(19) United States
(12) Patent Application Publication
(10) Pub. No.: US 2011/0203018 A1
(43) Pub. Date: Aug. 18, 2011

(54) Use of Carboxamides on Cultivated Plants

(75) Inventors: Markus Gewehr, Kastellaun (DE); Dirk Voeste, Limburgerhof (DE); Juergen Logemann, Schifferstadt (DE); Maarten Hendrik Stuiver, Neustadt (DE); Pilar Puente, Speyer (DE); Robert John Gladwin, Limburgerhof (DE)

(73) Assignee: BASF SE, Ludwigshafen (DE)

(21) Appl. No.: 13/124,940

(22) PCT Filed: Oct. 21, 2009
(86) PCT No.: PCT/EP2009/063781

§ 371 Date: Apr. 19, 2011

(30) Foreign Application Priority Data
Oct. 21, 2008 (EP) 08167079.6

(51) Int. Cl. A01H 5/00; A01N 43/40; A01N 43/56; A01N 37/22; A01N 43/32; A01N 43/08; A01N 43/78; A01P 3/00; A01C 1/06; A01N 65/00

(52) U.S. Cl. 800/298; 514/355; 514/406; 514/357; 514/617; 514/433; 514/471; 514/365; 504/100; 424/93.7

(57) ABSTRACT

The present invention relates to a method of controlling pests and/or increasing the health of a plant as compared to a corresponding control plant by treating the cultivated plant, parts of a plant, seed, or their locus of growth with a carboxamide compound.

This application claims the priority benefit of application EP 08167079.6, filed 21 Oct. 2008. The entire contents of each of the above-referenced applications is incorporated herein by reference.

The present invention relates to a method of controlling pests and/or increasing the health of a plant as compared to a corresponding control plant by treating the cultivated plant, parts of a plant, seed, or their locus of growth with a carboxamide compound selected from the group consisting of boscalid, N-(3′,4′,5′-trifluorobiphenyl-2-yl)-3-difluoromethyl-1-methyl-1H-pyrazole-4-carboxamide, bixafen, penflufen (N-[2-(1,3-dimethylbutyl)-phenyl]-1,3-dimethyl-5-fluoro-1H-pyrazole-4-carboxamide), fluopyram, sedaxane, isopyrazam, penthiopyrad, benodanil, carboxin, fenfuram, flutolanil, furametpyr, mepronil, oxycarboxin and thifluzamide.

One typical problem arising in the field of pest control lies in the need to reduce the dosage rates of the active ingredient in order to reduce or avoid unfavorable environmental or toxicological effects whilst still allowing effective pest control.

In regard to the instant invention the term pests embrace harmful fungi. The term harmful fungi includes, but is not limited to the following genera and species:

Albugo spp. (white rust) on ornamentals, vegetables (e.g. A. candida) and sunflowers (e.g. A. tragopogonis); Alternaria spp. (Alternaria leaf spot) on vegetables, rape (A. brassicola or brassicae), sugar beets (A. tenuis), fruits, rice, soybeans, potatoes (e.g. A. solani or A. alternata), tomatoes (e.g. A. solani or A. alternata) and wheat; Aphanomyces spp. on sugar beets and vegetables; Ascochyta spp. on cereals and vegetables, e.g. A. tritici (anthracnose) on wheat and A. hordei on barley; Bipolaris and Drechslera spp. (teleomorph: Cochliobolus spp.), e.g. Southern leaf blight (D. maydis) or Northern leaf blight (B. zeicola) on corn, e.g. spot blotch (B. sorokiniana) on cereals and e.g. B. oryzae on rice and turfs; Blumeria (formerly Elysiphe) graminis (powdery mildew) on cereals (e.g. on wheat or barley); Botrytis cinerea (teleomorph: Botryotinia fuckeliana: grey mold) on fruits and berries (e.g. strawberries), vegetables (e.g. lettuce, carrots, celery and cabbages), rape, flowers, vines, forestry plants and wheat; Bremia lactucae (downy mildew) on lettuce; Ceratocystis (syn. Ophiostoma) spp. (rot or wilt) on broad-leaved trees and evergreens, e.g. C. ulmi (Dutch elm disease) on elms; Cercospora spp. (Cercospora leaf spots) on corn (e.g. Gray leaf spot: C. zeae-maydis), rice, sugar beets (e.g. C. beticola), sugar cane, vegetables, coffee, soybeans (e.g. C. sojina or C. kikuchii) and rice; Cladosporium spp. on tomatoes (e.g. C. fulvum: leaf mold) and cereals, e.g. C. herbarum (black ear) on wheat; Claviceps purpurea (ergot) on cereals; Cochliobolus (anamorph: Helminthosporium of Bipolaris) spp. (leaf spots) on corn (C. carbonum), cereals (e.g. C. sativus, anamorph: B. sorokiniana) and rice (e.g. C. miyabeanus, anamorph: H. oryzae); Colletotrichum (teleomorph: Glomerella) spp. (anthracnose) on cotton (e.g. C. gossypii), corn (e.g. C. graminicola: Anthracnose stalk rot), soft fruits, potatoes (e.g. C. coccodes: black dot), beans (e.g. C. lindemuthianum) and soybeans (e.g. C. truncatum or C. gloeosporiades); Corticium spp., e.g. C. sasakii (sheath blight) on rice; Corynespora cassiicola (leaf spots) on soybeans and ornamentals; Cycloconium spp., e.g. C. oleaginum on olive trees; Cylindrocarpon spp. (e.g. fruit tree canker or young vine decline, teleomorph: Nectria or Neonectria spp.) on fruit trees, vines (e.g. C. liriodendri, teleomorph: Neonectria liriodendri: Black Foot Disease) and ornamentals; Dematophora (teleomorph: Rosellinia) necatrix (root and stem rot) on soybeans; Diaporthe spp., e.g. D. phaseolorum (damping off) on soybeans; Drechslera (syn. Helminthosporium, teleomorph: Pyrenophora) spp. on corn, cereals, such as barley (e.g. D. teres, net blotch) and wheat (e.g. D. tritici-repentis: tan spot), rice and turf; Esca (dieback, apoplexy) on vines, caused by Formitiporia (syn. Phellinus) punctata, F. mediterranea, Phaeomoniella chlamydospora (earlier Phaeoacremonium chlamydosporum), Phaeoacremonium aleophilum and/or Botryosphaeria obtusa; Elsinoe spp. on pome fruits (E. pyri), soft fruits (E. veneta: anthracnose) and vines (E. ampelina: anthracnose); Entyloma oryzae (leaf smut) on rice; Epicoccum spp. (black mold) on wheat; Erysiphe spp. (powdery mildew) on sugar beets (E. betae), vegetables (e.g. E. pisi), such as cucurbits (e.g. E. cichoracearum), cabbages, rape (e.g. E. cruciferarum); Eutypa lata (Eutypa canker or dieback, anamorph: Cytosporina lata, syn. Libertella blepharis) on fruit trees, vines and ornamental woods; Exserohilum (syn. Helminthosporium) spp. on corn (e.g. E. turcicum); Fusarium (teleomorph: Gibberella) spp. (wilt, root or stem rot) on various plants, such as F. gramnearum or F. culmorum (root rot, scab or head blight) on cereals (e.g. wheat or barley), F. oxysporum on tomatoes, F. solani on soybeans and F. verticillioides on corn; Gaeumannomyces graminis (take-all) on cereals (e.g. wheat or barley) and corn; Gibberella spp. on cereals (e.g. G. zeae) and rice (e.g. G. fujikuror: Bakanae disease); Glomerella cingulata on vines, pome fruits and other plants and G. gossypii on cotton; Grainstaining complex on rice; Guignardia bidwellii (black rot) on vines; Gymnosporangium spp. on rosaceous plants and junipers, e.g. G. sabinae (rust) on pears; Helminthosporium spp. (syn. Drechslera, teleomorph: Cochliobolus) on corn, cereals and rice; Hemileia spp., e.g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e.g. wheat or barley); Microsphaera diffusa (powdery mildew) on soybeans; Monilinia spp., e.g. M. laxa, M. fructicola and M. fructigena (bloom and twig blight, brown rot) on stone fruits and other rosaceous plants; Mycosphaerella spp. on cereals, bananas, soft fruits and ground nuts, such as e.g. M. graminicola (anamorph: Septoria tritici, Septoria blotch) on wheat or M. fijiensis (black Sigatoka disease) on bananas; Peronospora spp. (downy mildew) on cabbage (e.g. P. brassicae), rape (e.g. P. parasitica), onions (e.g. P. destructor), tobacco (P. tabacina) and soybeans (e.g. P. manshurica); Phakopsora pachyrhizi and P. meibomiae (soybean rust) on soybeans; Phialophora spp. e.g. on vines (e.g. P. tracheiphila and P. tetraspora) and soybeans (e.g. P. gregata: stem rot); Phoma lingam (root and stem rot) on rape and cabbage and P. betae (root rot, leaf spot and damping-off) on sugar beets; Phomopsis spp. on sunflowers, vines (e.g. P. viticola: can and leaf spot) and soybeans (e.g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e.g. P. capsici), soybeans (e.g. P. megasperma, syn. P. sojae), potatoes and tomatoes (e.g. P. infestans: late blight) and broad-leaved trees (e.g. P. ramorum: sudden oak death); Plasmodiophora brassicae (club root) on cabbage, rape, radish and other plants; Plasmopara spp., e.g. P. viticola (grapevine downy mildew) on vines and P. halstedii on sunflowers; Podosphaera spp. (powdery mildew) on rosaceous plants, hop, pome and soft fruits, e.g. P. leucotricha on apples; Polymyxa spp., e.g. on cereals, such as barley and wheat (P. graminis) and sugar beets (P. betae) and thereby transmitted viral diseases; Pseudocercosporella herpotrichoides (eyespot, teleomorph: Tapesia yallundae) on cereals, e.g. wheat or barley; Pseudoperonospora (downy mildew) on various plants, e.g. P. cubensis on cucurbits or P. humili on hop; Pseudopezicula tracheiphila (red fire disease or ,rotbrenner', anamorph: Phialophora) on vines; Puccinia spp. (rusts) on various plants, e.g. P. triticina (brown or leaf rust), P. striiformis (stripe or yellow rust), P. hordei (dwarf rust), P. graminis (stem or black rust) or P. recondita (brown or leaf rust) on cereals, such as e.g. wheat, barley or rye, and asparagus (e.g. P. asparagi); Pyrenophora (anamorph: Drechslera) tritici-repentis (tan spot) on wheat or P. teres (net blotch) on barley; Pyricularia spp., e.g. P. oryzae (teleomorph: Magnaporthe grisea, rice blast) on rice and P. grisea on turf and cereals; Pythium spp. (damping-off) on turf, rice, corn, wheat, cotton, rape, sunflowers, soybeans, sugar beets, vegetables and various other plants (e.g. P. ultimum or P. aphanidermatum); Ramularia spp., e.g. R. collo-cygni (Ramularia leaf spots, Physiological leaf spots) on barley and R. beticola on sugar beets; Rhizoctonia spp. on cotton, rice, potatoes, turf, corn, rape, potatoes, sugar beets, vegetables and various other plants, e.g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secalis (scald) on barley, rye and triticale; Sarocladium oryzae and S. attenuatum (sheath rot) on rice; Sclerotinia spp. (stem rot or white mold) on vegetables and field crops, such as rape, sunflowers (e.g. S. sclerotiorum) and soybeans (e.g. S. rolfsii or S. sclerotiorum); Septoria spp. on various plants, e.g. S. glycines (brown spot) on soybeans, S. tritici (Septoria blotch) on wheat and S. (syn. Stagonospora) nodorum (Stagonospora blotch) on cereals; Uncinula (syn. Erysiphe) necator (powdery mildew, anamorph: Oidium tuckeri) on vines; Setospaeria spp. (leaf blight) on corn (e.g. S. turcicum, syn. Helminthosporium turcicum) and turf; Sphacelotheca spp. (smut) on corn, (e.g. S. reiliana: head smut), sorghum and sugar cane; Sphaerotheca fuliginea (powdery mildew) on cucurbits; Spongospora subterranea (powdery scab) on potatoes and thereby transmitted viral diseases; Stagonospora spp. on cereals, e.g. S. nodorum (Stagonospora blotch, teleomorph: Leptosphaeria [syn. Phaeosphaeria] nodorum) on wheat; Synchytrium endobioticum on potatoes (potato wart disease); Taphrina spp., e.g. T. deformans (leaf curl disease) on peaches and T. pruni (plum pocket) on plums; Thielaviopsis spp. (black root rot) on tobacco, pome fruits, vegetables, soybeans and cotton, e.g. T. basicola (syn. Chalara elegans); Tilletia spp. (common bunt or stinking smut) on cereals, such as e.g. T. tritici (syn. T. caries, wheat bunt) and T. controversa (dwarf bunt) on wheat; Typhula incarnate (grey snow mold) on barley or wheat; Urocystis spp., e.g. U. occulta (stem smut) on rye; Uromyces spp. (rust) on vegetables, such as beans (e.g. U. appendiculatus, syn. U. phaseoli) and sugar beets (e.g. U. betae); Ustilago spp. (loose smut) on cereals (e.g. U. nuda and U. avaenae), corn (e.g. U. maydis: corn smut) and sugar cane; Ventura spp. (scab) on apples (e.g. V. inaequalis) and pears; and Verticillium spp. (wilt) on various plants, such as fruits and ornamentals, vines, soft fruits, vegetables and field crops, e.g. V. dahliae on strawberries, rape, potatoes and tomatoes.

Another problem underlying the present invention is the desire for compositions that improve the health of a plant, a process which is commonly and hereinafter referred to as “plant health”. The term plant health comprises various sorts of improvements of plants that are not connected to the control of pests and which do not embrace the reduction of negative consequences of harmful fungi. The term “plant health” is to be understood to denote a condition of the plant and/or its products which is determined by several indicators alone or in combination with each other such as yield (e.g. increased biomass and/or increased content of valuable ingredients), plant vigor (e.g. improved plant growth and/or greener leaves (“greening effect”), quality (e.g. improved content or composition of certain ingredients) and tolerance to abiotic and/or biotic stress. The above identified indicators for the health condition of a plant may be interdependent or may result from each other.

It was therefore an objective of the present invention to provide a method, which solves the problems as outlined above and which especially reduces the dosage rate and/or promotes the health of a plant.

Surprisingly, it has now been found that the use of carboxamide compounds as defined above in cultivated plants displays a synergistic effect between the trait of the cultivated plant and the applied carboxamide.

Synergistic in the present context means that
a) the use of a carboxamide compound as defined above in combination with a cultivated plant exceeds the additive effect, to be expected on the harmful fungi to be controlled and thus extends the range of action of the carboxamide compound and of the active principle expressed by the cultivated plant, and/or
b) such use results in an increased plant health effect in such cultivated plants compared to the plant health effects that are possible with the carboxamide compound, when applied to the non-cultivated plant; and/or
c) the carboxamide compound induces “side effects” in the cultivated plant which increases plant health, as compared to the respective control plant, additionally to the primary mode of action, meaning the fungicidal activity; and/or
d) the carboxamide compound induces “side effects” additionally to the primary mode of action, meaning the fungicidal activity in the control plant which are detrimental to the plant health compared to a control plant which is not treated with said compound. In combination with the cultivated plant these negative side effects are reduced, nullified or converted to an increase of the plant health of the cultivated plant compared to a cultivated plant not treated with said compound.

Thus, the term “synergistic”, is to be understood in this context as synergistic fungicidal activity and/or the synergistic increase of plant health.

Especially, it has been found that the application of at least one carboxamide compound as defined above to cultivated plants leads to a synergistically enhanced action against harmful fungi compared to the control rates that are possible with the carboxamide compound as defined above in non-cultivated plants and/or leads to an synergistic increase in the health of a plant when applied to a cultivated plant, parts of a plant, plant propagation material, or to their locus of growth.

Thus, the present invention relates to a method of controlling harmful fungi and/or increasing the health of a cultivated plant by treating a cultivated plant, parts of a plant, plant propagation material, or to their locus of growth with a carboxamide compound selected from the group consisting of boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam, penthiopyrad, benodanil, carboxin, fenfuram, flutolanil, furametpyr, mepronil, oxycarboxin, thifluzamide, preferably with a carboxamide compound selected from the group consisting of boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad.

The carboxamide compounds are known as fungicides (cf., for example, EP-A 545 099, EP-A 589 301, EP-A 737682, EP-A 824099, WO 99/09013, WO 03/010149, WO 03/070705, WO 03/074491, WO 2004/005242, WO 2004/035589, WO 2004/067515, WO 06/087343). For instance, the commercially available compounds may be found in The Pesticide Manual, 13th Edition, British Crop Protection Council (2003) among other publications.

The term “plant propagation material” is to be understood to denote all the generative parts of a plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants, including seedlings and young plants, which are to be transplanted after germination or after emergence from soil. These young plants may also be protected before transplantation by a total or partial treatment by immersion or pouring. Preferably, the term plant propagation material denotes seeds.

In a preferred embodiment, the present invention relates to a method of controlling harmful fungi and/or increasing the health of a cultivated plant by treating plant propagation material, preferably seeds with a carboxamide compound selected from boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam, penthiopyrad, benodanil, carboxin, fenfuram, flutolanil, furametpyr, mepronil, oxycarboxin, thifluzamide, more preferably with a carboxamide compound selected from the group consisting of boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad, most preferably with boscalid, N-(3′,4′,5′-trifluorobiphenyl-2-yl)-3-difluoromethyl-1-methyl-1H-pyrazole-4-carboxamide, penflufen, fluopyram, sedaxane and penthiopyrad.

The present invention also comprises plant propagation material, preferably seed, of a cultivated plant treated with a carboxamide as defined above, preferably boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam, penthiopyrad, benodanil, carboxin, fenfuram, flutolanil, furametpyr, mepronil, oxycarboxin, thifluzamide, preferably with a carboxamide compound selected from the group consisting of boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad, most preferably with boscalid, N-(3′,4′,5′-trifluorobiphenyl-2-yl)-3-difluoromethyl-1-methyl-1H-pyrazole-4-carboxamide, penflufen, fluopyram, sedaxane and penthiopyrad.

In another preferred embodiment, the present invention relates to a method of controlling harmful fungi and/or increasing the health of a cultivated plant by treating the cultivated plant, part(s) of such plant or at its locus of growth with a carboxamide compound selected, preferably boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam, penthiopyrad, benodanil, carboxin, fenfuram, flutolanil, furametpyr, mepronil, oxycarboxin, thifluzamide, more preferably with a carboxamide compound selected from the group consisting of boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad, most preferably from boscalid, N-(3′,4′,5′-trifluorobiphenyl-2-yl)-3-difluoromethyl-1-methyl-1H-pyrazole-4-carboxamide bixafen, fluopyram, isopyrazam and penthiopyrad.

In another embodiment, the present invention relates to a composition comprising a pesticide and a cultivated plant or parts or cells thereof, wherein the pesticide is a carboxamide compound, preferably selected from the group consisting of boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam, penthiopyrad, benodanil, carboxin, fenfuram, flutolanil, furametpyr, mepronil, oxycarboxin, thifluzamide, more preferably with a carboxamide compound selected from the group consisting of boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad, most preferably from boscalid, N-(3′,4′,5′-trifluorobiphenyl-2-yl)-3-difluoromethyl-1-methyl-1H-pyrazole-4-carboxamide bixafen, fluopyram, isopyrazam and penthiopyrad. Said compositions may include other pesticides and other carboxamide s or several of the carboxamide s of the group described in the previous sentence. Said compositions may include substances used in plant protection, and in particular in formulation of plant protection products. The composition of the invention may comprise live plant material or plant material unable to propagate or both. The composition may contain plant material from more than one plant. In a preferred embodiment, the ratio of plant material from at least one cultivated plant to pesticide on a weight per weight basis is greater then 10 to 1, preferably greater than 100 to 1 or more preferably greater than 1000 to 1, even more preferably greater than 10 000 to 1. In some cases a ratio of greater than 100000 or million to one is utmostly preferred.

In one embodiment, under “agricultural composition” is to be understood, that such a composition is in agreement with the laws regulating the content of fungicides, plant nutrients, herbicides etc. Preferably such a composition is without any harm to the protected plants and/or the animals (humans included) fed therewith.

In another embodiment, the present invention relates to a method for the production of an agricultural product comprising the application of a pesticide to cultivated plants with at least one modification, parts of such plants, plant propagation materials, or at their locus of growth, and producing the agricultural product from said plants parts of such plants or plant propagation materials, wherein the pesticide is a carboxamide compound preferably selected from the group consisting of boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam, penthiopyrad, benodanil, carboxin, fenfuram, flutolanil, furametpyr, mepronil, oxycarboxin, thifluzamide, more preferably with a carboxamide compound selected from the group consisting of boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad, most preferably from boscalid, N-(3′,4′,5′-trifluorobiphenyl-2-yl)-3-difluoromethyl-1-methyl-1H-pyrazole-4-carboxamide bixafen, fluopyram, isopyrazam and penthiopyrad.

In one embodiment of the invention the term “agricultural product” is defined as the output of the cultivation of the soil, for example grain, forage, fruit, fiber, flower, pollen, leaves, tuber, root, beet or seed.

In one embodiment of the invention the term “agricultural product” is defined according to USDA's (U.S. Department of Agriculture) definition of “agricultural products”. Preferably under “agricultural product” are understood “food and fiber” products, which cover a broad range of goods from unprocessed bulk commodities like soybeans, feed corn, wheat, rice, and raw cotton to highly-processed, high-value foods and beverages like sausages, bakery goods, ice cream, beer and wine, and condiments sold in retail stores and restaurants. In one embodiment “agricultural product” are products found in Chapters 4, 6-15, 17-21, 23-24, Chapter 33, and Chapter 52 of the U.S. Harmonized Tariff Schedule (from December 1993, occurred as a result of the Uruguay Round Agreements) based on the international Harmonized Commodity Coding and Classification System (Harmonized System) which has been established by the World Customs Organization). Agricultural products according to the invention within these chapters preferably fall into the following categories: grains, animal feeds, and grain products (like bread and pasta); oilseeds and oilseed products (like soybean oil and olive oil); horticultural products including all fresh and processed fruits, vegetables, tree nuts, as well as nursery products, unmanufactured tobacco; and tropical products like sugar, cocoa and coffee. In one embodiment “agricultural product” is a product selected from the group of products as found in the U.S. Harmonized Tariff Schedule under the items: 0409, 0601 to 0604, 0701 to 0714, 0801 to 0814, 0901 to 0910, 1001 to 1008, 1101 to 1109, 1201 to 1214, 1301 to 1302, 14 01 to 1404, 1507 to 1522, 1701 to 1704, 1801 to 1806, 1901 to 1905, 2001 to 2009, 2101 to 2106, 2302 to 2309, 2401 to 2403, 3301, 5201 to 5203.

The term “cultivated plant(s)” refers to “modified plant(s)” and “transgenic plant(s)”.

In one embodiment of the invention, the term “cultivated plants” refers to “modified plants”.

In one embodiment of the invention, the term “cultivated plants” refers to “transgenic plants”.

“Modified plants” are those which have been modified by conventional breeding techniques. The term “modification” means in relation to modified plants a change in the genome, epigenome, transcriptome or proteome of the modified plant, as compared to the control, wild type, mother or parent plant whereby the modification confers a trait (or more than one trait) or confers the increase of a trait (or more than one trait) as listed below.

The modification may result in the modified plant to be a different, for example a new plant variety than the parental plant.

“Transgenic plants” are those, which genetic material has been modified by the use of recombinant DNA techniques that under natural circumstances can not readily be obtained by cross breeding, mutations or natural recombination, whereby the modification confers a trait (or more than one trait) or confers the increase of a trait (or more than one trait) as listed below as compared to the wild-type plant.

In one embodiment, one or more genes have been integrated into the genetic material of a genetically modified plant in order to improve certain properties of the plant, preferably increase a trait as listed below as compared to the wild-type plant. Such genetic modifications also include but are not limited to targeted post-translational modification of protein(s), or to post-transcriptional modifications of oligo- or polypeptides e.g. by glycosylation or polymer additions such as prenylated, acetylated, phosphorylated or farnesylated moieties or PEG moieties.

In one embodiment under the term “modification” when referring to a transgenic plant or parts thereof is understood that the activity, expression level or amount of a gene product or the metabolite content is changed, e.g. increased or decreased, in a specific volume relative to a corresponding volume of a control, reference or wild-type plant or plant cell, including the de novo creation of the activity or expression.

In one embodiment the activity of a polypeptide is increased or generated by expression or overexpression of the gene coding for said polypeptide which confers a trait or confers the increase of a trait as listed below as compared to the control plant. The term “expression” or “gene expression” means the transcription of a specific gene or specific genes or specific genetic construct. The term “expression” or “gene expression” in particular means the transcription of a gene or genes or genetic construct into structural RNA (rRNA, tRNA), regulatory RNA (e.g. miRNA, RNAi, RNAa) or mRNA with or without subsequent translation of the latter into a protein. In another embodiment the term “expression” or “gene expression” in particular means the transcription of a gene or genes or genetic construct into structural RNA (rRNA, tRNA) or mRNA with or without subsequent translation of the latter into a protein. In yet another embodiment it means the transcription of a gene or genes or genetic construct into mRNA.

The process includes transcription of DNA and processing of the resulting mRNA product. The term “increased expression” or “overexpression” as used herein means any form of expression that is additional to the original wild-type expression level.

The term “expression of a polypeptide” is understood in one embodiment to mean the level of said protein or polypeptide, preferably in an active form, in a cell or organism.

In one embodiment the activity of a polypeptide is decreased by decreased expression of the gene coding for said polypeptide which confers a trait or confers the increase of a trait as listed below as compared to the control plant. Reference herein to “decreased expression” or “reduction or substantial elimination” of expression is taken to mean a decrease in endogenous gene expression and/or polypeptide levels and/or polypeptide activity relative to control plants. It comprises further reducing, repressing, decreasing or deleting of an expression product of a nucleic acid molecule.

The terms “reduction”, “repression”, “decrease” or “deletion” relate to a corresponding change of a property in an organism, a part of an organism such as a tissue, seed, root, tuber, fruit, leave, flower etc. or in a cell. Under “change of a property” it is understood that the activity, expression level or amount of a gene product or the metabolite content is changed in a specific volume or in a specific amount of protein relative to a corresponding volume or amount of protein of a control, reference or wild type. Preferably, the overall activity in the volume is reduced, decreased or deleted in cases if the reduction, decrease or deletion is related to the reduction, decrease or deletion of an activity of a gene product, independent whether the amount of gene product or the specific activity of the gene product or both is reduced, decreased or deleted or whether the amount, stability or translation efficacy of the nucleic acid sequence or gene encoding for the gene product is reduced, decreased or deleted.

The terms “reduction”, “repression”, “decrease” or “deletion” include the change of said property in only parts of the subject of the present invention, for example, the modification can be found in compartment of a cell, like an organelle, or in a part of a plant, like tissue, seed, root, leave, tuber, fruit, flower etc. but is not detectable if the overall subject, i.e. complete cell or plant, is tested. Preferably, the “reduction”, “repression”, “decrease” or “deletion” is found cellular, thus the term “reduction, decrease or deletion of an activity” or “reduction, decrease or deletion of a metabolite content” relates to the cellular reduction, decrease or deletion compared to the wild type cell. In addition the terms “reduction”, “repression”, “decrease” or “deletion” include the change of said property only during different growth phases of the organism used in the inventive process, for example the reduction, repression, decrease or deletion takes place only during the seed growth or during blooming. Furthermore the terms include a transitional reduction, decrease or deletion for example because the used method, e.g. the antisense, RNAi, snRNA, dsRNA, siRNA, miRNA, ta-siRNA, cosuppression molecule, or ribozyme, is not stable integrated in the genome of the organism or the reduction, decrease, repression or deletion is under control of a regulatory or inducible element, e.g. a chemical or otherwise inducible promoter, and has therefore only a transient effect. Methods to achieve said reduction, decrease or deletion in an expression product are known in the art, for example from the international patent application WO 2008/034648, particularly in paragraphs [0020.1.1.1], [0040.1.1.1], [0040.2.1.1] and [0041.1.1.1].

Reducing, repressing, decreasing or deleting of an expression product of a nucleic acid molecule in modified plants is known. Examples are canola i.e. double nill oilseed rape with reduced amounts of erucic acid and sinapins.

Such a decrease can also be achieved for example by the use of recombinant DNA technology, such as antisense or regulatory RNA (e.g. miRNA, RNAi, RNAa) or siRNA approaches. In particular RNAi, snRNA, dsRNA, siRNA, miRNA, ta-siRNA, cosuppression molecule, ribozyme, or antisense nucleic acid molecule, a nucleic acid molecule conferring the expression of a dominant-negative mutant of a protein or a nucleic acid construct capable to recombine with and silence, inactivate, repress or reduces the activity of an endogenous gene may be used to decrease the activity of a polypeptide in a transgenic plant or parts thereof or a plant cell thereof used in one embodiment of the methods of the invention. Examples of transgenic plants with reduced, repressed, decreased or deleted expression product of a nucleic acid molecule are Carica papaya (Papaya plants) with the event name X17-2 of the University of Florida, Prunus domestica (Plum) with the event name C5 of the United States Department of Agriculture—Agricultural Research Service, or those listed in rows T9-48 and T9-49 of table 9 below. Also known are plants with increased resistance to nematodes for example by reducing, repressing, decreasing or deleting of an expression product of a nucleic acid molecule, e.g. from the PCT publication WO 2008/095886.

The reduction or substantial elimination is in increasing order of preference at least 10%, 20%, 30%, 40% or 50%, 60%, 70%, 80%, 85%, 90%, or 95%, 96%, 97%, 98%, 99% or more reduced compared to that of control plants. Reference herein to an “endogenous” gene not only refers to the gene in question as found in a plant in its natural form (i.e., without there being any human intervention), but also refers to that same gene (or a substantially homologous nucleic acid/gene) in an isolated form subsequently (re)introduced into a plant (a transgene). For example, a transgenic plant containing such a transgene may encounter a substantial reduction of the transgene expression and/or substantial reduction of expression of the endogenous gene.

The terms “control” or “reference” are exchangeable and can be a cell or a part of a plant such as an or ganelle like a chloroplast or a tissue, in particular a plant, which was not modified or treated according to the herein described process according to the invention. Accordingly, the plant used as control or reference corresponds to the plant as much as possible and is as identical to the subject matter of the invention as possible. Thus, the control or reference is treated identically or as identical as possible, saying that only conditions or properties might be different which do not influence the quality of the tested property other than the treatment of the present invention.

It is possible that control or reference plants are wild-type plants. However, “control” or “reference” may refer to plants carrying at least one genetic modification, when the plants employed in the process of the present invention carry at least one genetic modification more than said control or reference plants. In one embodiment control or reference plants may be transgenic but differ from transgenic plants employed in the process of the present invention only by said modification contained in the transgenic plants employed in the process of the present invention.

The term “wild type” or “wild-type plants” refers to a plant without said genetic modification. These terms can refer to a cell or a part of a plant such as an organelle like a chloroplast or a tissue, in particular a plant, which lacks said genetic modification but is otherwise as identical as possible to the plants with at least one genetic modification employed in the present invention. In a particular embodiment the “wild-type” plant is not transgenic.

Preferably, the wild type is identically treated according to the herein described process according to the invention. The person skilled in the art will recognize if wild-type plants will not require certain treatments in advance to the process of the present invention, e.g. non-transgenic wild-type plants will not need selection for transgenic plants for example by treatment with a selecting agent such as a herbicide.

The control plant may also be a nullizygote of the plant to be assessed. The term “nullizygotes” refers to a plant that has undergone the same production process as a transgenic, yet has not acquired the same genetic modification as the corresponding transgenic. If the starting material of said production process is transgenic, then nullizygotes are also transgenic but lack the additional genetic modification introduced by the production process. In the process of the present invention the purpose of wild-type and nullizygotes is the same as the one for control and reference or parts thereof. All of these serve as controls in any comparison to provide evidence of the advantageous effect of the present invention.

Preferably, any comparison is carried out under analogous conditions. The term “analogous conditions” means that all conditions such as, for example, culture or growing conditions, soil, nutrient, water content of the soil, temperature, humidity or surrounding air or soil, assay conditions (such as buffer composition, temperature, substrates, pathogen strain, concentrations and the like) are kept identical between the experiments to be compared. The person skilled in the art will recognize if wild-type, control or reference plants will not require certain treatments in advance to the process of the present invention, e.g. non-transgenic wild-type plants will not need selection for transgenic plants for example by treatment with herbicide.

In case that the conditions are not analogous the results can be normalized or standardized based on the control.

The “reference”, “control”, or “wild type” is preferably a plant, which was not modified or treated according to the herein described process of the invention and is in any other property as similar to a plant, employed in the process of the present invention of the invention as possible. The reference, control or wild type is in its genome, transcriptome, proteome or metabolome as similar as possible to a plant, employed in the process of the present invention of the present invention. Preferably, the term “reference-” “control-” or “wild-type-” plant, relates to a plant, which is nearly genetically identical to the organelle, cell, tissue or organism, in particular plant, of the present invention or a part thereof preferably 90% or more, e.g. 95%, more preferred are 98%, even more preferred are 99.00%, in particular 99.10%, 99.30%, 99.50%, 99.70%, 99.90%, 99.99%, 99.999% or more. Most preferable the “reference”, “control”, or “wild type” is a plant, which is genetically identical to the plant, cell, a tissue or organelle used according to the process of the invention except that the responsible or activity conferring nucleic acid molecules or the gene product encoded by them have been amended, manipulated, exchanged or introduced in the organelle, cell, tissue, plant, employed in the process of the present invention.

Preferably, the reference and the subject matter of the invention are compared after standardization and normalization, e.g. to the amount of total RNA, DNA, or protein or activity or expression of reference genes, like housekeeping genes, such as ubiquitin, actin or ribosomal proteins.

The genetic modification carried in the organelle, cell, tissue, in particular plant used in the process of the present invention is in one embodiment stable e.g. due to a stable transgenic expression or to a stable mutation in the corresponding endogenous gene or to a modulation of the expression or of the behaviour of a gene, or transient, e.g. due to an transient transformation or temporary addition of a modulator such as an agonist or antagonist or inducible, e.g. after transformation with a inducible construct carrying a nucleic acid molecule under control of a inducible promoter and adding the inducer, e.g. tetracycline.

Preferred plants according to the invention, from which “modified plants” and/or “transgenic plants” are selected, are selected from the group consisting of cereals, such as maize (corn), wheat, barley sorghum, rice, rye, millet, triticale, oat, pseudocereals (such as buckwheat and quinoa), alfalfa, apples, banana, beet, broccoli, Brussels sprouts, cabbage, canola (rapeseed), carrot, cauliflower, cherries, chickpea, Chinese cabbage, Chinese mustard, collard, cotton, cranberries, creeping bentgrass, cucumber, eggplant, flax, grape, grapefruit, kale, kiwi, kohlrabi, melon, mizuna, mustard, papaya, peanut, pears, pepper, persimmons, pigeon pea, pineapple, plum, plum, potato, raspberry, rutabaga, soybean, squash, strawberries, sugar beet, sugarcane, sunflower, sweet corn, tobacco, tomato, turnip, walnut, watermelon and winter squash;

preferably the plants are selected from the group consisting of alfalfa, barley, canola (rapeseed), cotton, maize (corn), papaya, potato, rice, sorghum, soybean, squash, sugar beet, sugarcane, tomato and cereals (such as wheat, barley, rye and oat), most preferably the plant is selected from the group consisting of soybean, maize (corn), rice, cotton, oilseed rape, tomatoes, potatoes and cereals such as wheat, barley, rye and oat.

In another embodiment of the invention the cultivated plant is a gymnosperm plant, especially a spruce, pine or fir.

In one embodiment, the cultivated plant is selected from the families Aceraceae, Anacardiaceae, Apiaceae, Asteraceae, Brassicaceae, Cactaceae, Cucurbitaceae, Euphor-biaceae, Fabaceae, Malvaceae, Nymphaeaceae, Papaveraceae, Rosaceae, Salicaceae, Solanaceae, Arecaceae, Bromeliaceae, Cyperaceae, Iridaceae, Liliaceae, Orchidaceae, Gentianaceae, Labiaceae, Magnoliaceae, Ranunculaceae, Carifolaceae, Rubiaceae, Scrophulariaceae, Caryophyllaceae, Ericaceae, Polygonaceae, Violaceae, Juncaceae or Poaceae and preferably from a plant selected from the group of the families Apiaceae, As-teraceae, Brassicaceae, Cucurbitaceae, Fabaceae, Papaveraceae, Rosaceae, Solanaceae, Liliaceae or Poaceae.

Preferred are crop plants and in particular plants selected from the families and genera mentioned above for example preferred the species Anacardium occidentale, Calendula officinalis, Carthamus tinctorius, Cichofium intybus, Cynara scolymus, Helianthus annus, Tagetes lucida, Tagetes erecta, Tagetes tenuifolia; Daucus carota; Corylus avellana, Corylus colurna, Borago officinalis; Brassica napus, Brassica rapa ssp., Sinapis arvensis Brassica juncea, Brassica juncea var. juncea, Brassica juncea var. crispifolia, Brassica juncea var. foliosa, Brassica nigra, Brassica sinapioides, Melanosinapis communis, Brassica oleracea, Arabidopsis thaliana, Anana comosus, Ananas ananas, Bromelia comosa, Carica papaya, Cannabis sative, Ipomoea batatus, Ipomoea pandurata, Convolvulus batatas, Convolvulus tiliaceus, Ipomoea fas-tigiata, Ipomoea tiliacea, Ipomoea triloba, Convolvulus panduratus, Beta vulgaris, Beta vul-garis var. altissima, Beta vulgaris var. vulgaris, Beta maritima, Beta vulgaris var. perennis, Beta vulgaris var. conditiva, Beta vulgaris var. esculenta, Cucurbita maxima, Cucurbita mixta, Cucurbita pepo, Cucurbita moschata, Olea europaea, Manihot utilissima, Janipha manihot, Jatropha manihot, Manihot aipil, Manihot dulcis, Manihot manihot, Manihot melanobasis, Manihot esculenta, Ricinus communis, Pisum sativum, Pisum arvense, Pisum humile, Medicago sativa, Medicago falcata, Medicago varia, Glycine max Dolichos soja, Glycine gracilis, Glycine hispida, Phaseolus max, Soja hispida, Soja max, Cocos nucifera, Pelargonium grossularioides, Oleum cocoas, Laurus nobilis, Persea americana, Arachis hypogaea, Linum usitatissimum, Linum humile, Linum austriacum, Linum bienne, Linum angustifolium, Linum catharticum, Linum flavum, Linum grandiflorum, Adenolinum grandfflo-rum, Linum lewisii, Linum narbonense, Linum perenne, Linum perenne var. Linum pratense, Linum trigynum, Punica granatum, Gossypium hirsutum, Gossypium arboreum, Gossypium barbadense, Gossypium herbaceum, Gossypium thurberi, Musa nana, Musa acuminata, Musa paradisiaca, Musa spp., Elaeis guineensis, Papaver orientale, Papaver rhoeas, Papaver dubium, Sesamum indicum, Piper aduncum, Piper amalago, Piper angustifolium, Piper auritum, Piper betel, Piper cubeba, Piper longum, Piper nigrum, Piper ret-rofractum, Artanthe adunca, Artanthe elongata, Peperomia elongata, Piper elongatum, Steffensia elongata, Hordeum vulgare, Hordeum jubatum, Hordeum murinum, Hordeum secalinum, Hordeum distichon Hordeum aegiceras, Hordeum hexastichon, Hordeum hexa-stichum, Hordeum irregulare, Hordeum sativum, Hordeum secalinum, Avena sativa, Avena fatua, Avena byzantina, Avena fatua var. sativa, Avena hybrida, Sorghum bicolor, Sorghum halepense, Sorghum saccharatum, Sorghum vulgare, Andropogon drummondii, Holcus bi-color, Holcus sorghum, Sorghum aethiopicum, Sorghum arundinaceum, Sorghum caf-frorum, Sorghum cernuum, Sorghum dochna, Sorghum drummondii, Sorghum durra, Sor-ghum guineense, Sorghum lanceolatum, Sorghum nervosum, Sorghum saccharatum, Sorghum subglabrescens, Sorghum verticilliflorum, Sorghum vulgare, Holcus halepensis, Sorghum miliaceum millet, Panicum militaceum, Zea mays, Triticum aestivum, Triticum durum, Triticum turgidum, Triticum hybernum, Triticum macha, Triticum sativum or Tritcum vulgare, Cofea spp., Coffea arabica, Coffea canephora, Coffea liberca, Capsicum annuum, Capsi-cum annuum var. glabriusculum, Capsicum frutescens, Capsicum annuum, Nicotiana tabacum, Solanum tuberosum, Solanum melongena, Lycopersicon esculentum, Lycopersicon lycopersicum, Lycopersicon pyriforme, Solanum integrifolium, Solanum lycopersicum Theobroma cacao and Camellia sinensis.

Anacardiaceae such as the genera Pistacia, Mangifera, Anacardium e.g. the species Pistacia vera [pistachios, Pistazie], Mangifer indica [Mango] or Anacardium occi-dentate [Cashew], Asteraceae such as the genera Calendula, Carthamus, Centaurea, Cichorium, Cynara, Helianthus, Lactuca, Locusta, Tagetes, Valeriana e.g. the species Calendula officinalis [Marigold], Carthamus tinctorius [safflower], Centaurea cyanus [corn-flower], Cichorium intybus [blue daisy], Cynara scolymus [Artichoke], Helianthus annus [sunflower], Lactuca sativa, Lactuca crispa, Lactuca esculenta, Lactuca scariola L. ssp. sativa, Lactuca scariola L. var. integrata, Lactuca scariola L. var. integrifolia, Lactuca sativa subsp. romana, Locusta communis, Valeriana locusta [lettuce], Tagetes lucida, Tagetes erecta or Tagetes tenuifolia [Marigold]; Apiaceae such as the genera Daucus e.g. the species Daucus carota [carrot]; Betulaceae such as the genera Corylus e.g. the species Corylus avellana or Corylus colurna [hazelnut]; Boraginaceae such as the genera Borago e.g. the species Borago officinalis [borage]; Brassicaceae such as the genera Brassica, Melanosinapis, Sinapis, Arabadopsis e.g. the species Brassica napus, Brassica rapa ssp. [canola, oilseed rape, turnip rape], Sinapis arvensis Brassica juncea, Brassica juncea var juncea, Brassica juncea var. crispifolia, Brassica juncea var. foliosa, Brassica nigra, Bras-sica sinapioides, Melanosinapis communis [mustard], Brassica oleracea [fodder beet] or Arabidopsis thaliana; Bromeliaceae such as the genera Anana, Bromelia e.g. the species Anana comosus, Ananas ananas or Bromelia comosa [pineapple]; Caricaceae such as the genera Carica e.g. the species Carica papaya [papaya]; Cannabaceae such as the genera Cannabis e.g. the species Cannabis sative [hemp], Convolvulaceae such as the genera Ipomea, Convolvulus e.g. the species Ipomoea batatas, Ipomoea pandurata, Convolvulus batatas, Convolvulus tiliaceus, Ipomoea fastigiata, Ipomoea tiliacea, Ipomoea triloba or Convolvulus panduratus [sweet potato, Man of the Earth, wild potato], Chenopodiaceae such as the genera Beta, i.e. the species Beta vulgaris, Beta vulgaris var. altissima, Beta vulgaris var Vulgaris, Beta maritima, Beta vulgaris var. perennis, Beta vulgaris var conditiva or Beta vulgaris var esculenta [sugar beet]; Cucurbitaceae such as the genera Cucubita e.g. the species Cucurbita maxima, Cucurbita mixta, Cucurbita pepo or Cucurbita mo-schata [ pumpkin, squash]; Elaeagnaceae such as the genera Elaeagnus e.g. the species Olea europaea [olive]; Ericaceae such as the genera Kalmia e.g. the species Kalmia latifolia, Kalmia angustifolia, Kalmia microphylla, Kalmia polifolia, Kalmia occidentalis, Cistus chamaerhodendros or Kalmia lucida [American laurel, broad-leafed laurel, calico bush, spoon wood, sheep laurel, alpine laurel, bog laurel, western bog-laurel, swamp-laurel]; Euphorbiaceae such as the genera Manihot, Janipha, Jatropha, Ricinus e.g. the species Manihot utilissima, Janipha manihot, Jatropha manihot., Manihot aipil, Manihot dulcis, Manihot manihot, Manihot melanobasis, Manihot esculenta [manihot, arrowroot, tapioca, cassava] or Ricinus communis [castor bean, Castor Oil Bush, Castor Oil Plant, Palma Christi, Wonder Tree]; Fabaceae such as the genera Pisum, Albizia, Cathormion, Feuillea, Inga, Pithecolobium, Acacia, Mimosa, Medicajo, Glycine, Dolichos, Phaseolus, Soja e.g. the species Pisum sativum, Pisum arvense, Pisum humile [pea], Albizia berteriana, Albizia julibrissin, Albizia lebbeck, Acacia berteriana, Acacia littoralis, Albizia berteriana, Albizzia berteriana, Cathormion berteriana, Feuillea berteriana, Inga fragrans, Pithecellabium berterianum, Pithecellobium fragrans, Pithecolobium berterianum, Pseudalbizzia berteriana, Acacia julthrissin, Acacia nemu, Albizia nemu, Feuilleea julibrissin, Mimosa julthrissin, Mimosa speciosa, Sericanrda julibrissin, Acacia lebbeck, Acacia macrophylla, Albizia lebbek, Feuilleea lebbeck, Mimosa lebbeck, Mimosa speciosa [bastard logwood, silk tree, East Indian Walnut], Medicago sativa, Medicago falcata, Medicago varia [alfalfa] Glycine max Dolichos soja, Glycine gracilis, Glycine hispida, Phaseolus max, Soja hispida or Soja max [soy-bean]; Geraniaceae such as the genera Pelargonium, Cocos, Oleum e.g. the species Cocos nucifera, Pelargonium grossularioides or Oleum cocois [coconut]; Gramineae such as the genera Saccharum e.g. the species Saccharum officinarum; Juglandaceae such as the genera Juglans, Wallia e.g. the species Juglans regia, Juglans ailanthifolia, Juglans sie-boldiana, Juglans cinerea, Wallia cinerea, Juglans bixbyi, Juglans californica, Juglans hind-sii, Juglans intermedia, Juglans jamaicensis, Juglans major, Juglans microcarpa, Juglans nigra or Wallia nigra [walnut, black walnut, common walnut, persian walnut, white walnut, butternut, black walnut]; Lauraceae such as the genera Persea, Laurus e.g. the species laurel Laurus nobilis [bay, laurel, bay laurel, sweet bay], Persea americana, Persea gratissima or Persea persea [avocado]; Leguminosae such as the genera Arachis e.g. the species Arachis hypogaea [peanut]; Linaceae such as the genera Linum, Adenolinum e.g. the species Linum usitatissimum, Linum humile, Linum austriacum, Linum bienne, Linum angustifolium, Linum catharticum, Linum flavum, Linum grandiflorum, Adeno-linum grandiflorum, Linum lewisii, Linum narbonense, Linum perenne, Linum perenne var. lewisii, Linum pratense or Linum trigynum [flax, linseed]; Lythrarieae such as the genera Punica e.g. the species Punica granatum [pomegranate]; Malvaceae such as the genera Gossypium e.g. the species Gossypium hirsutum, Gossypium arboreum, Gossypium barbadense, Gossypium herbaceum or Gossypium thurberi [cotton]; Musaceae such as the genera Musa e.g. the species Musa nana, Musa acuminata, Musa paradisiaca, Musa spp. [banana]; Onagraceae such as the genera Camissonia, Oenothera e.g. the species Oeno-thera biennis or Camissonia brevipes [primrose, evening primrose]; Palmae such as the genera Elacis e.g. the species Elaeis guineensis [oil plam]; Papaveraceae such as the genera Papaver e.g. the species Papaver orientale, Papaver rhoeas, Papaver dubium [poppy, oriental poppy, corn poppy, field poppy, shirley poppies, field poppy, long-headed poppy, long-pod poppy]; Pedaliaceae such as the genera Sesamum e.g. the species Sesamum indicum [sesame]; Piperaceae such as the genera Piper, Artanthe, Peperomia, Steffensia e.g. the species Piper aduncum, Piper amalago, Piper angustifolium, Piper auritum, Piper betel, Piper cubeba, Piper longum, Piper nigrum, Piper retrofractum, Artanthe adunca, Ar-tanthe elongate, Peperomia elongate, Piper elongatum, Steffensia elongata. [Cayenne pepper, wild pepper]; Poaceae such as the genera Hordeum, Secale, Avena, Sorghum, Andropogon, Holcus, Panicum, Oryza, Zea, Triticum e.g. the species Hordeum vulgare, Hordeum jubatum, Hordeum murinum, Hordeum secalinum, Hordeum distichon Hordeum aegiceras, Hordeum hexastichon, Hordeum hexastichum, Hordeum irregulare, Hordeum sativum, Hordeum secalinum [barley, pearl barley, foxtail barley, wall barley, meadow bar-ley], Secale cereale [rye], Avena sativa, Avena fatua, Avena byzantine, Avena fatua var. sativa, Avena hybrida [oat], Sorghum bicolor, Sorghum halepense, Sorghum saccharatum, Sorghum vulgare, Andropogon drummondii, Holcus bicolor, Holcus sorghum, Sorghum aethiopicum, Sorghum arundinaceum, Sorghum caffrorum, Sorghum cernuum, Sorghum dochna, Sorghum drummondii, Sorghum durra, Sorghum guineense, Sorghum lanceolaturn, Sorghum nervosum, Sorghum saccharatum, Sorghum subglabrescens, Sorghum ver-ticilliflorum, Sorghum vulgare, Holcus halepensis, Sorghum mlliaceum millet, Panicum mili-taceum [Sorghum, millet], Oryza sativa, Oryza latifolia [rice], Zea mays [corn, maize] Triticum aestivum, Triticum durum, Triticum turgidum, Triticum hybernum, Triticum macha, Triti-cum sativum or Triticum vulgare [wheat, bread wheat, common wheat], Proteaceae such as the genera Macadamia e.g. the species Macadamia intergrifolia [macadamia]; Rubiaceae such as the genera Coffea e.g. the species Cofea spp., Coffea arabica, Coffea canephora or Coffea liberica [coffee]; Scrophulariaceae such as the genera Verbascum e.g. the species Verbascum blattaria, Verbascum chaixii, Verbascum densiflorum, Verbascum lagurus, Verbascum longifolium, Verbascum lychnitis, Verbascum nigrum, Verbascum olympicum, Verbascum phlomoides, Verbascum phoenicum, Verbascum pulverulentum or Verbascum thapsus [mullein, white moth mullein, nettle-leaved mullein, dense-flowered mullein, silver mullein, long-leaved mullein, white mullein, dark mullein, greek mullein, orange mullein, purple mullein, hoary mullein, great mullein]; Solanaceae such as the genera Capsicum, Nicotiana, Solanum, Lycopersicon e.g. the species Capsicum annuum, Capsicum annuum var. glabriusculum, Capsicum frutescens [pepper], Capsicum annuum [paprika], Nicotiana tabacum, Nicotiana alata, Nicotiana attenuata, Nicotiana glauca, Nicotiana langsdorffii, Nicotiana obtusifolia, Nicotiana quadrivalvis, Nicotiana repanda, Nicotiana rustica, Nicotiana sylvestris [tobacco], Solanum tuberosum [potato], Solanum melongena [egg-plant], Lycopersicon esculentum, Lycopersicon lycopersicum., Lycopersicon pyriforme, Solanum in-tegrifolium or Solanum lycopersicum [tomato]; Sterculiaceae such as the genera Theobroma e.g. the species Theobroma cacao [cacao]; Theaceae such as the genera Camellia e.g. the species Camellia sinensis [tea].

In one embodiment, the cultivated plant is selected from the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Annona spp., Apium graveolens, Arachis spp, Artocarpus spp., Asparagus offianalis, Avena spp., Averrhoa carambola, Bambusa sp., Benincasa hispida, Bertholletia excelsea, Beta vulgaris, Brassica spp. Cadaba farinosa, Canna indica, Capsicum spp., Carex elata, Carissa macrocarpa, Carya spp., Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Crataegus spp., Crocus sativus, Cucurbita spp., Cucumis spp., Cynara spp., Daucus carota, Desmodium spp., Dimocarpus Iongan, Dioscorea spp., Diospyros spp., Echinochloa spp., Elaeis (e.g. Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Hemerocallis fulva, Hibiscus spp., Hordeum spp., Lathyrus spp., Lens culinaris, Litchi chinensis, Lotus spp., Luffa acutangula, Lupinus spp., Luzula sylvatica, Lycopersicon spp. Macrotyloma spp., Malus spp., Malpighia emarginata, Mammea americana, Manilkara zapota, Medicago sativa, Melllotus spp., Mentha spp., Miscanthus sinensis, Momordica spp., Morus nigra, Musa spp., Nicotiana spp., Olea spp., Opuntia spp., Ornithopus spp., Oryza spp, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pisum spp., Poa spp., Populus spp., Prosopis spp., Prunus spp., Psidium spp., Pyrus communis, Quercus spp., Raphanus sativus, Rheum rhabarbarum, Ribes spp., Rubus spp., Saccharum spp., Salix sp., Sambucus spp., Secale cereale, Sesamum spp., Sinapis sp., Solanum spp., Spinacia spp., Syzygium spp., Tagetes spp., Tamarindus indica, Theobroma cacao, Trifolium spp., Tripsacum daclyloides, Triticosecale rimpaui, Triticum spp. (e.g. Triticum monococcum), Tropaeolum minus, Tropaeolum majus, Vaccinium spp., Vida spp., Vigna spp., Viola odorata, Vitis spp., Zizania palustris, Ziziphus spp., amongst others.

The cultivated plants are plants, which comprise at least one trait. The term “trait” refers to a property, which is present in the plant either by genetic engineering or by conventional breeding techniques. Each trait has to be assessed in relation to its respective control. Examples of traits are:
herbicide tolerance,
insect resistance by expression of bacterial toxins,
fungal resistance or viral resistance or bacterial resistance,
antibiotic resistance,
stress tolerance,
maturation alteration,
content modification of chemicals present in the cultivated plant, preferably increasing the content of fine chemicals advantageous for applications in the field of the food and/or feed industry, the cosmetics industry and/or the pharmaceutical industry,
modified nutrient uptake, preferably an increased nutrient use efficiency and/or resistance to conditions of nutrient deficiency,
improved fiber quality,
plant vigor,
modified colour,
fertility restoration,
and male sterility.

Principally, cultivated plants may also comprise combinations of the aforementioned traits, e.g. they may be tolerant to the action of herbicides and express bacterial toxins.

Principally, all cultivated plants may also provide combinations of the aforementioned properties, e.g. they may be tolerant to the action of herbicides and express bacterial toxins.

In the detailed description below, the term “plant” refers to a cultivated plant.

In one embodiment of the invention, the term “increased plant health” means an increase, as compared to the respective control, in a trait selected from the group consisting of: yield (e.g. increased biomass and/or seed yield), plant vigor (e.g. improved plant growth and/or early vigour and/or “greening effect”, meaning greener leaves, preferably leaves with a higher greenness index), early vigour, greening effect (preservation of green surface of a leaf), quality (e.g. improved content or composition of certain ingredients), tolerance to environmental stress, herbicide tolerance, insect resistance, fungal resistance or viral resistance or bacterial resistance, antibiotic resistance, content of fine chemicals advantageous for applications in the field of the food and/or feed industry, the cosmetics industry or the pharmaceutical industry, nutrient use efficiency, nutrient use uptake, fiber quality, color, and male sterility and/or “increased plant health” is to be understood as an alteration or modification, compared to the respective control, in a trait selected from the group consisting of maturation, fertility restoration and color.

“Plant health” is defined as a condition of the plant which is determined by several aspects alone or in combination with each other. One indicator for the condition of the plant is its “yield”.

So, in a preferred embodiment of the invention, the term “increased plant health” means an increase in yield as compared to the respective control.

In one embodiment, term “increased plant health” means any combination of 2, 3, 4, 5, 6 or more of the above mentioned traits.

In one embodiment of the invention, the term “increased plant health” means that the same effect as in the control plant can be achieved in the cultivated plant by reduced application rates and/or reduced application dosages.

The term “yield” in general means a measurable produce of economic value, typically related to a specified crop, to an area, and to a period of time. Individual plant parts directly contribute to yield based on their number, size and/or weight, or the actual yield is the yield per square meter for a crop and year, which is determined by dividing total production (includes both harvested and appraised production) by planted square meters. The term “yield” of a plant may relate to vegetative biomass (root and/or shoot biomass), to reproductive organs, and/or to propagules (such as seeds) of that plant. In one embodiment yield is to be understood as any plant product of economic value that is produced by the plant such as fruits, vegetables, nuts, grains, seeds, wood or even flowers. The plant products may in addition be further utilized and/or processed after harvesting. According to the present invention, “increased yield” of a plant, in particular of an agricultural, horticultural, silvicultural and/or ornamental plant means that the yield of a product of the respective plant is increased by a measurable amount over the yield of the same product of the control plant produced under the same conditions. In one embodiment of the invention increased yield is characterized, among others, by the following improved properties of the plant and/or its products compared with a control, such as increased weight, increased height, increased biomass such as higher overall fresh weight, higher grain yield, more tillers, larger leaves, increased shoot growth, increased protein content, increased oil content, increased starch content and/or increased pigment content.

Another indicator for the condition of the plant is its “plant vigor”.

According to the present invention, “increased plant vigor” of a plant, in particular of an agricultural, horticultural, silvicultural and/or ornamental plant means that the vigor of a plant is increased by a measurable amount over the vigor of the control plant under the same conditions.

In one embodiment of the invention the plant vigor becomes manifest in at least one aspects selected from the group consisting of improved vitality of the plant, improved plant growth, improved plant development, improved visual appearance, improved plant stand (less plant verse/lodging), better harvestability, improved emergence, enhanced nodulation in particular rhizobial nodulation, bigger size, bigger leaf blade, increased plant weight, increased plant height, increased tiller number, increased shoot growth, increased root growth (extensive root system), increased yield when grown on poor soils or unfavorable climate, enhanced photosynthetic activity, enhanced pigment content (for example chlorophyll content), earlier flowering, shorter flowering period, earlier fruiting, earlier and improved germination, earlier grain maturity, improved self-defense mechanisms, improved stress tolerance and resistance of the plants against biotic and abiotic stress factors such as fungi, bacteria, viruses, insects, heat stress, cold stress, drought stress, UV stress and/or salt stress, less non-productive tillers, less dead basal leaves, less input needed (such as fertilizers, water or pesticides), greener leaves (“greening effect”), less premature stress-induced ripening and less fruit abscission, complete maturation under shortened vegetation periods, longer and better grain-filling, less seeds needed, easier harvesting (for example by induction of leaf defoliation), faster and more uniform ripening, induction of young fruit abscission (“fruit thinning”), improved storability, longer shelf-life, easier and more cost effective storage conditions, longer panicles, delay of senescence, stronger and/or more productive tillers, better extractability of ingredients, improved quality of seeds (for being seeded in the following seasons for seed production) and/or reduced production of ethylene and/or the inhibition of its reception by the plant as compared with the control plant. The improvement of the plant vigor according to the present invention compared with the control, particularly means that the improvement of any one or several or all of the above mentioned plant characteristics are improved independently of the pesticidal action of the composition or active ingredients.

“Early vigour” refers to active healthy well-balanced growth especially during early stages of plant growth, and may result from increased plant fitness due to, for example, the plants being better adapted to their environment (i.e. optimizing the use of energy resources and partitioning between shoot and root). Plants having early vigour also show increased seedling survival and a better establishment of the crop, which often results in highly uniform fields (with the crop growing in uniform manner, i.e. with the majority of plants reaching the various stages of development at substantially the same time), and often better and higher yield. Therefore, early vigour may be determined by measuring various factors, such as thousand kernel weight, percentage germination, percentage emergence, seedling growth, seedling height, root length, root and shoot biomass and many more.

Another indicator for the condition of the plant is the “quality” of a plant and/or its products.

According to the present invention, “enhanced quality” means that certain crop characteristics such as the content or composition of certain ingredients are increased or improved by a measurable or noticeable amount over the same factor of the control plant produced under the same conditions.

In one embodiment of the invention the quality of a product of the respective plant becomes manifest in in at least one aspects selected from the group consisting of improved nutrient content, improved protein content, improved content of fatty acids, improved metabolite content, improved carotenoid content, improved sugar content, improved amount of essential and/or non-essential amino acids, improved nutrient composition, improved protein composition, improved composition of fatty acids, improved metabolite composition, improved carotenoid composition, improved sugar composition, improved amino acids composition, improved or optimal fruit color, improved texture of fruits, improved leaf color, higher storage capacity and/or higher processability of the harvested products as compared to the control.

In one embodiment of the invention the quality of a product of the respective plant becomes manifest in in at least one aspects selected from the group consisting of improved nutrient yield, improved protein yield, improved yield of fatty acids, improved metabolite yield, improved carotenoid yield, improved sugar yield and/or improved yield of essential and/or non-essential amino acids of the harvested products as compared to the control. In one embodiment of the invention, the nutrient yield, protein yield, yield of fatty acids, metabolite yield, carotenoid yield, sugar yield and/or yield of essential and/or non-essential amino acids is calculated as a function of seed and/or biomass yield in relation to the respective nutrient, protein, fatty acids, metabolite, carotenoid, sugar and/or essential and/or non-essential amino acids.

The terms “increase”, “improve” or “enhance” are interchangeable and shall mean in the sense of the application at least a 0.5%, 1%, 2%, 3%, 4%, 5%, 6%, 7%, 8%, 9% or 10%, preferably at least 15% or 20%, more preferably at least 25%, 30%, 35% or 40% more of the respective trait, characteristic, aspect, property, feature or attribute as disclosed in this specification, for example yield and/or growth in comparison to control plants as defined herein.

In one embodiment of the invention the increased seed yield manifest itself as one or more of the following: a) an increase in seed biomass (total seed weight) which may be on an individual seed basis and/or per plant and/or per square meter; b) increased number of flowers per plant; c) increased number of (filled) seeds; d) increased seed filling rate (which is expressed as the ratio between the number of filled seeds divided by the total number of seeds); e) increased harvest index, which is expressed as a ratio of the yield of harvestable parts, such as seeds, divided by the total biomass; and f) increased thousand kernel weight (TKW), which is extrapolated from the number of filled seeds counted and their total weight. An increased TKW may result from an increased seed size and/or seed weight, and may also result from an increase in embryo and/or endosperm size.

In one embodiment of the invention the increase in seed yield is also manifested as an increase in seed size and/or seed volume. Furthermore, an increase in seed yield is also manifest itself as an increase in seed area and/or seed length and/or seed width and/or seed perimeter. In a further embodiment increased yield also results in modified architecture, or may occur because of modified architecture.

In one embodiment the beneficial effect of the present invention may manifest itself not in the seed yield per se, but in the seed quality and the quality of the agricultural products produced from the plants treated according to the invention. Seed quality may relate to different parameters known in the art, such as enhanced nutrient or fine chemical content, e.g. amounts of vitamins or fatty acids and their composition; colouring or shape of the seed; germination rate or seed vigour; or reduced amounts of toxins, e.g. fungal toxins, and/or of substances hard to digest or indigestible, e.g. phytate, lignin.

The “greenness index” as used herein is calculated from digital images of plants. For each pixel belonging to the plant object on the image, the ratio of the green value versus the red value (in the RGB model for encoding colour) is calculated. The greenness index is expressed as the percentage of pixels for which the green-to-red ratio exceeds a given threshold. Under normal growth conditions, under salt stress growth conditions, and under reduced nutrient availability growth conditions, the greenness index of plants is measured in the last imaging before flowering. In contrast, under drought stress growth conditions, the greenness index of plants is measured in the first imaging after drought. Similarly the measurements may be done after exposure to other abiotic stress treatments, e.g. temperature.

Another indicator for the condition of the plant is the plant's tolerance or resistance to biotic and/or abiotic stress factors. Biotic and abiotic stress, especially over longer terms, can have harmful effects on plants. Biotic stress is caused by living organisms while abiotic stress is caused for example by environmental extremes or conditions unfavourable for an optimal growth of the plant.

According to the present invention, “enhanced tolerance or resistance to biotic and/or abiotic stress factors” means (1.) that certain negative factors caused by biotic and/or abiotic stress are diminished in a measurable or noticeable amount as compared to control plants exposed to the same conditions, and (2.) that the negative effects are not diminished by a direct action of the composition on the stress factors, for example by its fungicidal or insecticidal action which directly destroys the microorganisms or pests, but rather by a stimulation of the plants' own defensive reactions (“priming”) against said stress factors (“induced resistance”) or by the above mentioned synergistic effect.

Biotic stress can be caused by living organisms, such as pests (for example insects, arachnides, nematodes), competing plants (for example weeds), microorganisms (such as phythopathogenic fungi and/or bacteria) and/or viruses. Abiotic stress can be caused for example by extremes in temperature such as heat or cold (heat stress, cold stress), strong variations in temperature, temperatures unusual for the specific season, drought (drought stress), extreme wetness, high salinity (salt stress), radiation (for example by increased UV radiation due to the decreasing ozone layer), increased ozone levels (ozone stress), organic pollution (for example by phythotoxic amounts of pesticides) and inorganic pollution (for example by heavy metal contaminants). Both biotic as well as abiotic stress factors may in addition lead to secondary stresses such as oxidative stress.

As a result of biotic and/or abiotic stress factors, the quantity and the quality of the stressed plants, their crops and fruits decrease.

In one embodiment of the invention enhanced tolerance or resistance to biotic of the respective plant becomes manifest in in at least one aspects selected from the group consisting of tolerance or resistance to pests (for example insects, arachnides, nematodes), competing plants (for example weeds), microorganisms (such as phythopathogenic fungi and/or bacteria) and/or viruses.

In one embodiment of the invention enhanced tolerance or resistance to abiotic of the respective plant becomes manifest in at least one aspects selected from the group consisting of tolerance or resistance to extremes in temperature such as heat or cold (heat stress, cold stress), strong variations in temperature, temperatures unusual for the specific season, drought (drought stress), extreme wetness, high salinity (salt stress), radiation (for example by increased UV radiation due to the decreasing ozone layer), increased ozone levels (ozone stress), organic pollution (for example by phythotoxic amounts of pesticides) and inorganic pollution (for example by heavy metal contaminants).

The above identified indicators for the health condition of a plant may be interdependent and may result from each other. For example, an increased resistance to biotic and/or abiotic stress may lead to a better plant vigor, e.g. to better and bigger crops, and thus to an increased yield. Inversely, a more developed root system may result in an increased resistance to biotic and/or abiotic stress. However, these interdependencies and interactions are neither all known nor fully understood.

In one embodiment of the present invention, plant yield is increased by increasing the environmental stress tolerance(s) of a plant, in particular the tolerance to abiotic stress. Generally, the term “increased tolerance to stress” can be defined as survival of plants, and/or higher yield production, under stress conditions as compared to a control plant: For example, the plant of the invention is better adapted to the stress conditions. “Improved adaptation” to environmental stress like e.g. drought, heat, nutrient depletion, freezing and/or chilling temperatures refers herein to an improved plant performance resulting in an increased yield, particularly with regard to one or more of the yield related traits as defined in more detail above.

During its life-cycle, a plant is generally confronted with a diversity of environmental conditions. Any such conditions, which may, under certain circumstances, have an impact on plant yield, are herein referred to as “stress” condition. Environmental stresses may generally be divided into biotic and abiotic (environmental) stresses. Unfavourable nutrient conditions are sometimes also referred to as “environmental stress”. In one embodiment the present invention does also contemplate solutions for this kind of environmental stress, e.g. referring to increased nutrient use efficiency.

For the purposes of the description of the present invention, the terms “enhanced tolerance to stress”, “enhanced resistance to environmental stress”, “enhanced tolerance to environmental stress”, “improved adaptation to environmental stress” and other variations and expressions similar in its meaning are used interchangeably and refer, without limitation, to an improvement in tolerance to one or more environmental stress(es) as described herein and as compared to a corresponding control plant.

The term abiotic stress tolerance(s) refers for example low temperature tolerance, drought tolerance or improved water use efficiency (WUE), heat tolerance, salt stress tolerance and others. Stress tolerance in plants like low temperature, drought, heat and salt stress tolerance can have a common theme important for plant growth, namely the availability of water. Plants are typically exposed during their life cycle to conditions of reduced environmental water content. The protection strategies are similar to those of chilling tolerance.

Accordingly, in one embodiment of the present invention, said yield-related trait relates to an increased water use efficiency of the plant of the invention and/or an increased tolerance to drought conditions of the plant of the invention. Water use efficiency (WUE) is a parameter often correlated with drought tolerance. An increase in biomass at low water availability may be due to relatively improved efficiency of growth or reduced water consumption. In selecting traits for improving crops, a decrease in water use, without a change in growth would have particular merit in an irrigated agricultural system where the water input costs were high. An increase in growth without a corresponding jump in water use would have applicability to all agricultural systems. In many agricultural systems where water supply is not limiting, an increase in growth, even if it came at the expense of an increase in water use also increases yield.

In one embodiment of the present invention, an increased plant yield is mediated by increasing the “nutrient use efficiency of a plant”, e.g. by improving the nutrient use efficiency of nutrients including, but not limited to, phosphorus, potassium, and nitrogen. An increased nutrient use efficiency is in one embodiment an enhanced nitrogen uptake, assimilation, accumulation or utilization. These complex processes are associated with absorption, translocation, assimilation, and redistribution of nitrogen in the plant.

It has to be emphasized that the above mentioned effects of the method according to the invention, i.e. enhanced health of the plant, are also present when the plant is not under biotic stress for example when the plant is not under fungal- or pest pressure. It is evident that a plant suffering from fungal or insecticidal attack produces a smaller biomass and a smaller crop yield as compared to a plant which has been subjected to curative or preventive treatment against the pathogenic fungus or pest and which can grow without the damage caused by the biotic stress factor. However, the method according to the invention leads to an enhanced plant health even in the absence of any biotic stress and in particular of any phytopathogenic fungi or pest. This means that the positive effects of the method of the invention cannot be explained just by the pesticidal activities of the compounds of the invention, but are based on further activity profiles.

The term “plant” as used herein encompasses whole plants and progeny of the plants and plant parts, including seeds, shoots, stems, leaves, roots (including tubers), flowers, and tissues and organs. For the purposes of the invention, as a rule the plural is intended to encompass the singular and vice versa.

Tolerance to herbicides can be obtained by creating insensitivity at the site of action of the herbicide by expression of a target enzyme which is resistant to herbicide; rapid metabolism (conjugation or degradation) of the herbicide by expression of enzymes which inactivate herbicide; or poor uptake and translocation of the herbicide. Examples are the expression of enzymes which are tolerant to the herbicide in comparison to wild type enzymes, such as the expression of 5-enolpyruvylshikimate-3-phosphate synthase (EPSPS), which is tolerant to glyphosate (see e.g. Heck et. al, Crop Sci. 45, 2005, 329-339; Funke et. al, PNAS 103, 2006, 13010-13015; U.S. Pat. No. 5,188,642, U.S. Pat. No. 4,940,835, U.S. Pat. No. 5,633,435, U.S. Pat. No. 5,804,425, U.S. Pat. No. 5,627,061), the expression of glutamine synthase which is tolerant to glufosinate and bialaphos (see e.g. U.S. Pat. No. 5,646,024, U.S. Pat. No. 5,561,236) and DNA constructs coding for dicamba-degrading enzymes (see e.g. U.S. Pat. No. 7,105,724). Gene constructs can be obtained, for example, from micro-organism or plants, which are tolerant to said herbicides, such as the Agrobacterium strain CP4 EPSPS which is resistant to glyphosate; Streptomyces bacteria which are resistance to glufosinate; Arabidopsis, Daucus carota, Pseudomonoas spp. or Zea mais with chimeric gene sequences coding for HDDP (see e.g. WO 1996/38567, WO 2004/55191); Arabidopsis thaliana which is resistant to protox inhibitors (see e.g. US 2002/0073443).

Preferably, the herbicide tolerant plant can be selected from cereals such as wheat, barley, rye, oat; canola, sorghum, soybean, rice, oil seed rape, sugar beet, sugarcane, grapes, lentils, sunflowers, alfalfa, pome fruits; stone fruits; peanuts; coffee; tea; strawberries; turf; vegetables, such as tomatoes, potatoes, cucurbits and lettuce, more preferably, the plant is selected from soybean, maize (corn), rice, cotton, oilseed rape in particular canola, tomatoes, potatoes, sugarcane and cereals such as wheat, barley, rye and oat.

Examples of commercial available transgenic plants with tolerance to herbicides, are the corn varieties “Roundup Ready Corn”, “Roundup Ready 2” (Monsanto), “Agrisure GT”, “Agrisure GT/CB/LL”, “Agrisure GT/RW”, “Agrisure 3000GT” (Syngenta), “YieldGard VT Rootworm/RR2” and “YieldGard VT Triple” (Monsanto) with tolerance to glyphosate; the corn varieties “Liberty Link” (Bayer), “Herculex I”, “Herculex RW”, “Herculex Xtra” (Dow, Pioneer), “Agrisure GT/CB/LL” and “Agrisure CB/LL/RW” (Syngenta) with tolerance to glufosinate; the soybean varieties “Roundup Ready Soybean” (Monsanto) and “Optimum GAT” (DuPont, Pioneer) with tolerance to glyphosate; the cotton varieties “Roundup Ready Cotton” and “Roundup Ready Flex” (Monsanto) with tolerance to glyphosate; the cotton variety “FiberMax Liberty Link” (Bayer) with tolerance to glufosinate; the cotton variety “BXN” (Calgene) with tolerance to bromoxynil; the canola varieties “Navigator” and “Compass” (Rhone-Poulenc) with bromoxynil tolerance; the canola variety “Roundup Ready Canola” (Monsanto) with glyphosate tolerance; the canola variety “InVigor” (Bayer) with glufosinate tolerance; the rice variety “Liberty Link Rice” (Bayer) with glulfosinate tolerance and the alfalfa variety “Roundup Ready Alfalfa” with glyphosate tolerance. Further transgenic plants with herbicide tolerance are commonly known, for instance alfalfa, apple, eucalyptus, flax, grape, lentils, oil seed rape, peas, potato, rice, sugar beet, sunflower, tobacco, tomato turf grass and wheat with tolerance to glyphosate (see e.g. U.S. Pat. No. 5,188,642, U.S. Pat. No. 4,940,835, U.S. Pat. No. 5,633,435, U.S. Pat. No. 5,804,425, U.S. Pat. No. 5,627,061); beans, soybean, cotton, peas, potato, sunflower, tomato, tobacco, corn, sorghum and sugarcane with tolerance to dicamba (see e.g. U.S. Pat. No. 7,105,724 and U.S. Pat. No. 5,670,454); pepper, apple, tomato, millet, sunflower, tobacco, potato, corn, cucumber, wheat and sorghum with tolerance to 2,4-D (see e.g. U.S. Pat. No. 6,153,401, U.S. Pat. No. 6,100,446, WO 2005107437, U.S. Pat. No. 5,608,147 and U.S. Pat. No. 5,670,454); sugarbeet, potato, tomato and tobacco with tolerance to glufosinate (see e.g. U.S. Pat. No. 5,646,024, U.S. Pat. No. 5,561,236); canola, barley, cotton, lettuce, melon, millet, oats, potato, rice, rye, sorghum, soybean, sugarbeet, sunflower, tobacco, tomato and wheat with tolerance to acetolactate synthase (ALS) inhibiting herbicides, such as triazolopyrimidine sulfonamides, sulfonylureas and imidazolinones (see e.g. U.S. Pat. No. 5,013,659, WO 2006060634, U.S. Pat. No. 4,761,373, U.S. Pat. No. 5,304,732, U.S. Pat. No. 6,211,438, U.S. Pat. No. 6,211,439 and U.S. Pat. No. 6,222,100); cereals, sugar cane, rice, corn, tobacco, soybean, cotton, rapeseed, sugar beet and potato with tolerance to HPPD inhibitor herbicides (see e.g. WO 2004/055191, WO 199638567, WO 1997049816 and U.S. Pat. No. 6,791,014); wheat, soybean, cotton, sugar beet, rape, rice, sorghum and sugar cane with tolerance to protoporphyrinogen oxidase (PPO) inhibitor herbicides (see e.g. US 2002/0073443, US 20080052798, Pest Management Science, 61, 2005, 277-285). The methods of producing such transgenic plants are generally known to the person skilled in the art and are described, for example, in the publications mentioned above.

Plants, which are capable of synthesising one or more selectively acting bacterial toxins, comprise for example at least one toxin from toxin-producing bacteria, especially those of the genus Bacillus, in particular plants capable of synthesising one or more insecticidal proteins from Bacillus cereus or Bacillus popliae; or insecticidal proteins from Bacillus thuringiensis, such as delta-endotoxins, e.g. CryIA(b), CryIA(c), CryIF, CryIF(a2), CryIIA(b), CryIIIA, CryIIIB(b1) or Cry9c, or vegetative insecticidal proteins (VIP), e.g. VIP1, VIP2, VIP3 or VIP3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsine inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases.

In one embodiment a plant is capable of producing a toxin, lectin or inhibitor if it contains at least one cell comprising a nucleic acid sequence encoding said toxin, lectin, inhibitor or inhibitor producing enzyme, and said nucleic acid sequence is transcribed and translated and if appropriate the resulting protein processed and/or secreted in a constitutive manner or subject to developmental, inducible or tissue-specific regulation.

In the context of the present invention there are to be understood delta.-endotoxins, for example CryIA(b), CryIA(c), CryIF, CryIF(a2), CryIIA(b), CryIIIA, CryIIIB(b1) or Cry9c, or vegetative insecticidal proteins (VIP), for example VIP1, VIP2, VIP3 or VIP3A, expressly also hybrid toxins, truncated toxins and modified toxins. Hybrid toxins are produced recombinantly by a new combination of different domains of those proteins (see, for example, WO 02/15701). An example for a truncated toxin is a truncated CryIA(b), which is expressed in the Bt11 maize from Syngenta Seed SAS, as described below. In the case of modified toxins, one or more amino acids of the naturally occurring toxin are replaced. In such amino acid replacements, preferably non-naturally present protease recognition sequences are inserted into the toxin, such as, for example, in the case of CryIIIA055, a cathepsin-D-recognition sequence is inserted into a CryIIIA toxin (see WO 2003/018810).

Examples of such toxins or transgenic plants capable of synthesising such toxins are disclosed, for example, in EP-A-0 374 753, WO 93/07278, WO 95/34656, EP-A-0 427 529, EP-A-451 878 and WO 2003/052073.

The processes for the preparation of such transgenic plants are generally known to the person skilled in the art and are described, for example, in the publications mentioned above. CryI-type deoxyribonucleic acids and their preparation are known, for example, from WO 95/34656, EP-A-0 367 474, EP-A-0 401 979 and WO 1990/13651.

The toxin contained in the transgenic plants imparts to the plants tolerance to harmful insects. Such insects can occur in any taxonomic group of insects, but are especially commonly found in the beetles (Coleoptera), two-winged insects (Diptera) and butterflies (Lepidoptera).

Preferably, the plant capable of expression of bacterial toxins is selected from cereals such as wheat, barley, rye, oat; canola, cotton, eggplant, lettuce, sorghum, soybean, rice, oil seed rape, sugar beet, sugarcane, grapes, lentils, sunflowers, alfalfa, pome fruits; stone fruits; peanuts; coffee; tea; strawberries; turf; vegetables, such as tomatoes, potatoes, cucurbits and lettuce, more preferably, the plant is selected from cotton, soybean, maize (corn), rice, tomatoes, potatoes, oilseed rape and cereals such as wheat, barley, rye and oat, most preferably from cotton, soybean, maize and cereals such as wheat, barley, rye and oat.

Examples of commercial available transgenic plants capable of expression of bacterial toxins are the corn varieties “YieldGard corn rootworm” (Monsanto), “YieldGard VT” (Monsanto), “Herculex RW” (Dow, Pioneer), “Herculex Rootworm” (Dow, Pioneer) and “Agrisure CRW” (Syngenta) with resistance against corn rootworm; the corn varieties “YieldGard corn borer” (Monsanto), “YieldGard VT Pro” (Monsanto), “Agrisure CB/LL” (Syngenta), “Agrisure 3000GT” (Syngenta), “Hercules I”, “Hercules II” (Dow, Pioneer), “KnockOut” (Novartis), “NatureGard” (Mycogen) and “StarLink” (Aventis) with resistance against corn borer, the corn varieties “Herculex I” (Dow, Pioneer) and “Herculex Xtra” (Dow, Pioneer) with resistance against western bean cutworm, corn borer, black cutworm and fall armyworm; the corn variety “YieldGard Plus” (Monsanto) with resistance against corn borer and corn rootworm; the cotton variety “Bollgard I” “(Monsanto) with resistance against tobacco budworm; the cotton varieties “Bollgard II” (Monsanto), “WideStrike” (Dow) and “VipCot” (Syngenta) with resistance against tobacco budworm, cotton bollworm, fall armyworm, beet armyworm, cabbage looper, soybean lopper and pink bollworm; the potato varieties “NewLeaf”, “NewLeaf Y” and “NewLeaf Plus” (Monsanto) with tobacco hornworm resistance and the eggplant varieties “Bt brinjal”, “Dumaguete Long Purple”, “Mara” with resistance against brinjal fruit and shoot borer, bruit borer and cotton bollworm (see e.g. U.S. Pat. No. 5,128,130). Further transgenic plants with insect resistance are commonly known, such as yellow stemborer resistant rice (see e.g. Molecular Breeding, Volume 18, 2006, Number 1), lepidopteran resistant lettuce (see e.g. U.S. Pat. No. 5,349,124), resistant soybean (see e.g. U.S. Pat. No. 7,432,421) and rice with resistance against Lepidopterans, such as rice stemborer, rice skipper, rice cutworm, rice caseworm, rice leaffolder and rice armyworm (see e.g. WO 2001021821). The methods of producing such transgenic plants are generally known to the person skilled in the art and are described, for example, in the publications mentioned above.

Preferably, plants, which are capable of synthesising antipathogenic substances are selected from soybean, maize (corn), rice, tomatoes, potato, banana, papaya, tobacco, grape, plum and cereals such as wheat, barley, rye and oat, most preferably from soybean, maize (corn), rice, cotton, tomatoes, potato, banana, papaya, oil seed rape and cereals such as wheat, barley, rye and oat.

Plants, which are capable of synthesising antipathogenic substances having a selective action are for example plants expressing the so-called “pathogenesis-related proteins” (PRPs, see e.g. EP-A-0 392 225) or so-called “antifungal proteins” (AFPs, see e.g. U.S. Pat. No. 6,864,068). A wide range of antifungal proteins with activity against plant pathogenic fungi have been isolated from certain plant species and are common knowledge. Examples of such antipathogenic substances and transgenic plants capable of synthesising such antipathogenic substances are known, for example, from EP-A-0 392 225, WO 93/05153, WO 95/33818, and EP-A-0 353 191. Transgenic plants which are resistant against fungal, viral and bacterial pathogens are produced by introducing plant resistance genes. Numerous resistant genes have been identified, isolated and were used to improve plant resistant, such as the N gene which was introduced into tobacco lines that are susceptible to Tobacco Mosaic Virus (TMV) in order to produce TMV-resistant tobacco plants (see e.g. U.S. Pat. No. 5,571,706), the Prf gene, which was introduced into plants to obtain enhanced pathogen resistance (see e.g. WO 199802545) and the Rps2 gene from Arabidopsis thaliana, which was used to create resistance to bacterial pathogens including Pseudomonas syringae (see e.g. WO 199528423). Plants exhibiting systemic acquired resistance response were obtained by introducing a nucleic acid molecule encoding the TIR domain of the N gene (see e.g. U.S. Pat. No. 6,630,618). Further examples of known resistance genes are the Xa21 gene, which has been introduced into a number of rice cultivars (see e.g. U.S. Pat. No. 5,952,485, U.S. Pat. No. 5,977,434, WO 1999/09151, WO 1996/22375), the Rcg1 gene for colletotrichum resistance (see e.g. US 2006/225152), the prp1 gene (see e.g. U.S. Pat. No. 5,859,332, WO 2008/017706), the ppv-cp gene to introduce resistance against plum pox virus (see e.g. US PP15, 154Ps), the P1 gene (see e.g. U.S. Pat. No. 5,968,828), genes such as Blb1, Blb2, Blb3 and RB2 to introduce resistance against Phytophthora infestans in potato (see e.g. U.S. Pat. No. 7,148,397), the LRPKmI gene (see e.g. WO1999064600), the P1 gene for potato virus Y resistance (see e.g. U.S. Pat. No. 5,968,828), the HA5-1 gene (see e.g. U.S. Pat. No. 5,877,403 and U.S. Pat. No. 6,046,384), the PIP gene to introduce a broad resistant to viruses, such as potato virus X (PVX), potato virus Y (PVY), potato leafroll virus (PLRV) (see e.g. EP 0707069) and genes such as Arabidopsis N116, ScaM4 and ScaM5 genes to obtain fungal resistance (see e.g. U.S. Pat. No. 6,706,952 and EP 1018553). The methods of producing such transgenic plants are generally known to the person skilled in the art and are described, for example, in the publications mentioned above.

Antipathogenic substances which can be expressed by such transgenic plants include, for example, ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins”” (PRPs; see e.g. EP-A-0 392 225); antipathogenic substances produced by microorganisms, for example peptide antibiotics or heterocyclic antibiotics (see e.g. WO 1995/33818) or protein or polypeptide factors involved in plant pathogen defense (so-called “plant disease resistance genes”, as described in WO 2003/000906).

Antipathogenic substances produced by the plants are able to protect the plants against a variety of pathogens, such as fungi, viruses and bacteria. Useful plants of elevated interest in connection with present invention are cereals, such as wheat, barley, rye and oat; soybean; maize; rice; alfalfa, cotton, sugar beet, sugarcane, tobacco, potato, banana, oil seed rape; pome fruits; stone fruits; peanuts; coffee; tea; strawberries; turf; vines and vegetables, such as tomatoes, potatoes, cucurbits, papaya, melon, lenses and lettuce, more preferably selected from soybean, maize (corn), alfalfa, cotton, potato, banana, papaya, rice, tomatoes and cereals such as wheat, barley, rye and oat, most preferably from soybean, maize (corn), rice, cotton, potato, tomato, oilseed rape and cereals such as wheat, barley, rye and oat.

Transgenic plants with resistance against fungal pathogens, are, for examples, soybeans with resistance against Asian soybean rust (see e.g. WO 2008/017706); plants such as alfalfa, corn, cotton, sugar beet, oilseed, rape, tomato, soybean, wheat, potato and tobacco with resistance against Phytophtora infestans (see e.g. U.S. Pat. No. 5,859,332, U.S. Pat. No. 7,148,397, EP 1334979); corn with resistance against leaf blights, ear rots and stalk rots (such as anthracnose leaf bligh, anthracnose stalk rot, diplodia ear rot, Fusarium verticilioides, Gibberella zeae and top dieback, see e.g. US 2006/225152); apples with resistance against apple scab (Venturia inaequalis, see e.g. WO 1999064600); plants such as rice, wheat, barley, rye, corn, oats, potato, melon, soybean and sorghum with resistance against fusarium diseases, such as Fusarium graminearum, Fusarium sporotrichioides, Fusarium lateritium, Fusarium pseudograminearum Fusarium sambucinum, Fusarium culmorum, Fusarium poae, Fusarium acuminatum, Fusarium equiseti (see e.g. U.S. Pat. No. 6,646,184, EP 1477557); plants, such as corn, soybean, cereals (in particular wheat, rye, barley, oats, rye, rice), tobacco, sorghum, sugarcane and potatoes with broad fungal resistance (see e.g. U.S. Pat. No. 5,689,046, U.S. Pat. No. 6,706,952, EP 1018553 and U.S. Pat. No. 6,020,129).

Transgenic plants with resistance against bacterial pathogens and which are covered by the present invention, are, for examples, rice with resistance against Xylella fastidiosa (see e.g. U.S. Pat. No. 6,232,528); plants, such as rice, cotton, soybean, potato, sorghum, corn, wheat, barley, sugarcane, tomato and pepper, with resistance against bacterial blight (see e.g. WO 2006/42145, U.S. Pat. No. 5,952,485, U.S. Pat. No. 5,977,434, WO 1999/09151, WO 1996/22375); tomato with resistance against Pseudomonas syringae (see e.g. Can. J. Plant Path., 1983, 5: 251-255).

Transgenic plants with resistance against viral pathogens, are, for examples, stone fruits, such as plum, almond, apricot, cherry, peach, nectarine, with resistance against plum pox virus (PPV, see e.g. US PP15, 154Ps, EP 0626449); potatoes with resistance against potato virus Y (see e.g. U.S. Pat. No. 5,968,828); plants such as potato, tomato, cucumber and leguminosaes which are resistant against tomato spotted wilt virus (TSWV, see e.g. EP 0626449, U.S. Pat. No. 5,973,135); corn with resistance against maize streak virus (see e.g. U.S. Pat. No. 6,040,496); papaya with resistance against papaya ring spot virus (PRSV, see e.g. U.S. Pat. No. 5,877,403, U.S. Pat. No. 6,046,384); cucurbitaceae, such as cucumber, melon, watermelon and pumpkin, and solanaceae, such as potato, tobacco, tomato, eggplant, paprika and pepper, with resistance against cucumber mosaic virus (CMV, see e.g. U.S. Pat. No. 6,849,780); cucurbitaceae, such as cucumber, melon, watermelon and pumpkin, with resistance against watermelon mosaic virus and zucchini yellow mosaic virus (see e.g. U.S. Pat. No. 6,015,942); potatoes with resistance against potato leafroll virus (PLRV, see e.g. U.S. Pat. No. 5,576,202); potatoes with a broad resistance to viruses, such as potato virus X (PVX), potato virus Y (PVY), potato leafroll virus (PLRV) (see e.g. EP 0707069).

TABLE I
Further examples of deregulated or commercially available transgenic
plants with modified genetic material capable of expression of
antipathogenic substances are

| Crop | Event | Company |
|---|---|---|
| Carica papaya (Papaya) | 55-1/63-1 | Cornell University |
| Carica papaya (Papaya) | X17-2 | University of Florida |
| Cucurbita pepo (Squash) | CZW-3 | Asgrow (USA); Seminis |
| | | Vegetable Inc. (Canada) |
| Cucurbita pepo (Squash) | ZW20 | Upjohn (USA); Seminis |
| | | Vegetable Inc. (Canada) |
| Prunus domestica (Plum) | C5 | United States Department |
| | | of Agriculture - Agricultural |
| | | Research Service |
| Solanum tuberosum L. | RBMT15-101, | Monsanto Company |
| (Potato) | SEMT15-02, | |
| | SEMT15-15 | |
| Solanum tuberosum L. | RBMT21-129, | Monsanto Company |
| (Potato) | RBMT21-350, | |
| | RBMT22-082 | |

Transgenic plants with resistance against nematodes and which may be used in the methods of the present invention are, for examples, soybean plants with resistance to soybean cyst nematodes. Methods have been proposed for the genetic transformation of plants in order to confer increased resistance to plant parasitic nematodes. U.S. Pat. Nos. 5,589,622 and 5,824,876 are directed to the identification of plant genes expressed specifically in or adjacent to the feeding site of the plant after attachment by the nematode.

Also known in the art are transgenic plants with reduced feeding structures for parasitic nematodes, e.g. plants resistant to herbicides except of those parts or those cells that are nematode feeding sites and treating such plant with a herbicide to prevent, reduce or limit nematode feeding by damaging or destroying feeding sites (e.g. U.S. Pat. No. 5,866,777).

Use of RNAi to target essential nematode genes has been proposed, for example, in PCT Publication WO 2001/96584, WO 2001/17654, US 2004/0098761, US 2005/0091713, US 2005/0188438, US 2006/0037101, US 2006/0080749, US 2007/0199100, and US 2007/0250947.

Transgenic nematode resistant plants have been disclosed, for example in the PCT publications WO 2008/095886 and WO 2008/095889.

Plants which are resistant to antibiotics, such as kanamycin, neomycin and ampicillin. The naturally occurring bacterial nptII gene expresses the enzyme that blocks the effects of the antibiotics kanamycin and neomycin. The ampicillin resistance gene ampR (also known as blaTEM1) is derived from the bacterium Salmonella paratyphi and is used as a marker gene in the transformation of micro-organisms and plants. It is responsible for the synthesis of the enzyme beta-lactamase, which neutralises antibiotics in the penicillin group, including ampicillin. Transgenic plants with resistance against antibiotics, are, for examples potato, tomato, flax, canola, oilseed rape and corn (see e.g. Plant Cell Reports, 20, 2001, 610-615. Trends in Plant Science, 11, 2006, 317-319. Plant Molecular Biology, 37, 1998, 287-296. Mol Gen Genet., 257, 1998, 606-13.). Plant Cell Reports, 6, 1987, 333-336. Federal Register (USA), Vol. 60, No. 113, 1995, page 31139. Federal Register (USA), Vol. 67, No. 226, 2002, page 70392. Federal Register (USA), Vol. 63, No. 88, 1998, page 25194. Federal Register (USA), Vol. 60, No. 141, 1995, page 37870. Canadian Food Inspection Agency, FD/OFB-095-264-A, October 1999, FD/OFB-099-127-A, October 1999. Preferably, the plant is selected from soybean, maize (corn), rice, cotton, oilseed rape, potato, sugarcane, alfalfa, tomatoes and cereals, such as wheat, barley, rye and oat, most preferably from soybean, maize (corn), rice, cotton, oilseed rape, tomato, potato and cereals such as wheat, barley, rye and oat.

Plants which are tolerant to stress conditions (see e.g. WO 2000/04173, WO 2007/131699, CA 2521729 and US 2008/0229448) are plants, which show increased tolerance to abiotic stress conditions such as drought, high salinity, high light intensities, high UV irradiation, chemical pollution (such as high heavy metal concentration), low or high temperatures, limited supply of nutrients (i.e. nitrogen, phosphorous) and population stress. Preferably, transgenic plants with resistance to stress conditions, are selected from rice, corn, soybean, sugarcane, alfalfa, wheat, tomato, potato, barley, rapeseed, beans, oats, sorghum and cotton with tolerance to drought (see e.g. WO 2005/048693, WO 2008/002480 and WO 2007/030001); corn, soybean, wheat, cotton, rice, rapeseed and alfalfa with tolerance to low temperatures (see e.g. U.S. Pat. No. 4,731,499 and WO 2007/112122); rice, cotton, potato, soybean, wheat, barley, rye, sorghum, alfalfa, grape, tomato, sunflower and tobacco with tolerance to high salinity (see e.g. U.S. Pat. No. 7,256,326, U.S. Pat. No. 7,034,139, WO 2001/030990). The methods of producing such transgenic plants are generally known to the person skilled in the art and are described, for example, in the publications mentioned above. Preferably, the plant is selected from soybean, maize (corn), rice, cotton, sugarcane, alfalfa, sugar beet, potato, oilseed rape, tomatoes and cereals such as wheat, barley, rye and oat, most preferably from soybean, maize (corn), rice, cotton, oilseed rape, tomato, potato, sugarcane and cereals such as wheat, barley, rye and oat.

Altered maturation properties, are for example delayed ripening, delayed softening and early maturity. Preferably, transgenic plants with modified maturation properties, are, selected from tomato, melon, raspberry, strawberry, muskmelon, pepper and papaya with delayed ripening (see e.g. U.S. Pat. No. 5,767,376, U.S. Pat. No. 7,084,321, U.S. Pat. No. 6,107,548, U.S. Pat. No. 5,981,831, WO 1995035387, U.S. Pat. No. 5,952,546, U.S. Pat. No. 5,512,466, WO 1997001952, WO 1992/008798, Plant Cell. 1989, 53-63. Plant Molecular Biology, 50, 2002). The methods of producing such transgenic plants are generally known to the person skilled in the art and are described, for example, in the publications mentioned above. Preferably, the plant is selected from fruits, such as tomato, vine, melon, papaya, banana, pepper, raspberry and strawberry; stone fruits, such as cherry, apricot and peach; pome fruits, such as apple and pear; and citrus fruits, such as citron, lime, orange, pomelo, grapefruit, and mandarin; more preferably from tomato, vine, apple, banana, orange and strawberry, most preferably tomatoes.

Content modification is synthesis of modified chemical compounds (if compared to the corresponding control plant) or synthesis of enhanced amounts of chemical (if compounds compared to the corresponding control plant) and corresponds to an increased or reduced amount of vitamins, amino acids, proteins and starch, different oils and a reduced amount of nicotine.

Commercial examples are the soybean varieties “Vistive II” and “Visitive III” with low-linolenic/medium oleic content; the corn variety “Mavera high-value corn” with increased lysine content; and the soybean variety “Mavera high value soybean” with yielding 5% more protein compared to conventional varieties when processed into soybean meal. Further transgenic plants with altered content are, for example, potato and corn with modified amylopectin content (see e.g. U.S. Pat. No. 6,784,338, US 20070261136); canola, corn, cotton, grape, catalpa, cattail, rice, soybean, wheat, sunflower, balsam pear and vernonia with a modified oil content (see e.g. U.S. Pat. No. 7,294,759, U.S. Pat. No. 7,157,621, U.S. Pat. No. 5,850,026, U.S. Pat. No. 6,441,278, U.S. Pat. No. 6,380,462, U.S. Pat. No. 6,365,802, U.S. Pat. No. 6,974,898, WO 2001/079499, US 2006/0075515 and U.S. Pat. No. 7,294,759); sunflower with increased fatty acid content (see e.g. U.S. Pat. No. 6,084,164); soybeans with modified allergens content (so called “hypoallergenic soybean, see e.g. U.S. Pat. No. 6,864,362); tobacco with reduced nicotine content (see e.g. US 20060185684, WO 2005000352 and WO 2007064636); canola and soybean with increased lysine content (see e.g. Bio/Technology 13, 1995, 577-582); corn and soybean with altered composition of methionine, leucine, isoleucine and valine (see e.g. U.S. Pat. No. 6,946,589, U.S. Pat. No. 6,905,877); soybean with enhanced sulfur amino acid content (see e.g. EP 0929685, WO 1997041239); tomato with increased free amino acid contents, such as asparagine, aspartic acid, serine, threonine, alanine, histidine and glutamic acid (see e.g. U.S. Pat. No. 6,727,411); corn with enhanced amino acid content (see e.g. WO 05077117); potato, corn and rice with modified starch content (see e.g. WO 1997044471 and U.S. Pat. No. 7,317,146); tomato, corn, grape, alfalfa, apple, beans and peas with modified flavonoid content (see e.g. WO 2000/04175); corn, rice, sorghum, cotton, soybeans with altered content of phenolic compounds (see e.g. US 20080235829). The methods of producing such transgenic plants are generally known to the person skilled in the art and are described, for example, in the publications mentioned above. Preferably, the plant is selected from soybean, maize (corn), rice, cotton, sugarcane, potato, tomato, oilseed rape, flax and cereals such as wheat, barley, rye and oat, most preferably soybean, maize (corn), rice, oilseed rape, potato, tomato, cotton and cereals such as wheat, barley, rye and oat.

Enhanced nutrient utilization is e.g. assimilation or metabolism of nitrogen or phosphorous. Preferably, transgenic plants with enhanced nitrogen assimilatory and utilization capacities are selected from for example, canola, corn, wheat, sunflower, rice, tobacco, soybean, cotton, alfalfa, tomato, wheat, potato, sugar beet, sugar cane and rapeseed (see e.g. WO 1995/009911, WO 1997/030163, U.S. Pat. No. 6,084,153, U.S. Pat. No. 5,955,651 and U.S. Pat. No. 6,864,405). Plants with improved phosphorous uptake are, for example, tomato and potato (see e.g. U.S. Pat. No. 7,417,181). The methods of producing such transgenic plants are generally known to the person skilled in the art and are described, for example, in the publications mentioned above. Preferably, the plant is selected from soybean, maize (corn), rice, cotton, sugarcane, alfalfa, potato, oilseed rape and cereals such as wheat, barley, rye and oat, most preferably from soybean, maize (corn), rice, cotton, oilseed rape, tomato, potato and cereals such as wheat, barley,

Transgenic plants with male sterility are preferably selected from canola, corn, tomato, rice, Indian mustard, wheat, soybean and sunflower (see e.g. U.S. Pat. No. 6,720,481, U.S. Pat. No. 6,281,348, U.S. Pat. No. 5,659,124, U.S. Pat. No. 6,399,856, U.S. Pat. No. 7,345,222, U.S. Pat. No. 7,230,168, U.S. Pat. No. 6,072,102, EP1 135982, WO 2001/092544 and WO 1996/040949). The methods of producing such transgenic plants are generally known to the person skilled in the art and are described, for example, in the publications mentioned above. Preferably, the plant is selected from soybean, maize (corn), rice, cotton, oilseed rape, tomato, potato and cereals such as wheat, barley.

TABLE II
Further examples of deregulated or commercially available transgenic
plants with modified genetic material being male sterile are

| Crop | Event | Company |
|---|---|---|
| Brassica napus (Argentine | MS1, RF1 => PGS1 | Bayer CropScience (formerly Plant |
| Canola) | | Genetic Systems) |
| Brassica napus (Argentine | MS1, RF2 => PGS2 | Bayer CropScience (formerly Plant |
| Canola) | | Genetic Systems) |
| Brassica napus (Argentine | MS8xRF3 | Bayer CropScience (Aventis Crop- |
| Canola) | | Science(AgrEvo)) |
| Brassica napus (Argentine | PHY14, PHY35 | Bayer CropScience (formerly Plant |
| Canola) | | Genetic Systems) |
| Brassica napus (Argentine | PHY36 | Bayer CropScience (formerly Plant |
| Canola) | | Genetic Systems) |
| Cichorium intybus (Chicory) | RM3-3, RM3-4, | Bejo Zaden BV |
| | RM3-6 | |
| Zea mays L. (Maize) | 676, 678, 680 | Pioneer Hi-Bred International Inc. |
| Zea mays L. (Maize) | MS3 | Bayer CropScience (Aventis Crop- |
| | | Science(AgrEvo)) |
| Zea mays L. (Maize) | MS6 | Bayer CropScience (Aventis Crop- |
| | | Science(AgrEvo)) |

Plants, which produce higher quality fiber are e.g. transgenic cotton plants. The such improved quality of the fiber is related to improved micronaire of the fiber, increased strength, improved staple length, improved length uniformity and color of the fibers (see e.g. WO 1996/26639, U.S. Pat. No. 7,329,802, U.S. Pat. No. 6,472,588 and WO 2001/17333). The methods of producing such transgenic plants are generally known to the person skilled in the art and are described, for example, in the publications mentioned above.

As set forth above, cultivated plants may comprise one or more traits, e.g. selected from the group consisting of herbicide tolerance, insect resistance, fungal resistance, viral resistance, bacterial resistance, stress tolerance, maturation alteration, content modification, modified nutrient uptake and male sterility (see e.g. WO 2005033319 and U.S. Pat. No. 6,376,754).

Examples of commercial available transgenic plants with two combined properties are the corn varieties “YieldGard Roundup Ready” and YieldGard Roundup Ready 2″ (Monsanto) with glyphosate tolerance and resistance to corn borer; the corn variety “Agrisure CB/LL” (Syntenta) with glufosinate tolerance and corn borer resistance; the corn variety “Yield Gard VT Rootworm/RR2” with glyphosate tolerance and corn rootworm resistance; the corn variety “Yield Gard VT Triple” with glyphosate tolerance and resistance against corn rootworm and corn borer; the corn variety “Herculex I” with glufosinate tolerance and lepidopteran resistance (CryIF), i.e. against western bean cutworm, corn borer, black cutworm and fall armyworm; the corn variety “YieldGard Corn Rootworm/Roundup Ready 2” (Monsanto) with glyphosate tolerance and corn rootworm resistance; the corn variety “Agrisure GT/RW” (Syngenta) with gluphosinate tolerance and lepidopteran resistance (Cry3A), i.e. against western corn rootworm, northern corn rootworm and Mexican corn rootworm; the corn variety “Herculex RW” (Dow, Pioneer) with glufosinate tolerance and lepidopteran resistance (Cry34/35Ab1), i.e. against western corn rootworm, northern corn rootworm and Mexican corn rootworm; the corn variety “Yield Gard VT Rootworm/RR2” with glyphosate tolerance and corn rootworm resistance; the soybean variety “Optimum GAT” (DuPont, Pioneer) with glyphosate tolerance and ALS herbicide tolerance; the corn variety “Mavera high-value corn” with glyphosate tolerance, resistance to corn rootworm and European corn borer and high lysine trait.

Examples of commercial available transgenic plants with three traits are the corn variety “Herculex I/Roundup Ready 2” with glyphosate tolerance, gluphosinate tolerance and lepidopteran resistance (Cry1F), i.e. against western bean cutworm, corn borer, black cutworm and fall armyworm; the corn variety “YieldGard Plus/Roundup Ready 2” (Monsanto) with glyphosate tolerance, corn rootworm resistance and corn borer resistance; the corn variety “Agrisure GT/CB/LL” (Syngenta) with tolerance to glyphosate tolerance, tolerance to gluphosinate and corn borer resistance; the corn variety “Herculex Xtra” (Dow, Pioneer) with glufosinate tolerance and lepidopteran resistance (Cry1F+Cry34/35Ab1), i.e. against western corn rootworm, northern corn rootworm, Mexican corn rootworm, western bean cutworm, corn borer, black cutworm and fall armyworm; the corn varieties “Agrisure CB/LL/RW” (Syngenta) with glufosinate tolerance, corn borer resistance (Cry1Ab) and lepidopteran resistance (Cry3A), i.e. against western corn rootworm, northern corn rootworm and Mexican corn rootworm; the corn variety “Agrisure 3000GT” (Syngenta) with glyphosate tolerance+corn borer resistance (Cry1Ab) and lepidopteran resistance (Cry3A), i.e. against western corn rootworm, northern corn rootworm and Mexican corn rootworm. The methods of producing such transgenic plants are generally known to the person skilled in the art. An example of a commercial available transgenic plant with four traits is “Hercules Quad-Stack” with glyphosate tolerance, glufosinate tolerance, corn borer resistance and corn rootworm resistance.

In one embodiment of the invention the cultivated plant is selected from the group of plants as mentioned in the paragraphs and tables of this disclosure, preferably as mentioned above. Preferably, the cultivated plants are plants, which comprise at least one trait selected from herbicide tolerance, insect resistance for example by expression of one or more bacterial toxins, fungal resistance or viral resistance or bacterial resistance by expression of one or more antipathogenic substances, stress tolerance, nutrient uptake, nutrient use efficiency, content modification of chemicals present in the cultivated plant compared to the corresponding control plant.

More preferably, the cultivated plants are plants, which comprise at least one trait selected from herbicide tolerance, insect resistance by expression of one or more bacterial toxins, fungal resistance or viral resistance or bacterial resistance by expression of one or more antipathogenic substances, stress tolerance, content modification of one or more chemicals present in the cultivated plant compared to the corresponding control plant.

Most preferably, the cultivated plants are plants, which are tolerant to the action of herbicides and plants, which express one or more bacterial toxins, which provides resistance against one or more animal pests (such as insects or arachnids or nematodes), wherein the bacterial toxin is preferably a toxin from Bacillus thuriginensis. Herein, the cultivated plant is preferably selected from soybean, maize (corn), rice, cotton, sugarcane, alfalfa, potato, oilseed rape, tomatoes and cereals such as wheat, barley, rye and oat, most preferably from soybean, maize (corn), cotton, rice and cereals such as wheat, barley, rye and oat.

Utmost preference is given to cultivated plants, which are tolerant to the action of herbicides.

In another utmost preference, the cultivated plants are plants, which are given in table A. Sources: AgBios database and GMO-compass database (AG BIOS, P.O. Box 475, 106 St. John St. Merickville, Ontario KOG1NO, Canada, access: http://www.agbios.com/dbase.php, also see BioTechniques, Volume 35, No. 3, September 2008, p. 213, and http://www.gmo-compass.org/eng/gmo/db/).

TABLE A

| | | Trait category | | | |
|---|---|---|---|---|---|
| | | (sub- | Transgenic | | |
| No | Crop | category) | event | Company | Description |
| A-1 | Agrostis | Herbicide | ASR368 | Scotts | ARS368 was developed by introducing the |
| | stolonifera | tolerance | | Seeds | CP4 EPSPS coding sequences into the |
| | (creeping | (Glyphosate | | | creeping bentgrass line B99061R using microprojectile |
| | bentgrass) | tolerance) | | | bombardment. Glyphosate tolerance |
| | | | | | derived inserting a modified EPSPS |
| | | | | | encoding gene from Agrobacterium tumefaciens. |
| A-2 | Beta vulgaris | Herbicide | A5-15 | Danisco | Soil bacterium Agrobacteriums sp. strain |
| | (sugar | tolerance | | Seeds/DLF | CP4. The cp4 epsps gene encodes for a |
| | beet) | (Glyphosate | | Trifolium | version of EPSPS that is highly tolerant to |
| | | tolerance) | | | inhibition by glyphosate and therefore leads |
| | | | | | to increased tolerance to glyphosate- |
| | | | | | containing herbicides. |
| A-3 | Beta vulgaris | Herbicide | GTSB77 | Novartis | Glyphosate herbicide tolerant sugar beet |
| | (sugar | tolerance | | Seeds; | produced by inserting a gene encoding the |
| | beet) | (Glyphosate | | Monsanto | enzyme 5-enolypyruvylshikimate-3- |
| | | tolerance) | | Company | phosphate synthase (EPSPS) from the CP4 |
| | | | | | strain of Agrobacterium tumefaciens. |
| A-4 | Beta vulgaris | Herbicide | H7-1 | Monsanto | Glyphosate herbicide tolerant sugar beet |
| | (sugar | tolerance | | Company | produced by inserting a gene encoding the |
| | beet) | (Glyphosate | | | enzyme 5-enolypyruvylshikimate-3- |
| | | tolerance) | | | phosphate synthase (EPSPS) from the CP4 |
| | | | | | strain of Agrobacterium tumefaciens. |
| A-5 | Beta vulgaris | Herbicide | T120-7 | Bayer | Introduction of the PPT-acetyltransferase |
| | (sugar | tolerance | | CropScience | (PAT) encoding gene from Streptomyces |
| | beet) | (Glyphosate | | (Aventis | viridochromogenes, an aerobic soil bacteria. |
| | | tolerance) | | CropScience | PPT normally acts to inhibit glutamine synthetase, |
| | | | | (AgrEvo)) | causing a fatal accumulation of |
| | | | | | ammonia. Acetylated PPT is inactive. |
| A-6 | Brassica | Herbicide | GT200 | Monsanto | Glyphosate herbicide tolerant canola produced |
| | napus (Argentine | tolerance | | Company | by inserting genes encoding the enzymes |
| | canola) | (Glyphosate | | | 5-enolypyruvylshikimate-3-phosphate |
| | | tolerance) | | | synthase (EPSPS) from the CP4 strain of |
| | | | | | Agrobacterium tumefaciens and glyphosate |
| | | | | | oxidase from Ochrobactrum anthropi. |
| A-7 | Brassica | Herbicide | GT73, RT73 | Monsanto | Glyphosate herbicide tolerant canola produced |
| | napus (Argentine | tolerance | | Company | by inserting genes encoding the enzymes |
| | canola) | (Glyphosate | | | 5-enolypyruvylshikimate-3-phosphate |
| | | tolerance) | | | synthase (EPSPS) from the CP4 strain of |
| | | | | | Agrobacterium tumefaciens and glyphosate |
| | | | | | oxidase from Ochrobactrum anthropi. |
| A-8 | Brassica | Herbicide | HCN10 | Aventis | Introduction of the PPT-acetyltransferase |
| | napus (Argentine | tolerance | | CropScience | (PAT) encoding gene from Streptomyces |
| | canola) | (Glyphosate | | | viridochromogenes, an aerobic soil bacteria. |
| | | tolerance) | | | PPT normally acts to inhibit glutamine synthetase, |
| | | | | | causing a fatal accumulation of |
| | | | | | ammonia. Acetylated PPT is inactive. |
| A-9 | Brassica | Herbicide | HCN92 | Bayer | Introduction of the PPT-acetyltransferase |
| | napus (Argentine | tolerance | | CropScience | (PAT) encoding gene from Streptomyces |
| | canola) | (Glyphosate | | (Aventis | viridochromogenes, an aerobic soil bacteria. |
| | | tolerance) | | CropScience | PPT normally acts to inhibit glutamine synthetase, |
| | | | | (AgrEvo)) | causing a fatal accumulation of |
| | | | | | ammonia. Acetylated PPT is inactive. |
| A-10 | Brassica | Herbicide | T45 | Bayer | Introduction of the PPT-acetyltransferase |
| | napus (Argentine | tolerance | (HCN28) | CropScience | (PAT) encoding gene from Streptomyces |
| | canola) | (Glyphosate | | (Aventis | viridochromogenes, an aerobic soil bacteria. |
| | | tolerance) | | CropScience | PPT normally acts to inhibit glutamine synthetase, |
| | | | | (AgrEvo)) | causing a fatal accumulation of |
| | | | | | ammonia. Acetylated PPT is inactive. |
| A-11 | Brassica | Herbicide | ZSR500/502 | Monsanto | Introduction of a modified 5-enol- |
| | rapa (Polish | tolerance | | Company | pyruvylshikimate-3-phosphate synthase |
| | canola) | (Glyphosate | | | (EPSPS) and a gene from Achromobacter sp |
| | | tolerance) | | | that degrades glyphosate by conversion to |
| | | | | | aminomethylphosphonic acid (AMPA) and |
| | | | | | glyoxylate by interspecific crossing with |
| | | | | | GT73. |
| A-12 | Glycine max L. | Herbicide | GTS 40-3-2 | Monsanto | Glyphosate tolerant soybean variety produced |
| | (soybean) | tolerance | | Company | by inserting a modified 5- |
| | | (Glyphosate | | | enolpyruvylshikimate-3-phosphate synthase |
| | | tolerance) | | | (EPSPS) encoding gene from the soil bacterium |
| | | | | | Agrobacterium tumefaciens. |
| A-13 | Glycine max L. | Herbicide | MON40-3-2 | Monsanto | The cp4 epsps gene from soil bacterium |
| | (soybean) | tolerance | | Company | Agrobacterium ssp. strain CP4 was introduced. |
| | | (Glyphosate | | | The cp4 epsps gene encodes for a |
| | | tolerance) | | | version of EPSPS that is highly tolerant to |
| | | | | | inhibition by glyphosate and therefore leads |
| | | | | | to increased tolerance to glyphosate- |
| | | | | | containing herbicides. |
| A-14 | Glycine max L. | Herbicide | MON89788 | Monsanto | Glyphosate-tolerant soybean produced by |
| | (soybean) | tolerance | | Company | inserting a modified 5-enolpyruvylshikimate- |
| | | (Glyphosate | | | 3-phosphate synthase (EPSPS) encoding |
| | | tolerance) | | | aroA (epsps) gene from Agrobacterium tumefaciens |
| | | | | | CP4. |
| A-15 | Glycine max L. | Herbicide | DP356043 | Pioneer Hi- | Soybean event with two herbicide tolerance |
| | (soybean) | tolerance | | Bred International | genes: glyphosate N-acetlytransferase, |
| | | (Glyphosate | | Inc. | which detoxifies glyphosate, and a modified |
| | | tolerance, | | | acetolactate synthase (ALS) gene which is |
| | | ALH-inhibitor | | | tolerant to ALS-inhibitng herbicides. |
| | | tolerance) | | | |
| A-16 | Gossypium | Herbicide | GHB614 | Bayer Crop- | Glyphosate herbicide tolerant cotton produced |
| | hirsutum L. | tolerance | | Science | by inserting a double-mutated form of |
| | (cotton) | (Glyphosate | | USA LP | the enzyme 5-enolpyruvyl shikimate-3- |
| | | tolerance) | | | phosphate synthase (EPSPS) from Zea |
| | | | | | mays. |
| A-17 | Gossypium | Herbicide | MON1445 | Monsanto | Introduction of cp4 epsps gene from soil |
| | hirsutum L. | tolerance | | Company | bacterium Agrobacterium ssp. strain CP4. |
| | (cotton) | (Glyphosate | | | The cp4 epsps gene encodes for a version |
| | | tolerance) | | | of EPSPS that is highly tolerant to inhibition |
| | | | | | by glyphosate. |
| A-18 | Gossypium | Herbicide | MON1445/1698 | Monsanto | Glyphosate herbicide tolerant cotton produced |
| | hirsutum L. | tolerance | | Company | by inserting a naturally glyphosate |
| | (cotton) | (Glyphosate | | | tolerant form of the enzyme 5-enolpyruvyl |
| | | tolerance) | | | shikimate-3-phosphate synthase (EPSPS) |
| | | | | | from A. tumefaciens strain CP4. |
| A-19 | Gossypium | Herbicide | MON88913 | Monsanto | Glyphosate herbicide tolerant cotton produced |
| | hirsutum L. | tolerance | | Company | by inserting two genes encoding the |
| | (cotton) | (Glyphosate | | | enzyme 5-enolypyruvylshikimate-3- |
| | | tolerance) | | | phosphate synthase (EPSPS) from the CP4 |
| | | | | | strain of Agrobacterium tumefaciens. |
| A-20 | Medicago | Herbicide | MON- | Monsanto | Containing glyphosate-tolerant form of the |
| | sativa (alfalfa) | tolerance | ØØ1Ø1-8, | and Forage | plant enzyme 5-enolpyruvylshikimate-3- |
| | | (Glyphosate | MON- | Genetics | phosphate synthase (EPSPS), isolated from |
| | | tolerance) | ØØ163-7 | International | the soil bacterium Agrobacterium tumefaciens |
| | | | (J101, J163) | | strain CP4. The novel form of this enzyme |
| | | | | | is termed hereafter CP4 EPSPS. |
| A-21 | Triticum | Herbicide | MON71800 | Monsanto | Glyphosate tolerant wheat variety produced |
| | aestivum | tolerance | | Company | by inserting a modified 5- |
| | (wheat) | (Glyphosate | | | enolpyruvylshikimate-3-phosphate synthase |
| | | tolerance) | | | (EPSPS) encoding gene from the soil bacterium |
| | | | | | Agrobacterium tumefaciens, strain CP4. |
| A-22 | Zea mays L. | Herbicide | NK603 | Monsanto | Introduction, by particle bombardment, of a |
| | (corn, | tolerance | | Company | modified 5-enolpyruvyl shikimate-3- |
| | maize) | (Glyphosate | | | phosphate synthase (EPSPS), an enzyme |
| | | tolerance) | | | involved in the shikimate biochemical pathway |
| | | | | | for the production of the aromatic amino |
| | | | | | acids. |
| A-23 | Zea mays L. | Herbicide | GA21 | Syngenta | Introduction, by particle bombardment, of a |
| | (corn, | tolerance | | Seeds, Inc. | modified 5-enolpyruvyl shikimate-3- |
| | maize) | (Glyphosate | | (formerly | phosphate synthase (EPSPS), an enzyme |
| | | tolerance) | | Zeneca | involved in the shikimate biochemical pathway |
| | | | | Seeds) | for the production of the aromatic amino |
| | | | | | acids. |
| A-24 | Zea mays L. | Herbicide | MON832 | Monsanto | Introduction, by particle bombardment, of |
| | (corn, | tolerance | | Company | glyphosate oxidase (GOX) and a modified 5- |
| | maize) | (Glyphosate | | | enolpyruvyl shikimate-3-phosphate synthase |
| | | tolerance) | | | (EPSPS), an enzyme involved in the shikimate |
| | | | | | biochemical pathway for the production |
| | | | | | of the aromatic amino acids. |
| A-25 | Zea mays L. | Herbicide | Event 98140 | Pioneer Hi- | Maize event expressing tolerance to glyphosate |
| | (corn, | tolerance | | Bred International | herbicide, via expression of a modified |
| | maize) | (Glyphosate | | Inc. | bacterial glyphosate N- |
| | | tolerance/ | | | acetlytransferase, and ALS-inhibiting herbicides, |
| | | ALS- | | | vial expression of a modified form of |
| | | inhibitor tol- | | | the maize acetolactate synthase enzyme. |
| A-26 | Brassica | Herbicide | GS40/ | Bayer Crop- | Introduction of pat-gene from Soil bacterium |
| | napus (Argentine | tolerance | 90pHoe6/ | Science | (Streptomyces viridochromogenes). The pat |
| | canola) | (Glufosinate | Ac | | gene codes for the enzyme Phosphinothricin- |
| | | tolerance) | | | Acetyltransferase (PAT) and leads |
| | | | | | to increased tolerance to glufosinate- |
| | | | | | containing herbicides. |
| A-27 | Brassica | Herbicide | Liberator | Bayer Crop- | Introduction of pat-gene from Soil bacterium |
| | napus (Argentine | tolerance | pHoe6/Ac | Science | (Streptomyces viridochromogenes). The pat |
| | canola) | (Glufosinate | | | gene codes for the enzyme Phosphinothricin- |
| | | tolerance) | | | Acetyltransferase (PAT) and leads |
| | | | | | to increased tolerance to glufosinate- |
| | | | | | containing herbicides. |
| A-28 | Brassica | Herbicide | TOPAS 19/2 | Bayer Crop- | Introduction of pat-gene from Soil bacterium |
| | napus (Argentine | tolerance | | Science | (Streptomyces viridochromogenes). The pat |
| | canola) | (Glufosinate | | | gene codes for the enzyme Phosphinothricin- |
| | | tolerance) | | | Acetyltransferase (PAT) and leads |
| | | | | | to increased tolerance to glufosinate- |
| | | | | | containing herbicides. |
| A-29 | Zea mays L. | Herbicide | T14, T25 | Bayer | Glufosinate herbicide tolerant maize produced |
| | (corn, | tolerance | (ACS- | CropScience | by inserting the phosphinothricin N- |
| | maize) | (Glufosinate | ZMØØ2-1/ | (Aventis | acetyltransferase (PAT) encoding gene from |
| | | tolerance) | ACS- | CropScience | the aerobic actinomycete Streptomyces viridochromogenes. |
| | | | ZMØØ3-2) | (AgrEvo)) | |
| A-30 | Brassica | Herbicide | PHY14, | Aventis | Male sterility was via insertion of the barnase |
| | napus (Argentine | tolerance | PHY35 | CropScience | ribonuclease gene from Bacillus amyloliquefaciens; |
| | canola) | (Glufosinate | | (formerly | fertility restoration by insertion of the |
| | | ammonium | | Plant Genetic | barstar RNase inhibitor; PPT resistance was |
| | | tolerance) | | Systems) | via PPT-acetyltransferase (PAT) from Streptomyces |
| | | | | | hygroscopicus. |
| A-31 | Brassica | Herbicide | PHY36 | Aventis | Male sterility was via insertion of the barnase |
| | napus (Argentine | tolerance | | CropScience | ribonuclease gene from Bacillus amyloliquefaciens; |
| | canola) | (Glufosinate | | (formerly | fertility restoration by insertion of the |
| | | ammonium | | Plant Genetic | barstar RNase inhibitor; PPT resistance was |
| | | tolerance) | | Systems) | via PPT-acetyltransferase (PAT) from Streptomyces |
| | | | | | hygroscopicus. |
| A-32 | Brassica | Herbicide | HCR-1 | Bayer | Introduction of the glufosinate ammonium |
| | rapa (Polish | tolerance | | CropScience | herbicide tolerance trait from transgenic B. napus |
| | canola) | (Glufosinate | | (Aventis | line T45. This trait is mediated by the |
| | | ammonium | | CropScience | phosphinothricin acetyltransferase (PAT) |
| | | tolerance) | | (AgrEvo)) | encoding gene from S. viridochromogenes. |
| A-33 | Cichorium | Herbicide | RM3-3, | Bejo Zaden | Male sterility produced by inssertion of the |
| | intybus | tolerance | RM3-4, | BV | barnase ribunoclease gene from Bacillus |
| | (Chicory) | (Glufosinate | RM3-6 | | amyloliquefaciens; PPT resistance was introduced |
| | | ammonium | | | by the bar gene from |
| | | tolerance) | | | S. hygroscopicus, which encodes the PAT |
| | | | | | enzyme. |
| A-34 | Glycine max L. | Herbicide | A2704-12, | Bayer | Glufosinate ammonium herbicide tolerant |
| | (soybean) | tolerance | A2704-21, | CropScience | soybean produced by inserting a modified |
| | | (Glufosinate | A5547-35 | (Aventis | phosphinothricin acetyltransferase (PAT) |
| | | ammonium | | CropScience | encoding gene from the soil bacterium Streptomyces |
| | | tolerance) | | (AgrEvo)) | viridochromogenes. |
| A-35 | Glycine max L. | Herbicide | A5547-127 | Bayer | Glufosinate ammonium herbicide tolerant |
| | (soybean) | tolerance | | CropScience | soybean produced by inserting a modified |
| | | (Glufosinate | | (Aventis | phosphinothricin acetyltransferase (PAT) |
| | | ammonium | | CropScience | encoding gene from the soil bacterium Streptomyces |
| | | tolerance) | | (AgrEvo)) | viridochromogenes. |
| A-36 | Glycine max L. | Herbicide | GU262 | Bayer | Glufosinate ammonium herbicide tolerant |
| | (soybean) | tolerance | | CropScience | soybean produced by inserting a modified |
| | | (Glufosinate | | (Aventis | phosphinothricin acetyltransferase (PAT) |
| | | ammonium | | CropScience | encoding gene from the soil bacterium Streptomyces |
| | | tolerance) | | (AgrEvo)) | viridochromogenes. |
| A-37 | Glycine max L. | Herbicide | W62, W98 | Bayer | Glufosinate ammonium herbicide tolerant |
| | (soybean) | tolerance | | CropScience | soybean produced by inserting a modified |
| | | (Glufosinate | | (Aventis | phosphinothricin acetyltransferase (PAT) |
| | | ammonium | | CropScience | encoding gene from the soil bacterium Streptomyces |
| | | tolerance) | | (AgrEvo)) | hygroscopicus. |
| A-38 | Gossypium | Herbicide | LLCotton25 | Bayer | Glufosinate ammonium herbicide tolerant |
| | hirsutum L. | tolerance | | CropScience | cotton produced by inserting a modified |
| | (cotton) | (Glufosinate | | (Aventis | phosphinothricin acetyltransferase (PAT) |
| | | ammonium | | CropScience | encoding gene from the soil bacterium Streptomyces |
| | | tolerance) | | (AgrEvo)) | hygroscopicus. |
| A-39 | Oryza sativa | Herbicide | LL RICE 62 | Bayer Crop- | Introduction of pat gene from soil bacterium |
| | (rice) | tolerance | | Science | (Streptomyces viridochromogenes). The pat |
| | | (Glufosinate | | | gene codes for the enzyme Phosphinothricin- |
| | | ammonium | | | Acetyltransferase (PAT) and leads |
| | | tolerance) | | | to increased tolerance to glufosinate- |
| | | | | | containing herbicides. |
| A-40 | Oryza sativa | Herbicide | LLrice06 | Bayer Crop- | Glufosinate ammonium herbicide tolerant |
| | (rice) | tolerance | LLrice 62 | Science | rice produced by inserting a modified |
| | | (Glufosinate | | | phosphinothricin acetyltransferase (PAT) |
| | | ammonium | | | encoding gene from the soil bacterium Streptomyces |
| | | tolerance) | | | hygroscopicus). |
| A-41 | Oryza sativa | Herbicide | LLrice601 | Bayer Crop- | Glufosinate ammonium herbicide tolerant |
| | (rice) | tolerance | | Science | rice produced by inserting a modified |
| | | (Glufosinate | | | phosphinothricin acetyltransferase (PAT) |
| | | ammonium | | | encoding gene from the soil bacterium Streptomyces |
| | | tolerance) | | | hygroscopicus). |
| A-42 | Zea mays L. | Herbicide | 676, 678, | Pioneer Hi- | Male-sterile and glufosinate ammonium herbicide |
| | (corn, | tolerance | 680 | Bred International | tolerant maize produced by inserting |
| | maize) | (Glufosinate | | Inc. | genes encoding DNA adenine methylase |
| | | ammonium | | | and phosphinothricin acetyltransferase |
| | | tolerance) | | | (PAT) from Escherichia coli and Streptomyces |
| | | | | | viridochromogenes, respectively. |
| A-43 | Zea mays L. | Herbicide | B16 (DLL25) | Dekalb Genetics | Glufosinate ammonium herbicide tolerant |
| | (corn, | tolerance | | Corporation | maize produced by inserting the gene encoding |
| | maize) | (Glufosinate | | | phosphinothricin acetyltransferase |
| | | ammonium | | | (PAT) from Streptomyces hygroscopicus. |
| | | tolerance) | | | |
| A-44 | Brassica | Herbicide | NS738, | Pioneer Hi- | Selection of somaclonal variants with altered |
| | napus (Argentine | tolerance | NS1471, | Bred International | acetolactate synthase (ALS) enzymes, following |
| | canola) | (Imidazolinone | NS1473 | Inc. | chemical mutagenesis. Two lines |
| | | tolerance) | | | (P1, P2) were initially selected with modifications |
| | | | | | at different unlinked loci. NS738 contains |
| | | | | | the P2 mutation only. |
| A-45 | Helianthus | Herbicide | X81359 | BASF | The tolerance to imidazolinone herbicides in |
| | annuus | tolerance | | | X81359 is due to a naturally occurring mutation |
| | (sunflower) | (Imidazolinone | | | in the AHAS gene discovered in a wild |
| | | tolerance) | | | population of Helianthus annus. This trait |
| | | | | | was introduced into X81359 using conventional |
| | | | | | plant breeding techniques. |
| A-46 | Lens culinaris | Herbicide | RH44 | BASF | Trait developed using chemically induced |
| | (lentil) | tolerance | | | seed mutagenesis and whole plant selection |
| | | (Imidazolinone | | | procedures. This rice line expresses a mutated |
| | | tolerance) | | | form of the acetohydroxyacid synthase |
| | | | | | (AHAS) enzyme, which renders the plant |
| | | | | | tolerant to levels of imazethapyr used in |
| | | | | | weed control. |
| A-47 | Oryza sativa | Herbicide | CFX51 | BASF | Tolerance to the imidazolinone herbicide, |
| | (rice) | tolerance | | | imazethapyr, induced by chemical |
| | | (Imidazolinone | | | mutagenesis of the acetolactate synthase |
| | | tolerance) | | | (ALS) enzyme using ethyl methanesulfonate |
| | | | | | (EMS). |
| A-48 | Oryza sativa | Herbicide | IMINTA-1, | BASF | Tolerance to imidazolinone herbicides induced |
| | (rice) | tolerance | IMINTA-4 | | by chemical mutagenesis of the acetolactate |
| | | (Imidazolinone | | | synthase (ALS) enzyme using sodium |
| | | tolerance) | | | azide. |
| A-49 | Oryza sativa | Herbicide | PWC16 | BASF | Tolerance to the imidazolinone herbicide, |
| | (rice) | tolerance | | | imazethapyr, induced by chemical |
| | | (Imidazolinone | | | mutagenesis of the acetolactate synthase |
| | | tolerance) | | | (ALS) enzyme using ethyl methanesulfonate |
| | | | | | (EMS). |
| A-50 | Triticum | Herbicide | AP205CL | BASF Inc. | Selection for a mutagenized version of the |
| | aestivum | tolerance | | | enzyme acetohydroxyacid synthase (AHAS), |
| | (wheat) | (Imidazolinone | | | also known as acetolactate synthase (ALS) |
| | | tolerance) | | | or acetolactate pyruvate-lyase. |
| A-51 | Triticum | Herbicide | AP602CL | BASF Inc. | Selection for a mutagenized version of the |
| | aestivum | tolerance | | | enzyme acetohydroxyacid synthase (AHAS), |
| | (wheat) | (Imidazolinone | | | also known as acetolactate synthase (ALS) |
| | | tolerance) | | | or acetolactate pyruvate-lyase. |
| A-52 | Triticum | Herbicide | BW255-2, | BASF Inc. | Selection for a mutagenized version of the |
| | aestivum | tolerance | BW238-3 | | enzyme acetohydroxyacid synthase (AHAS), |
| | (wheat) | (Imidazolinone | | | also known as acetolactate synthase (ALS) |
| | | tolerance) | | | or acetolactate pyruvate-lyase. |
| A-53 | Triticum | Herbicide | BW7 | BASF Inc. | Tolerance to imidazolinone herbicides induced |
| | aestivum | tolerance | | | by chemical mutagenesis of the acetohydroxyacid |
| | (wheat) | (Imidazolinone | | | synthase (AHAS) gene using |
| | | tolerance) | | | sodium azide. |
| A-54 | Triticum | Herbicide | SWP965001 | Cyanamid | Selection for a mutagenized version of the |
| | aestivum | tolerance | | Crop Protection | enzyme acetohydroxyacid synthase (AHAS), |
| | (wheat) | (Imidazolinone | | | also known as acetolactate synthase (ALS) |
| | | tolerance) | | | or acetolactate pyruvate-lyase. |
| A-55 | Triticum | Herbicide | Teal 11A | BASF Inc. | Selection for a mutagenized version of the |
| | aestivum | tolerance | | | enzyme acetohydroxyacid synthase (AHAS), |
| | (wheat) | (Imidazolinone | | | also known as acetolactate synthase (ALS) |
| | | tolerance) | | | or acetolactate pyruvate-lyase. |
| A-56 | Zea mays L. | Herbicide | 3751IR | Pioneer Hi- | Selection of somaclonal variants by culture |
| | (corn, | tolerance | | Bred International | of embryos on imidazolinone containing media. |
| | maize) | (Imidazolinone | | Inc. | |
| | | tolerance) | | | |
| A-57 | Zea mays L. | Herbicide | EXP1910IT | Syngenta | Tolerance to the imidazolinone herbicide, |
| | (corn, | tolerance | | Seeds, Inc. | imazethapyr, induced by chemical |
| | maize) | (Imidazolinone | | (formerly | mutagenesis of the acetolactate synthase |
| | | tolerance) | | Zeneca | (ALS) enzyme using ethyl methanesulfonate |
| | | | | Seeds) | (EMS). |
| A-58 | Zea mays L. | Herbicide | IT | Pioneer Hi- | Tolerance to the imidazolinone herbicide, |
| | (corn, | tolerance | | Bred International | imazethapyr, was obtained by in vitro selection |
| | maize) | (Imidazolinone | | Inc. | of somaclonal variants. |
| | | tolerance) | | | |
| A-59 | Gossypium | Herbicide | 19-51A | DuPont | Introduction of a variant form of acetolactate |
| | hirsutum L. | tolerance | | Canada Agricultural | synthase (ALS). |
| | (cotton) | (sulfonyl | | Products | |
| | | urea toler- | | | |
| A-60 | University of | Herbicide | CDC-FL001- | Linum | In addition to its native ALS gene, CDC Triffid |
| | Saskatchewan, | tolerance | 2 (FP967) | usitatissimum L. | contains an als gene from a chlorsulfuron |
| | Crop | (sulfonyl | | (flax, | tolerant line of A. thaliana. This variant als |
| | Dev. Centre | urea tolerance) | | linseed) | gene differs from the wild type A. thaliana |
| | | | | | gene by one nucleotide and the resulting |
| | | | | | ALS enzyme differs by one amino acid from |
| | | | | | the wild type ALS enzyme. The inserted als |
| | | | | | gene is linked to its native promoter and terminator. |
| A-61 | Brassica | Herbicide | OXY-235 | Aventis | Tolerance to the herbicides bromoxynil and |
| | napus (Argentine | tolerance | | CropScience | ioxynil by incorporation of the nitrilase gene |
| | canola) | (Bromoxynil | | (formerly | from Klebsiella pneumoniae. |
| | | and loxynil | | Rhone Poulenc | |
| | | tolerance) | | Inc.) | |
| A-62 | Gossypium | Herbicide | BXN | Calgene Inc. | Bromoxynil herbicide tolerant cotton produced |
| | hirsutum L. | tolerance | | | by inserting a nitrilase encoding gene |
| | (cotton) | (Bromoxynil | | | from Klebsiella pneumoniae. |
| | | and loxynil | | | |
| | | tolerance) | | | |
| A-63 | Nicotiana | Herbicide | C/F/93/08- | Societe National | Tolerance to the herbicides bromoxynil and |
| | tabacum L. | tolerance | 02 | d'Exploitation | ioxynil by incorporation of the nitrilase gene |
| | (tobacco) | (Bromoxynil | | des Tabacset | from Klebsiella pneumoniae. |
| | | and loxynil | | Allumettes | |
| | | tolerance) | | | |
| A-64 | Zea mays L. | Herbicide | DK404SR | BASF Inc. | Somaclonal variants with a modified acetyl- |
| | (corn, | tolerance | | | CoA-carboxylase (ACCase) were selected |
| | maize) | (Cyclo- | | | by culture of embryos on sethoxydim enriched |
| | | hexanone | | | medium. |
| A-65 | Gossypium | insect resistance | 281-24-236 | DOW Agro- | Insect-resistant cotton produced by inserting |
| | hirsutum L. | (Lepidoptera | (DAS- | Sciences | the cry1F gene from Bacillus thuringiensis- |
| | (cotton) | resistance) | 24236-5) | LLC | var. aizawai. The PAT encoding gene from |
| | | | | | Streptomyces viridochromogenes was introduced |
| | | | | | as a selectable marker. |
| A-66 | Gossypium | Insect resistance | 281-24-236 | Dow Agro- | Introduction of cry1A(c) + cry1F-gene from |
| | hirsutum L. | (Lepidoptera | x 3006-210- | Sciences | Bacillus thuringiensis ssp. These genes encoding |
| | (cotton) | resistance) | 23 | | the Bt-toxins Cry1A(c) and Cry1F, |
| | | | | | which confer resistance to lepidopteran |
| | | | | | pests of cotton, such as tobacco budworm |
| | | | | | (Heliothis virescens), cotton bollworm (Helicoverpa |
| | | | | | zea), beet armyworm (Spodoptera |
| | | | | | exigua), pink bollworm (Pectinophora gossypiella), |
| | | | | | and soybean looper (Pseudoplusia |
| | | | | | includens). |
| A-67 | Gossypium | Insect resistance | 3006-210-23 | DOW Agro- | Insect-resistant cotton produced by inserting |
| | hirsutum L. | (Lepidoptera | (DAS- | Sciences | the cry1Ac gene from Bacillus thuringiensis- |
| | (cotton) | resistance) | 21Ø23-5) | LLC | subsp. Kurstaki. The PAT encoding gene |
| | | | | | from Streptomyces viridochromogenes was |
| | | | | | introduced as a selectable marker. |
| A-68 | Gossypium | Insect resistance | COT102 | Syngenta | Insect-resistant cotton produced by inserting |
| | hirsutum L. | (Lepidoptera | (SYN- | Seeds, Inc. | the vip3A(a) gene from Bacillus thuringiensis |
| | (cotton) | resistance) | IR1Ø2-7) | | AB88. The APH4 encoding gene from E. coli |
| | | | | | was introduced as a selectable marker. |
| A-69 | Gossypium | Insect resistance | DAS-21Ø23- | DOW Agro- | WideStrike ™, a stacked insect-resistant cotton |
| | hirsutum L. | (Lepidoptera | 5 x DAS- | Sciences | derived from conventional cross- |
| | (cotton) | resistance) | 24236-5 | LLC | breeding of parental lines 3006-210-23 |
| | | | | | (OECD identifier: DAS-21Ø23-5) and 281- |
| | | | | | 24-236 (OECD identifier: DAS-24236-5). |
| A-70 | Gossypium | Insect resistance | Event-1 | JK Agri | Insect-resistant cotton produced by inserting |
| | hirsutum L. | (Lepidoptera | | Genetics Ltd | the cry1Ac gene from Bacillus thuringiensis |
| | (cotton) | resistance) | | (India) | subsp. Kurstaki HD-73 (B.t.k.). |
| A-71 | Gossypium | Insect resistance | MON531/757/ | Monsanto | Insect-resistant cotton produced by inserting |
| | hirsutum L. | (Lepidoptera | 1076 | Company | the cry1Ac gene from Bacillus thuringiensis |
| | (cotton) | resistance) | | | subsp. kurstaki HD-73 (B.t.k.). |
| A-72 | Gossypium | Insect resistance | 15985 | Monsanto | Insect resistant cotton derived by transformation |
| | hirsutum L. | (Lepidoptera | (MON- | Company | of the DP50B parent variety, which |
| | (cotton) | resistance) | 15985-7) | | contained event 531 (expressing Cry1Ac |
| | | | | | protein), with purified plasmid DNA containing |
| | | | | | the cry2Ab gene from B. thuringiensis |
| | | | | | subsp. Kurstaki. |
| A-73 | Lycopersicon | Insect resistance | 5345 | Monsanto | Resistance to lepidopteran pests through the |
| | esculentum | (Lepidoptera | | Company | introduction of the cry1Ac gene from Bacillus |
| | (tomato) | resistance) | | | thuringiensis subsp. Kurstaki. |
| A-74 | Zea mays L. | Insect resistance | MIR162 | Syngenta | Insect-resistant maize event expressing a |
| | (corn, | (Lepidoptera | | Seeds, Inc. | Vip3A protein from Bacillus thuringiensis and |
| | maize) | resistance) | | | the Escherichia coli PMI selectable marker. |
| A-75 | Zea mays L. | Insect resistance | MON89034 | Monsanto | Maize event expressing two different insecticidal |
| | (corn, | (Lepidoptera | | Company | proteins from Bacillus thuringiensis |
| | maize) | resistance) | | | providing resistance to number of lepidopteran |
| | | | | | pests. |
| A-76 | Zea mays L. | Insect resistance, | MON- | Monsanto | Stacked insect resistant and herbicide tolerant |
| | (corn, | Altered | ØØ81Ø-6 x | Company | corn hybrid derived from conventional |
| | maize) | composition | LY038 | | cross-breeding of the parental lines NK603 |
| | | (Lepidoptera | | | (OECD identifier: MON-ØØ6Ø3-6) and |
| | | resistance & | | | MON810 (OECD identifier: MON-ØØ81Ø-6). |
| | | enhanced | | | |
| | | lysine content) | | | |
| A-77 | Zea mays L. | Insect resistance | MON863 x | Monsanto | Stacked insect resistant corn hybrid derived |
| | (corn, | (Corn | MON810 | Company | from conventional cross-breeding of the parental |
| | maize) | root worm | (MON- | | lines MON863 (OECD identifier: MON- |
| | | resistance & | ØØ863-5, | | ØØ863-5) and MON810 (OECD identifier: |
| | | European | MON- | | MON-ØØ81Ø-6) |
| | | corn borer | ØØ81Ø-6) | | |
| | | resistance) | | | |
| A-78 | Zea mays L. | Insect resistance | MIR604 | Syngenta | Corn rootworm resistant maize produced by |
| | (corn, | (Corn | | Seeds, Inc. | transformation with a modified cry3A gene. |
| | maize) | Rootworm | | | The phosphomannose isomerase gene from |
| | | resistance) | | | E. coli was used as a selectable marker. |
| A-79 | Zea mays L. | Insect resistance | MON863 | Monsanto | Corn root worm resistant maize produced by |
| | (corn, | (Corn | | Company | inserting the cry3Bb1 gene from Bacillus |
| | maize) | Rootworm | | | thuringiensis subsp. kumamotoensis. |
| | | resistance) | | | |
| A-80 | Zea mays L. | Insect resistance | 176 | Syngenta | Insect-resistant maize produced by inserting |
| | (corn, | (European | | Seeds, Inc. | the cry1Ab gene from Bacillus thuringiensis |
| | maize) | Corn | | | subsp. Kurstaki. The genetic modification |
| | | Borer resistance) | | | affords resistance to attack by the European |
| | | | | | corn borer (ECB). |
| A-81 | Zea mays L. | Insect resistance | MON80100 | Monsanto | Insect-resistant maize produced by inserting |
| | (corn, | (European | | Company | the cry1Ab gene from Bacillus thuringiensis |
| | maize) | Corn | | | subsp. Kurstaki. The genetic modification |
| | | Borer resistance) | | | affords resistance to attack by the European |
| | | | | | corn borer (ECB). |
| A-82 | Zea mays L. | Insect resistance | MON810 | Monsanto | Insect-resistant maize produced by inserting |
| | (corn, | (European | | Company | a truncated form of the cry1Ab gene from |
| | maize) | Corn | | | Bacillus thuringiensis subsp. kurstaki HD-1. |
| | | Borer resistance) | | | The genetic modification affords resistance |
| | | | | | to attack by the European corn borer (ECB). |
| A-83 | Zea mays L. | Insect resistance, | MON810 x | Monsanto | Stacked insect resistant and enhanced lysine |
| | (corn, | Altered | LY038 | Company | content maize derived from conventional |
| | maize) | composition | | | cross-breeding of the parental lines |
| | | (European | | | MON810 (OECD identifier: MON-ØØ81Ø-6) |
| | | Corn Borer | | | and LY038 (OECD identifier: REN-ØØØ38- |
| | | resistance & | | | 3). |
| | | enhanced | | | |
| | | lysine level) | | | |
| A-84 | Solanum | Insect resistance | ATBT04-6, | Monsanto | Colorado potato beetle resistant potatoes |
| | tuberosum L. | (Colorado | ATBT04-27, | Company | produced by inserting the cry3A gene from |
| | (potato) | potato | ATBT04-30, | | Bacillus thuringiensis (subsp. Tenebrionis). |
| | | beetle) | ATBT04-31, | | |
| | | | ATBT04-36, | | |
| | | | SPBT02-5, | | |
| | | | SPBT02-7 | | |
| A-85 | Solanum | Insect resistance | BT6, BT10, | Monsanto | Colorado potato beetle resistant potatoes |
| | tuberosum L. | (Colorado | BT12, BT16, | Company | produced by inserting the cry3A gene from |
| | (potato) | potato | BT17, BT18, | | Bacillus thuringiensis (subsp. Tenebrionis). |
| | | beetle) | BT23 | | |
| A-86 | Solanum | Insect resistance | RBMT15- | Monsanto | Colorado potato beetle and potato virus Y |
| | tuberosum L. | (Colorado | 101, | Company | (PVY) resistant potatoes produced by inserting |
| | (potato) | potato | SEMT15-02, | | the cry3A gene from Bacillus thuringiensis |
| | | beetle) | SEMT15-15 | | (subsp. Tenebrionis) and the coat protein |
| | | | | | encoding gene from PVY. |
| A-87 | Solanum | Insect resistance | RBMT21- | Monsanto | Colorado potato beetle and potato leafroll |
| | tuberosum L. | (Colorado | 129, | Company | virus (PLRV) resistant potatoes produced by |
| | (potato) | potato | RBMT21- | | inserting the cry3A gene from Bacillus thuringiensis |
| | | beetle) | 350, | | (subsp. Tenebrionis) and the replicase |
| | | | RBMT22- | | encoding gene from PLRV. |
| | | | 082 | | |
| A-88 | Gossypium | Herbicide | MON- | Monsanto | Stacked insect resistant and herbicide tolerant |
| | hirsutum L. | tolerance, | ØØ531-6 x | Company | cotton derived from conventional cross- |
| | (cotton) | Insect resistance | MON- | | breeding of the parental lines MON531 |
| | | (Glyphosate | Ø1445-2 | | (OECD identifier: MON-ØØ531-6) and |
| | | tolerance & | | | MON1445 (OECD identifier: MON-Ø1445-2). |
| | | lepidopteran | | | |
| | | resistance) | | | |
| A-89 | Gossypium | Herbicide | LLCotton25 x | Bayer | Stacked herbicide tolerant and insect resistant- |
| | hirsutum L. | tolerance, | MON15985 | CropScience | cotton combining tolerance to glufosinate |
| | (cotton) | Insect resistance | | (Aventis | ammonium herbicide from LLCotton25 |
| | | (Glufosinate | | CropScience | (OECD identifier: ACS-GHØØ1-3) with resistance |
| | | ammonium | | (AgrEvo)) | to insects from MON15985 (OECD |
| | | tolerance & | | | identifier: MON-15985-7). |
| | | lepidopteran | | | |
| | | resistance) | | | |
| A-90 | Gossypium | Herbicide | DAS-21Ø23- | DOW Agro- | Stacked insect-resistant and glyphosate- |
| | hirsutum L. | tolerance, | 5 x DAS- | Sciences | tolerant cotton derived from conventional |
| | (cotton) | Insect resistance | 24236-5 x | LLC and | cross-breeding of WideStrike cotton (OECD |
| | | (Glyphosate | MON88913 | Pioneer Hi- | identifier: DAS-21Ø23-5 x DAS-24236-5) |
| | | tolerance & | (DAS- | Bred International | with MON88913, known as RoundupReady |
| | | lepidopteran | 24236-5, | Inc. | Flex (OECD identifier: MON-88913-8). |
| | | resistance) | DAS-21Ø23- | | |
| | | | 5, MON- | | |
| | | | 88913-8) | | |
| A-91 | Gossypium | Herbicide | MON15985 x | Monsanto | Stacked insect resistant and glyphosate tolerant |
| | hirsutum L. | tolerance, | MON88913 | Company | cotton produced by conventional cross- |
| | (cotton) | Insect resistance | (MON- | | breeding of the parental lines MON88913 |
| | | (Glyphosate | 15985-7, | | (OECD identifier: MON-88913-8) and 15985 |
| | | tolerance & | MON-88913- | | (OECD identifier: MON-15985-7). Glyphosate |
| | | lepidopteran | 8) | | tolerance is derived from |
| | | resistance) | | | MON88913 which contains two genes encoding |
| | | | | | the enzyme 5-enolypyruvylshikimate- |
| | | | | | 3-phosphate synthase (EPSPS) from the |
| | | | | | CP4 strain of Agrobacterium tumefaciens. |
| | | | | | Insect resistance is derived MON15985 |
| | | | | | which was produced by transformation of the |
| | | | | | DP50B parent variety, which contained event |
| | | | | | 531 (expressing Cry1Ac protein), with purified |
| | | | | | plasmid DNA containing the cry2Ab |
| | | | | | gene from B. thuringiensis subsp. Kurstaki. |
| A-92 | Gossypium | Herbicide | MON-15985- | Monsanto | Stacked insect resistant and herbicide tolerant |
| | hirsutum L. | tolerance, | 7 x MON- | Company | cotton derived from conventional cross- |
| | (cotton) | Insect resistance | Ø1445-2 | | breeding of the parental lines 15985 (OECD |
| | | (Glyphosate | | | identifier: MON-15985-7) and MON1445 |
| | | tolerance & | | | (OECD identifier: MON-Ø1445-2). |
| | | lepidopteran | | | |
| | | resistance) | | | |
| A-93 | Gossypium | Herbicide | 31807/31808 | Calgene Inc. | Insect-resistant and bromoxynil herbicide |
| | hirsutum L. | tolerance, | | | tolerant cotton produced by inserting the |
| | (cotton) | Insect resistance | | | cry1Ac gene from Bacillus thuringiensis and |
| | | (Oxynil | | | a nitrilase encoding gene from Klebsiella |
| | | tolerance & | | | pneumoniae. |
| | | lepidopteran | | | |
| | | resistance) | | | |
| A-94 | Gossypium | Herbicide | DAS-21Ø23- | DOW Agro- | WideStrike ™/Roundup Ready ® cotton, a |
| | hirsutum L. | tolerance, | 5 x DAS- | Sciences | stacked insect-resistant and glyphosate- |
| | (cotton) | Insect resistance | 24236-5 x | LLC | tolerant cotton derived from conventional |
| | | (Glyphosate | MON- | | cross-breeding of WideStrike cotton (OECD |
| | | tolerance & | Ø1445-2 | | identifier: DAS-21Ø23-5 x DAS-24236-5) |
| | | lepidopteran | | | with MON1445 (OECD identifier: MON- |
| | | resistance) | | | Ø1445-2). |
| A-95 | Zea mays L. | Herbicide | TC1507 x | DOW Agro- | Stacked insect resistant and herbicide tolerant |
| | (corn, | tolerance, | DAS-59122- | Sciences | maize produced by conventional cross |
| | maize) | Insect resistance | 7 (DAS- | LLC and | breeding of parental lines TC1507 (OECD |
| | | (Glufosinate | Ø15Ø7-1, | Pioneer Hi- | unique identifier: DAS-Ø15Ø7-1) with DAS- |
| | | tolerance & | DAS-59122- | Bred International | 59122-7 (OECD unique identifier: DAS- |
| | | Coleoptera | 7) | Inc. | 59122-7). Resistance to lepidopteran insects |
| | | and lepidoptera | | | is derived from TC1507 due the presence of |
| | | resistance) | | | the cry1F gene from Bacillus thuringiensis |
| | | | | | var. aizawai. Corn rootworm-resistance is |
| | | | | | derived from DAS-59122-7 which contains |
| | | | | | the cry34Ab1 and cry35Ab1 genes from Bacillus |
| | | | | | thuringiensis strain PS149B1. Tolerance |
| | | | | | to glufosinate ammonium herbcicide is |
| | | | | | derived from TC1507 from the phosphinothricin |
| | | | | | N-acetyltransferase encoding gene |
| | | | | | from Streptomyces viridochromogenes. |
| A-96 | Zea mays L. | Herbicide | MON810 x | Monsanto | Stacked insect resistant and glyphosate tolerant |
| | (corn, | tolerance, | MON88017 | Company | maize derived from conventional cross- |
| | maize) | Insect resistance | | | breeding of the parental lines MON810 |
| | | (Glyphosate | | | (OECD identifier: MON-ØØ81Ø-6) and |
| | | tolerance & | | | MON88017 (OECD identifier: MON-88Ø17- |
| | | Coleoptera | | | 3). European corn borer (ECB) resistance is |
| | | and lepidoptera | | | derived from a truncated form of the cry1Ab |
| | | resistance) | | | gene from Bacillus thuringiensis subsp. |
| | | | | | kurstaki HD-1 present in MON810. Corn |
| | | | | | rootworm resistance is derived from the |
| | | | | | cry3Bb1 gene from Bacillus thuringiensis |
| | | | | | subspecies kumamotoensis strain EG4691 |
| | | | | | present in MON88017. Glyphosate tolerance |
| | | | | | is derived from a 5-enolpyruvylshikimate-3- |
| | | | | | phosphate synthase (EPSPS) encoding |
| | | | | | gene from Agrobacterium tumefaciens strain |
| | | | | | CP4 present in MON88017. |
| A-97 | Zea mays L. | Herbicide | MON89034 x | Monsanto | Stacked insect resistant and glyphosate tolerant |
| | (corn, | tolerance, | MON88017 | Company | maize derived from conventional cross- |
| | maize) | Insect resistance | (MON- | | breeding of the parental lines MON89034 |
| | | (Glyphosate | 89Ø34-3, | | (OECD identifier: MON-89Ø34-3) and |
| | | tolerance & | MON- | | MON88017 (OECD identifier: MON-88Ø17- |
| | | Coleoptera | 88Ø17-3) | | 3). Resistance to Lepiopteran insects is derived |
| | | and lepidoptera | | | from two crygenes present in |
| | | resistance) | | | MON89043. Corn rootworm resistance is |
| | | | | | derived from a single cry genes and glyphosate |
| | | | | | tolerance is derived from the 5- |
| | | | | | enolpyruvylshikimate-3-phosphate synthase |
| | | | | | (EPSPS) encoding gene from Agrobacterium |
| | | | | | tumefaciens present in MON88017. |
| A-98 | Zea mays L. | Herbicide | DAS-59122- | DOW Agro- | Stacked insect resistant and herbicide tolerant |
| | (corn, | tolerance, | 7 x TC1507 | Sciences | maize produced by conventional cross |
| | maize) | Insect resistance | x NK603 | LLC and | breeding of parental lines DAS-59122-7 |
| | | (Glyphosate | | Pioneer Hi- | (OECD unique identifier: DAS-59122-7) and |
| | | tolerance & | | Bred International | TC1507 (OECD unique identifier: DAS- |
| | | Glufosinate | | Inc. | Ø15Ø7-1) with NK603 (OECD unique identifier: |
| | | ammonium | | | MON-ØØ6Ø3-6). Corn rootworm- |
| | | tolerance & | | | resistance is derived from DAS-59122-7 |
| | | Coleoptera | | | which contains the cry34Ab1 and cry35Ab1 |
| | | and lepidoptera | | | genes from Bacillus thuringiensis strain |
| | | resistance) | | | PS149B1. Lepidopteran resistance and tolerance |
| | | | | | to glufosinate ammonium herbicide is |
| | | | | | derived from TC1507. Tolerance to glyphosate |
| | | | | | herbcicide is derived from NK603. |
| A-99 | Zea mays L. | Herbicide | BT11 x | Syngenta | Stacked insect resistant and herbicide tolerant |
| | (corn, | tolerance, | MIR604 | Seeds, Inc. | maize produced by conventional cross |
| | maize) | Insect resistance | (SYN- | | breeding of parental lines BT11 (OECD |
| | | (Glufosinate | BTØ11-1, | | unique identifier: SYN-BTØ11-1) and |
| | | ammonium | SYN-IR6Ø4- | | MIR604 (OECD unique identifier: SYN- |
| | | tolerance & | 5) | | IR6Ø5-5). Resistance to the European Corn |
| | | Coleoptera | | | Borer and tolerance to the herbicide glufosinate |
| | | resistance) | | | ammonium (Liberty) is derived from |
| | | | | | BT11, which contains the cry1Ab gene from |
| | | | | | Bacillus thuringiensis subsp. Kurstaki, and |
| | | | | | the phosphinothricin N-acetyltransferase |
| | | | | | (PAT) encoding gene from S. viridochromogenes. |
| | | | | | Corn rootworm-resistance is derived |
| | | | | | from MIR604 which contains the |
| | | | | | mcry3A gene from Bacillus thuringiensis. |
| A-100 | Zea mays L. | Herbicide | DAS-59122- | DOW Agro- | Stacked insect resistant and herbicide tolerant |
| | (corn, | tolerance, | 7 x NK603 | Sciences | maize produced by conventional cross |
| | maize) | Insect resistance | | LLC and | breeding of parental lines DAS-59122-7 |
| | | (Glyphosate | | Pioneer Hi- | (OECD unique identifier: DAS-59122-7) with |
| | | tolerance & | | Bred International | NK603 (OECD unique identifier: MON- |
| | | Coleoptera | | Inc. | ØØ6Ø3-6). Corn rootworm-resistance is |
| | | resistance) | | | derived from DAS-59122-7 which contains |
| | | | | | the cry34Ab1 and cry35Ab1 genes from Bacillus |
| | | | | | thuringiensis strain PS149B1. Tolerance |
| | | | | | to glyphosate herbcicide is derived |
| | | | | | from NK603. |
| A-101 | Zea mays L. | Herbicide | MIR604 x | Syngenta | Stacked insect resistant and herbicide tolerant |
| | (corn, | tolerance, | GA21 | Seeds, Inc. | maize produced by conventional cross |
| | maize) | Insect resistance | | | breeding of parental lines MIR604 (OECD |
| | | (Glyphosate | | | unique identifier: SYN-IR6Ø5-5) and GA21 |
| | | tolerance & | | | (OECD unique identifier: MON-ØØØ21-9). |
| | | Coleoptera | | | Corn rootworm-resistance is derived from |
| | | resistance) | | | MIR604 which contains the mcry3A gene |
| | | | | | from Bacillus thuringiensis. Tolerance to |
| | | | | | glyphosate herbcicide is derived from GA21. |
| A-102 | Zea mays L. | Herbicide | MON863 x | Monsanto | Stacked insect resistant and herbicide tolerant |
| | (corn, | tolerance, | NK603 | Company | corn hybrid derived from conventional |
| | maize) | Insect resistance | (MON- | | cross-breeding of the parental lines MON863 |
| | | (Glyphosate | ØØ863-5, | | (OECD identifier: MON-ØØ863-5) and NK603 |
| | | tolerance & | MON- | | (OECD identifier: MON-ØØ6Ø3-6). |
| | | Coleoptera | ØØ6Ø3-6) | | |
| | | resistance) | | | |
| A-103 | Zea mays L. | Herbicide | MON863 x | Monsanto | Stacked insect resistant and herbicide tolerant |
| | (corn, | tolerance, | MON810 x | Company | corn hybrid derived from conventional |
| | maize) | Insect resistance | NK603 | | cross-breeding of the stacked hybrid MON- |
| | | (Glyphosate | | | ØØ863-5 x MON-ØØ81Ø-6 and NK603 |
| | | tolerance & | | | (OECD identifier: MON-ØØ6Ø3-6). |
| | | Coleoptera | | | |
| | | resistance & | | | |
| | | lepidoptera | | | |
| | | resistance) | | | |
| A-104 | Zea mays L. | Herbicide | DAS-59122-7 | DOW Agro- | Corn rootworm-resistant maize produced by |
| | (corn, | tolerance, | | Sciences | inserting the cry34Ab1 and cry35Ab1 genes |
| | maize) | Insect resistance | | LLC and | from Bacillus thuringiensis strain PS149B1. |
| | | (Glufosinate | | Pioneer Hi- | The PAT encoding gene from Streptomyces |
| | | ammonium | | Bred International | viridochromogenes was introduced as a selectable |
| | | tolerance & | | Inc. | marker. |
| | | Corn root | | | |
| | | worm resistance) | | | |
| A-105 | Zea mays L. | Herbicide | MON88017 | Monsanto | Corn rootworm-resistant maize produced by |
| | (corn, | tolerance, | | Company | inserting the cry3Bb1 gene from Bacillus |
| | maize) | Insect resistance | | | thuringiensis subspecies kumamotoensis |
| | | (Glyphosate | | | strain EG4691. Glyphosate tolerance derived |
| | | tolerance & | | | by inserting a 5-enolpyruvylshikimate-3- |
| | | Corn root | | | phosphate synthase (EPSPS) encoding |
| | | worm resistance) | | | gene from Agrobacterium tumefaciens strain |
| | | | | | CP4. |
| A-106 | Zea mays L. | Herbicide | DAS-59122-7 | Dow Agro- | |
| | (corn, | tolerance, | | Sciences | |
| | maize) | Insect resistance | | | |
| | | (Glufosinate | | | |
| | | ammonium | | | |
| | | tolerance & | | | |
| | | Corn root | | | |
| | | worm resistance) | | | |
| A-107 | Zea mays L. | Herbicide | BT11 | Syngenta | Insect-resistant and herbicide tolerant maize |
| | (corn, | tolerance, | (X4334CBR, | Seeds, Inc. | produced by inserting the cry1Ab gene from |
| | maize) | Insect resistance | X4734CBR) | | Bacillus thuringiensis subsp. Kurstaki, and |
| | | (Glufosinate | | | the phosphinothricin N-acetyltransferase |
| | | ammonium | | | (PAT) encoding gene from S. viridochromogenes. |
| | | tolerance & | | | |
| | | European | | | |
| | | corn borer | | | |
| | | resistance) | | | |
| A-108 | Zea mays L. | Herbicide | CBH-351 | Aventis | Insect-resistant and glufosinate ammonium |
| | (corn, | tolerance, | | CropScience | herbicide tolerant maize developed by inserting |
| | maize) | Insect resistance | | | genes encoding Cry9C protein from Bacillus |
| | | (Glufosinate | | | thuringiensis subsp tolworthi and |
| | | ammonium | | | phosphinothricin acetyltransferase (PAT) |
| | | tolerance & | | | from Streptomyces hygroscopicus. |
| | | European | | | |
| | | corn borer | | | |
| | | resistance) | | | |
| A-109 | Zea mays L. | Herbicide | DBT418 | Dekalb Genetics | Insect-resistant and glufosinate ammonium |
| | (corn, | tolerance, | | Corporation | herbicide tolerant maize developed by inserting |
| | maize) | Insect resistance | | | genes encoding Cry1AC protein from |
| | | (Glufosinate | | | Bacillus thuringiensis subsp kurstaki and |
| | | ammonium | | | phosphinothricin acetyltransferase (PAT) |
| | | tolerance & | | | from Streptomyces hygroscopicus |
| | | European | | | |
| | | corn borer | | | |
| | | resistance) | | | |
| A-110 | Zea mays L. | Herbicide | TC1507 | Mycogen | Insect-resistant and glufosinate ammonium |
| | (corn, | tolerance, | | (c/o Dow | herbicide tolerant maize produced by inserting |
| | maize) | Insect resistance | | Agro- | the cry1F gene from Bacillus thuringiensis |
| | | (Glufosinate | | Sciences); | var. aizawai and the phosphinothricin N- |
| | | ammonium | | Pioneer (c/o | acetyltransferase encoding gene from Streptomyces |
| | | tolerance & | | Dupont) | viridochromogenes. |
| | | European | | | |
| | | corn borer | | | |
| | | resistance) | | | |
| A-111 | Zea mays L. | Herbicide | MON802 | Monsanto | Insect-resistant and glyphosate herbicide |
| | (corn, | tolerance, | | Company | tolerant maize produced by inserting the |
| | maize) | Insect resistance | | | genes encoding the Cry1Ab protein from |
| | | (Glyphosate | | | Bacillus thuringiensis and the 5- |
| | | tolerance & | | | enolpyruvylshikimate-3-phosphate synthase |
| | | European | | | (EPSPS) from A. tumefaciens strain CP4. |
| | | corn borer | | | |
| | | resistance) | | | |
| A-112 | Zea mays L. | Herbicide | MON809 | Pioneer Hi- | Resistance to European corn borer (Ostrinia |
| | (corn, | tolerance, | | Bred International | nubilalis) by introduction of a synthetic |
| | maize) | Insect resistance | | Inc. | cry1Ab gene. Glyphosate resistance via introduction |
| | | (Glyphosate | | | of the bacterial version of a plant |
| | | tolerance & | | | enzyme, 5-enolpyruvyl shikimate-3- |
| | | European | | | phosphate synthase (EPSPS). |
| | | corn borer | | | |
| | | resistance) | | | |
| A-113 | Zea mays L. | Herbicide | BT11 x | Syngenta | Stacked insect resistant and herbicide tolerant |
| | (corn, | tolerance, | MIR162 | Seeds, Inc. | maize produced by conventional cross |
| | maize) | Insect resistance | (SYN- | | breeding of parental lines BT11 (OECD |
| | | (Glufosinate | BTØ11-1, | | unique identifier: SYN-BTØ11-1) and |
| | | ammonium | SYN-IR162- | | MIR162 (OECD unique identifier: SYN- |
| | | tolerance & | 4) | | IR162-4). Resistance to the European Corn |
| | | lepidopteran | | | Borer and tolerance to the herbicide glufosinate |
| | | resistance) | | | ammonium (Liberty) is derived from |
| | | | | | BT11, which contains the cry1Ab gene from |
| | | | | | Bacillus thuringiensis subsp. Kurstaki, and |
| | | | | | the phosphinothricin N-acetyltransferase |
| | | | | | (PAT) encoding gene from S. viridochromogenes. |
| | | | | | Resistance to other lepidopteran |
| | | | | | pests, including H. zea, S. frugiperda, A. ipsilon, |
| | | | | | and S. albicosta, is derived from |
| | | | | | MIR162, which contains the vip3Aa gene |
| | | | | | from Bacillus thuringiensis strain AB88. |
| A-114 | Zea mays L. | Herbicide | DAS-06275-8 | DOW Agro- | Lepidopteran insect resistant and glufosinate |
| | (corn, | tolerance, | | Sciences | ammonium herbicide-tolerant maize variety |
| | maize) | Insect resistance | | LLC | produced by inserting the cry1F gene from |
| | | (Glufosinate | | | Bacillus thuringiensis var aizawai and the |
| | | ammonium | | | phosphinothricin acetyltransferase (PAT) |
| | | tolerance & | | | from Streptomyces hygroscopicus. |
| | | lepidopteran | | | |
| | | resistance) | | | |
| A-115 | Zea mays L. | Herbicide | BT11 x | Syngenta | Stacked insect resistant and herbicide tolerant |
| | (corn, | tolerance, | GA21 (SYN- | Seeds, Inc. | maize produced by conventional cross |
| | maize) | Insect resistance | BTØ11-1, | | breeding of parental lines BT11 (OECD |
| | | (Glufosinate | MON- | | unique identifier: SYN-BTØ11-1) and GA21 |
| | | ammonium | ØØØ21-9) | | (OECD unique identifier: MON-ØØØ21-9). |
| | | tolerance & | | | |
| | | Glyphosate | | | |
| | | tolerance & | | | |
| | | Lepidoptera | | | |
| | | resistance) | | | |
| A-116 | Zea mays L. | Herbicide | BT11 x | Syngenta | Stacked insect resistant and herbicide tolerant |
| | (corn, | tolerance, | MIR604 x | Seeds, Inc. | maize produced by conventional cross |
| | maize) | Insect resistance | GA21 (SYN- | | breeding of parental lines BT11 (OECD |
| | | (Glufosinate | BTØ11-1, | | unique identifier: SYN-BTØ11-1), MIR604 |
| | | ammonium | SYN-IR6Ø4- | | (OECD unique identifier: SYN-IR6Ø5-5) and |
| | | tolerance & | 5, MON- | | GA21 (OECD unique identifier: MON- |
| | | Glyphosate | ØØØ21-9) | | ØØØ21-9). Resistance to the European |
| | | tolerance & | | | Corn Borer and tolerance to the herbicide |
| | | Lepidoptera | | | glufosinate ammonium (Liberty) is derived |
| | | resistance) | | | from BT11, which contains the cry1Ab gene |
| | | | | | from Bacillus thuringiensis subsp. Kurstaki, |
| | | | | | and the phosphinothricin N-acetyltransferase |
| | | | | | (PAT) encoding gene from S. viridochromogenes. |
| | | | | | Corn rootworm-resistance is derived |
| | | | | | from MIR604 which contains the |
| | | | | | mcry3A gene from Bacillus thuringiensis. |
| | | | | | Tolerance to glyphosate herbcicide is derived |
| | | | | | from GA21 which contains a a modified |
| | | | | | EPSPS gene from maize. |
| A-117 | Zea mays L. | Herbicide | TC1507 x | DOW Agro- | Stacked insect resistant and herbicide tolerant |
| | (corn, | tolerance, | NK603 | Sciences | corn hybrid derived from conventional |
| | maize) | Insect resistance | (DAS- | LLC | cross-breeding of the parental lines 1507 |
| | | (Glufosinate | Ø15Ø7-1 x | | (OECD identifier: DAS-Ø15Ø7-1) and NK603 |
| | | ammonium | MON- | | (OECD identifier: MON-ØØ6Ø3-6). |
| | | tolerance & | ØØ6Ø3-6) | | |
| | | Glyphosate | | | |
| | | tolerance & | | | |
| | | Lepidoptera | | | |
| | | resistance) | | | |
| A-118 | Zea mays L. | Herbicide | GA21 x | Monsanto | Stacked insect resistant and herbicide tolerant |
| | (corn, | tolerance, | MON810 | Company | corn hybrid derived from conventional |
| | maize) | Insect resistance | | | cross-breeding of the parental lines GA21 |
| | | (Glyphosate | | | (OECD identifider: MON-ØØØ21-9) and |
| | | tolerance & | | | MON810 (OECD identifier: MON-ØØ81Ø-6). |
| | | lepidopteran | | | |
| | | resistance) | | | |
| A-119 | Zea mays L. | Herbicide | MON89034 | Monsanto | Stacked insect resistant and herbicide tolerant |
| | (corn, | tolerance, | x NK603 | Company | maize produced by conventional cross |
| | maize) | Insect resistance | (MON- | | breeding of parental lines MON89034 |
| | | (Glyphosate | 89Ø34-3, | | (OECD identifier: MON-89Ø34-3) with |
| | | tolerance & | MON- | | NK603 (OECD unique identifier: MON- |
| | | lepidopteran | ØØ6Ø3-6) | | ØØ6Ø3-6). Resistance to Lepiopteran insects |
| | | resistance) | | | is derived from two crygenes present |
| | | | | | in MON89043. Tolerance to glyphosate |
| | | | | | herbcicide is derived from NK603. |
| A-120 | Zea mays L. | Herbicide | NK603 x | Monsanto | Stacked insect resistant and herbicide tolerant |
| | (corn, | tolerance, | MON810 | Company | corn hybrid derived from conventional |
| | maize) | Insect resistance | (MON- | | cross-breeding of the parental lines NK603 |
| | | (Glyphosate | ØØ6Ø3-6, | | (OECD identifier: MON-ØØ6Ø3-6) and |
| | | tolerance & | MON- | | MON810 (OECD identifier: MON-ØØ81Ø-6). |
| | | lepidopteran | ØØ81Ø-6) | | |
| | | resistance) | | | |
| A-121 | Zea mays L. | Herbicide | T25 x | Bayer | Stacked insect resistant and herbicide tolerant |
| | (corn, | tolerance, | MON810 | CropScience | corn hybrid derived from conventional |
| | maize) | Insect resistance | (ACS- | (Aventis | cross-breeding of the parental lines T25 |
| | | (Glufosinate | ZMØØ3-2, | CropScience | (OECD identifier: ACS-ZMØØ3-2) and |
| | | ammonium | MON- | (AgrEvo)) | MON810 (OECD identifier: MON-ØØ81Ø-6). |
| | | tolerance & | ØØ81Ø-6) | | |
| | | lepidopteran | | | |
| | | resistance) | | | |
| A-122 | Brassica | Herbicide | MS1, RF1 | Bayer | Introduction of the PPT-acetyltransferase |
| | napus (Argentine | tolerance | (PGS1) | CropScience | (PAT) encoding gene from Streptomyces |
| | canola) | (Gluphosinate | | (Aventis | viridochromogenes, an aerobic soil bacteria. |
| | | tolerance), | | CropScience | PPT normally acts to inhibit glutamine synthetase, |
| | | Male | | (AgrEvo)) | causing a fatal accumulation of |
| | | sterility | | | ammonia. Acetylated PPT is inactive. |
| A-123 | Brassica | Herbicide | MS1, RF2 | Aventis | Introduction of the PPT-acetyltransferase |
| | napus (Argentine | tolerance | (PGS2) | CropScience | (PAT) encoding gene from Streptomyces |
| | canola) | (Gluphosinate | | (formerly | viridochromogenes, an aerobic soil bacteria. |
| | | tolerance), | | Plant Genetic | PPT normally acts to inhibit glutamine synthetase, |
| | | Male | | Systems) | causing a fatal accumulation of |
| | | sterility | | | ammonia. Acetylated PPT is inactive. |
| A-124 | Brassica | Herbicide | MS8xRF3 | Bayer | Male-sterility, fertility restoration, pollination |
| | napus (Argentine | tolerance | | CropScience | control system displaying glufosinate herbicide |
| | canola) | (Gluphosinate | | (Aventis | tolerance. MS lines contained the |
| | | tolerance), | | CropScience | barnase gene from Bacillus amyloliquefaciens, |
| | | Male | | (AgrEvo)) | RF lines contained the barstar gene |
| | | sterility | | | from the same bacteria, and both lines contained |
| | | | | | the phosphinothricin N- |
| | | | | | acetyltransferase (PAT) encoding gene from |
| | | | | | Streptomyces hygroscopicus. |
| A-125 | Zea mays L. | Herbicide | MS3 (ACS- | Bayer | Male sterility caused by expression of the |
| | (corn, | tolerance | ZMØØ1-9) | CropScience | barnase ribonuclease gene from Bacillus |
| | maize) | (Gluphosinate | | (Aventis | amyloliquefaciens; PPT resistance was via |
| | | tolerance), | | CropScience | PPT-acetyltransferase (PAT). |
| | | Male | | (AgrEvo)) | |
| | | sterility | | | |
| A-126 | Zea mays L. | Herbicide | MS6 (ACS- | Bayer | Male sterility caused by expression of the |
| | (corn, | tolerance | ZMØØ5-4) | CropScience | barnase ribonuclease gene from Bacillus |
| | maize) | (Gluphosinate | | (Aventis | amyloliquefaciens; PPT resistance was via |
| | | tolerance), | | CropScience | PPT-acetyltransferase (PAT). |
| | | Male | | (AgrEvo)) | |
| | | sterility | | | |
| A-127 | Glycine max L. | Herbicide | 305423 x | Pioneer Hi- | Plants produced by introducing gm-fad2-1- |
| | (soybean) | tolerance, | 40-3-2 | Bred | gene and cp4 epsps-gene. Function of the |
| | | Altered composition | | | gm-fad2-1 gene fragment from soybean |
| | | (Glyphosate | | | (Glycine max): through the introduction of a |
| | | tolerance & | | | copy of its natural gene, the production of |
| | | high oleic | | | the enzyme D12-desaturase in the soybean |
| | | acid content) | | | is blocked (antisense). This enzyme is instrumental |
| | | | | | in the transformation of oleic acid |
| | | | | | to linoleic acid. The result: the soybeans |
| | | | | | have a significantly higher content of oleic |
| | | | | | acid and, conversely, less linoleic acid. At |
| | | | | | high temperatures, such as in the case of the |
| | | | | | tempering of fats or of frying, a portion of the |
| | | | | | linoleic acid will be transformed into trans fat |
| | | | | | acids, which are regarded as questionable in |
| | | | | | regards to health. In the processing of oils |
| | | | | | from 305423x40-3-2-Soybean, fewer trans |
| | | | | | fat acids are produced. |
| A-128 | Carica papaya | Fungal and | 55-1/63-1 | Cornell University | Papaya ringspot virus (PRSV) resistant papaya |
| | (papaya) | virus resistance | | | produced by inserting the coat protein |
| | | (papaya | | | (CP) encoding sequences from this plant |
| | | ringspot | | | potyvirus. |
| | | virus | | | |
| | | (PRSV) resistance) | | | |
| A-129 | Carica papaya | Fungal and | X17-2 | University of | Papaya ringspot virus (PRSV) resistant papaya |
| | (papaya) | virus resistance | | Florida | produced by inserting the coat protein |
| | | (papaya | | | (CP) encoding sequences from PRSV isolate |
| | | ringspot | | | H1K with a thymidine inserted after the initiation |
| | | virus | | | codon to yield a frameshift. Also contains |
| | | (PRSV) resistance) | | | nptII as a selectable marker |
| A-130 | Cucurbita | Fungal and | CZW-3 | Asgrow | Cucumber mosiac virus (CMV), zucchini |
| | pepo | virus resistance | | (USA); Seminis | yellows mosaic (ZYMV) and watermelon |
| | (squash) | (cucumber | | Vegetable | mosaic virus (WMV) 2 resistant squash |
| | | mosaiv | | Inc. | (Curcurbita pepo) produced by inserting the |
| | | virus | | (Canada) | coat protein (CP) encoding sequences from |
| | | (CMV), zucchini | | | each of these plant viruses into the host genome. |
| | | yellow | | | |
| | | mosaic virus | | | |
| | | (ZYMV), | | | |
| | | watermelon | | | |
| | | mosaic virus | | | |
| | | (WMV) resistance) | | | |
| A-131 | Cucurbita | Fungal and | ZW20 | Upjohn | Zucchini yellows mosaic (ZYMV) and watermelon |
| | pepo | virus resistance | | (USA); Seminis | mosaic virus (WMV) 2 resistant |
| | (squash) | (zucchini | | Vegetable | squash (Curcurbita pepo) produced by inserting |
| | | yellow | | Inc. | the coat protein (CP) encoding sequences |
| | | mosaic virus | | (Canada) | from each of these plant potyviruses |
| | | (ZYMV), | | | into the host genome. |
| | | watermelon | | | |
| | | mosaic virus | | | |
| | | (WMV) resistance) | | | |
| A-132 | Prunus domestica | Fungal and | C5 | United | The coat protein gene of the plum pox virus |
| | (plum tree) | virus resistance | | States Department | containing the 35S promoter and the nos |
| | | (Plum | | of | terminator, from plasmid pBIPCP was sub- |
| | | pox virus | | Agriculture- | cloned into HindIII-digested pGA482GG and |
| | | resistant | | Agricultural | the resulting plasmid was designated |
| | | resistance) | | Research | pGA482GG/PPV-CP-33. This plasmid was |
| | | | | Service | used to electrotransform A. tumefaciens |
| | | | | | strain C58/Z707 and used for transformation |
| | | | | | of plum tissue. |
| A-133 | Brassica | Altered composition | 23-18-17, | Monsanto | High laurate (12:0) and myristate (14:0) canola |
| | napus (Argentine | (oil | 23-198 | Company | produced by inserting a thioesterase |
| | canola) | profile alteration) | | | encoding gene from the California bay laurel |
| | | | | | (Umbellularia californica). |
| A-134 | Brassica | Altered composition | 46A12, | Pioneer Hi- | Combination of chemical mutagenesis, to |
| | napus (Argentine | (oil | 46A16 | Bred International | achieve the high oleic acid trait, and traditional |
| | canola) | profile alteration) | | Inc. | breeding with registered canola varieties. |
| A-135 | Brassica | Altered composition | 45A37, | Pioneer Hi- | High oleic acid and low linolenic acid canola |
| | napus (Argentine | (oleic acid | 46A40 | Bred International | produced through a combination of chemical |
| | canola) | and linolenic | | Inc. | mutagenesis to select for a fatty acid desaturase |
| | | acid profile | | | mutant with elevated oleic acid, and |
| | | alteration) | | | traditional back-crossing to introduce the low |
| | | | | | linolenic acid trait. |
| A-136 | Dianthus | Altered composition | Carnation | Florigene | Introduction of gene acc from carnations |
| | caryophyllus | (increased | Moon- | Ltd | (Dyanthus caryophyllus). By shortening the |
| | (carnation) | shelf-life) | shadow 2 | | gene Aminocyclopropane Cyclase (ACC) |
| | | | | | synthase, the plant produces less Ethene (a |
| | | | | | plant hormone responsible for plant maturation) |
| | | | | | and retards ripening. |
| A-137 | Glycine max L. | Altered composition | OT96-15 | Agriculture & | Low linolenic acid soybean produced |
| | (soybean) | (linolenic | | Agri-Food | through traditional cross-breeding to incorporate |
| | | acid | | Canada | the novel trait from a naturally occurring |
| | | profile alteration) | | | fan1 gene mutant that was selected for low |
| | | | | | linolenic acid. |
| A-138 | Glycine max L. | Altered composition | G94-1, G94- | DuPont | High oleic acid soybean produced by inserting |
| | (soybean) | (oil | 19, G168 | Canada Agricultural | a second copy of the fatty acid desaturase |
| | | profile alteration) | | Products | (GmFad2-1) encoding gene from soybean, |
| | | | | | which resulted in “silencing” of the |
| | | | | | endogenous host gene. |
| A-139 | Glycine max L. | Altered composition | DP-305423 | Pioneer Hi- | High oleic acid soybean produced by inserting |
| | (soybean) | (increased | | Bred International | additional copies of a portion of the |
| | | oleic | | Inc. | omega-6 desaturase encoding gene, gm- |
| | | acid content) | | | fad2-1 resulting in silencing of the endogenous |
| | | | | | omega-6 desaturase gene (FAD2-1). |
| A-140 | Nicotiana | Altered composition | Vector 21-41 | Vector Tobacco | Reduced nicotine content through introduction |
| | tabacum L. | (Nicotine | | Inc. | of a second copy of the tobacco quinolinic |
| | (tobacco) | reduction) | | | acid phosphoribosyltransferase |
| | | | | | (QTPase) in the antisense orientation. The |
| | | | | | NPTII encoding gene from E. coli was introduced |
| | | | | | as a selectable marker to identify |
| | | | | | transformants. |
| A-141 | Solanum | Altered composition | EH92-527-1 | BASF Plant | Introduction of GBSS gene from potato (Solanum |
| | tuberosum L. | (starch with | | Science | tuberosum). GBSS (granule bound |
| | (potato) | increased | | | starch synthase) is one of the key enzymes |
| | | amylopectin | | | in the biosynthesis of starch and catalyses |
| | | content) | | | the formation of amylose. This gene was |
| | | | | | inactivated by antisense technology. Thus, |
| | | | | | the starch produced has little or no amylose |
| | | | | | and consists of branched amylopectin, which |
| | | | | | modifies the physical properties of the starch |
| | | | | | and is advantageous for the starch processing |
| | | | | | industry. |
| A-142 | Zea mays L. | Altered composition | LY038 | Monsanto | Altered amino acid composition, specifically |
| | (corn, | (enhanced | | Company | elevated levels of lysine, through the introduction |
| | maize) | lysin | | | of the cordapA gene, derived from |
| | | level) | | | Corynebacterium glutamicum, encoding the |
| | | | | | enzyme dihydrodipicolinate synthase |
| | | | | | (cDHDPS). |
| A-143 | Zea mays L. | Altered composition | Event 3272 | Syngenta | Maize line expressing a heat stable alpha- |
| | (corn, | (modified | | Seeds, Inc. | amylase gene amy797E for use in the dry- |
| | maize) | amylase | | | grind ethanol process. The phosphomannose |
| | | content) | | | isomerase gene from E. coli was used |
| | | | | | as a selectable marker. |
| A-144 | Cucumis | Altered | A, B | Agritope Inc. | Delayed ripening by introduction of a gene |
| | melo | maturation | | | that results in degradation of a precursor of |
| | (melon) | (delayed | | | the plant hormone, ethylene. Accomplished |
| | | ripening) | | | by introduction of a bacteriophage encoded |
| | | | | | enzyme, S-adenosylmethionine hydrolase, |
| | | | | | capable of degrading and thus reducing |
| | | | | | SAM. The conversion of SAM to 1- |
| | | | | | aminocyclopropane-1-carboxylic acid (ACC) |
| | | | | | is the first step in ethylene biosynthesis and |
| | | | | | the lack of sufficient pools of SAM results in |
| | | | | | significantly reduced synthesis of this phyto- |
| | | | | | hormone, which is known to play a key role |
| | | | | | in fruit ripening. |
| A-145 | Dianthus | Altered | 66 | Florigene | Delayed senescence and sulfonylurea herbicide |
| | caryophyllus | maturation | | Pty Lt | tolerant carnations produced by inserting |
| | (carnation) | (Increased | | | a truncated copy of the carnation amino- |
| | | shelf-life; | | | cyclopropane cyclase (ACC) synthase encoding |
| | | Sulfonylurea | | | gene in order to suppress expression |
| | | herbicide | | | of the endogenous unmodified gene, which |
| | | tolerance) | | | is required for normal ethylene biosynthesis. |
| | | | | | Tolerance to sulfonyl urea herbicides was via |
| | | | | | the introduction of a chlorsulfuron tolerant |
| | | | | | version of the acetolactate synthase (ALS) |
| | | | | | encoding gene from tobacco. |
| A-146 | Lycopersicon | Altered | B, Da, F | Zeneca | Delayed softening tomatoes produced by |
| | esculentum | maturation | | Seeds | inserting a truncated version of the polygalacturonase |
| | (tomato) | (Delayed | | | (PG) encoding gene in the |
| | | sofenting) | | | sense or anti-sense orientation in order to |
| | | | | | reduce expression of the endogenous PG |
| | | | | | gene, and thus reduce pectin degradation. |
| A-147 | Lycopersicon | Altered | FLAVR | Calgene Inc. | Delayed softening tomatoes produced by |
| | esculentum | maturation | SAVR | | inserting an additional copy of the polygalacturonase |
| | (tomato) | (Delayed | | | (PG) encoding gene in the anti- |
| | | sofenting) | | | sense orientation in order to reduce expression |
| | | | | | of the endogenous PG gene and thus |
| | | | | | reduce pectin degradation. |
| A-148 | Lycopersicon | Altered | 8338 | Monsanto | Introduction of a gene sequence encoding |
| | esculentum | maturation | | Company | the enzyme 1-amino-cyclopropane-1- |
| | (tomato) | (fruit ripening | | | carboxylic acid deaminase (ACCd) that metabolizes |
| | | alteration) | | | the precursor of the fruit ripening |
| | | | | | hormone ethylene. |
| A-149 | Lycopersicon | Altered | 1345-4 | DNA plant | Delayed ripening tomatoes produced by inserting |
| | esculentum | maturation | | technology | an additional copy of a truncated |
| | (tomato) | (fruit ripening | | corporation | gene encoding 1-aminocyclopropane-1- |
| | | alteration) | | | carboxyllic acid (ACC) synthase, which resulted |
| | | | | | in downregulation of the endogenous |
| | | | | | ACC synthase and reduced ethylene accumulation. |
| A-150 | Lycopersicon | Altered | 35 1 N | Agritopoe | Introduction of a gene sequence encoding |
| | esculentum | maturation | | Inc. | the enzyme S-adenosylmethionine hydrolase |
| | (tomato) | (fruit ripening | | | that metabolizes the precursor of the fruit |
| | | alteration) | | | ripening hormone ethylene. |
| A-151 | Dianthus | Altered morphology | 4, 11, 15, 16 | Florigene | Modified colour and sulfonylurea herbicide |
| | caryophyllus | (coloration; | | Pty Lt | tolerant carnations produced by inserting two |
| | (carnation) | Sulfonylurea | | | anthocyanin biosynthetic genes whose expression |
| | | herbicide | | | results in a violet/mauve colouration. |
| | | tolerance) | | | Tolerance to sulfonyl urea herbicides |
| | | | | | was via the introduction of a chlorsulfuron |
| | | | | | tolerant version of the acetolactate synthase |
| | | | | | (ALS) encoding gene from tobacco. |
| A-152 | Dianthus | Altered morphology | 959A, 988A, | Florigene | Introduction of two anthocyanin biosynthetic |
| | caryophyllus | (coloration; | 1226A, | Pty Lt | genes to result in a violet/mauve colouration; |
| | (carnation) | Sulfonylurea | 1351A, | | Introduction of a variant form of acetolactate |
| | | herbicide | 1363A, | | synthase (ALS). |
| | | tolerance) | 1400A | | |
| A-153 | Dianthus | Altered morphology | Carnation | Florigene | Genes dfr, bp40 from Petunia (Petunia hybrida). |
| | caryophyllus | (modified | Moonaqua | Ltd | The genes have been transferred to a |
| | (carnation) | flower color) | | | white-flowering carnation. They lead to a |
| | | | | | modified synthesis pathway, producing a |
| | | | | | blue-violet flower dye. |
| A-154 | Dianthus | Altered morphology | Carnation | Florigene | Introduction of three genes: petunia DFR |
| | caryophyllus | (modified | Moonlite | Ltd | gene, coding for dihydroflavonol-4-reductase |
| | (carnation) | flower color) | | | and derived from Petunia × hybrida; petunia |
| | | | | | F3′5′H gene, coding for flavonoid |
| | | | | | 3′5′hydroxylase, derived from Petunia × hybrida; |
| | | | | | and ALS gene (SuRB), coding for a |
| | | | | | mutant acetolactate synthase proteil (ALS), |
| | | | | | derived from Nicotiana tabacum. |
| A-155 | Dianthus | Altered morphology | Carnation | Florigene | Genes dfr, bp40 from Petunia (Petunia hybrida). |
| | caryophyllus | (modified | Moondust | Ltd | The genes have been transferred to a |
| | (carnation) | flower color) | | | white-flowering carnation. They lead to a |
| | | | | | modified synthesis pathway, producing a |
| | | | | | blue-violet flower dye. |
| A-156 | Dianthus | Altered morphology | Carnation | Florigene | Introduction of gene acc from carnations |
| | caryophyllus | (modified | Moon- | Ltd | (Dyanthus caryophyllus). By shortening the |
| | (carnation) | flower color) | shadow 1 | | gene Aminocyclopropane Cyclase (ACC) |
| | | | | | synthase, the plant produces less Ethene (a |
| | | | | | plant hormone responsible for plant maturation) |
| | | | | | and retards ripening. |
| A-157 | Gossypium | Insect resistance | COT67B | Syngenta | COT67B cotton has been genetically modified |
| | hirsutum L. | (resistance | | Seeds, Inc. | for protection against feeding damage |
| | (Cotton) | to lepidopteran | | 7500 Olson | caused by larvae of a number of insect pest |
| | | pests) | | Memorial | species, including: Helicoverpa zea, cotton |
| | | | | Highway | bollworm; and Heliothis virescens, tobacco |
| | | | | Golden Valley | budworm. Protection against these pests is |
| | | | | MN USA | achieved through expression in the plant of |
| | | | | | an insecticidal Cry protein, Cry1Ab, encoded |
| | | | | | by the full-length cry1Ab gene derived from |
| | | | | | Bacillus thuringiensis subspecies Kurstaki |
| | | | | | HD-1. |

In a further utmost preference, the cultivated plants are plants comprising one or more genes as given in Table B. Sources: AgBios database (AG BIOS, P.O. Box 475, 106 St. John St. Merickville, Ontario KOG1NO, Canada, access: http://www.agbios.com/dbase.php)

TABLE B

| No | Crop | Gene |
|---|---|---|
| B-1 | alfalfa | CP4 epsps |
| | (Medicago sativa) | |
| B-2 | canola | als |
| B-3 | canola | bar |
| B-4 | canola | bxn |
| B-5 | canola | CP4 epsps |
| B-6 | canola | CP4 epsps + goxv247 |
| B-7 | canola | goxv247 |
| B-8 | canola | pat |
| B-9 | corn (Zea mays L.) | Accase |
| B-10 | corn (Zea mays L.) | als |
| B-11 | corn (Zea mays L.) | CP4 epsps |
| B-12 | corn (Zea mays L.) | CP4 epsps + Cry1Ab |
| B-13 | corn (Zea mays L.) | CP4 epsps + Cry1Ab + Cry3Bb1 |
| B-14 | corn (Zea mays L.) | CP4 epsps + Cry1Ab + goxv247 |
| B-15 | corn (Zea mays L.) | CP4 epsps + Cry1Ab + mCry3A |
| B-16 | corn (Zea mays L.) | CP4 epsps + Cry1Fa2 |
| B-17 | corn (Zea mays L.) | CP4 epsps + Cry34Ab1 + Cry35Ab1 |
| B-18 | corn (Zea mays L.) | CP4 epsps + Cry34Ab1 + Cry35Ab1 + |
| | | Cry1Fa2 |
| B-19 | corn (Zea mays L.) | CP4 epsps + Cry34Ab1 + Cry35Ab1 + |
| | | Cry1Fa2 + pat |
| B-20 | corn (Zea mays L.) | CP4 epsps + goxv247 |
| B-21 | corn (Zea mays L.) | CP4 epsps + pat |
| B-22 | corn (Zea mays L.) | Cry1A.105 |
| B-23 | corn (Zea mays L.) | Cry1Ab |
| B-24 | corn (Zea mays L.) | Cry1Ab + mCry3A |
| B-25 | corn (Zea mays L.) | Cry1Ab + mCry3A + pat |
| B-26 | corn (Zea mays L.) | Cry1Ab + pat |
| B-27 | corn (Zea mays L.) | Cry1Ab + vip3Aa20 + pat |
| B-28 | corn (Zea mays L.) | Cry1Ac |
| B-29 | corn (Zea mays L.) | Cry1F |
| B-30 | corn (Zea mays L.) | Cry1Fa2 |
| B-31 | corn (Zea mays L.) | Cry1Fa2 + pat |
| B-32 | corn (Zea mays L.) | Cry34Ab1 |
| B-33 | corn (Zea mays L.) | Cry34Ab1 + Cry35Ab1 |
| B-34 | corn (Zea mays L.) | Cry34Ab1 + Cry35Ab1 + |
| | | Cry1Fa2 + pat |
| B-35 | corn (Zea mays L.) | Cry35Ab1 |
| B-36 | corn (Zea mays L.) | Cry3A |
| B-37 | corn (Zea mays L.) | Cry3Bb1 |
| B-38 | corn (Zea mays L.) | Cry9C |
| B-39 | corn (Zea mays L.) | goxv247 |
| B-40 | corn (Zea mays L.) | mCry3A |
| B-41 | corn (Zea mays L.) | mcry3A |
| B-42 | corn (Zea mays L.) | pat |
| B-43 | corn (Zea mays L.) | vip3A |
| B-44 | cotton | ALS |
| B-45 | cotton | als |
| B-46 | cotton | bxn |
| B-47 | cotton | CP4 epsps |
| B-48 | cotton | CP4 epsps + Cry1Ac |
| B-49 | cotton | CP4 epsps + Cry1Ac + Cry1F |
| B-50 | cotton | CP4 epsps + Cry1Ac + |
| | | Cry1F + pat |
| B-51 | cotton | CP4 epsps + Cry1Ac + Cry2Ab |
| B-52 | cotton | Cr1Ac + Cry2Ab |
| B-53 | cotton | Cr1Ac + Cry2Ab |
| B-54 | cotton | Cry1A.105 |
| B-55 | cotton | Cry1Ac |
| B-56 | cotton | Cry1Ac + bxn |
| B-57 | cotton | Cry1Ac + Cry1F |
| B-58 | cotton | Cry1Ac + pat |
| B-59 | cotton | Cry1F |
| B-60 | cotton | Cry1F + pat |
| B-61 | cotton | Cry2Ab |
| B-62 | cotton | Cry3Bb1 |
| B-63 | cotton | pat |
| B-64 | cotton | vip3A(a) |
| B-65 | papaya | prsv-cp |
| B-66 | potato | CP4 epsps |
| B-67 | potato | Cry3A |
| B-68 | rice | ALS |
| B-69 | soybean | ALS |
| B-70 | soybean | CP4 epsps |
| B-71 | soybean | pat |
| B-72 | squash | cmv-cp |
| B-73 | squash | wmv2-cp |
| B-74 | squash | zymv-cp |
| B-75 | sugar beet | CP4 epsps |
| B-76 | sugar beet | CP4 epsps + goxv247 |
| B-77 | sugar beet | goxy247 |
| B-78 | sugar beet | pat |
| B-79 | sunflower | als |
| B-80 | tobacco | bxn |
| B-81 | tomato | ACC |
| B-82 | tomato | Cry1Ac |
| B-83 | wheat | ALS |
| B-84 | wheat | CP4 epsps |

Preferably, the cultivated plants are plants, which comprise at least one trait selected from herbicide tolerance,

insect resistance by expression of bacertial toxins,
fungal resistance or viral resistance or bacterial resistance by expression of antipathogenic substances stress tolerance,
content modification of chemicals present in the cultivated plant compared to the corresponding wild-type plant.

More preferably, the cultivated plants are plants, which comprise at least one trait selected from herbicide tolerance,

insect resistance by expression of bacertial toxins,
fungal resistance or viral resistance or bacterial resistance by expression of antipathogenic substances content modification of chemicals present in the cultivated plant compared to the corresponding wild-type plant.

Most preferably, the cultivated plants are plants, which are tolerant to the action of herbicides and plants, which express bacterial toxins, which provides resistance against animal pests (such as insects or arachnids or nematodes), wherein the bacterial toxin is preferably a toxin from Bacillus thuriginensis. Herein, the plant is preferably selected from cereals (wheat, barley, rye, oat), soybean, rice, vine and fruit and vegetables such as tomato, potato and pome fruits, most preferably from soybean and cereals such as wheat, barley, rye and oat.

Thus, in one preferred embodiment, the present invention relates to a method of controlling harmful fungi and/or increasing the health of cultivated plants by treating cultivated plants, parts of such plants, plant propagation materials, or at their locus of growth with a carboxamide compound as defined above, wherein the plant is a plant, which is rendered tolerant to herbicides, more preferably to herbicides such as glutamine synthetase inhibitors, 5-enol-pyrovyl-shikimate-3-phosphate-synthase inhibitors, acetolactate synthase (ALS) inhibitors, protoporphyrinogen oxidase (PPO) inhibitors, auxine type herbicides, most preferably to herbicides such as glyphosate, glufosinate, imazapyr, imazapic, imazamox, imazethapyr, imazaquin, imazamethabenz methyl, dicamba and 2,4-D.

In a more preferred embodiment, the present invention relates to a method of controlling harmful fungi and/or increasing the health of cultivated plants by treating cultivated plants, parts of such plants, plant propagation materials, or at their locus of growth with a carboxamide compound selected from boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad, wherein the plant corresponds to row of table 1.

In a more preferred embodiment, the present invention relates to a method of controlling harmful fungi and/or increasing the health of cultivated plants by treating plant propagation materials, preferably seeds with a carboxamide compound as defined above, preferably with a carboxamide compound selected from boscalid, N-(3′,4′,5′-trifluorobiphenyl-2-yl)-3-difluoromethyl-1-methyl-1H-pyrazole-4-carboxamide, penflufen, fluopyram sedaxane, penthiopyrad carboxin, fenfuram, flutolanil; mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide, penflufen, fluopyram, sedaxane and penthiopyrad, wherein the plant corresponds to row of table 1.

In another more preferred embodiment, the present invention relates to a method of controlling harmful fungi and/or increasing the health of cultivated plants by treating cultivated plants, parts of such plants or at their locus of growth with a carboxamide compound selected from boscalid, N-(3′,4′,5′-trifluorobiphenyl-2-yl)-3-difluoromethyl-1-methyl-1′-1-pyrazole-4-carboxamide bixafen, fluopyram, isopyrazam, penthiopyrad, flutolanil, furametpyr, mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide bixafen, fluopyram, isopyrazam and penthiopyrad, wherein the plant corresponds to a row of table 1.

In a most preferred embodiment, the present invention relates to a method of controlling harmful fungi and/or increasing the health of plants by treating cultivated plants, parts of such plants or at their locus of growth with a carboxamide compound, wherein the plant corresponds to a row of table 1 and the carboxamide compound is boscalid.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 1 and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 1 and the carboxamide compound is bixafen.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 1 and the carboxamide compound is fluopyram.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 1 and the carboxamide compound is isopyrazam.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 1 and the carboxamide compound is penthiopyrad.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 1 and the carboxamide compound is boscalid.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 1 and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 1 and the carboxamide compound is penflufen.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 1 and the carboxamide compound is fluopyram.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 1 and the carboxamide compound is sedaxane.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 1 and the carboxamide compound is penthiopyrad.

TABLE 1

| No | detailed description | plant | Literature/commercial plants |
|---|---|---|---|
| T1-1 | imidazolinone tolerance | canola | B* |
| T1-2 | imidazolinone tolerance | maize | A*, B* |
| T1-3 | imidazolinone tolerance | rice | A*, C* |
| T1-4 | imidazolinone tolerance | millet | A* |
| T1-5 | imidazolinone tolerance | barley | A* |
| T1-6 | imidazolinone tolerance | wheat | A* |
| T1-7 | imidazolinone tolerance | sorghum | A* |
| T1-8 | imidazolinone tolerance | oats | A* |
| T1-9 | imidazolinone tolerance | rye | A* |
| T1-10 | imidazolinone tolerance | sugar beet | WO 1998/02526/WO 1998/02527 |
| T1-11 | imidazolinone tolerance | lentils | US2004/0187178 |
| T1-12 | imidazolinone tolerance | sunflowers | B* |
| T1-13 | imidazolinone tolerance | wheat | D* |
| T1-14 | glyphosate tolerance | alfalfa | E*; “Roundup Ready Alfalfa” |
| T1-15 | glyphosate tolerance | apple | E* |
| T1-16 | glyphosate tolerance | barley | E* |
| T1-17 | glyphosate tolerance | canola | E*; V* |
| T1-18 | glyphosate tolerance | maize | E*; W* |
| T1-19 | glyphosate tolerance | cotton | E*; X* |
| T1-20 | glyphosate tolerance | flax | E* |
| T1-21 | glyphosate tolerance | grape | E* |
| T1-22 | glyphosate tolerance | lentil | E* |
| T1-23 | glyphosate tolerance | oil seed rape | E* |
| T1-24 | glyphosate tolerance | pea | E* |
| T1-25 | glyphosate tolerance | potato | E* |
| T1-26 | glyphosate tolerance | rice | “Roundup Ready Rice” (Monsanto) |
| T1-27 | glyphosate tolerance | soybean | E*; Y* |
| T1-28 | glyphosate tolerance | sugar beet | E* |
| T1-29 | glyphosate tolerance | sunflower | E* |
| T1-30 | glyphosate tolerance | tobacco | E* |
| T1-31 | glyphosate tolerance | tomato | E* |
| T1-32 | glyphosate tolerance | turf grass | E* |
| T1-33 | glyphosate tolerance | wheat | E* |
| T1-34 | gluphosinate tolerance | canola | F*; U* |
| T1-35 | gluphosinate tolerance | maize | F*; Z* |
| T1-36 | gluphosinate tolerance | cotton | F*; “FiberMax Liberty Link” (Bayer), |
| T1-37 | gluphosinate tolerance | potato | F* |
| T1-38 | gluphosinate tolerance | rice | F*, G*; “Liberty Link Rice” (Bayer), |
| T1-39 | gluphosinate tolerance | sugar beet | F* |
| T1-40 | gluphosinate tolerance | soybean | U.S. Pat. No. 6,376,754 |
| T1-41 | gluphosinate tolerance | tobacco | F* |
| T1-42 | gluphosinate tolerance | tomato | F* |
| T1-43 | dicamba tolerance | bean | U.S. Pat. No. 7,105,724 |
| T1-44 | dicamba tolerance | maize | U.S. Pat. No. 7,105,724, |
| | | | WO 2008/051633 |
| T1-45 | dicamba tolerance | cotton | U.S. Pat. No. 7,105,724, |
| | | | U.S. Pat. No. 5,670,454 |
| T1-46 | dicamba tolerance | pea | U.S. Pat. No. 7,105,724 |
| T1-47 | dicamba tolerance | potato | U.S. Pat. No. 7,105,724 |
| T1-48 | dicamba tolerance | sorghum | U.S. Pat. No. 7,105,724 |
| T1-49 | dicamba tolerance | soybean | U.S. Pat. No. 7,105,724, |
| | | | U.S. Pat. No. 5,670,454 |
| T1-50 | dicamba tolerance | sunflower | U.S. Pat. No. 7,105,724 |
| T1-51 | dicamba tolerance | tobacco | U.S. Pat. No. 7,105,724 |
| T1-52 | dicamba tolerance | tomato | U.S. Pat. No. 7,105,724, |
| | | | U.S. Pat. No. 5,670,454 |
| T1-53 | bromoxynil tolerance | canola | “Navigator”, “Compass” (Rhone- |
| | | | Poulenc) |
| T1-54 | bromoxynil tolerance | cotton | “BXN” (calgene) |
| T1-55 | 2,4-D tolerance | apple | H* |
| T1-56 | 2,4-D tolerance | maize | H* |
| T1-57 | 2,4-D tolerance | cotton | U.S. Pat. No. 5,670,454 |
| T1-58 | 2,4-D tolerance | cucumber | H* |
| T1-59 | 2,4-D tolerance | pepper | H* |
| T1-60 | 2,4-D tolerance | potato | H* |
| T1-61 | 2,4-D tolerance | sorghum | H* |
| T1-62 | 2,4-D tolerance | soybean | H* |
| T1-63 | 2,4-D tolerance | sunflower | H* |
| T1-64 | 2,4-D tolerance | tobacco | H* |
| T1-65 | 2,4-D tolerance | tomato | H* |
| T1-66 | 2,4-D tolerance | wheat | H* |
| T1-67 | HPPD inhibitor tolerance (K*) | barley | I* |
| T1-68 | HPPD inhibitor tolerance (K*) | maizef | I* |
| T1-69 | HPPD inhibitor tolerance (K*) | cotton | I* |
| T1-70 | HPPD inhibitor tolerance (K*) | potato | I* |
| T1-71 | HPPD inhibitor tolerance (K*) | rapeseed | I* |
| T1-72 | HPPD inhibitor tolerance (K*) | rice | I* |
| T1-73 | HPPD inhibitor tolerance (K*) | soybean | I* |
| T1-74 | HPPD inhibitor tolerance (K*) | sutarbeet | I* |
| T1-75 | HPPD inhibitor tolerance (K*) | sugarcane | I* |
| T1-76 | HPPD inhibitor tolerance (K*) | tobacco | I* |
| T1-77 | HPPD inhibitor tolerance (K*) | wheat | I* |
| T1-78 | Protox inhibitor tolerance (L*) | cotton | M* |
| T1-79 | Protox inhibitor tolerance (L*) | rape | M* |
| T1-80 | Protox inhibitor tolerance (L*) | rice | M* |
| T1-81 | Protox inhibitor tolerance (L*) | sorghum | M* |
| T1-82 | Protox inhibitor tolerance (L*) | soybean | M* |
| T1-83 | Protox inhibitor tolerance (L*) | sugarbeet | M* |
| T1-84 | Protox inhibitor tolerance (L*) | sugar cane | M* |
| T1-85 | Protox inhibitor tolerance (L*) | wheat | M* |
| T1-86 | imidazolinone tolerance | soybean | N* |

A* refers to U.S. Pat. No. 4,761,373, U.S. Pat. No. 5,304,732, U.S. Pat. No. 5,331,107, U.S. Pat. No. 5,718,079, U.S. Pat. No. 6,211,438, U.S. Pat. No. 6,211,439 and U.S. Pat. No. 6,222,100.
B* refers to Tan et. al, Pest Manag. Sci 61, 246-257 (2005).
C* refers to imidazolinone-herbicide resistant rice plants with specific mutation of the acetohydroxyacid synthase gene: S653N (see e.g. US 2003/0217381), S654K (see e.g. US 2003/0217381), A122T (see e.g. WO 2004/106529) S653(At)N, S654(At)K, A122(At)T and other resistant rice plants as described in WO 2000/27182, WO 2005/20673 and WO 2001/85970 or U.S. Pat. Nos. U.S. Pat. No. 5,545,822, U.S. Pat. No. 5,736,629, U.S. Pat. No. 5,773,703, U.S. Pat. No. 5,773,704, U.S. Pat. No. 5,952,553, U.S. Pat. No. 6,274,796, wherein plants with mutation S653A and A122T are most preferred.
D* refers to WO 2004/106529, WO 2004/16073, WO 2003/14357, WO 2003/13225 and WO 2003/14356.
E* refers to U.S. Pat. No. 5,188,642, U.S. Pat. No. 4,940,835, U.S. Pat. No. 5,633,435, U.S. Pat. No. 5,804,425 and U.S. Pat. No. 5,627,061.
F* refers to U.S. Pat. No. 5,646,024 and U.S. Pat. No. 5,561,236.
G* refers to U.S. Pat. No. 6,333,449, U.S. Pat. No. 6,933,111 and U.S. Pat. No. 6,468,747.
H* refers to U.S. Pat. No. 6,153,401, U.S. Pat. No. 6,100,446, WO 2005/107437, U.S. Pat. No. 5,670,454 and U.S. Pat. No. 5,608,147.
I* refers to WO 2004/055191, WO 199638567 and U.S. Pat. No. 6,791,014.
K* refers to HPPD inhibitor herbicides, such as isoxazoles (e.g. isoxaflutole), diketonitriles, trikeones (e.g. sulcotrione and mesotrione), pyrazolinates.
L* refers to protoporphyrinogen oxidase (PPO) inhibiting herbicides.
M* refers to US 2002/0073443, US 20080052798, Pest Management Science, 61, 2005, 277-285.
N* refers to the herbicide tolerant soybean plants presented under the name of Cultivance on the XVI Congresso Brasileiro de Sementes, 31st Aug. to 3^{rd} Sep. 2009 at Estação Embratel Convention Center - Curitiba/PR, Brazil
U* “InVigor” (Bayer)
V* “Roundup Ready Canola” (Monsanto)
W* “Roundup Ready Corn”, “Roundup Ready 2” (Monsanto), “Agrisure GT”, “Agrisure GT/CB/LL”, “Agrisure GT/RW”, ^{“}Agrisure 3000GT” (Syngenta), “YieldGard VT Rootworm/RR2”, “YieldGard VT Triple” (Monsanto)
X* “Roundup Ready Cotton”, “Roundup Ready Flex” (Monsanto]
Y* “Roundup Ready Soybean” (Monsanto), “Optimum GAT” (DuPont, Pioneer)
Z* “Liberty Link” (Bayer), “Herculex I”, “Herculex RW”, “Herculex Xtra”(Dow, Pioneer), “Agrisure GT/CB/LL”, “Agrisure CB/LL/RW” (Syngenta),

A subset of especially preferred herbicide tolerant plants is given in table 2. In this subset, there are further preferred embodiments:

In a more preferred embodiment, the present invention relates to a method of controlling harmful fungi and/or increasing the health of cultivated plants by treating plant propagation materials, preferably seeds with a carboxamide compound as defined above, preferably with a carboxamide compound selected from boscalid, N-(3′,4′,5′-trifluorobiphenyl-2-yl)-3-difluoromethyl-1-methyl-1H-pyrazole-4-carboxamide, penflufen, fluopyram sedaxane, penthiopyrad carboxin, fenfuram, flutolanil; mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide, penflufen, fluopyram, sedaxane and penthiopyrad, wherein the plant corresponds to a row of table 2.

In another more preferred embodiment, the present invention relates to a method of controlling harmful fungi and/or increasing the health of cultivated plants by treating cultivated plants, parts of such plants or at their locus of growth with a carboxamide compound selected from boscalid, N-(3′,4′,5′-trifluorobiphenyl-2-yl)-3-difluoromethyl-1-methyl-1H-pyrazole-4-carboxamide bixafen, fluopyram, isopyrazam, penthiopyrad, flutolanil, furametpyr, mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide bixafen, fluopyram, isopyrazam and penthiopyrad, wherein the plant corresponds to a row of table 2.

In a most preferred embodiment, the present invention relates to a method of controlling harmful fungi and/or increasing the health of plants by treating cultivated plants, parts of such plants or at their locus of growth with a carboxamide compound, wherein the plant corresponds to a row of table 2 and the carboxamide compound is boscalid.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 2 and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 2 and the carboxamide compound is bixafen.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 2 and the carboxamide compound is fluopyram.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 2 and the carboxamide compound is isopyrazam.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 2 and the carboxamide compound is penthiopyrad.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 2 and the carboxamide compound is boscalid.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 2 and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 2 and the carboxamide compound is penflufen.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 2 and the carboxamide compound is fluopyram.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 2 and the carboxamide compound is sedaxane.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 2 and the carboxamide compound is penthiopyrad.

In a utmost preferred embodiment, the present invention relates to a method of controlling harmful fungi and/or increasing the health of plants by treating cultivated plants, parts of such plants or at their locus of growth with a carboxamide compound, wherein the plant is selected from T2-3, T2-5, T2-10, T2-11, T2-16, T2-17 and T2-23 of table 2 and the carboxamide compound is boscalid.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T2-3, T2-5, T2-10, T2-11, T2-16, T2-17 and T2-23 of table 2 and the carboxamide compound is N-(3′,4′,5′-trifluorobiphenyl-2-yl)-3-difluoromethyl-1-methyl-1H-pyrazole-4-carboxamide.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T2-3, T2-5, T2-10, T2-11, T2-16, T2-17 and T2-23 of table 2 and the carboxamide compound is bixafen.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T2-3, T2-5, T2-10, T2-11, T2-16, T2-17 and T2-23 of table 2 and the carboxamide compound is fluopyram.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T2-3, T2-5, T2-10, T2-11, T2-16, T2-17 and T2-23 of table 2 and the carboxamide compound is isopyrazam.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T2-3, T2-5, T2-10, T2-11, T2-16, T2-17 and T2-23 of table 2 and the carboxamide compound is penthiopyrad.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T2-3, T2-5, T2-10, T2-11, T2-16, T2-17 and T2-23 of table 2 and the carboxamide compound is boscalid.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T2-3, T2-5, T2-10, T2-11, T2-16, T2-17 and T2-23 of table 2 and the carboxamide compound is N-(3′,4′,5′-trifluorobiphenyl-2-yl)-3-difluoromethyl-1-methyl-1H-pyrazole-4-carboxamide.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T2-3, T2-5, T2-10, T2-11, T2-16, T2-17 and T2-23 of table 2 and the carboxamide compound is penflufen.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T2-3, T2-5, T2-10, T2-11, T2-16, 12-17 and T2-23 of table 2 and the carboxamide compound is fluopyram.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T2-3, T2-5, T2-10, T2-11, T2-16, 12-17 and T2-23 of table 2 and the carboxamide compound is sedaxane.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T2-3, T2-5, T2-10, T2-11, T2-16, T2-17 and T2-23 of table 2 and the carboxamide compound is penthiopyrad.

TABLE 2

| No | detailed description | plant | Literature/commercial plants |
|---|---|---|---|
| T2-1 | imidazolinone tolerance | canola | B* |
| T2-2 | imidazolinone tolerance | maize | A*, B* |
| T2-3 | imidazolinone tolerance | rice | C* |
| T2-4 | imidazolinone tolerance | sunflowers | B* |
| T2-5 | imidazolinone tolerance | wheat | D* |
| T2-6 | glyphosate tolerance | alfalfa | E*; “Roundup Ready Alfalfa” |
| T2-7 | glyphosate tolerance | canola | E*; U* |
| T2-8 | glyphosate tolerance | maize | E*; V* |
| T2-9 | glyphosate tolerance | cotton | E*; W* |
| T2-10 | glyphosate tolerance | rice | E*; “Roundup Ready Rice” (Monsanto) |
| T2-11 | glyphosate tolerance | soybean | E*; X* |
| T2-12 | glyphosate tolerance | sugar beet | E* |
| T2-13 | glufosinate tolerance | canola | F*; “InVigor” (Bayer) |
| T2-14 | glufosinate tolerance | maize | F*; Y* |
| T2-15 | glufosinate tolerance | cotton | F*; “FiberMax Liberty Link” (Bayer), |
| T2-16 | glufosinate tolerance | rice | F*, G*; “Liberty Link Rice” (Bayer), |
| T2-17 | glufosinate tolerance | soybean | I* |
| T2-18 | dicamba tolerance | cotton | U.S. Pat. No. 7,105,724 |
| T2-19 | dicamba tolerance | soybean | U.S. Pat. No. 7,105,724 |
| T2-20 | bromoxynil tolerance | canola | Z* |
| T2-21 | bromoxynil tolerance | cotton | “BXN” (Calgene) |
| T2-22 | 2,4-D tolerance | maize | H* |
| T2-23 | imidazolinone tolerance | soybean | N* |

A* refers to U.S. Pat. No. 4,761,373, U.S. Pat. No. 5,304,732, U.S. Pat. No. 5,331,107, U.S. Pat. No. 5,718,079, U.S. Pat. No. 6,211,438, U.S. Pat. No. 6,211,439 and U.S. Pat. No. 6,222,100.
B* refers to Tan et. al, Pest Manag. Sci 61, 246-257 (2005).
C* refers to imidazolinone-herbicide resistant rice plants with specific mutation of the acetohydroxyacid synthase gene: S653N (see e.g. US 2003/0217381), S654K (see e.g. US 2003/0217381), A122T (see e.g. WO 04/106529) S653(At)N, S654(At)K, A122(At)T and other resistant rice plants as described in WO 2000/27182, WO 2005/20673 and WO 2001/85970 or U.S. Pat. Nos. U.S. Pat. No. 5,545,822, U.S. Pat. No. 5,736,629, U.S. Pat. No. 5,773,703, U.S. Pat. No. 5,773,704, U.S. Pat. No. 5,952,553, U.S. Pat. No. 6,274,796, wherein plants with mutation S653A and A122T are most preferred.
D* refers to WO 04/106529, WO 04/16073, WO 03/14357, WO 03/13225 and WO 03/14356.
E* refers to U.S. Pat. No. 5,188,642, U.S. Pat. No. 4,940,835, U.S. Pat. No. 5,633,435, U.S. Pat. No. 5,804,425 and U.S. Pat. No. 5,627,061.
F* refers to U.S. Pat. No. 5,646,024 and U.S. Pat. No. 5,561,236.
G* refers to U.S. Pat. No. 6,333,449, U.S. Pat. No. 6,933,111 and U.S. Pat. No. 6,468,747.
H* refers to U.S. Pat. No. 6,153,401, U.S. Pat. No. 6,100,446, WO 2005/107437 and U.S. Pat. No. 5,608,147.
I* refers to Federal Register (USA), Vol. 61, No.160, 1996, page 42581. Federal Register (USA), Vol. 63, No.204, 1998, page 56603.
N* refers to the herbicide tolerant soybean plants presented under the name of Cultivance on the XVI Congresso Brasileiro de Sementes, 31st Aug. to 3^{rd} Sep. 2009 at Estação Embratel Convention Center - Curitiba/PR, Brazil
U* “Roundup Ready Canola” (Monsanto)
V* “Roundup Ready Corn”, “Roundup Ready 2” (Monsanto), “Agrisure GT”, “Agrisure GT/CB/LL”, “Agrisure GT/RW”, _{“}Agrisure 3000GT” (Syngenta), “YieldGard VT Rootworm/RR2”, “YieldGard VT Triple” (Monsanto)
W* “Roundup Ready Cotton”, “Roundup Ready Flex” (Monsanto)
X* “Roundup Ready Soybean” (Monsanto), “Optimum GAT” (DuPont, Pioneer)
Y* “Liberty Link” (Bayer), “Herculex I”, “Herculex RW”, “Herculex Xtra”(Dow, Pioneer), “Agrisure GT/CB/LL”, “Agrisure CB/LL/RW” (Syngenta)
Z* “Navigator”, “Compass” (Rhone-Poulenc)

In a further one preferred embodiment, the present invention relates to a method of controlling harmful fungi and/or increasing the health of cultivated plants by treating cultivated plants, parts of such plants, plant propagation materials, or at their locus of growth with a carboxamide compound selected from boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad wherein the plant is a plant, which express at least one insecticidal toxin, preferably a toxin from Bacillus species, more preferably from Bacillus thuringiensis.

In a more preferred embodiment, the present invention relates to a method of controlling harmful fungi and/or increasing the health of cultivated plants by treating plant propagation materials, preferably seeds with a carboxamide compound as defined above, preferably with a carboxamide compound selected from boscalid, N-(3′,4′,5′-trifluorobiphenyl-2-yl)-3-difluoromethyl-1-methyl-1H-pyrazole-4-carboxamide, penflufen, fluopyram sedaxane, penthiopyrad carboxin, fenfuram, flutolanil; mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide, penflufen, fluopyram, sedaxane and penthiopyrad, wherein the plant corresponds to a row of table 3.

In another more preferred embodiment, the present invention relates to a method of controlling harmful fungi and/or increasing the health of cultivated plants by treating cultivated plants, parts of such plants or at their locus of growth with a carboxamide compound selected from boscalid, N-(3′,4′,5′-trifluorobiphenyl-2-yl)-3-difluoromethyl-1-methyl-1H-pyrazole-4-carboxamide bixafen, fluopyram, isopyrazam, penthiopyrad, flutolanil, furametpyr, mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide bixafen, fluopyram, isopyrazam and penthiopyrad, wherein the plant corresponds to a row of table 3.

In a most preferred embodiment, the present invention relates to a method of controlling harmful fungi and/or increasing the health of plants by treating cultivated plants, parts of such plants or at their locus of growth with a carboxamide compound, wherein the plant corresponds to a row of table 3 and the carboxamide compound is boscalid.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 3 and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 3 and the carboxamide compound is bixafen.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 3 and the carboxamide compound is fluopyram.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 3 and the carboxamide compound is isopyrazam.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 3 and the carboxamide compound is penthiopyrad.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 3 and the carboxamide compound is boscalid.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 3 and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 3 and the carboxamide compound is penflufen.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 3 and the carboxamide compound is fluopyram.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 3 and the carboxamide compound is sedaxane.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 3 and the carboxamide compound is penthiopyrad.

In a utmost preferred embodiment, the present invention relates to a method of controlling harmful fungi and/or increasing the health of plants by treating cultivated plants, parts of such plants or at their locus of growth with a carboxamide compound, wherein the plant is selected from T3-13, T3-14, T3-15, T3-16, T3-17, T3-18, T3-19, T3-20, T3-23 and T3-25 of table 3 and the carboxamide compound is boscalid.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T3-13, T3-14, T3-15, T3-16, T3-17, T3-18, 13-19, T3-20, T3-23 and T3-25 of table 3 and the carboxamide compound is N-(3′,4′,5′-trifluorobiphenyl-2-yl)-3-difluoromethyl-1-methyl-1H-pyrazole-4-carboxamide.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T3-13, T3-14, T3-15, T3-16, T3-17, T3-18, T3-19, T3-20, T3-23 and T3-25 of table 3 and the carboxamide compound is bixafen.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T3-13, T3-14, T3-15, T3-16, T3-17, T3-18, T3-19, T3-20, T3-23 and T3-25 of table 3 and the carboxamide compound is fluopyram.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T3-13, T3-14, T3-15, T3-16, T3-17, T3-18, T3-19, T3-20, T3-23 and T3-25 of table 3 and the carboxamide compound is isopyrazam.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T3-13, T3-14, T3-15, T3-16, T3-17, T3-18, T3-19, T3-20, T3-23 and T3-25 of table 3 and the carboxamide compound is penthiopyrad.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T3-13, T3-14, T3-15, T3-16, T3-17, T3-18, T3-19, T3-20, T3-23 and T3-25 of table 3 and the carboxamide compound is boscalid.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T3-13, T3-14, T3-15, T3-16, T3-17, T3-18, T3-19, T3-20, T3-23 and T3-25 of table 3 and the carboxamide compound is N-(3′,4′,5′-trifluorobiphenyl-2-yl)-3-difluoromethyl-1-methyl-1H-pyrazole-4-carboxamide.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T3-13, T3-14, T3-15, T3-16, T3-17, T3-18, T3-19, T3-20, T3-23 and T3-25 of table 3 and the carboxamide compound is penflufen.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T3-13, T3-14, T3-15, T3-16, T3-17, T3-18, T3-19, T3-20, T3-23 and T3-25 of table 3 and the carboxamide compound is fluopyram.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T3-13, T3-14, T3-15, T3-16, T3-17, T3-18, T3-19, T3-20, T3-23 and T3-25 of table 3 and the carboxamide compound is sedaxane.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T3-13, T3-14, T3-15, T3-16, T3-17, T3-18, T3-19, T3-20, T3-23 and T3-25 of table 3 and the carboxamide compound is penthiopyrad.

TABLE 3

| No | detailed description | plant | Literature/commercial plants |
|---|---|---|---|
| T3-1 | corn rootworm resistance | maize | B* |
| T3-2 | corn borer resistance | maize | C* |
| T3-3 | western bean cutworm resis- | maize | D* |
| | tance | | |
| T3-4 | black cutworm resistance | maize | E* |
| T3-5 | fall armyworm resistance | maize | _{“}Herculex I” (Dow, Pioneer), _{“}Herculex |
| | | | Xtra” (Dow, Pioneer) |
| T3-6 | tobacco budworm resistance | cotton | “Bollgard I” (Monsanto), “Bollgard II” |
| | | | (Monsanto), _{“}WideStrike” (Dow), _{“}VipCot” |
| | | | (Syngenta) |
| T3-7 | cotton bollworm resistance | cotton | “Bollgard II” (Monsanto), _{“}WideStrike” |
| | | | (Dow), _{“}VipCot” (Syngenta) |
| T3-8 | fall armyworm resistance | cotton | “Bollgard II” (Monsanto), _{“}WideStrike” |
| | | | (Dow), _{“}VipCot” (Syngenta) |
| T3-9 | beet armyworm resistance | cotton | “Bollgard II” (Monsanto), _{“}WideStrike” |
| | | | (Dow), _{“}VipCot” (Syngenta) |
| T3-10 | cabbage looper resistance | cotton | “Bollgard II” (Monsanto), _{“}WideStrike” |
| | | | (Dow), _{“}VipCot” (Syngenta) |
| T3-11 | soybean lopper resistance | cotton | “Bollgard II” (Monsanto), _{“}WideStrike” |
| | | | (Dow), _{“}VipCot” (Syngenta) |
| T3-12 | pink bollworm resistance | cotton | “Bollgard II” (Monsanto), _{“}WideStrike” |
| | | | (Dow), _{“}VipCot” (Syngenta) |
| T3-13 | rice stemborer resistance | rice | A* |
| T3-14 | striped rice borer resistance | rice | A* |
| T3-15 | rice leaf roller resistance | rice | A* |
| T3-16 | yellow stemborer resistance | rice | A* |
| T3-17 | rice skipper resistance | rice | A* |
| T3-18 | rice caseworm resistance | rice | A* |
| T3-19 | rice cutworm resistance | rice | A* |
| T3-20 | rice armyworm resistance | rice | A* |
| T3-21 | brinjal fruit and shoot borer | eggplant | U.S. Pat. No. 5,128,130, “Bt brinjal”, |
| | resistance | | “Dumaguete Long Purple”, “Mara” |
| T3-22 | cotton bollworm resistance | eggplant | U.S. Pat. No. 5,128,130, “Bt brinjal”, |
| | | | “Dumaguete Long Purple”, “Mara” |
| T3-23 | tobacco hornworm resistance | potato | D* |
| T3-24 | lepidopteran resistance | lettuce | U.S. Pat. No. 5,349,124 |
| T3-25 | lepidopteran resistance | soybean | U.S. Pat. No. 7,432,421 |

A* refers to _{“}Zhuxian B”, WO 2001/021821, Molecular Breeding, Volume 18, Number 1/August 2006.
B* “YieldGard corn rootworm” (Monsanto), “YieldGard Plus” (Monsanto), “YieldGard VT” (Monsanto), “Herculex RW” (Dow, Pioneer), “Herculex Rootworm” (Dow, Pioneer), “Agrisure 0CRW” (Syngenta)
C* “YieldGard corn borer” (Monsanto), _{“}YieldGard Plus” (Monsanto), _{“}YieldGard VT Pro” (Monsanto), “Agrisure CB/LL” (Syngenta), “Agrisure 3000GT” (Syngenta), “Hercules I”, “Hercules II” (Dow, Pioneer), “KnockOut” (Novartis), _{“}NatureGard” (Mycogen), _{“}StarLink” (Aventis)
D* “NewLeaf” (Monsanto), “NewLeaf Y” (Monsanto), “NewLeaf Plus” (Monsanto), U.S. Pat. No. 6,100,456

In a further one preferred embodiment, the present invention relates to a method of controlling harmful fungi and/or increasing the health of cultivated plants by treating cultivated plants, parts of such plants, plant propagation materials, or at their locus of growth with a carboxamide compound selected from boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad, wherein the plant is a plant, which shows increased resistance against fungal, viral and bacterial diseases, more preferably a plant, which expresses antipathogenic substances, such as antifungal proteins, or which has systemic acquired resistance properties.

In a more preferred embodiment, the present invention relates to a method of controlling harmful fungi and/or increasing the health of cultivated plants by treating plant propagation materials, preferably seeds with a carboxamide compound selected from boscalid, N-(3′,4′,5′-trifluorobiphenyl-2-yl)-3-difluoromethyl-1-methyl-1H-pyrazole-4-carboxamide, penflufen, fluopyram sedaxane, penthiopyrad carboxin, fenfuram, flutolanil; mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide, penflufen, fluopyram, sedaxane and penthiopyrad, wherein the plant corresponds to row of table 4.

In another more preferred embodiment, the present invention relates to a method of controlling harmful fungi and/or increasing the health of cultivated plants by treating cultivated plants, parts of such plants or at their locus of growth with a carboxamide compound selected from boscalid, N-(3′,4′,5′-trifluorobiphenyl-2-yl)-3-difluoromethyl-1-methyl-1H-pyrazole-4-carboxamide bixafen, fluopyram, isopyrazam, penthiopyrad, flutolanil, furametpyr, mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide bixafen, fluopyram, isopyrazam and penthiopyrad, wherein the plant corresponds to row of table 4.

In a most preferred embodiment, the present invention relates to a method of controlling harmful fungi and/or increasing the health of plants by treating cultivated plants, parts of such plants or at their locus of growth with a carboxamide compound, wherein the plant corresponds to a row of table 4 and the carboxamide compound is boscalid.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 4 and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 4 and the carboxamide compound is bixafen.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 4 and the carboxamide compound is fluopyram.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 4 and the carboxamide compound is isopyrazam.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 4 and the carboxamide compound is penthiopyrad.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 4 and the carboxamide compound is boscalid.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 4 and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 4 and the carboxamide compound is penflufen.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 4 and the carboxamide compound is fluopyram.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 4 and the carboxamide compound is sedaxane.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 4 and the carboxamide compound is penthiopyrad.

TABLE 4

| No | detailed description | plant | Literature |
|---|---|---|---|
| T4-1. | fungal resistance | apple | A*, B*, C* |
| T4-2. | fungal resistance | barley | A*, B*, C* |
| T4-3. | fungal resistance | banana | A*, B*, C* |
| T4-4. | fungal resistance | bean | B*, C* |
| T4-5. | fungal resistance | maize | A*, B*, C* |
| T4-6. | fungal resistance | cotton | A*, C* |
| T4-7. | fungal resistance | cucumber | B*, C* |
| T4-8. | fungal resistance | grape | C* |
| T4-9. | fungal resistance | oat | A*, C* |
| T4-10. | fungal resistance | pepper | B*, C* |
| T4-11. | fungal resistance | potato | A*, B*, C* |
| T4-12. | fungal resistance | rape | B*, C* |
| T4-13. | fungal resistance | rice | A*, B*, C* |
| T4-14. | fungal resistance | rye | A*, B*, C* |
| T4-15. | fungal resistance | sorghum | B*, C* |
| T4-16. | fungal resistance | soybean | A*, B*, C* |
| T4-17. | fungal resistance | sugarcane | B*, C* |
| T4-18. | fungal resistance | tobacco | A*, B*, C* |
| T4-19. | fungal resistance | tomato | A*, B*, C* |
| T4-20. | fungal resistance | wheat | A*, B*, C* |
| T4-21. | bacterial resistance | apple | D* |
| T4-22. | bacterial resistance | barley | D* |
| T4-23. | bacterial resistance | banana | D* |
| T4-24. | bacterial resistance | bean | D* |
| T4-25. | bacterial resistance | maize | |
| T4-26. | bacterial resistance | cotton | D* |
| T4-27. | bacterial resistance | cucumber | D* |
| T4-28. | bacterial resistance | grape | D*, U.S. Pat. |
| | | | No. 6,172,280 |
| T4-29. | bacterial resistance | oat | D* |
| T4-30. | bacterial resistance | pepper | D* |
| T4-31. | bacterial resistance | potato | D* |
| T4-32. | bacterial resistance | rape | D* |
| T4-33. | bacterial resistance | rice | D* |
| T4-34. | bacterial resistance | rye | D* |
| T4-35. | bacterial resistance | sorghum | D* |
| T4-36. | bacterial resistance | soybean | D* |
| T4-37. | bacterial resistance | sugarcane | D* |
| T4-38. | bacterial resistance | tobacco | D* |
| T4-39. | bacterial resistance | tomato | D* |
| T4-40. | bacterial resistance | wheat | D* |
| T4-41. | viral resistance | apple | C* |
| T4-42. | viral resistance | barley | C* |
| T4-43. | viral resistance | banana | C* |
| T4-44. | viral resistance | bean | C* |
| T4-45. | viral resistance | maize | C* |
| T4-46. | viral resistance | cotton | C* |
| T4-47. | viral resistance | cucumber | C* |
| T4-48. | viral resistance | oat | C* |
| T4-49. | viral resistance | pepper | C* |
| T4-50. | viral resistance | potato | C* |
| T4-51. | viral resistance | rape | C* |
| T4-52. | viral resistance | rice | C* |
| T4-53. | viral resistance | rye | C* |
| T4-54. | viral resistance | sorghum | C* |
| T4-55. | viral resistance | soybean | C* |
| T4-56. | viral resistance | sugarcane | C* |
| T4-57. | viral resistance | tobacco | C* |
| T4-58. | viral resistance | tomato | C* |
| T4-59. | viral resistance | wheat | C* |
| T4-60. | fungal resistance | potato | E* |

A* refers to U.S. Pat. No. 5,689,046 and U.S. Pat. No. 6,020,129.
B* refers to U.S. Pat. No. 6,706,952 and EP 1018553.
C* refers to U.S. Pat. No. 6,630,618.
D* refers to WO 1995/005731 and U.S. Pat. No. 5,648,599.
E* refers to the potato plant variety submitted for variety registration with the Community Plant Variety Office (CPVO), 3, boulevard Maréchal Foch, BP 10121, FR - 49101 Angers Cedex 02, France and having the CPVO file number 20082800

In a more preferred embodiment, the present invention relates to a method of controlling harmful fungi and/or increasing the health of cultivated plants by treating cultivated plants, parts of such plants, plant propagation materials, or at their locus of growth with a carboxamide compound selected from boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad, wherein the plant is a plant, which is listed in table 5.

In a more preferred embodiment, the present invention relates to a method of controlling harmful fungi and/or increasing the health of cultivated plants by treating plant propagation materials, preferably seeds with a carboxamide compound selected from boscalid, N-(3′,4′,5′-trifluorobiphenyl-2-yl)-3-difluoromethyl-1-methyl-1H-pyrazole-4-carboxamide, penflufen, fluopyram sedaxane, penthiopyrad carboxin, fenfuram, flutolanil; mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide, penflufen, fluopyram, sedaxane and penthiopyrad, wherein the plant corresponds to a row of table 5.

In another more preferred embodiment, the present invention relates to a method of controlling harmful fungi and/or increasing the health of cultivated plants by treating cultivated plants, parts of such plants or at their locus of growth with a carboxamide compound selected from boscalid, N-(3′,4′,5′-trifluorobiphenyl-2-yl)-3-difluoromethyl-1-methyl-1H-pyrazole-4-carboxamide bixafen, fluopyram, isopyrazam, penthiopyrad, flutolanil, furametpyr, mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide bixafen, fluopyram, isopyrazam and penthiopyrad, wherein the plant corresponds to a row of table 5.

In a most preferred embodiment, the present invention relates to a method of controlling harmful fungi and/or increasing the health of plants by treating cultivated plants, parts of such plants or at their locus of growth with a carboxamide compound, wherein the plant corresponds to a row of table 5 and the carboxamide compound is boscalid.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 5 and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 5 and the carboxamide compound is bixafen.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 5 and the carboxamide compound is fluopyram.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 5 and the carboxamide compound is isopyrazam.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 5 and the carboxamide compound is penthiopyrad.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 5 and the carboxamide compound is boscalid.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 5 and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 5 and the carboxamide compound is penflufen.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 5 and the carboxamide compound is fluopyram.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 5 and the carboxamide compound is sedaxane.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 5 and the carboxamide compound is penthiopyrad.

In a utmost preferred embodiment, the present invention relates to a method of controlling harmful fungi and/or increasing the health of plants by treating cultivated plants, parts of such plants or at their locus of growth with a carboxamide compound, wherein the plant is selected from T5-2, T5-5, T5-6, T5-9, T5-10, T5-11, T5-13 and T5-14 of table 5 and the carboxamide compound is boscalid.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T5-2, T5-5, T5-6, T5-9, T5-10, T5-11, T5-13 and T5-14 of table 5 and the carboxamide compound is N-(3′,4′,5′-trifluorobiphenyl-2-yl)-3-difluoromethyl-1-methyl-1H-pyrazole-4-carboxamide.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T5-2, T5-5, T5-6, T5-9, T5-10, T5-11, T5-13 and T5-14 of table 5 and the carboxamide compound is bixafen.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T5-2, T5-5, T5-6, T5-9, T5-10, T5-11, T5-13 and T5-14 of table 5 and the carboxamide compound is fluopyram.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T5-2, T5-5, T5-6, T5-9, T5-10, T5-11, T5-13 and T5-14 of table 5 and the carboxamide compound is isopyrazam.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T5-2, T5-5, T5-6, T5-9, T5-10, T5-11, T5-13 and T5-14 of table 5 and the carboxamide compound is penthiopyrad.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T5-2, T5-5, T5-6, T5-9, T5-10, T5-11, T5-13 and T5-14 of table 5 and the carboxamide compound is boscalid.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T5-2, T5-5, T5-6, T5-9, T5-10, T5-11, T5-13 and T5-14 of table 5 and the carboxamide compound is N-(3′,4′,5′-trifluorobiphenyl-2-yl)-3-difluoromethyl-1-methyl-1H-pyrazole-4-carboxamide.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T5-2, T5-5, T5-6, T5-9, T5-10, T5-11, T5-13 and T5-14 of table 5 and the carboxamide compound is penflufen.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T5-2, T5-5, T5-6, T5-9, T5-10, T5-11, T5-13 and T5-14 of table 5 and the carboxamide compound is fluopyram.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T5-2, T5-5, T5-6, T5-9, T5-10, T5-11, T5-13 and T5-14 of table 5 and the carboxamide compound is sedaxane.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T5-2, T5-5, T5-6, T5-9, T5-10, T5-11, T5-13 and T5-14 of table 5 and the carboxamide compound is penthiopyrad.

TABLE 5

| No | detailed description | plant | Literature/commercial plants |
|---|---|---|---|
| T5-1 | broad fungal resistance | maize | A*, B*, C* |
| T5-2 | broad fungal resistance | soybean | A*, B*, C* |
| T5-3 | asian soybean rust resistance | soybean | WO 2008/017706 |
| T5-4 | resistance against anthracnose leaf bligh, | maize | US 2006/225152 |
| | anthracnose stalk rot (colletotrichum | | |
| | graminicola), diplodia ear rot, fusarium | | |
| | verticilioides, gibberella zeae, top dieback | | |
| T5-5 | resistance against anthracnose leaf bligh, | maize | US 2006/225152 |
| | anthracnose stalk rot (colletotrichum | | |
| | graminicola), diplodia ear rot, fusarium | | |
| | verticilioides, gibberella zeae, top dieback | | |
| T5-6 | fusarium resistance | wheat | U.S. Pat. No. 6,646,184, EP 1477557 |
| T5-7 | apple scab resistance | apple | WO 1999/064600 |
| T5-8 | plum pox virus resistance | plum | US PP15154PS |
| T5-9 | potato virus X resistance | potato | U.S. Pat. No. 5,968,828, EP 0707069 |
| T5-10 | potato virus Y resistance | potato | EP 0707069; “NewLeaf Y” (Monsanto) |
| T5-11 | potato leafroll virus resistance | potato | EP 0707069, U.S. Pat. No. 5,576,202; |
| | | | “New-Leaf Plus” (Monsanto) |
| T5-12 | papaya ring spot virus resistance | papaya | U.S. Pat. No. 5,877,403, U.S. Pat. No. 6,046,384 |
| T5-13 | bacterial blight resistance | rice | D* |
| T5-14 | fungal resistance | potato | E* |

A* refers to U.S. Pat. No. 5,689,046 and U.S. Pat. No. 6,020,129.
B* refers to U.S. Pat. No. 6,706,952 and EP 1018553.
C* refers to U.S. Pat. No. 6,630,618.
D* refers to WO 2006/42145, U.S. Pat. No. 5,952,485, U.S. Pat. No. 5,977,434, WO 1999/09151 and WO 1996/22375.
E* refers to the potato plant variety submitted for variety registration with the Community Plant Variety Office (CPVO), 3, boulevard Maréchal Foch, BP 10121, FR - 49101 Angers Cedex 02, France and having the CPVO file number 20082800.

In a further one preferred embodiment, the present invention relates to a method of controlling harmful fungi and/or increasing the health of cultivated plants by treating cultivated plants, parts of such plants, plant propagation materials, or at their locus of growth with a carboxamide compound selected from boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad, wherein the plant is a plant, which is tolerant to abiotic stress, preferably drought, high salinity, high light intensities, high UV irradiation, chemical pollution (such as high heavy metal concentration), low or high temperatures, limited supply of nutrients and population stress, most preferably drought, high salinity, low temperatures and limited supply of nitrogen.

In a more preferred embodiment, the present invention relates to a method of controlling harmful fungi and/or increasing the health of cultivated plants by treating plant propagation materials, preferably seeds with a carboxamide compound selected from boscalid, N-(3′,4′,5′-trifluorobiphenyl-2-yl)-3-difluoromethyl-1-methyl-1H-pyrazole-4-carboxamide, penflufen, fluopyram sedaxane, penthiopyrad carboxin, fenfuram, flutolanil; mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide, penflufen, fluopyram, sedaxane and penthiopyrad, wherein the plant corresponds to a row of table 6.

In another more preferred embodiment, the present invention relates to a method of controlling harmful fungi and/or increasing the health of cultivated plants by treating cultivated plants, parts of such plants or at their locus of growth with a carboxamide compound selected from boscalid, N-(3′,4′,5′-trifluorobiphenyl-2-yl)-3-difluoromethyl-1-methyl-1H-pyrazole-4-carboxamide bixafen, fluopyram, isopyrazam, penthiopyrad, flutolanil, furametpyr, mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide bixafen, fluopyram, isopyrazam and penthiopyrad, wherein the plant corresponds to a row of table 6.

In a most preferred embodiment, the present invention relates to a method of controlling harmful fungi and/or increasing the health of plants by treating cultivated plants, parts of such plants or at their locus of growth with a carboxamide compound, wherein the plant corresponds to a row of table 6 and the carboxamide compound is boscalid.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 6 and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 6 and the carboxamide compound is bixafen.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 6 and the carboxamide compound is fluopyram.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 6 and the carboxamide compound is isopyrazam.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 6 and the carboxamide compound is penthiopyrad.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 6 and the carboxamide compound is boscalid.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 6 and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 6 and the carboxamide compound is penflufen.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 6 and the carboxamide compound is fluopyram.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 6 and the carboxamide compound is sedaxane.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 6 and the carboxamide compound is penthiopyrad.

TABLE 6

| No | detailed description | plant | Literature |
|---|---|---|---|
| T6-1 | drought tolerance | alfalfa | A*, B*, F* |
| T6-2 | drought tolerance | barley | A*, B*, C* |
| T6-3 | drought tolerance | canola | A*, B*, F* |
| T6-4 | drought tolerance | maize | A*, B*, C*, F* |
| T6-5 | drought tolerance | cotton | A*, B*, C*, F* |
| T6-6 | drought tolerance | pomefruit | A*, B* |
| T6-7 | drought tolerance | potato | A*, B*, C* |
| T6-8 | drought tolerance | rapeseed | A*, B*, C* |
| T6-9 | drought tolerance | rice | A*, B*, C*, F* |
| T6-10 | drought tolerance | soybean | A*, B*, F* |
| T6-11 | drought tolerance | sugarbeet | A*, B* |
| T6-12 | drought tolerance | sugarcane | A*, B*, F* |
| T6-13 | drought tolerance | sunflower | A*, B* |
| T6-14 | drought tolerance | tomato | A*, B*, C* |
| T6-15 | drought tolerance | wheat | A*, B*, C*, F* |
| T6-16 | tolerance to high salinity | alfalfa | A*, B* |
| T6-17 | tolerance to high salinity | barley | A*, B* |
| T6-18 | tolerance to high salinity | canola | A*, B* |
| T6-19 | tolerance to high salinity | maize | A*, D* |
| T6-20 | tolerance to high salinity | cotton | A*, D* |
| T6-21 | tolerance to high salinity | pomefruit | A*, D* |
| T6-22 | tolerance to high salinity | potato | A*, D* |
| T6-23 | tolerance to high salinity | rapeseed | A*, D* |
| T6-24 | tolerance to high salinity | rice | A*, D*, U.S. Pat. No. 7,034,139, WO |
| | | | 2001/30990 |
| T6-25 | tolerance to high salinity | soybean | A*, D* |
| T6-26 | tolerance to high salinity | sugarbeet | A*, D* |
| T6-27 | tolerance to high salinity | sugarcane | A*, D* |
| T6-28 | tolerance to high salinity | sunflower | A*, D* |
| T6-29 | tolerance to high salinity | tomato | A*, D* |
| T6-30 | tolerance to high salinity | wheat | A*, D* |
| T6-31 | low temperature tolerance | alfalfa | A*, E* |
| T6-32 | low temperature tolerance | barley | A* |
| T6-33 | low temperature tolerance | canola | A* |
| T6-34 | low temperature tolerance | maize | A*, E* |
| T6-35 | low temperature tolerance | cotton | A*, E* |
| T6-36 | low temperature tolerance | pomefruit | A* |
| T6-37 | low temperature tolerance | potato | A* |
| T6-38 | low temperature tolerance | rapeseed | A*, E* |
| T6-39 | low temperature tolerance | rice | A*, E* |
| T6-40 | low temperature tolerance | soybean | A*, E* |
| T6-41 | low temperature tolerance | sugarbeet | A* |
| T6-42 | low temperature tolerance | sugarcane | A* |
| T6-43 | low temperature tolerance | sunflower | A* |
| T6-44 | low temperature tolerance | tomato | A* |
| T6-45 | low temperature tolerance | wheat | A*, E* |
| T6-46 | low nitrogen supply tolerance | alfalfa | A* |
| T6-47 | low nitrogen supply tolerance | barley | A* |
| T6-48 | low nitrogen supply tolerance | canola | A* |
| T6-49 | low nitrogen supply tolerance | maize | A* |
| T6-50 | low nitrogen supply tolerance | cotton | A* |
| T6-51 | low nitrogen supply tolerance | pomefruit | A* |
| T6-52 | low nitrogen supply tolerance | potato | A* |
| T6-53 | low nitrogen supply tolerance | rapeseed | A* |
| T6-54 | low nitrogen supply tolerance | rice | A* |
| T6-55 | low nitrogen supply tolerance | soybean | A* |
| T6-56 | low nitrogen supply tolerance | sugarbeet | A* |
| T6-57 | low nitrogen supply tolerance | sugarcane | A* |
| T6-58 | low nitrogen supply tolerance | sunflower | A* |
| T6-59 | low nitrogen supply tolerance | tomato | A* |
| T6-60 | low nitrogen supply tolerance | wheat | A* |

A* referes to WO 2000/04173, WO 2007/131699 and US 2008/0229448.
B* referes to WO 2005/48693.
C* referes to WO 2007/20001.
D* referes to U.S. Pat. No. 7,256,326.
E* referes to U.S. Pat. No. 4,731,499.
F* refers to WO 2008/002480.

In a more preferred embodiment, the present invention relates to a method of controlling harmful fungi and/or increasing the health of cultivated plants by treating cultivated plants, parts of such plants, plant propagation materials, or at their locus of growth with a carboxamide compound selected from boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad, wherein the plant is a plant, which is listed in table 7.

In a more preferred embodiment, the present invention relates to a method of controlling harmful fungi and/or increasing the health of cultivated plants by treating plant propagation materials, preferably seeds with a carboxamide compound selected from boscalid, N-(3′,4′,5′-trifluorobiphenyl-2-yl)-3-difluoromethyl-1-methyl-1H-pyrazole-4-carboxamide, penflufen, fluopyram sedaxane, penthiopyrad carboxin, fenfuram, flutolanil; mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide, penflufen, fluopyram, sedaxane and penthiopyrad, wherein the plant corresponds to a row of table 7.

In another more preferred embodiment, the present invention relates to a method of controlling harmful fungi and/or increasing the health of cultivated plants by treating cultivated plants, parts of such plants or at their locus of growth with a carboxamide compound selected from boscalid, N-(3′,4′,5′-trifluorobiphenyl-2-yl)-3-difluoromethyl-1-methyl-1H-pyrazole-4-carboxamide bixafen, fluopyram, isopyrazam, penthiopyrad, flutolanil, furametpyr, mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide bixafen, fluopyram, isopyrazam and penthiopyrad, wherein the plant corresponds to a row of table 7.

In a most preferred embodiment, the present invention relates to a method of controlling harmful fungi and/or increasing the health of plants by treating cultivated plants, parts of such plants or at their locus of growth with a carboxamide compound, wherein the plant corresponds to a row of table 7 and the carboxamide compound is boscalid.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 7 and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 7 and the carboxamide compound is bixafen.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 7 and the carboxamide compound is fluopyram.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 7 and the carboxamide compound is isopyrazam.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 7 and the carboxamide compound is penthiopyrad.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 7 and the carboxamide compound is boscalid.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 7 and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 7 and the carboxamide compound is penflufen.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 7 and the carboxamide compound is fluopyram.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 7 and the carboxamide compound is sedaxane.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 7 and the carboxamide compound is penthiopyrad.

In a utmost preferred embodiment, the present invention relates to a method of controlling harmful fungi and/or increasing the health of plants by treating cultivated plants, parts of such plants or at their locus of growth with a carboxamide compound, wherein the plant is selected from T7-5, T7-6, T7-7, T7-8 and T7-9 of table 7 and the carboxamide compound is boscalid.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T7-5, T7-6, T7-7, T7-8 and T7-9 of table 7 and the carboxamide compound is N-(3′,4′,5′-trifluorobiphenyl-2-yl)-3-difluoromethyl-1-methyl-1H-pyrazole-4-carboxamide.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T7-5, T7-6, T7-7, T7-8 and T7-9 of table 7 and the carboxamide compound is bixafen.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T7-5, T7-6, T7-7, T7-8 and T7-9 of table 7 and the carboxamide compound is fluopyram.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T7-5, T7-6, T7-7, T7-8 and T7-9 of table 7 and the carboxamide compound is isopyrazam.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T7-5, T7-6, T7-7, T7-8 and T7-9 of table 7 and the carboxamide compound is penthiopyrad.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T7-5, T7-6, T7-7, T7-8 and T7-9 of table 7 and the carboxamide compound is boscalid.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T7-5, T7-6, T7-7, T7-8 and T7-9 of table 7 and the carboxamide compound is N-(3′,4′,5′-trifluorobiphenyl-2-yl)-3-difluoromethyl-1-methyl-1H-pyrazole-4-carboxamide.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T7-5, T7-6, T7-7, T7-8 and T7-9 of table 7 and the carboxamide compound is penflufen.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T7-5, T7-6, T7-7, T7-8 and T7-9 of table 7 and the carboxamide compound is fluopyram.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T7-5, T7-6, T7-7, T7-8 and T7-9 of table 7 and the carboxamide compound is sedaxane.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T7-5, T7-6, T7-7, T7-8 and T7-9 of table 7 and the carboxamide compound is penthiopyrad.

TABLE 7

| No | detailed description | plant | Literature |
|---|---|---|---|
| T7-1 | drought tolerance | maize | A*, B*, C* |
| T7-2 | drought tolerance | canola | A*, B*, C* |
| T7-3 | drought tolerance | cotton | A*, B*, C* |
| T7-4 | drought tolerance | rapeseed | A*, B*, C* |
| T7-5 | drought tolerance | rice | A*, B*, C* |
| T7-6 | drought tolerance | soybean | A*, B* |
| T7-7 | drought tolerance | wheat | A*, B*, C* |
| T7-8 | tolerance to high salinity | rice | A*, D*, U.S. Pat. No. |
| | | | 7,034,139, WO |
| | | | 2001/30990 |
| T7-9 | tolerance to high salinity | tomato | A*, D* |
| T7-10 | low nitrogen supply | canola | A* |
| | tolerance | | |
| T7-11 | low nitrogen supply | maize | A* |
| | tolerance | | |

A* referes to WO 2000/04173, WO 2007/131699 and US 2008/0229448.
B* referes to WO 2005/48693.
C* referes to WO 2007/20001.
D* referes to U.S. Pat. No. 7,256,326.
E* referes to U.S. Pat. No. 4,731,499.

In a further one preferred embodiment, the present invention relates to a method of controlling harmful fungi and/or increasing the health of cultivated plants by treating cultivated plants, parts of such plants, plant propagation materials, or at their locus of growth with a carboxamide compound selected from boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad, wherein the plant is a plant, which shows improved maturation, preferably fruit ripening, early maturity and delayed softening.

In a more preferred embodiment, the present invention relates to a method of controlling harmful fungi and/or increasing the health of cultivated plants by treating cultivated plants, parts of such plants, plant propagation materials, or at their locus of growth a carboxamide compound selected from boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad, wherein the plant is a plant, which corresponds to a row of table 8 or 8a.

In a more preferred embodiment, the present invention relates to a method of controlling harmful fungi and/or increasing the health of cultivated plants by treating plant propagation materials, preferably seeds with a carboxamide compound selected from boscalid, N-(3′,4′,5′-trifluorobiphenyl-2-yl)-3-difluoromethyl-1-methyl-1H-pyrazole-4-carboxamide, penflufen, fluopyram sedaxane, penthiopyrad carboxin, fenfuram, flutolanil; mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide, penflufen, fluopyram, sedaxane and penthiopyrad, wherein the plant corresponds to a row of table 8 or 8a.

In another more preferred embodiment, the present invention relates to a method of controlling harmful fungi and/or increasing the health of cultivated plants by treating cultivated plants, parts of such plants or at their locus of growth with a carboxamide compound selected from boscalid, N-(3′,4′,5′-trifluorobiphenyl-2-yl)-3-difluoromethyl-1-methyl-1H-pyrazole-4-carboxamide bixafen, fluopyram, isopyrazam, penthiopyrad, flutolanil, furametpyr, mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide bixafen, fluopyram, isopyrazam and penthiopyrad, wherein the plant corresponds to a row of table 8 or 8a.

In a most preferred embodiment, the present invention relates to a method of controlling harmful fungi and/or increasing the health of plants by treating cultivated plants, parts of such plants or at their locus of growth with a carboxamide compound, wherein the plant corresponds to a row of table 8 or 8a and the carboxamide compound is boscalid.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 8 or 8a and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 8 or 8a and the carboxamide compound is bixafen.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 8 or 8a and the carboxamide compound is fluopyram.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 8 or 8a and the carboxamide compound is isopyrazam.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 8 or 8a and the carboxamide compound is penthiopyrad.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 8 or 8a and the carboxamide compound is boscalid.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 8 or 8a and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 8 or 8a and the carboxamide compound is penflufen.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 8 or 8a and the carboxamide compound is fluopyram.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 8 or 8a and the carboxamide compound is sedaxane.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 8 or 8a and the carboxamide compound is penthiopyrad.

In a utmost preferred embodiment, the present invention relates to a method of controlling harmful fungi and/or increasing the health of plants by treating cultivated plants, parts of such plants or at their locus of growth with a carboxamide compound, wherein the plant is T8-1 of table 8 and the carboxamide compound is boscalid.

In another utmost preferred embodiment, the present invention relates to a method of controlling harmful fungi and/or increasing the health of plants by treating cultivated plants, parts of such plants or at their locus of growth with a carboxamide compound, wherein the plant is T8-1 of table 8 and the carboxamide compound is N-(3′,4′,5′-trifluorobiphenyl-2-yl)-3-difluoromethyl-1-methyl-1H-pyrazole-4-carboxamide.

In another utmost preferred embodiment, the present invention relates to a method of controlling harmful fungi and/or increasing the health of plants by treating cultivated plants, parts of such plants or at their locus of growth with a carboxamide compound, wherein the plant is T8-1 of table 8 and the carboxamide compound is bixafen.

In another utmost preferred embodiment, the present invention relates to a method of controlling harmful fungi and/or increasing the health of plants by treating cultivated plants, parts of such plants or at their locus of growth with a carboxamide compound, wherein the plant is T8-1 of table 8 and the carboxamide compound is fluopyram.

In another utmost preferred embodiment, the present invention relates to a method of controlling harmful fungi and/or increasing the health of plants by treating cultivated plants, parts of such plants or at their locus of growth with a carboxamide compound, wherein the plant is T8-1 of table 8 and the carboxamide compound is isopyrazam.

In another utmost preferred embodiment, the present invention relates to a method of controlling harmful fungi and/or increasing the health of plants by treating cultivated plants, parts of such plants or at their locus of growth with a carboxamide compound, wherein the plant is T8-1 of table 8 and the carboxamide compound is penthiopyrad.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is T8-1 of table 8 and the carboxamide compound is boscalid.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is T8-1 of table 8 and the carboxamide compound is N-(3′,4′,5′-trifluorobiphenyl-2-yl)-3-difluoromethyl-1-methyl-1H-pyrazole-4-carboxamide.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is T8-1 of table 8 and the carboxamide compound is penflufen.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is T8-1 of table 8 and the carboxamide compound is fluopyram.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is T8-1 of table 8 and the carboxamide compound is sedaxane.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is T8-1 of table 8 and the carboxamide compound is penthiopyrad.

TABLE 8

| No | detailed description | plant | Literature |
|---|---|---|---|
| T8-1 | fruit ripening | tomato | U.S. Pat. No. 5,952,546, |
| | | | U.S. Pat. No. 5,512,466, WO |
| | | | 1997/001952, WO 1995/035387 |
| | | | WO 1992/008798, |
| | | | Plant Cell. 1989; 1(1): 53-63. |
| T8-2 | fruit ripening | papaya | U.S. Pat. No. 5,767,376, |
| | | | U.S. Pat. No. 7,084,321 |
| T8-3 | fruit ripening | pepper | Plant Molecular Biology, |
| | | | Volume 50, 2002, Number 3 |
| T8-4 | fruit ripening | melon | WO 1995/035387 |
| T8-5 | fruit ripening | strawberry | WO 1995/035387 |
| T8-6 | fruit ripening | raspberry | WO 1995/035387 |

TABLE 8a

| No | Plant | Event | Company |
|---|---|---|---|
| T8a-1 | Cucumis melo (Melon) | A, B | Agritope Inc. |
| T8a-2 | Lycopersicon esculentum | 66 | Florigene |
| | (Tomato) | | Pty Ltd. |
| T8a-3 | Lycopersicon esculentum | 1345-4 | DNA Plant |
| | (Tomato) | | Technology |
| | | | Corporation |
| T8a-4 | Lycopersicon esculentum | 35 1 N | Agritope Inc. |
| | (Tomato) | | |
| T8a-5 | Lycopersicon esculentum | 8338 | Monsanto |
| | (Tomato) | | Company |
| T8a-6 | Lycopersicon esculentum | B, Da, F | Zeneca Seeds |
| | (Tomato) | | |
| T8a-7 | Lycopersicon esculentum | FLAVR SAVR | Calgene Inc. |
| | (Tomato) | | |

In a further one preferred embodiment, the present invention relates to a method of controlling harmful fungi and/or increasing the health of cultivated plants by treating cultivated plants, parts of such plants, plant propagation materials, or at their locus of growth with a carboxamide compound selected from boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad, wherein the plant is a transgenic plant, which has modified content in comparison to wildtype plants, preferably increased vitamin content, altered oil content, nicotine reduction, increased or reduced amino acid content, protein alteration, modified starch content, enzyme alteration, altered flavonoid content and reduced allergens (hypoallergenic plants), most preferably increased vitamin content, altered oil content, nicotine reduction, increased lysine content, amylase alteration, amylopectin alteration.

In a more preferred embodiment, the present invention relates to a method of controlling harmful fungi and/or increasing the health of cultivated plants by treating cultivated plants, parts of such plants, plant propagation materials, or at their locus of growth with a carboxamide compound selected from boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad, wherein the plant is a plant, which corresponds to a row of table 9.

In a more preferred embodiment, the present invention relates to a method of controlling harmful fungi and/or increasing the health of cultivated plants by treating plant propagation materials, preferably seeds with carboxamide compound selected from boscalid, N-(3′,4′,5′-trifluorobiphenyl-2-yl)-3-difluoromethyl-1-methyl-1H-pyrazole-4-carboxamide, penflufen, fluopyram sedaxane, penthiopyrad carboxin, fenfuram, flutolanil; mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide, penflufen, fluopyram, sedaxane and penthiopyrad, wherein the plant corresponds to a row of table 9.

In another more preferred embodiment, the present invention relates to a method of controlling harmful fungi and/or increasing the health of cultivated plants by treating cultivated plants, parts of such plants or at their locus of growth with a carboxamide compound selected from boscalid, N-(3′,4′,5′-trifluorobiphenyl-2-yl)-3-difluoromethyl-1-methyl-1H-pyrazole-4-carboxamide bixafen, fluopyram, isopyrazam, penthiopyrad, flutolanil, furametpyr, mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide, bixafen, fluopyram, isopyrazam and penthiopyrad, wherein the plant corresponds to a row of table 9.

In a most preferred embodiment, the present invention relates to a method of controlling harmful fungi and/or increasing the health of plants by treating cultivated plants, parts of such plants or at their locus of growth with a carboxamide compound, wherein the plant corresponds to a row of table 9 and the carboxamide compound is boscalid.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 9 and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 9 and the carboxamide compound is bixafen.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 9 and the carboxamide compound is fluopyram.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 9 and the carboxamide compound is isopyrazam.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 9 and the carboxamide compound is penthiopyrad.

In another most preferred embodiment, the present invention relates to a method of controlling harmful fungi and/or increasing the health of plants by treating cultivated plants, parts of such plants or at their locus of growth with a carboxamide compound, wherein the plant corresponds to row T9-48 of table 9 and the carboxamide compound is selected from the group consisting of boscalid, N-(3′,4′,5′-trifluorobiphenyl-2-yl)-3-difluoromethyl-1-methyl-1H-pyrazole-4-carboxamide, bixafen, fluopyram, isopyrazam and penthiopyrad.

In another most preferred embodiment, the present invention relates to a method of controlling harmful fungi and/or increasing the health of plants by treating cultivated plants, parts of such plants or at their locus of growth with a carboxamide compound, wherein the plant corresponds to row T9-49 of table 9 and the carboxamide compound is selected from the group consisting of boscalid, N-(3′,4′,5′-trifluorobiphenyl-2-yl)-3-difluoromethyl-1-methyl-1′-1-pyrazole-4-carboxamide, bixafen, fluopyram, isopyrazam and penthiopyrad.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 9 and the carboxamide compound is boscalid.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 9 and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 9 and the carboxamide compound is penflufen.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 9 and the carboxamide compound is fluopyram.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 9 and the carboxamide compound is sedaxane.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 9 and the carboxamide compound is penthiopyrad.

TABLE 9

| No | detailed description | plant | Literature/commercial plants |
|---|---|---|---|
| T9-1 | increased Vitamin A content | tomato | U.S. Pat. No. 6,797,498 |
| T9-2 | increased Vitamin A content | rice | “Golden rice”. |
| | | | Science 287, 303-305. |
| T9-3 | increased Vitamin E content | canola | U.S. Pat. No. 7,348,167, US 11/170,711 (application) |
| T9-4 | increased Vitamin E content | barley | US 11/170,711 (application) |
| T9-5 | increased Vitamin E content | maize | US 11/170,711 (application) |
| T9-6 | increased Vitamin E content | rice | US 11/170,711 (application) |
| T9-7 | increased Vitamin E content | rye | US 11/170,711 (application) |
| T9-8 | increased Vitamin E content | potato | U.S. Pat. No. 7,348,167 |
| T9-9 | increased Vitamin E content | soybean | U.S. Pat. No. 7,348,167 |
| T9-10 | increased Vitamin E content | sunflower | U.S. Pat. No. 7,348,167 |
| T9-11 | increased Vitamin E content | wheat | US 11/170,711 (application) |
| T9-12 | decreased nicotine content | tobacco | US 2006/0185684, WO 2005/000352, WO |
| | | | 2007/064636 |
| T9-13 | amylase alteration | maize | “AmylaseTM” |
| T9-14 | amylopectin alteration | potato | U.S. Pat. No. 6,784,338, WO 1997/044471 |
| T9-15 | amylopectin alteration | maize | US 20070261136 |
| T9-16 | modified oil content | balsam pear | A* |
| T9-17 | modified oil content | canola | U.S. Pat. No. 5,850,026, U.S. Pat. No. 6,441,278, U.S. Pat. No. 5,723,761 |
| T9-18 | modified oil content | catalpa | A* |
| T9-19 | modified oil content | cattail | A* |
| T9-20 | modified oil content | maize | A*, US 2006/0075515, U.S. Pat. No. 7,294,759 |
| T9-21 | modified oil content | cotton | U.S. Pat. No. 6,974,898, WO 2001/079499 |
| T9-22 | modified oil content | grape | A* |
| T9-23 | modified oil content | rapeseed | U.S. Pat. No. 5,723,761 |
| T9-24 | modified oil content | rice | A* |
| T9-25 | modified oil content | soybean | A*, U.S. Pat. No. 6,380,462, U.S. Pat. No. 6,365,802, |
| | | | “Vistive II”, “Vistsive III” |
| T9-26 | modified oil content | safflower | U.S. Pat. No. 6,084,164 |
| T9-27 | modified oil content | sunflower | A*, U.S. Pat. No. 6,084,164 |
| T9-28 | modified oil content | wheat | A* |
| T9-29 | modified oil content | vernonia | A* |
| T9-30 | hypoallergenic modification | soybean | U.S. Pat. No. 6,864,362 |
| T9-31 | increased lysine content | canola | Bio/Technology 13, 577-582 (1995) |
| T9-32 | increased lysine content | maize | “Mavera high value corn” |
| T9-33 | increased lysine content | soybean | Bio/Technology 13, 577-582 (1995) |
| T9-34 | altered starch content | maize | U.S. Pat. No. 7,317,146, EP 1105511 |
| T9-35 | altered starch content | rice | U.S. Pat. No. 7,317,146, EP 1105511 |
| T9-36 | altered starch content | wheat | EP 1105511 |
| T9-37 | altered starch content | barley | EP 1105511 |
| T9-38 | altered starch content | rye | EP 1105511 |
| T9-39 | altered starch content | oat | EP 1105511 |
| T9-40 | altered fllavonoid content | alfalfa | WO 2000/04175 |
| T9-41 | altered fllavonoid content | apple | WO 2000/04175 |
| T9-42 | altered fllavonoid content | bean | WO 2000/04175 |
| T9-43 | altered fllavonoid content | maize | WO 2000/04175 |
| T9-44 | altered fllavonoid content | grape | WO 2000/04175 |
| T9-45 | altered fllavonoid content | pea | WO 2000/04175 |
| T9-46 | altered fllavonoid content | tomato | WO 2000/04175 |
| T9-47 | increased protein content | soybean | “Mavera high value soybeans” |
| T9-48 | amylopectin alteration | potato | B* |
| T9-49 | altered starch content | potato | C* |

A* refers to U.S. Pat. No. 7,294,759 and U.S. Pat. No. 7,157,621.
B* refers to the potato plant variety submitted for variety registration with the Community Plant Variety Office (CPVO), 3, boulevard Marechal Foch, BP 10121, FR-49101 Angers Cedex 02, France and having the CPVO file number 20031520.
C* refers to the potato plant variety submitted for variety registration with the Community Plant Variety Office (CPVO), 3, boulevard Marechal Foch, BP 10121, FR-49101 Angers Cedex 02, France and having the CPVO file number 20082534.

In a more preferred embodiment, the present invention relates to a method of controlling harmful fungi and/or increasing the health of cultivated plants by treating cultivated plants, parts of such plants, plant propagation materials, or at their locus of growth with a carboxamide compound selected from boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad, wherein the plant is a plant, which corresponds to a row of table 10.

In a more preferred embodiment, the present invention relates to a method of controlling harmful fungi and/or increasing the health of cultivated plants by treating plant propagation materials, preferably seeds with a carboxamide compound selected from boscalid, N-(3′,4′,5′-trifluorobiphenyl-2-yl)-3-difluoromethyl-1-methyl-1H-pyrazole-4-carboxamide, penflufen, fluopyram sedaxane, penthiopyrad carboxin, fenfuram, flutolanil; mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide, penflufen, fluopyram, sedaxane and penthiopyrad, wherein the plant corresponds to a row of table 10.

In another more preferred embodiment, the present invention relates to a method of controlling harmful fungi and/or increasing the health of cultivated plants by treating cultivated plants, parts of such plants or at their locus of growth with a carboxamide compound selected from boscalid, N-(3′,4′,5′-trifluorobiphenyl-2-yl)-3-difluoromethyl-1-methyl-1H-pyrazole-4-carboxamide bixafen, fluopyram, isopyrazam, penthiopyrad, flutolanil, furametpyr, mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide bixafen, fluopyram, isopyrazam and penthiopyrad, wherein the plant corresponds to a row of table 10.

In a most preferred embodiment, the present invention relates to a method of controlling harmful fungi and/or increasing the health of plants by treating cultivated plants, parts of such plants or at their locus of growth with a carboxamide compound, wherein the plant corresponds to a row of table 10 and the carboxamide compound is boscalid.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 10 and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 10 and the carboxamide compound is bixafen.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 10 and the carboxamide compound is fluopyram.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 10 and the carboxamide compound is isopyrazam.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 10 and the carboxamide compound is penthiopyrad.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 10 and the carboxamide compound is boscalid.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 10 and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 10 and the carboxamide compound is penflufen.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 10 and the carboxamide compound is fluopyram.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 10 and the carboxamide compound is sedaxane.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 10 and the carboxamide compound is penthiopyrad.

In a utmost preferred embodiment, the present invention relates to a method of controlling harmful fungi and/or increasing the health of plants by treating cultivated plants, parts of such plants or at their locus of growth with a carboxamide compound, wherein the plant is selected from T10-1, T10-2, T10-6 and T10-10 of table 10 and the carboxamide compound is boscalid.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T10-1, T10-2, T10-6 and T10-10 of table 10 and the carboxamide compound is N-(3′,4′,5′-trifluorobiphenyl-2-yl)-3-difluoromethyl-1-methyl-1H-pyrazole-4-carboxamide.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T10-1, T10-2, T10-6 and T10-10 of table 10 and the carboxamide compound is bixafen.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T10-1, T10-2, T10-6 and T10-10 of table 10 and the carboxamide compound is fluopyram.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T10-1, T10-2, T10-6 and T10-10 of table 10 and the carboxamide compound is isopyrazam.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T10-1, T10-2, T10-6 and T10-10 of table 10 and the carboxamide compound is penthiopyrad.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T10-1, T10-2, T10-6 and T10-10 of table 10 and the carboxamide compound is boscalid.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T10-1, T10-2, T10-6 and T10-10 of table 10 and the carboxamide compound is N-(3′,4′,5′-trifluorobiphenyl-2-yl)-3-difluoromethyl-1-methyl-1H-pyrazole-4-carboxamide.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T10-1, T10-2, T10-6 and T10-10 of table 10 and the carboxamide compound is penflufen.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T10-1, T10-2, T10-6 and T10-10 of table 10 and the carboxamide compound is fluopyram.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T10-1, T10-2, T10-6 and T10-10 of table 10 and the carboxamide compound is sedaxane.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T10-1, T10-2, T10-6 and T10-10 of table 10 and the carboxamide compound is penthiopyrad.

TABLE 10

| No | detailed description | plant | Literature/commercial plants |
|---|---|---|---|
| T10-1 | increased Vitamin A content | tomato | U.S. Pat. No. 6,797,498 |
| T10-2 | increased Vitamin A content | rice | “Golden rice”. |
| | | | Science 287, 303-305. |
| T10-3 | increased Vitamin E content | canola | U.S. Pat. No. 7,348,167, US 11/170,711 (application) |
| T10-4 | decreased nicotine content | tobacco | US 20060185684, WO 2005/000352, WO |
| | | | 2007/064636 |
| T10-5 | amylase alteration | maize | “AmylaseTM” |
| T10-6 | amylopectin alteration | potato | U.S. Pat. No. 6,784,338, WO 1997/044471 |
| T10-7 | modified oil content | canola | U.S. Pat. No. 5,850,026, U.S. Pat. No. 6,441,278, U.S. Pat. No. 5,723,761 |
| T10-8 | modified oil content | rapeseed | U.S. Pat. No. 5,723,761 |
| T10-9 | modified oil content | safflower | U.S. Pat. No. 6,084,164 |
| T10-10 | modified oil content | soybean | A*, U.S. Pat. No. 6,380,462, U.S. Pat. No. 6,365,802; |
| | | | “Vistive II”, “Vistsive III” |
| T10-11 | increased protein content | soybean | “Mavera high value soybeans” |
| T10-12 | increased lysine content | maize | “Mavera high value corn” |

A* refers to U.S. Pat. No. 7,294,759 and U.S. Pat. No. 7,157,621.

In a further one preferred embodiment, the present invention relates to a method of controlling harmful fungi and/or increasing the health of cultivated plants by treating cultivated plants, parts of such plants, plant propagation materials, or at their locus of growth with a carboxamide compound selected from boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad, wherein the plant is a plant, which shows improved nutrient utilization, preferably the uptake, assimilation and metabolism of nitrogen and phosphorous.

In a more preferred embodiment, the present invention relates to a method of controlling harmful fungi and/or increasing the health of cultivated plants by treating cultivated plants, parts of such plants, plant propagation materials, or at their locus of growth with a carboxamide compound selected from boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad, wherein the plant is a plant, which corresponds to a row of table 11.

In a more preferred embodiment, the present invention relates to a method of controlling harmful fungi and/or increasing the health of cultivated plants by treating plant propagation materials, preferably seeds with a carboxamide compound selected from boscalid, N-(3′,4′,5′-trifluorobiphenyl-2-yl)-3-difluoromethyl-1-methyl-1H-pyrazole-4-carboxamide, penflufen, fluopyram sedaxane, penthiopyrad carboxin, fenfuram, flutolanil; mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide, penflufen, fluopyram, sedaxane and penthiopyrad, wherein the plant corresponds to a row of table 11.

In another more preferred embodiment, the present invention relates to a method of controlling harmful fungi and/or increasing the health of cultivated plants by treating cultivated plants, parts of such plants or at their locus of growth with a carboxamide compound selected from boscalid, N-(3′,4′,5′-trifluorobiphenyl-2-yl)-3-difluoromethyl-1-methyl-1H-pyrazole-4-carboxamide, bixafen, fluopyram, isopyrazam, penthiopyrad, flutolanil, furametpyr, mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide bixafen, fluopyram, isopyrazam and penthiopyrad, wherein the plant corresponds to a row of table 11.

In a most preferred embodiment, the present invention relates to a method of controlling harmful fungi and/or increasing the health of plants by treating cultivated plants, parts of such plants or at their locus of growth with a carboxamide compound, wherein the plant corresponds to a row of table 11 and the carboxamide compound is boscalid.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 11 and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 11 and the carboxamide compound is bixafen.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 11 and the carboxamide compound is fluopyram.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 11 and the carboxamide compound is isopyrazam.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 11 and the carboxamide compound is penthiopyrad.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 11 and the carboxamide compound is boscalid.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 11 and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 11 and the carboxamide compound is penflufen.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 11 and the carboxamide compound is fluopyram.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 11 and the carboxamide compound is sedaxane.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 11 and the carboxamide compound is penthiopyrad.

In a utmost preferred embodiment, the present invention relates to a method of controlling harmful fungi and/or increasing the health of plants by treating cultivated plants, parts of such plants or at their locus of growth with a carboxamide compound, wherein the plant is selected from T11-3 and T11-4 of table 11 and the carboxamide compound is boscalid.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T11-3 and T11-4 of table 11 and the carboxamide compound is N-(3′,4′,5′-trifluorobiphenyl-2-yl)-3-difluoromethyl-1-methyl-1H-pyrazole-4-carboxamide.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T11-3 and T11-4 of table 11 and the carboxamide compound is bixafen.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T11-3 and T11-4 of table 11 and the carboxamide compound is fluopyram.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T11-3 and T11-4 of table 11 and the carboxamide compound is isopyrazam.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T11-3 and T11-4 of table 11 and the carboxamide compound is penthiopyrad.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T11-3 and T11-4 of table 11 and the carboxamide compound is boscalid.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T11-3 and T11-4 of table 11 and the carboxamide compound is N-(3′,4′,5′-trifluorobiphenyl-2-yl)-3-difluoromethyl-1-methyl-1H-pyrazole-4-carboxamide.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T11-3 and T11-4 of table 11 and the carboxamide compound is penflufen.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T11-3 and T11-4 of table 11 and the carboxamide compound is fluopyram.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T11-3 and T11-4 of table 11 and the carboxamide compound is sedaxane.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T11-3 and T11-4 of table 11 and the carboxamide compound is penthiopyrad.

TABLE 11

| No | detailed description | plant | Literature |
|---|---|---|---|
| T11-1 | nitrogen utilization (D*) | alfalfa | A*, B*, F* |
| T11-2 | nitrogen utilization (D*) | barley | A*, B* |
| T11-3 | nitrogen utilization (D*) | canola | A*, B*, F* |
| T11-4 | nitrogen utilization (D*) | maize | A*, B*, F* |
| T11-5 | nitrogen utilization (D*) | cotton | B*, F* |
| T11-6 | nitrogen utilization (D*) | potato | B*, E*, F* |
| T11-7 | nitrogen utilization (D*) | rapeseed | B* |
| T11-8 | nitrogen utilization (D*) | rice | A*, B*, F* |
| T11-9 | nitrogen utilization (D*) | soybean | A*, B*, F* |
| T11-10 | nitrogen utilization (D*) | sugarbeet | B*, E* |
| T11-11 | nitrogen utilization (D*) | sugarcane | B*, E* |
| T11-12 | nitrogen utilization (D*) | sunflower | B* |
| T11-13 | nitrogen utilization (D*) | tobacco | E*, F* |
| T11-14 | nitrogen utilization (D*) | tomato | B*, F* |
| T11-15 | nitrogen utilization (D*) | wheat | A*, B*, F* |
| T11-16 | phosphorous utilization (D*) | alfalfa | C* |
| T11-17 | phosphorous utilization (D*) | barley | C* |
| T11-18 | phosphorous utilization (D*) | canola | C* |
| T11-19 | phosphorous utilization (D*) | maize | C* |
| T11-20 | phosphorous utilization (D*) | cotton | C* |
| T11-21 | phosphorous utilization (D*) | potato | U.S. Pat. No. 7,417,181, C* |
| T11-22 | phosphorous utilization (D*) | rapeseed | C* |
| T11-23 | phosphorous utilization (D*) | rice | C* |
| T11-24 | phosphorous utilization (D*) | soybean | C* |
| T11-25 | phosphorous utilization (D*) | sugarbeet | C* |
| T11-26 | phosphorous utilization (D*) | sugarcane | C* |
| T11-27 | phosphorous utilization (D*) | sunflower | C* |
| T11-28 | phosphorous utilization (D*) | tomato | U.S. Pat. No. 7,417,181, C* |
| T11-29 | phosphorous utilization (D*) | wheat | C* |
| T11-30 | low nitrogen supply tolerance | canola | G* |
| T11-31 | low nitrogen supply tolerance | maize | G* |

A* refers to U.S. Pat. No. 6,084,153.
B* referes to U.S. Pat. No. 5,955,651 and U.S. Pat. No. 6,864,405.
C* refers to US 10/898,322 (application).
D* the term “utilization” refers to the improved nutrient uptake, assimilation or metabolism.
E* refers to WO 1995/009911.
F* refers to WO 1997/030163.
G* referes to WO 2000/04173, WO 2007/131699 and US 2008/0229448

In a further one preferred embodiment, the present invention relates to a method of controlling harmful fungi and/or increasing the health of plants by treating cultivated plants parts of such plants, plant propagation materials, or at their locus of growth with a carboxamide compound selected from boscalid, bixafen, penflufen, fluopyram, sedaxane, isopyrazam and penthiopyrad, wherein the plant is a plant selected from the group consisting of cotton, fiber plants (e.g. palms) and trees, preferably a cotton plant, which produces higher quality fiber, preferably improved micronaire of the fiber, increased strength, improved staple length, improved length unifomity and color of the fibers.

In a more preferred embodiment, the present invention relates to a method of controlling harmful fungi and/or increasing the health of cotton plants by treating cultivated plants parts of such plants, plant propagation materials, or at their locus of growth with a carboxamide compound selected from boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad.

In a more preferred embodiment, the present invention relates to a method of controlling harmful fungi and/or increasing the health of cotton plants by treating plant propagation materials, preferably seeds with a carboxamide compound selected from boscalid, N-(3′,4′,5′-trifluorobiphenyl-2-yl)-3-difluoromethyl-1-methyl-1H-pyrazole-4-carboxamide, penflufen, fluopyram sedaxane, penthiopyrad carboxin, fenfuram, flutolanil; mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide, penflufen, fluopyram, sedaxane and penthiopyrad.

In another more preferred embodiment, the present invention relates to a method of controlling harmful fungi and/or increasing the health of cotton plants by treating cultivated plants parts of such plants or at their locus of growth with a carboxamide compound selected from boscalid, N-(3′,4′,5′-trifluorobiphenyl-2-yl)-3-difluoromethyl-1-methyl-1H-pyrazole-4-carboxamide, bixafen, fluopyram, isopyrazam, penthiopyrad, flutolanil, furametpyr, mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide, bixafen, fluopyram, isopyrazam and penthiopyrad.

In a further one preferred embodiment, the present invention relates to a method of controlling harmful fungi and/or increasing the health of cultivated plants by treating cultivated plants, parts of such plants, plant propagation materials, or at their locus of growth with a carboxamide compound selected from boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad, wherein the plant is a plant, which is male sterile or has an other trait as mentioned in table 12a.

In a more preferred embodiment, the present invention relates to a method of controlling harmful fungi and/or increasing the health of cultivated plants by treating cultivated plants, parts of such plants, plant propagation materials, or at their locus of growth with a carboxamide compound selected from boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad, wherein the plant is a plant, which is listed in table 12 or 12a.

In a more preferred embodiment, the present invention relates to a method of controlling harmful fungi and/or increasing the health of cultivated plants by treating plant propagation materials, preferably seeds with a carboxamide compound selected from boscalid, N-(3′,4′,5′-trifluorobiphenyl-2-yl)-3-difluoromethyl-1-methyl-1H-pyrazole-4-carboxamide, penflufen, fluopyram sedaxane, penthiopyrad carboxin, fenfuram, flutolanil; mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide, penflufen, fluopyram, sedaxane and penthiopyrad, wherein the plant corresponds to a row of table 12 or 12a.

In another more preferred embodiment, the present invention relates to a method of controlling harmful fungi and/or increasing the health of cultivated plants by treating cultivated plants, parts of such plants or at their locus of growth with a carboxamide compound selected from boscalid, N-(3′,4′,5′-trifluorobiphenyl-2-yl)-3-difluoromethyl-1-methyl-1H-pyrazole-4-carboxamide, bixafen, fluopyram, isopyrazam, penthiopyrad, flutolanil, furametpyr, mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide bixafen, fluopyram, isopyrazam and penthiopyrad, wherein the plant corresponds to a row of table 12 or 12a.

In a most preferred embodiment, the present invention relates to a method of controlling harmful fungi and/or increasing the health of plants by treating cultivated plants, parts of such plants or at their locus of growth with a carboxamide compound, wherein the plant corresponds to a row of table 12 or 12a and the carboxamide compound is boscalid.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 12 or 12a and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 12 or 12a and the carboxamide compound is bixafen.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 12 or 12a and the carboxamide compound is fluopyram.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 12 or 12a and the carboxamide compound is isopyrazam.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 12 or 12a and the carboxamide compound is penthiopyrad.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 12 or 12a and the carboxamide compound is boscalid.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 12 or 12a and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 12 or 12a and the carboxamide compound is penflufen.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 12 or 12a and the carboxamide compound is fluopyram.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 12 or 12a and the carboxamide compound is sedaxane.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 12 or 12a and the carboxamide compound is penthiopyrad.

TABLE 12

| No | detailed description | plant | Literature |
|---|---|---|---|
| T12-1 | male sterility | canola | U.S. Pat. No. 6,720,481 |
| T12-2 | male sterility | maize | A*, B*, C* |
| T12-3 | male sterility | rice | B*, EP1135982 |
| T12-4 | male sterility | soybean | B*, C*, WO |
| | | | 1996/040949 |
| T12-5 | male sterility | sunflower | C* |
| T12-6 | male sterility | tomato | U.S. Pat. No. 7,345,222 |
| T12-7 | male sterility | wheat | B* |

A* refers to U.S. Pat. No. 6,281,348, U.S. Pat. No. 6,399,856, U.S. Pat. No. 7,230,168, U.S. Pat. No. 6,072,102.
B* refers to WO 2001/062889.
C* refers to WO 1996/040949.

TABLE 12a

| No | plant | Event | Company | Description |
|---|---|---|---|---|
| T12a-1 | Brassicanapus | MS1, RF1 => | Aventis Crop- | Male-sterility, fertility restoration, pollination control |
| | (Argentine | PGS1 | Science (formerly | system displaying glufosinate herbicide tolerance. |
| | Canola) | | Plant Genetic | MS lines contained the barnase gene from Bacillus |
| | | | Systems) | amyloliquefaciens, RF lines contained the barstar |
| | | | | gene from the same bacteria, and both lines contained |
| | | | | the phosphinothricin N-acetyltransferase |
| | | | | (PAT) encoding gene from Streptomyces hygroscopicus. |
| T12a-2 | Brassicanapus | MS1, RF2 => | Aventis Crop- | Male-sterility, fertility restoration, pollination control |
| | (Argentine | PGS2 | Science (formerly | system displaying glufosinate herbicide tolerance. |
| | Canola) | | Plant Genetic | MS lines contained the barnase gene from Bacillus |
| | | | Systems) | amyloliquefaciens, RF lines contained the barstar |
| | | | | gene from the same bacteria, and both lines contained |
| | | | | the phosphinothricin N-acetyltransferase |
| | | | | (PAT) encoding gene from Streptomyces hygroscopicus. |
| T12a-3 | Brassicanapus | MS8xRF3 | Bayer Crop- | Male-sterility, fertility restoration, pollination control |
| | (Argentine | | Science | system displaying glufosinate herbicide tolerance. |
| | Canola) | | (Aventis Crop- | MS lines contained the barnase gene from Bacillus |
| | | | Science | amyloliquefaciens, RF lines contained the barstar |
| | | | (AgrEvo)) | gene from the same bacteria, and both lines contained |
| | | | | the phosphinothricin N-acetyltransferase |
| | | | | (PAT) encoding gene from Streptomyces hygroscopicus. |
| T12a-4 | Brassicanapus | PHY14, | Aventis Crop- | Male sterility was via insertion of the barnase ribo- |
| | (Argentine | PHY35 | Science (formerly | nuclease gene from Bacillus amyloliquefaciens; |
| | Canola) | | Plant Genetic | fertility restoration by insertion of the barstar |
| | | | Systems) | RNase inhibitor; PPT resistance was via PPT- |
| | | | | acetyltransferase (PAT) from Streptomyces hygroscopicus. |
| T12a-4 | Brassicanapus | PHY36 | Aventis Crop- | Male sterility was via insertion of the barnase ribo- |
| | (Argentine | | Science (formerly | nuclease gene from Bacillus amyloliquefaciens; |
| | Canola) | | Plant Genetic | fertility restoration by insertion of the barstar |
| | | | Systems) | RNase inhibitor; PPT resistance was via PPT- |
| | | | | acetyltransferase (PAT) from Streptomyces hygroscopicus. |

In a further one preferred embodiment, the present invention relates to a method of controlling harmful fungi and/or increasing the health of cultivated plants by treating cultivated plants, parts of such plants, plant propagation materials, or at their locus of growth with a carboxamide compound selected from boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad, wherein the plant is resistant to antibiotics, more referably resistant to kanamycin, neomycin and ampicillin, most preferably resistant to kanamycin.

In a more preferred embodiment, the present invention relates to a method of controlling harmful fungi and/or increasing the health of cultivated plants by treating cultivated plants, parts of such plants, plant propagation materials, or at their locus of growth with a carboxamide compound selected from boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad, wherein the plant is a plant corresponding to a row of table 13.

In a more preferred embodiment, the present invention relates to a method of controlling harmful fungi and/or increasing the health of cultivated plants by treating plant propagation materials, preferably seeds with a carboxamide compound selected from boscalid, N-(3′,4′,5′-trifluorobiphenyl-2-yl)-3-difluoromethyl-1-methyl-1H-pyrazole-4-carboxamide, penflufen, fluopyram sedaxane, penthiopyrad carboxin, fenfuram, flutolanil; mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide, penflufen, fluopyram, sedaxane and penthiopyrad, wherein the plant corresponds to a row of table 13.

In another more preferred embodiment, the present invention relates to a method of controlling harmful fungi and/or increasing the health of cultivated plants by treating cultivated plants, parts of such plants or at their locus of growth with a carboxamide compound selected from boscalid, N-(3′,4′,5′-trifluorobiphenyl-2-yl)-3-difluoromethyl-1-methyl-1H-pyrazole-4-carboxamide bixafen, fluopyram, isopyrazam, penthiopyrad, flutolanil, furametpyr, mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide bixafen, fluopyram, isopyrazam and penthiopyrad, wherein the plant corresponds to a row of table 13.

In a most preferred embodiment, the present invention relates to a method of controlling harmful fungi and/or increasing the health of plants by treating cultivated plants, parts of such plants or at their locus of growth with a carboxamide compound, wherein the plant corresponds to a row of table 13 and the carboxamide compound is boscalid.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 13 and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 13 and the carboxamide compound is bixafen.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 13 and the carboxamide compound is fluopyram.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 13 and the carboxamide compound is isopyrazam.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 13 and the carboxamide compound is penthiopyrad.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 13 and the carboxamide compound is boscalid.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 13 and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 13 and the carboxamide compound is penflufen.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 13 and the carboxamide compound is fluopyram.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 13 and the carboxamide compound is sedaxane.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 13 and the carboxamide compound is penthiopyrad.

In a utmost preferred embodiment, the present invention relates to a method of controlling harmful fungi and/or increasing the health of plants by treating cultivated plants, parts of such plants or at their locus of growth with a carboxamide compound, wherein the plant is T13-6 of table 13 and the carboxamide compound is boscalid.

In another utmost preferred embodiment, the present invention relates to a method of controlling harmful fungi and/or increasing the health of plants by treating cultivated plants, parts of such plants or at their locus of growth with a carboxamide compound, wherein the plant is T13-6 of table 13 and the carboxamide compound is N-(3′,4′,5′-trifluorobiphenyl-2-yl)-3-difluoromethyl-1-methyl-1H-pyrazole-4-carboxamide.

In another utmost preferred embodiment, the present invention relates to a method of controlling harmful fungi and/or increasing the health of plants by treating cultivated plants, parts of such plants or at their locus of growth with a carboxamide compound, wherein the plant is T13-6 of table 13 and the carboxamide compound is bixafen.

In another utmost preferred embodiment, the present invention relates to a method of controlling harmful fungi and/or increasing the health of plants by treating cultivated plants, parts of such plants or at their locus of growth with a carboxamide compound, wherein the plant is T13-6 of table 13 and the carboxamide compound is fluopyram.

In another utmost preferred embodiment, the present invention relates to a method of controlling harmful fungi and/or increasing the health of plants by treating cultivated plants, parts of such plants or at their locus of growth with a carboxamide compound, wherein the plant is T13-6 of table 13 and the carboxamide compound is isopyrazam.

In another utmost preferred embodiment, the present invention relates to a method of controlling harmful fungi and/or increasing the health of plants by treating cultivated plants, parts of such plants or at their locus of growth with a carboxamide compound, wherein the plant is T13-6 of table 13 and the carboxamide compound is penthiopyrad.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is T13-6 of table 13 and the carboxamide compound is boscalid.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is T13-6 of table 13 and the carboxamide compound is N-(3′,4′,5′-trifluorobiphenyl-2-yl)-3-difluoromethyl-1-methyl-1 H-pyrazole-4-carboxamide.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is T13-6 of table 13 and the carboxamide compound is penflufen.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is T13-6 of table 13 and the carboxamide compound is fluopyram.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is T13-6 of table 13 and the carboxamide compound is sedaxane.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is T13-6 of table 13 and the carboxamide compound is penthiopyrad.

TABLE 13

| | | | Literature/ |
|---|---|---|---|
| No | detailed description | plant | commercial plants |
| T13-1 | kanamycin resistance | canola | A* |
| T13-2 | kanamycin resistance | cotton | A* |
| T13-3 | kanamycin resistance | flax | A* |
| T13-4 | kanamycin resistance | maize | A* |
| T13-5 | kanamycin resistance | oilseed rape | A* |
| T13-6 | kanamycin resistance | potato | A* |
| T13-7 | kanamycin resistance | rape seed | A* |
| T13-8 | kanamycin resistance | sugar beet | A* |
| T13-9 | kanamycin resistance | tomato | A*, B* |

A* refers to Plant Cell Reports, 20, 2001, 610-615. Trends in Plant Science, 11, 2006, 317-319. Plant Molecular Biology, 37, 1998, 287-296. Mol Gen Genet., 257, 1998, 606-13.
B* refers to Plant Cell Reports, 6, 1987, 333-336.

In a further one preferred embodiment, the present invention relates to a method of controlling harmful fungi and/or increasing the health of cultivated plants by treating cultivated plants, parts of such plants, plant propagation materials, or at their locus of growth with a carboxamide compound selected from boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad, wherein the plant has the trait of improved fiber quality.

In a more preferred embodiment, the present invention relates to a method of controlling harmful fungi and/or increasing the health of cultivated plants by treating cultivated plants, parts of such plants, plant propagation materials, or at their locus of growth with a carboxamide compound selected from boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad, wherein the plant is a cotton plant comprising the DP 104 B2RF event (“DP 104 B2RF—A new early maturing B2RF variety” presented at 2008 Beltwide Cotton Conferences by Tom R. Speed, Richard Sheetz, Doug Shoemaker, Monsanto/Delta and Pine Land, see http://www.monsanto.com/pdf/beltwide_{—}08/dp104b2rf_doc.pdf.

In a further one preferred embodiment, the present invention relates to a method of controlling harmful fungi and/or increasing the health of cultivated plants by treating cultivated plants, parts of such plants, plant propagation materials, or at their locus of growth with a carboxamide compound selected from boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam and penthiopyrad, wherein the plant is a transgenic plant, which has two traits stacked, more preferably two or more traits selected from the group consisting of herbicide tolerance, insect resistance, fungal resistance, viral resistance, bacterial resistance, stress tolerance, maturation alteration, content modification and modified nutrient uptake, most preferably the combination of herbicide tolerance and insect resistance, two herbicide tolerances, herbicide tolerance and stress tolerance, herbicide tolerance and modified content, two herbicide tolerances and insect resistance, herbicide tolerance, insect resistance and stress tolerance, herbicide tolerance, insect resistance and modified content.

In a more preferred embodiment, the present invention relates to a method of controlling harmful fungi and/or increasing the health of cultivated plants by treating plant propagation materials, preferably seeds with a carboxamide compound selected from boscalid, N-(3′,4′,5′-trifluorobiphenyl-2-yl)-3-difluoromethyl-1-methyl-1H-pyrazole-4-carboxamide, penflufen, fluopyram sedaxane, penthiopyrad carboxin, fenfuram, flutolanil; mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide, penflufen, fluopyram, sedaxane and penthiopyrad, wherein the plant corresponds to a row of table 14.

In another more preferred embodiment, the present invention relates to a method of controlling harmful fungi and/or increasing the health of cultivated plants by treating cultivated plants, parts of such plants or at their locus of growth with a carboxamide compound selected from boscalid, N-(3′,4′,5′-trifluorobiphenyl-2-yl)-3-difluoromethyl-1-methyl-1H-pyrazole-4-carboxamide bixafen, fluopyram, isopyrazam, penthiopyrad, flutolanil, furametpyr, mepronil, oxycarboxin, thifluzamide, more preferably with a carboxamide compound selected from boscalid, N-(3′,4′,5′-trifluorobiphenyl-2-yl)-3-difluoromethyl-1-methyl-1H-pyrazole-4-carboxamide bixafen, fluopyram, isopyrazam and penthiopyrad, wherein the plant corresponds to a row of table 14.

In a most preferred embodiment, the present invention relates to a method of controlling harmful fungi and/or increasing the health of plants by treating cultivated plants, parts of such plants or at their locus of growth with a carboxamide compound, wherein the plant corresponds to a row of table 14 and the carboxamide compound is boscalid.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 14 and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 14 and the carboxamide compound is bixafen.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 14 and the carboxamide compound is fluopyram.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 14 and the carboxamide compound is isopyrazam.

In another most preferred embodiment, the present invention relates to a method of controlling harmful fungi and/or increasing the health of plants by treating cultivated plants, parts of such plants or at their locus of growth with a carboxamide compound, wherein the plant corresponds to a row of table 14 and the carboxamide compound is penthiopyrad.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 14 and the carboxamide compound is boscalid.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 14 and the carboxamide compound is N-(3′,4′,5′-trifluorobiphenyl-2-yl)-3-difluoromethyl-1-methyl-1H-pyrazole-4-carboxamide.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 14 and the carboxamide compound is penflufen.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 14 and the carboxamide compound is fluopyram.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 14 and the carboxamide compound is sedaxane.

In another most preferred embodiment, the present invention relates to a method of controlling harmful fungi and/or increasing the health of cultivated plants by treating plant propagation materials, preferably seeds of cultivated plants of cultivated crops with a carboxamide compound, wherein the plant corresponds to a row of table 14 and the carboxamide compound is penthiopyrad.

In a utmost preferred embodiment, the present invention relates to a method of controlling harmful fungi and/or increasing the health of plants by treating cultivated plants, parts of such plants or at their locus of growth with a carboxamide compound, wherein the plant is selected from T14-1, T14-8, T14-13, T14-18, T14-19, T14-20, T14-21, T14-35, T14-36 and T14-37 of table 14 and the carboxamide compound is boscalid.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T14-1, T14-8, T14-13, T14-18, T14-19, T14-20, T14-21, T14-35, T14-36 and T14-37 of table 14 and the carboxamide compound is N-(3′,4′,5′-trifluorobiphenyl-2-yl)-3-difluoromethyl-1-methyl-1H-pyrazole-4-carboxamide.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T14-1, T14-8, T14-13, T14-18, T14-19, T14-20, T14-21, T14-35, T14-36 and T14-37 of table 14 and the carboxamide compound is bixafen.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T14-1, T14-8, T14-13, T14-18, T14-19, T14-20, T14-21, T14-35, T14-36 and T14-37 of table 14 and the carboxamide compound is fluopyram.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T14-1, T14-8, T14-13, T14-18, T14-19, T14-20, T14-21, T14-35, T14-36 and T14-37 of table 14 and the carboxamide compound is isopyrazam.

In another utmost preferred embodiment, the present invention relates to a method of controlling harmful fungi and/or increasing the health of plants by treating cultivated plants, parts of such plants or at their locus of growth with a carboxamide compound, wherein the plant is selected from T14-1, T14-8, T14-13, T14-18, T14-19, T14-20, T14-21, T14-35, T14-36 and T14-37 of table 14 and the carboxamide compound is penthiopyrad.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T14-1, T14-8, T14-13, T14-18, T14-19, T14-20, T14-21, T14-35, T14-36 and T14-37 of table 14 and the carboxamide compound is boscalid.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T14-1, T14-8, T14-13, T14-18, T14-19, T14-20, T14-21, T14-35, T14-36 and T14-37 of table 14 and the carboxamide compound is N-(3′,4′,5′-trifluorobiphenyl-2-yl)-3-difluoromethyl-1-methyl-1H-pyrazole-4-carboxamide.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T14-1, T14-8, T14-13, T14-18, T14-19, T14-20, T14-21, T14-35, T14-36 and T14-37 of table 14 and the carboxamide compound is penflufen.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T14-1, T14-8, T14-13, T14-18, T14-19, T14-20, T14-21, T14-35, T14-36 and T14-37 of table 14 and the carboxamide compound is fluopyram.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T14-1, T14-8, T14-13, T14-18, T14-19, T14-20, T14-21, T14-35, T14-36 and T14-37 of table 14 and the carboxamide compound is sedaxane.

In another utmost preferred embodiment, the present invention relates to a method of controlling harmful fungi and/or increasing the health of cultivated plants by treating plant propagation materials, preferably seeds of cultivated plants of cultivated crops with a carboxamide compound, wherein the plant is selected from T14-1, T14-8, T14-13, T14-18, T14-19, T14-20, T14-21, T14-35, T14-36 and T14-37 of table 14 and the carboxamide compound is penthiopyrad.

TABLE 14

| No | detailed description | plant | Literature/commercial plants |
|---|---|---|---|
| T14-1 | corn borer resistance + glyphosate tolerance | maize | “YieldGard Roundup Ready”, |
| | | | YieldGard Roundup Ready 2” (Monsanto) |
| T14-2 | corn borer resistance + glufosinate tolerance | maize | “Agrisure CB/LL” (Syntenta) |
| T14-3 | glyphosate tolerance + corn rootworm | maize | “Yield Gard VT Rootworm/RR2” |
| | resistance | | |
| T14-4 | glyphosate tolerance + corn root- | maize | “Yield Gard VT Triple” |
| | worm/corn borer resistance | | |
| T14-5 | glufosinate tolerance + lepidopteran | maize | “Herculex I” |
| | resistance (Cry1F; western bean cut- | | |
| | worm, corn borer, black cutworm, fall | | |
| | armyworm resistance) | | |
| T14-6 | glyphosate tolerance + corn rootworm | maize | “YieldGard Corn Rootworm/Roundup |
| | resistance | | Ready 2” (Monsanto) |
| T14-7 | glyphosate tolerance + gluphosinate | maize | “Herculex I/Roundup Ready 2”; |
| | tolerance + lepidopteran resistance | | |
| | (Cry1F; western bean cutworm, corn | | |
| | borer, black cutworm, fall armyworm | | |
| | resistance) | | |
| T14-8 | glyphosate tolerance + corn rootworm | maize | “YieldGard Plus/Roundup Ready 2” |
| | resistance + corn borer resistance | | (Monsanto) |
| T14-9 | gluphosinate tolerance + lepidopteran | maize | “Agrisure GT/RW” (Syngenta) |
| | resistance (Cry3A; western corn root- | | |
| | worm, northern corn rootworm, Mexican | | |
| | corn rootworm resistance) | | |
| T14-10 | glyphosate tolerance + gluphosinate | maize | “Agrisure GT/CB/LL” (Syngenta) |
| | tolerance + corn borer resistance | | |
| T14-11 | glufosinate tolerance + lepidopteran | maize | “Herculex RW” (Dow, Pioneer) |
| | resistance (Cry34/35Ab1; western corn | | |
| | rootworm, northern corn rootworm, | | |
| | Mexican corn rootworm resistance) | | |
| T14-12 | glufosinate tolerance + lepidopteran | maize | “Herculex Xtra” (Dow, Pioneer) |
| | resistance (Cry1F + Cry34/35Ab1; | | |
| | western corn rootworm, northern corn | | |
| | rootworm, Mecxican corn rootworm, | | |
| | western bean cutworm, corn borer, | | |
| | black cutworm, fall armyworm resistance) | | |
| T14-13 | glyphosate tolerance + glufosinate tolerance + | maize | “Herculex Quad-Stack” |
| | corn borer resistance + corn | | |
| | rootworm resistance | | |
| T14-14 | glyphosate tolerance + corn rootworm | maize | “Yield Gard VT Rootworm/RR2” |
| | resistance | | |
| T14-15 | glufosinate tolerance + corn borer resistance | maize | “Agrisure CB/LL/RW” (Syngenta) |
| | (Cry1Ab) + lepidopteran resistance | | |
| | (Cry3A; western corn rootworm, | | |
| | northern corn rootworm, Mexican corn | | |
| | rootworm resistance) | | |
| T14-16 | glyphosate tolerance + corn borer | maize | “Agrisure 3000GT” (Syngenta) |
| | resistance (Cry1Ab) + lepidopteran | | |
| | resistance (Cry3A; western corn | | |
| | rootworm, northern corn rootworm, | | |
| | Mexican corn rootworm resistance) | | |
| T14-17 | glyphosate tolerance + resistance to | maize | “Mavera high-value corn” (Monsanto) |
| | corn borer and corn rootworm + high | | |
| | lysine content | | |
| T14-18 | glyphosate tolerance + ALS herbicide | soy- | “Optimum GAT” (DuPont, Pioneer) |
| | tolerance (F*) | bean | |
| T14-19 | glyphosate tolerance + lepidoptera resistance | soy- | A*, U.S. Pat. No. 7,432,421 |
| | (Bt) | bean | |
| T14-20 | glyphosate tolerance + Dicamba tolerance | soy- | A*, U.S. Pat. No. 7,105,724 |
| | | bean | |
| T14-21 | glyphosate tolerance + modified oil content | soy- | A*, G* |
| | | bean | |
| T14-22 | glufosinate tolerance + modified oil content | soy- | G*, I* |
| | | bean | |
| T14-23 | glyphosate tolerance + dicamba tolerance | cotton | A*, U.S. Pat. No. 7,105,724, WO2008051633 |
| T14-24 | glufosinate tolerance + lepidopteran | cotton | D*, U.S. Pat. No. 5,646,024, U.S. Pat. No. 5,561,236 |
| | resistance | | |
| T14-25 | glyphosate tolerance + lepidopteran | cotton | A*, D* |
| | resistance | | |
| T14-26 | glufosinate tolerance + dicamba tolerance | cotton | U.S. Pat. No. 5,646,024, U.S. Pat. No. 5,561,236, |
| | | | U.S. Pat. No. 7,105,724, WO2008051633 |
| T14-27 | glyphosate tolerance + improved fiber | cotton | A*, E* |
| | quality | | |
| T14-28 | glufosinate tolerance + improved fiber | cotton | E*, U.S. Pat. No. 5,646,024, U.S. Pat. No. 5,561,236 |
| | quality | | |
| T14-29 | glyphosate tolerance + drought tolerance | cotton | A*, C* |
| T14-30 | glyphosate tolerance + dicamba tolerance + | cotton | A*, C*, U.S. Pat. No. 7,105,724, WO 2008/051633 |
| | drought tolerance | | |
| T14-31 | glufosinate tolerance + insect resistance | cotton | D*, U.S. Pat. No. 5,646,024, U.S. Pat. No. 5,561,236 |
| | (tobacco budworm, cotton boll- | | |
| | worm, fall armyworm, beet armyworm, | | |
| | cabbage looper, soybean lopper, pink | | |
| | bollworm resistance) | | |
| T14-32 | glyphosate tolerance + modified oil content | canola | A*, U.S. Pat. No. 5,850,026, U.S. Pat. No. 6,441,278, U.S. Pat. No. |
| | | | 5,723,761, WO 2005/033319 |
| T14-33 | glufosinate tolerance + modified oil content | canola | U.S. Pat. No. 5,646,024, U.S. Pat. No. 5,561,236, U.S. Pat. No. |
| | | | 5,850,026, U.S. Pat. No. 6,441,278, U.S. Pat. No. 5,723,761, |
| | | | WO 2005/033319 |
| T14-34 | glyphosate tolerance + insect resistance | canola | D*, A* |
| T14-35 | glufosinate tolerance + insect resistance | canola | D*, U.S. Pat. No. 5,646,024, U.S. Pat. No. 5,561,236 |
| T14-36 | IMI tolerance + Coleoptera resistance | rice | B*, WO 2001/021821 |
| T14-37 | IMI tolerance + Lepidoptera resistance | rice | B*, WO 2001/021821 |
| T14-38 | IMI tolerance + modified oil content | sun- | Tan et. al, Pest Manag. Sci 61, 246-257 |
| | | flower | (2005). |
| T14-39 | Coleoptera resistance, + | potato | H* |
| | Kanamycin resistance | | |
| T14-40 | Coleoptera resistance, + | potato | H* |
| | Kanamycin resistance + potato leaf | | |
| | roll virus resistance | | |
| T14-41 | Coleoptera resistance, + | potato | H* |
| | Kanamycin resistance + potato leaf | | |
| | roll virus resistance | | |

A* refers to U.S. Pat. No. 5,188,642, U.S. Pat. No. 4,940,835, U.S. Pat. No. 5,633,435, U.S. Pat. No. 5,804,425 and U.S. Pat. No. 5,627,061.
B* refers to imidazolinone-herbicide resistant rice plants with specific mutation of the acetohydroxyacid synthase gene: S653N (see e.g. US 2003/0217381), S654K (see e.g. US 2003/0217381), A122T (see e.g. WO 2004/106529) S653(At)N, S654(At)K, A122(At)T and other resistant rice plants as described in WO 2000/27182, WO 2005/20673 and WO 2001/85970 or US patents U.S. Pat. No. 5,545,822, U.S. Pat. No. 5,736,629, U.S. Pat. No. 5,773,703, U.S. Pat. No. 5,773,704, U.S. Pat. No. 5,952,553, U.S. Pat. No. 6,274,796, wherein plants with mutation S653A and A122T are most preferred.
C* referes to WO 2000/04173, WO 2007/131699, US 20080229448 and WO 2005/48693.
D* refers to WO 1993/07278 and WO 1995/34656.
E* refers to WO 1996/26639, U.S. Pat. No. 7,329,802, U.S. Pat. No. 6,472,588 and WO 2001/17333.
F* refers to sulfonylurea and imidazolinone herbicides, such as imazamox, imazethapyr, imazaquin, chlorimuron, flumetsulam, cloransulam, diclosulam and thifensulfuron.
G* refers to U.S. Pat. No. 6,380,462, U.S. Pat. No. 6,365,802, U.S. Pat. No. 7,294,759 and U.S. Pat. No. 7,157,621.
H* refers to Plant Cell Reports, 20, 2001, 610-615. Trends in Plant Science, 11, 2006, 317-319. Plant Molecular Biology, 37, 1998, 287-296. Mol Gen Genet., 257, 1998, 606-13. Federal Register (USA), Vol. 60, No. 113, 1995, page 31139. Federal Register (USA), Vol. 67, No. 226, 2002, page 70392. Federal Register (USA), Vol. 63, No. 88, 1998, page 25194. Federal Register (USA), Vol. 60, No. 141, 1995, page 37870. Canadian Food Inspection Agency, FD/OFB-095-264-A, October 1999, FD/OFB-099-127-A, October 1999.
I* refers to Federal Register (USA), Vol. 61, No. 160, 1996, page 42581. Federal Register (USA), Vol. 63, No. 204, 1998, page 56603.

Preferred embodiments of the invention are those methods of controlling harmful fungi and/or increasing the health of plants by treating cultivated plants, parts of such plants or at their locus of growth with a carboxamide compound, wherein the plant is a transgenic plant which is selected from the plants listed in table A.

In a more preferred embodiment, the present invention relates of methods of controlling harmful fungi and/or increasing the health of plants by treating cultivated plants, parts of such plants or at their locus of growth with a carboxamide compound, wherein the plant is selected from the plants listed in table A and the carboxamide compound is boscalid.

In a more preferred embodiment, the present invention relates of methods of controlling harmful fungi and/or increasing the health of plants by treating cultivated plants, parts of such plants or at their locus of growth with a carboxamide compound, wherein the plant is selected from the plants listed in table A and the carboxamide compound is N-(3′,4′,5′-trifluorobiphenyl-2-yl)-3-difluoromethyl-1-methyl-1H-pyrazole-4-carboxamide.

In a more preferred embodiment, the present invention relates of methods of controlling harmful fungi and/or increasing the health of plants by treating cultivated plants, parts of such plants or at their locus of growth with a carboxamide compound, wherein the plant is selected from the plants listed in table A and the carboxamide compound is bixafen.

In a more preferred embodiment, the present invention relates of methods of controlling harmful fungi and/or increasing the health of plants by treating cultivated plants, parts of such plants or at their locus of growth with a carboxamide compound, wherein the plant is selected from the plants listed in table A and the carboxamide compound is penflufen.

In a more preferred embodiment, the present invention relates of methods of controlling harmful fungi and/or increasing the health of plants by treating cultivated plants, parts of such plants or at their locus of growth with a carboxamide compound, wherein the plant is selected from the plants listed in table A and the carboxamide compound is fluopyram.

In a more preferred embodiment, the present invention relates of methods of controlling harmful fungi and/or increasing the health of plants by treating cultivated plants, parts of such plants or at their locus of growth with a carboxamide compound, wherein the plant is selected from the plants listed in table A and the carboxamide compound is sedaxane.

In a more preferred embodiment, the present invention relates of methods of controlling harmful fungi and/or increasing the health of plants by treating cultivated plants, parts of such plants or at their locus of growth with a carboxamide compound, wherein the plant is selected from the plants listed in table A and the carboxamide compound is isopyrazam.

In a more preferred embodiment, the present invention relates of methods of controlling harmful fungi and/or increasing the health of plants by treating cultivated plants, parts of such plants or at their locus of growth with a carboxamide compound, wherein the plant is selected from the plants listed in table A and the carboxamide compound is penthiopyrad.

Another preferred embodiment of the invention are those methods of controlling harmful fungi and/or increasing the health of plants by treating cultivated plants, parts of such plants or at their locus of growth with a carboxamide compound, wherein the plant is a transgenic plant which is selected from the plants listed in table B.

In a more preferred embodiment, the present invention relates of methods of controlling harmful fungi and/or increasing the health of plants by treating cultivated plants, parts of such plants or at their locus of growth with a carboxamide compound, wherein the plant is selected from the plants listed in table B and the carboxamide compound is boscalid.

In a more preferred embodiment, the present invention relates of methods of controlling harmful fungi and/or increasing the health of plants by treating cultivated plants, parts of such plants or at their locus of growth with a carboxamide compound, wherein the plant is selected from the plants listed in table B and the carboxamide compound is N-(3′,4′,5′-trifluorobiphenyl-2-yl)-3-difluoromethyl-1-methyl-1H-pyrazole-4-carboxamide.

In a more preferred embodiment, the present invention relates of methods of controlling harmful fungi and/or increasing the health of plants by treating cultivated plants, parts of such plants or at their locus of growth with a carboxamide compound, wherein the plant is selected from the plants listed in table B and the carboxamide compound is bixafen.

In a more preferred embodiment, the present invention relates of methods of controlling harmful fungi and/or increasing the health of plants by treating cultivated plants, parts of such plants or at their locus of growth with a carboxamide compound, wherein the plant is selected from the plants listed in table B and the carboxamide compound is penflufen.

In a more preferred embodiment, the present invention relates of methods of controlling harmful fungi and/or increasing the health of plants by treating cultivated plants, parts of such plants or at their locus of growth with a carboxamide compound, wherein the plant is selected from the plants listed in table B and the carboxamide compound is fluopyram.

In a more preferred embodiment, the present invention relates of methods of controlling harmful fungi and/or increasing the health of plants by treating cultivated plants, parts of such plants or at their locus of growth with a carboxamide compound, wherein the plant is selected from the plants listed in table B and the carboxamide compound is sedaxane.

In a more preferred embodiment, the present invention relates of methods of controlling harmful fungi and/or increasing the health of plants by treating cultivated plants, parts of such plants or at their locus of growth with a carboxamide compound, wherein the plant is selected from the plants listed in table B and the carboxamide compound is isopyrazam.

In a more preferred embodiment, the present invention relates of methods of controlling harmful fungi and/or increasing the health of plants by treating cultivated plants, parts of such plants or at their locus of growth with a carboxamide compound, wherein the plant is selected from the plants listed in table B and the carboxamide compound is penthiopyrad.

In another preferred embodiment, the present invention relates of methods of controlling harmful fungi and/or increasing the health of plants by treating cultivated plants, parts of such plants or at their locus of growth with a respiration complex III inhibitor, wherein the plant is selected from B-3, B-4, B-5, B-7, B-8, B-11, B-23, B-28,B-29, B-30, B-39, B-42, B-44, B-46, B-47, B-55, B-59, B-61, B-63, B-64, B-69, B-70, B-71 of table B.

In a most preferred embodiment, the present invention relates of methods of controlling harmful fungi and/or increasing the health of plants by treating cultivated plants, parts of such plants or at their locus of growth with a carboxamide compound, wherein the plant is selected from B-3, B-4, B-5, B-7, B-8, B-11, B-23, B-28,B-29, B-30, B-39, B-42, B-44, B-46, B-47, B-55, B-59, B-61, B-63, B-64, B-69, B-70, B-71 of table B and the carboxamide compound is boscalid.

In a most preferred embodiment, the present invention relates of methods of controlling harmful fungi and/or increasing the health of plants by treating cultivated plants, parts of such plants or at their locus of growth with a carboxamide compound, wherein the plant is selected from B-3, B-4, B-5, B-7, B-8, B-11, B-23, B-28,B-29, B-30, B-39, B-42, B-44, B-46, B-47, B-55, B-59, B-61, B-63, B-64, B-69, B-70, B-71 of table B and the carboxamide compound is N-(3′,4′,5′-trifluorobiphenyl-2-yl)-3-difluoromethyl-1-methyl-1 H-pyrazole-4-carboxamide.

In a most preferred embodiment, the present invention relates of methods of controlling harmful fungi and/or increasing the health of plants by treating cultivated plants, parts of such plants or at their locus of growth with a carboxamide compound, wherein the plant is selected from B-3, B-4, B-5, B-7, B-8, B-11, B-23, B-28,B-29, B-30, B-39, B-42, B-44, B-46, B-47, B-55, B-59, B-61, B-63, B-64, B-69, B-70, B-71 of table B and the carboxamide compound is bixafen.

In a most preferred embodiment, the present invention relates of methods of controlling harmful fungi and/or increasing the health of plants by treating cultivated plants, parts of such plants or at their locus of growth with a carboxamide compound, wherein the plant is selected from B-3, B-4, B-5, B-7, B-8, B-11, B-23, B-28,B-29, B-30, B-39, B-42, B-44, B-46, B-47, B-55, B-59, B-61, B-63, B-64, B-69, B-70, B-71 of table B and the carboxamide compound is penflufen.

In a most preferred embodiment, the present invention relates of methods of controlling harmful fungi and/or increasing the health of plants by treating cultivated plants, parts of such plants or at their locus of growth with a carboxamide compound, wherein the plant is selected from B-3, B-4, B-5, B-7, B-8, B-11, B-23, B-28,B-29, B-30, B-39, B-42, B-44, B-46, B-47, B-55, B-59, B-61, B-63, B-64, B-69, B-70, B-71 of table B and the carboxamide compound is fluopyram.

In a most preferred embodiment, the present invention relates of methods of controlling harmful fungi and/or increasing the health of plants by treating cultivated plants, parts of such plants or at their locus of growth with a carboxamide compound, wherein the plant is selected from B-3, B-4, B-5, B-7, B-8, B-11, B-23, B-28,B-29, B-30, B-39, B-42, B-44, B-46, B-47, B-55, B-59, B-61, B-63, B-64, B-69, B-70, B-71 of table B and the carboxamide compound is sedaxane.

In a most preferred embodiment, the present invention relates of methods of controlling harmful fungi and/or increasing the health of plants by treating cultivated plants, parts of such plants or at their locus of growth with a carboxamide compound, wherein the plant is selected from B-3, B-4, B-5, B-7, B-8, B-11, B-23, B-28,B-29, B-30, B-39, B-42, B-44, B-46, B-47, B-55, B-59, B-61, B-63, B-64, B-69, B-70, B-71 of table B and the carboxamide compound is isopyrazam.

In a most preferred embodiment, the present invention relates of methods of controlling harmful fungi and/or increasing the health of plants by treating cultivated plants, parts of such plants or at their locus of growth with a carboxamide compound, wherein the plant is selected from B-3, B-4, B-5, B-7, B-8, B-11, B-23, B-28,B-29, B-30, B-39, B-42, B-44, B-46, B-47, B-55, B-59, B-61, B-63, B-64, B-69, B-70, B-71 of table B and the carboxamide compound is penthiopyrad.

Further preferred embodiments of the invention are those methods of controlling harmful fungi and/or increasing the health of plants by treating cultivated plants, parts of such plants or at their locus of growth with a carboxamide compound, wherein the plant expresses one or more genes selected from aad, ACCase, ALS, AMY797E, APH4, bar, barnase, barstar, bla, bxn, cDHDPS, CP, cmv-cp, Cry1Ab, Cry1Ac, Cry1A.105, Cry1F, Cry1Fa2, Cry2Ab, Cry34Ab1, Cry35Ab1, Cry3A, Cry3Bb1, Cry9C, dam, DHFR, fad2, fan1, FH, flcry1Ab, GAT4601, GAT4602, gmFAD2-1, GM-HRA, goxv-247, gus, hel, mCry3A, nos, NPTII, pat, PG, pinII, PMI, prsv-cp, QTPASE, rep, SAMase, spc, TE, vip3A, vip3A(a), wmv2-cp and zymv-cp.

In a more preferred embodiment, the present invention relates of methods of controlling harmful fungi and/or increasing the health of plants by treating cultivated plants, parts of such plants or at their locus of growth with a carboxamide compound, wherein the carboxamide compound is boscalid and the plant expresses one or more genes selected from aad, ACCase, ALS, AMY797E, APH4, bar, barnase, barstar, bla, bxn, cDHDPS, CP, cmv-cp, Cry1Ab, Cry1Ac, Cry1A.105, Cry1F, Cry1Fa2, Cry2Ab, Cry34Ab1, Cry35Ab1, Cry3A, Cry3Bb1, Cry9C, dam, DHFR, fad2, fan1, FH, flcry1Ab, GAT4601, GAT4602, gmFAD2-1, GM-HRA, goxv-247, gus, hel, mCry3A, nos, NPTII, pat, PG, pinII, PMI, prsv-cp, QTPASE, rep, SAMase, spc, TE, vip3A, vip3A(a), wmv2-cp and zymv-cp.

In a more preferred embodiment, the present invention relates of methods of controlling harmful fungi and/or increasing the health of plants by treating cultivated plants, parts of such plants or at their locus of growth with a carboxamide compound, wherein the carboxamide compound is N-(3′,4′,5′-trifluorobiphenyl-2-yl)-3-difluoromethyl-1-methyl-1H-pyrazole-4-carboxamide and the plant expresses one or more genes selected from aad, ACCase, ALS, AMY797E, APH4, bar, barnase, barstar, bla, bxn, cDHDPS, CP, cmv-cp, Cry1Ab, Cry1Ac, Cry1A.105, Cry1F, Cry1Fa2, Cry2Ab, Cry34Ab1, Cry35Ab1, Cry3A, Cry3Bb1, Cry9C, dam, DHFR, fad2, fan1, FH, flcry1Ab, GAT4601, GAT4602, gmFAD2-1, GM-HRA, goxv-247, gus, hel, mCry3A, nos, NPTII, pat, PG, pinII, PMI, prsv-cp, QTPASE, rep, SAMase, spc, TE, vip3A, vip3A(a), wmv2-cp and zymv-cp.

In a more preferred embodiment, the present invention relates of methods of controlling harmful fungi and/or increasing the health of plants by treating cultivated plants, parts of such plants or at their locus of growth with a carboxamide compound, the carboxamide compound is bixafen and the plant expresses one or more genes selected from aad, ACCase, ALS, AMY797E, APH4, bar, barnase, barstar, bla, bxn, cDHDPS, CP, cmv-cp, Cry1Ab, Cry1Ac, Cry1A.105, Cry1F, Cry1Fa2, Cry2Ab, Cry34Ab1, Cry35Ab1, Cry3A, Cry3Bb1, Cry9C, dam, DHFR, fad2, fan1, FH, flcry1Ab, GAT4601, GAT4602, gmFAD2-1, GM-HRA, goxv-247, gus, hel, mCry3A, nos, NPTII, pat, PG, pinII, PMI, prsv-cp, QTPASE, rep, SAMase, spc, TE, vip3A, vip3A(a), wmv2-cp and zymv-cp.

In a more preferred embodiment, the present invention relates of methods of controlling harmful fungi and/or increasing the health of plants by treating cultivated plants, parts of such plants or at their locus of growth with a carboxamide compound, wherein the carboxamide compound is penflufen and the plant expresses one or more genes selected from aad, ACCase, ALS, AMY797E, APH4, bar, barnase, barstar, bla, bxn, cDHDPS, CP, cmv-cp, Cry1Ab, Cry1Ac, Cry1A.105, Cry1F, Cry1Fa2, Cry2Ab, Cry34Ab1, Cry35Ab1, Cry3A, Cry3Bb1, Cry9C, dam, DHFR, fad2, fan1, FH, flcry1Ab, GAT4601, GAT4602, gmFAD2-1, GM-HRA, goxv-247, gus, hel, mCry3A, nos, NPTII, pat, PG, pinII, PMI, prsv-cp, QTPASE, rep, SAMase, spc, TE, vip3A, vip3A(a), wmv2-cp and zymv-cp.

In a more preferred embodiment, the present invention relates of methods of controlling harmful fungi and/or increasing the health of plants by treating cultivated plants, parts of such plants or at their locus of growth with a carboxamide compound, wherein the carboxamide compound is fluopyram and the plant expresses one or more genes selected from aad, ACCase, ALS, AMY797E, APH4, bar, barnase, barstar, bla, bxn, cDHDPS, CP, cmv-cp, Cry1Ab, Cry1Ac, Cry1A.105, Cry1F, Cry1Fa2, Cry2Ab, Cry34Ab1, Cry35Ab1, Cry3A, Cry3Bb1, Cry9C, dam, DHFR, fad2, fan1, FH, flcry1Ab, GAT4601, GAT4602, gmFAD2-1, GM-HRA, goxv-247, gus, hel, mCry3A, nos, NPTII, pat, PG, pinII, PMI, prsv-cp, QTPASE, rep, SAMase, spc, TE, vip3A, vip3A(a), wmv2-cp and zymv-cp.

In a more preferred embodiment, the present invention relates of methods of controlling harmful fungi and/or increasing the health of plants by treating cultivated plants, parts of such plants or at their locus of growth with a carboxamide compound, wherein the carboxamide compound is sedaxane and the plant expresses one or more genes selected from aad, ACCase, ALS, AMY797E, APH4, bar, barnase, barstar, bla, bxn, cDHDPS, CP, cmv-cp, Cry1Ab, Cry1Ac, Cry1A.105, Cry1F, Cry1Fa2, Cry2Ab, Cry34Ab1, Cry35Ab1, Cry3A, Cry3Bb1, Cry9C, dam, DHFR, fad2, fan1, FH, flcry1Ab, GAT4601, GAT4602, gmFAD2-1, GM-HRA, goxv-247, gus, hel, mCry3A, nos, NPTII, pat, PG, pinII, PMI, prsv-cp, QTPASE, rep, SAMase, spc, TE, vip3A, vip3A(a), wmv2-cp and zymv-cp.

In a more preferred embodiment, the present invention relates of methods of controlling harmful fungi and/or increasing the health of plants by treating cultivated plants, parts of such plants or at their locus of growth with a carboxamide compound, wherein the carboxamide compound is isopyrazam and the plant expresses one or more genes selected from aad, ACCase, ALS, AMY797E, APH4, bar, barnase, barstar, bla, bxn, cDHDPS, CP, cmv-cp, Cry1Ab, Cry1Ac, Cry1A.105, Cry1F, Cry1Fa2, Cry2Ab, Cry34Ab1, Cry35Ab1, Cry3A, Cry3Bb1, Cry9C, dam, DHFR, fad2, fan1, FH, flcry1Ab, GAT4601, GAT4602, gmFAD2-1, GM-HRA, goxv-247, gus, hel, mCry3A, nos, NPTII, pat, PG, pinII, PMI, prsv-cp, QTPASE, rep, SAMase, spc, TE, vip3A, vip3A(a), wmv2-cp and zymv-cp.

In a more preferred embodiment, the present invention relates of methods of controlling harmful fungi and/or increasing the health of plants by treating cultivated plants, parts of such plants or at their locus of growth with a carboxamide compound, wherein the carboxamide compound is penthiopyrad and the plant expresses one or more genes selected from aad, ACCase, ALS, AMY797E, APH4, bar, barnase, barstar, bla, bxn, cDHDPS, CP, cmv-cp, Cry1Ab, Cry1Ac, Cry1A.105, Cry1F, Cry1Fa2, Cry2Ab, Cry34Ab1, Cry35Ab1, Cry3A, Cry3Bb1, Cry9C, dam, DHFR, fad2, fan1, FH, flcry1Ab, GAT4601, GAT4602, gmFAD2-1, GM-HRA, goxv-247, gus, hel, mCry3A, nos, NPTII, pat, PG, pinII, PMI, prsv-cp, QTPASE, rep, SAMase, spc, TE, vip3A, vip3A(a), wmv2-cp and zymv-cp.

Further preferred embodiments of the invention are those methods of controlling harmful fungi and/or increasing the health of plants by treating cultivated plants, parts of such plants or at their locus of growth with a carboxamide compound, wherein the plant expresses one or more genes selected from CP4 epsps, pat, bar, Cry1Ab, Cry1Ac, Cry3Bb1, Cry2Ab, Cry1F, Cry34Ab1 and Cry35Ab1.

In a more preferred embodiment, the present invention relates of methods of controlling harmful fungi and/or increasing the health of plants by treating cultivated plants, parts of such plants or at their locus of growth with a carboxamide compound, wherein the carboxamide compound is boscalid and the plant expresses one or more genes selected from CP4 epsps, pat, bar, Cry1Ab, Cry1Ac, Cry3Bb1, Cry2Ab, Cry1F, Cry34Ab1 and Cry35Ab1.

In a more preferred embodiment, the present invention relates of methods of controlling harmful fungi and/or increasing the health of plants by treating cultivated plants, parts of such plants or at their locus of growth with a carboxamide compound, wherein the carboxamide compound is N-(3′,4′,5′-trifluorobiphenyl-2-yl)-3-difluoromethyl-1-methyl-1H-pyrazole-4-carboxamide and the plant expresses one or more genes selected from CP4 epsps, pat, bar, Cry1Ab, Cry1Ac, Cry3Bb1, Cry2Ab, Cry1F, Cry34Ab1 and Cry35Ab1.

In a more preferred embodiment, the present invention relates of methods of controlling harmful fungi and/or increasing the health of plants by treating cultivated plants, parts of such plants or at their locus of growth with a carboxamide compound, wherein the carboxamide compound is bixafen and the plant expresses one or more genes selected from CP4 epsps, pat, bar, Cry1Ab, Cry1Ac, Cry3Bb1, Cry2Ab, Cry1F, Cry34Ab1 and Cry35Ab1.

In a more preferred embodiment, the present invention relates of methods of controlling harmful fungi and/or increasing the health of plants by treating cultivated plants, parts of such plants or at their locus of growth with a carboxamide compound, wherein the carboxamide compound is penflufen and the plant expresses one or more genes selected from CP4 epsps, pat, bar, Cry1Ab, Cry1Ac, Cry3Bb1, Cry2Ab, Cry1F, Cry34Ab1 and Cry35Ab1.

In a more preferred embodiment, the present invention relates of methods of controlling harmful fungi and/or increasing the health of plants by treating cultivated plants, parts of such plants or at their locus of growth with a carboxamide compound, wherein the carboxamide compound is fluopyram and the plant expresses one or more genes selected from CP4 epsps, pat, bar, Cry1Ab, Cry1Ac, Cry3Bb1, Cry2Ab, Cry1F, Cry34Ab1 and Cry35Ab1.

In a more preferred embodiment, the present invention relates of methods of controlling harmful fungi and/or increasing the health of plants by treating cultivated plants, parts of such plants or at their locus of growth with a carboxamide compound, wherein the carboxamide compound is sedaxane and the plant expresses one or more genes selected from CP4 epsps, pat, bar, Cry1Ab, Cry1Ac, Cry3Bb1, Cry2Ab, Cry1F, Cry34Ab1 and Cry35Ab1.

In a more preferred embodiment, the present invention relates of methods of controlling harmful fungi and/or increasing the health of plants by treating cultivated plants, parts of such plants or at their locus of growth with a carboxamide compound, wherein the carboxamide compound is isopyrazam and the plant expresses one or more genes selected from CP4 epsps, pat, bar, Cry1Ab, Cry1Ac, Cry3Bb1, Cry2Ab, Cry1F, Cry34Ab1 and Cry35Ab1.

In a more preferred embodiment, the present invention relates of methods of controlling harmful fungi and/or increasing the health of plants by treating cultivated plants, parts of such plants or at their locus of growth with a carboxamide compound, wherein the carboxamide compound is penthiopyrad and the plant expresses one or more genes selected from CP4 epsps, pat, bar, Cry1Ab, Cry1Ac, Cry3Bb1, Cry2Ab, Cry1F, Cry34Ab1 and Cry35Ab1.

Further preferred embodiments of the invention are those methods of controlling harmful fungi and/or increasing the health of plants by treating cultivated plants, parts of such plants or at their locus of growth with a carboxamide compound, wherein the plant is a transgenic plant which is selected from the plants listed in table C.

In a more preferred embodiment, the present invention relates of methods of controlling harmful fungi and/or increasing the health of plants by treating cultivated plants, parts of such plants or at their locus of growth with a carboxamide compound, wherein the plant is selected from the plants listed in table C and the carboxamide compound is boscalid.

In a more preferred embodiment, the present invention relates of methods of controlling harmful fungi and/or increasing the health of plants by treating cultivated plants, parts of such plants or at their locus of growth with a carboxamide compound, wherein the plant is selected from the plants listed in table C and the carboxamide compound is N-(3′,4′,5′-trifluorobiphenyl-2-yl)-3-difluoromethyl-1-methyl-1H-pyrazole-4-carboxamide.

In a more preferred embodiment, the present invention relates of methods of controlling harmful fungi and/or increasing the health of plants by treating cultivated plants, parts of such plants or at their locus of growth with a carboxamide compound, wherein the plant is selected from the plants listed in table C and the carboxamide compound is bixafen.

In a more preferred embodiment, the present invention relates of methods of controlling harmful fungi and/or increasing the health of plants by treating cultivated plants, parts of such plants or at their locus of growth with a carboxamide compound, wherein the plant is selected from the plants listed in table C and the carboxamide compound is penflufen.

In a more preferred embodiment, the present invention relates of methods of controlling harmful fungi and/or increasing the health of plants by treating cultivated plants, parts of such plants or at their locus of growth with a carboxamide compound, wherein the plant is selected from the plants listed in table C and the carboxamide compound is fluopyram.

In a more preferred embodiment, the present invention relates of methods of controlling harmful fungi and/or increasing the health of plants by treating cultivated plants, parts of such plants or at their locus of growth with a carboxamide compound, wherein the plant is selected from the plants listed in table C and the carboxamide compound is sedaxane.

In a more preferred embodiment, the present invention relates of methods of controlling harmful fungi and/or increasing the health of plants by treating cultivated plants, parts of such plants or at their locus of growth with a carboxamide compound, wherein the plant is selected from the plants listed in table C and the carboxamide compound is isopyrazam.

In a more preferred embodiment, the present invention relates of methods of controlling harmful fungi and/or increasing the health of plants by treating cultivated plants, parts of such plants or at their locus of growth with a carboxamide compound, wherein the plant is selected from the plants listed in table C and the carboxamide compound is penthiopyrad.

TABLE C
(source: Phillips McDougall AgriService,
Seed Service May 2009)

| No | seed name | crop | company |
|---|---|---|---|
| C-1 | Agrisure 3000GT | maize | Syngenta |
| C-2 | Agrisure CB/LL | maize | Syngenta |
| C-3 | Agrisure CB/LL/RW | maize | Syngenta |
| C-4 | Agrisure GT | maize | Syngenta |
| C-5 | Agrisure GT/CB/LL | maize | Syngenta |
| C-6 | Agrisure GT/RW | maize | Syngenta |
| C-7 | Agrisure RW | maize | Syngenta |
| C-8 | Bollgard | cotton | Monsanto |
| C-9 | Bollgard II | cotton | Monsanto |
| C-10 | Bollgard II RR Flex Cotton | cotton | Monsanto |
| C-11 | Bt-Xtra | maize | DeKalb |
| C-12 | Clearfield canola | canola | BASF |
| C-13 | Clearfield corn | maize | BASF |
| C-14 | Clearfield rice | rice | BASF |
| C-15 | Clearfield sunflower | sunflower | BASF |
| C-16 | Clearfield wheat | wheat | BASF |
| C-17 | Herculex 1 | maize | Dow/Pioneer |
| C-18 | Herculex Quad-Stack | maize | Dow/Pioneer |
| C-19 | Herculex RW | maize | Dow/Pioneer |
| C-20 | Herculex XTRA | maize | Dow/Pioneer |
| C-21 | Herculex Xtra | maize | Dow/Pioneer |
| C-22 | Knock Out | maize | Novartis |
| C-23 | Liberty Link | canola | AgrEvo |
| C-24 | Liberty Link | maize | Bayer |
| C-25 | Liberty Link | cotton | Bayer |
| C-26 | Maximiser | maize | Syngenta |
| C-27 | Nature Guard ® | maize | Dow |
| C-28 | New Leaf Potato | potato | Monsanto |
| C-29 | Optimum GAT | maize | DuPont |
| C-30 | Optimum GAT | cotton | DuPont |
| C-31 | Optimum GAT | soybean | DuPont |
| C-32 | Poast Compatibel | maize | BASF |
| C-33 | Roundup Ready 2 Yield | canola | Monsanto |
| C-34 | Roundup Ready 2 Yield | maize | Monsanto |
| C-35 | Roundup Ready 2 Yield | cotton | Monsanto |
| C-36 | Roundup Ready 2 Yield | soybean | Monsanto |
| C-37 | Roundup Ready Alfalfa | alfalfa | Monsanto |
| C-38 | Roundup Ready Bollgard | cotton | Monsanto |
| C-39 | Roundup Ready Bollgard II | cotton | Monsanto |
| C-40 | Roundup Ready Canola | canola | Monsanto |
| C-41 | Roundup Ready Corn | maize | Monsanto |
| C-42 | Roundup Ready Corn 2 | maize | Monsanto |
| C-43 | Roundup Ready Cotton | cotton | Monsanto |
| C-44 | Roundup Ready Flex | cotton | Monsanto |
| C-45 | Roundup Ready Flex Bollgard II | cotton | Monsanto |
| C-46 | Roundup Ready Soybean | soybean | Monsanto |
| C-47 | Roundup Ready Sugarbeet | sugarbeet | KWS/SES/Hilleshog |
| C-48 | Roundup Ready YieldGard | maize | Monsanto |
| | corn borer | | |
| C-49 | Roundup Ready YieldGard Plus | maize | Monsanto |
| C-50 | Roundup Ready, Herculex | maize | Dow/Pioneer |
| | XTRA | | |
| C-51 | StarLink | maize | Aventis |
| C-52 | Widestrike | cotton | Dow |
| C-53 | YieldGard | maize | Monsanto |
| C-54 | YieldGard corn borer and corn | maize | Monsanto |
| | rootworm | | |
| C-55 | YieldGard Corn Rootworm | maize | Monsanto |
| C-56 | YieldGard Plus RR Corn 2 | maize | Monsanto |
| C-57 | YieldGard rootworm RR Corn 2 | maize | Monsanto |
| C-58 | YieldGard | maize | Monsanto |

In an utmost preferred embodiment, the present invention relates to a method of controlling harmful fungi and/or increasing the health of cultivated plants by treating cultivated plants, parts of such plants or at their locus of growth with a carboxamide compound, wherein the plant and the carboxamide compound are selected as given in table D.

TABLE D

| No | Pesticide | crop | Gene |
|---|---|---|---|
| D-1 | Boscalid | canola | bar |
| D-2 | Boscalid | canola | bxn |
| D-3 | Boscalid | canola | CP4 |
| | | | epsps |
| D-4 | Boscalid | canola | goxv247 |
| D-5 | Boscalid | canola | pat |
| D-6 | Boscalid | maize | CP4 |
| | | | epsps |
| D-7 | Boscalid | maize | Cry1Ab |
| D-8 | Boscalid | maize | Cry1Ac |
| D-9 | Boscalid | maize | Cry1F |
| D-10 | Boscalid | maize | Cry1Fa2 |
| D-11 | Boscalid | maize | Cry34Ab1 |
| D-12 | Boscalid | maize | Cry35Ab1 |
| D-13 | Boscalid | maize | Cry3A |
| D-14 | Boscalid | maize | Cry3Bb1 |
| D-15 | Boscalid | maize | Cry9C |
| D-16 | Boscalid | maize | goxv247 |
| D-17 | Boscalid | maize | pat |
| D-18 | Boscalid | maize | vip3A |
| D-19 | Boscalid | cotton | ALS |
| D-20 | Boscalid | cotton | bxn |
| D-21 | Boscalid | cotton | CP4 |
| | | | epsps |
| D-22 | Boscalid | cotton | Cry1Ac |
| D-23 | Boscalid | cotton | Cry1F |
| D-24 | Boscalid | cotton | Cry2Ab |
| D-25 | Boscalid | cotton | pat |
| D-26 | Boscalid | cotton | vip3A(a) |
| D-27 | Boscalid | soybean | ALS |
| D-28 | Boscalid | soybean | CP4 |
| | | | epsps |
| D-29 | Boscalid | soybean | pat |
| D-30 | N-(3′,4′,5′-trifluorobiphenyl- | canola | bar |
| | 2-yl)-3-difluoromethyl-1-methyl-1H- | | |
| | pyrazole-4-carboxamide | | |
| D-31 | N-(3′,4′,5′-trifluorobiphenyl- | canola | bxn |
| | 2-yl)-3-difluoromethyl-1-methyl-1H- | | |
| | pyrazole-4-carboxamide | | |
| D-32 | N-(3′,4′,5′-trifluorobiphenyl- | canola | CP4 |
| | 2-yl)-3-difluoromethyl-1-methyl-1H- | | epsps |
| | pyrazole-4-carboxamide | | |
| D-33 | N-(3′,4′,5′-trifluorobiphenyl- | canola | goxv247 |
| | 2-yl)-3-difluoromethyl-1-methyl-1H- | | |
| | pyrazole-4-carboxamide | | |
| D-34 | N-(3′,4′,5′-trifluorobiphenyl- | canola | pat |
| | 2-yl)-3-difluoromethyl-1-methyl-1H- | | |
| | pyrazole-4-carboxamide | | |
| D-35 | N-(3′,4′,5′-trifluorobiphenyl- | maize | CP4 |
| | 2-yl)-3-difluoromethyl-1-methyl-1H- | | epsps |
| | pyrazole-4-carboxamide | | |
| D-36 | N-(3′,4′,5′-trifluorobiphenyl- | maize | Cry1Ab |
| | 2-yl)-3-difluoromethyl-1-methyl-1H- | | |
| | pyrazole-4-carboxamide | | |
| D-37 | N-(3′,4′,5′-trifluorobiphenyl- | maize | Cry1Ac |
| | 2-yl)-3-difluoromethyl-1-methyl-1H- | | |
| | pyrazole-4-carboxamide | | |
| D-38 | N-(3′,4′,5′-trifluorobiphenyl- | maize | Cry1F |
| | 2-yl)-3-difluoromethyl-1-methyl-1H- | | |
| | pyrazole-4-carboxamide | | |
| D-39 | N-(3′,4′,5′-trifluorobiphenyl- | maize | Cry1Fa2 |
| | 2-yl)-3-difluoromethyl-1-methyl-1H- | | |
| | pyrazole-4-carboxamide | | |
| D-40 | N-(3′,4′,5′-trifluorobiphenyl- | maize | Cry34Ab1 |
| | 2-yl)-3-difluoromethyl-1-methyl-1H- | | |
| | pyrazole-4-carboxamide | | |
| D-41 | N-(3′,4′,5′-trifluorobiphenyl- | maize | Cry35Ab1 |
| | 2-yl)-3-difluoromethyl-1-methyl-1H- | | |
| | pyrazole-4-carboxamide | | |
| D-42 | N-(3′,4′,5′-trifluorobiphenyl- | maize | Cry3A |
| | 2-yl)-3-difluoromethyl-1-methyl-1H- | | |
| | pyrazole-4-carboxamide | | |
| D-43 | N-(3′,4′,5′-trifluorobiphenyl- | maize | Cry3Bb1 |
| | 2-yl)-3-difluoromethyl-1-methyl-1H- | | |
| | pyrazole-4-carboxamide | | |
| D-44 | N-(3′,4′,5′-trifluorobiphenyl- | maize | Cry9C |
| | 2-yl)-3-difluoromethyl-1-methyl-1H- | | |
| | pyrazole-4-carboxamide | | |
| D-45 | N-(3′,4′,5′-trifluorobiphenyl- | maize | goxv247 |
| | 2-yl)-3-difluoromethyl-1-methyl-1H- | | |
| | pyrazole-4-carboxamide | | |
| D-46 | N-(3′,4′,5′-trifluorobiphenyl- | maize | pat |
| | 2-yl)-3-difluoromethyl-1-methyl-1H- | | |
| | pyrazole-4-carboxamide | | |
| D-47 | N-(3′,4′,5′-trifluorobiphenyl- | maize | vip3A |
| | 2-yl)-3-difluoromethyl-1-methyl-1H- | | |
| | pyrazole-4-carboxamide | | |
| D-48 | N-(3′,4′,5′-trifluorobiphenyl- | cotton | ALS |
| | 2-yl)-3-difluoromethyl-1-methyl-1H- | | |
| | pyrazole-4-carboxamide | | |
| D-49 | N-(3′,4′,5′-trifluorobiphenyl- | cotton | bxn |
| | 2-yl)-3-difluoromethyl-1-methyl-1H- | | |
| | pyrazole-4-carboxamide | | |
| D-50 | N-(3′,4′,5′-trifluorobiphenyl- | cotton | CP4 |
| | 2-yl)-3-difluoromethyl-1-methyl-1H- | | epsps |
| | pyrazole-4-carboxamide | | |
| D-51 | N-(3′,4′,5′-trifluorobiphenyl- | cotton | Cry1Ac |
| | 2-yl)-3-difluoromethyl-1-methyl-1H- | | |
| | pyrazole-4-carboxamide | | |
| D-52 | N-(3′,4′,5′-trifluorobiphenyl- | cotton | Cry1F |
| | 2-yl)-3-difluoromethyl-1-methyl-1H- | | |
| | pyrazole-4-carboxamide | | |
| D-53 | N-(3′,4′,5′-trifluorobiphenyl- | cotton | Cry2Ab |
| | 2-yl)-3-difluoromethyl-1-methyl-1H- | | |
| | pyrazole-4-carboxamide | | |
| D-54 | N-(3′,4′,5′-trifluorobiphenyl- | cotton | pat |
| | 2-yl)-3-difluoromethyl-1-methyl-1H- | | |
| | pyrazole-4-carboxamide | | |
| D-55 | N-(3′,4′,5′-trifluorobiphenyl- | cotton | vip3A(a) |
| | 2-yl)-3-difluoromethyl-1-methyl-1H- | | |
| | pyrazole-4-carboxamide | | |
| D-56 | N-(3′,4′,5′-trifluorobiphenyl- | soybean | ALS |
| | 2-yl)-3-difluoromethyl-1-methyl-1H- | | |
| | pyrazole-4-carboxamide | | |
| D-57 | N-(3′,4′,5′-trifluorobiphenyl- | soybean | CP4 |
| | 2-yl)-3-difluoromethyl-1-methyl-1H- | | epsps |
| | pyrazole-4-carboxamide | | |
| D-58 | N-(3′,4′,5′-trifluorobiphenyl- | soybean | pat |
| | 2-yl)-3-difluoromethyl-1-methyl-1H- | | |
| | pyrazole-4-carboxamide | | |

All embodiments of the carboxamide compound as defined above are also referred to herein after as carboxamide compound according to the present invention. They can also be converted into agrochemical compositions comprising a solvent or solid carrier and at least one carboxamide compounds according to the present invention.

An agrochemical composition comprises a fungicidally and/or plant health effective amount of a carboxamide compounds according to the present invention. The term “effective amount” denotes an amount of the composition or of the carboxamide compounds according to the present invention, which is sufficient to achieve the synergistic effects related to fungal control and/or plant health and which does not result in a substantial damage to the treated plants. Such an amount can vary in a broad range and is dependent on various factors, such as the fungal species to be controlled, the treated cultivated plant or material, the climatic conditions.

Examples of agrochemical compositions are solutions, emulsions, suspensions, dusts, powders, pastes and granules. The composition type depends on the particular intended purpose; in each case, it should ensure a fine and uniform distribution of the compound according to the invention.

More precise examples for composition types are suspensions (SC, OD, FS), pastes, pastilles, wettable powders or dusts (WP, SP, SS, WS, DP, DS) or granules (GR, FG, GG, MG), which can be water-soluble or wettable, as well as gel formulations for the treatment of plant propagation materials such as seeds (GF). Usually the composition types (e.g. SC, OD, FS, WG, SG, WP, SP, SS, WS, GF) are employed diluted. Composition types such as DP, DS, GR, FG, GG and MG are usually used undiluted.

The compositions are prepared in a known manner (cf. U.S. Pat. No. 3,060,084, EP-A 707 445 (for liquid concentrates), Browning: “Agglomeration”, Chemical Engineering, Dec. 4, 1967, 147-48, Perry's Chemical Engineer's Handbook, 4th Ed., McGraw-Hill, New York, 1963, S. 8-57 and ff. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman: Weed Control as a Science (J. Wiley & Sons, New York, 1961), Hance et al.: Weed Control Handbook (8th Ed., Blackwell Scientific, Oxford, 1989) and Mollet, H. and Grubemann, A.: Formulation technology (Wiley VCH Verlag, Weinheim, 2001).

The agrochemical compositions may also comprise auxiliaries which are customary in agrochemical compositions. The auxiliaries used depend on the particular application form and active substance, respectively.

Examples for suitable auxiliaries are solvents, solid carriers, dispersants or emulsifiers (such as further solubilizers, protective colloids, surfactants and adhesion agents), organic and anorganic thickeners, bactericides, anti-freezing agents, anti-foaming agents, if appropriate colorants and tackifiers or binders (e.g. for seed treatment formulations).

Suitable solvents are water, organic solvents such as mineral oil fractions of medium to high boiling point, such as kerosene or diesel oil, furthermore coal tar oils and oils of vegetable or animal origin, aliphatic, cyclic and aromatic hydrocarbons, e.g. toluene, xylene, paraffin, tetrahydronaphthalene, alkylated naphthalenes or their derivatives, alcohols such as methanol, ethanol, propanol, butanol and cyclohexanol, glycols, ketones such as cyclohexanone and gamma-butyrolactone, fatty acid dimethylamides, fatty acids and fatty acid esters and strongly polar solvents, e.g. amines such as N-methylpyrrolidone.

Solid carriers are mineral earths such as silicates, silica gels, talc, kaolins, limestone, lime, chalk, bole, loess, clays, dolomite, diatomaceous earth, calcium sulfate, magnesium sulfate, magnesium oxide, ground synthetic materials, fertilizers, such as, e.g., ammonium sulfate, ammonium phosphate, ammonium nitrate, ureas, and products of vegetable origin, such as cereal meal, tree bark meal, wood meal and nutshell meal, cellulose powders and other solid carriers.

Suitable surfactants (adjuvants, wtters, tackifiers, dispersants or emulsifiers) are alkali metal, alkaline earth metal and ammonium salts of aromatic sulfonic acids, such as ligninsoulfonic acid (Borresperse® types, Borregard, Norway) phenolsulfonic acid, naphthalenesulfonic acid (Morwet® types, Akzo Nobel, U.S.A.), dibutylnaphthalene-sulfonic acid (Nekal® types, BASF, Germany), and fatty acids, alkylsulfonates, alkylarylsulfonates, alkyl sulfates, laurylether sulfates, fatty alcohol sulfates, and sulfated hexa-, hepta- and octadecanolates, sulfated fatty alcohol glycol ethers, furthermore condensates of naphthalene or of naphthalenesulfonic acid with phenol and formaldehyde, polyoxy-ethylene octylphenyl ether, ethoxylated isooctylphenol, octylphenol, nonylphenol, alkylphenyl polyglycol ethers, tributylphenyl polyglycol ether, tristearylphenyl polyglycol ether, alkylaryl polyether alcohols, alcohol and fatty alcohol/ethylene oxide condensates, ethoxylated castor oil, polyoxyethylene alkyl ethers, ethoxylated polyoxypropylene, lauryl alcohol polyglycol ether acetal, sorbitol esters, lignin-sulfite waste liquors and proteins, denatured proteins, polysaccharides (e.g. methylcellulose), hydrophobically modified starches, polyvinyl alcohols (Mowiol® types, Clariant, Switzerland), polycarboxylates (Sokolan® types, BASF, Germany), polyalkoxylates, polyvinylamines (Lupasol® types, BASF, Germany), polyvinylpyrrolidone and the copolymers thereof.

Examples for thickeners (i.e. compounds that impart a modified flowability to compositions, i.e. high viscosity under static conditions and low viscosity during agitation) are polysaccharides and organic and anorganic clays such as Xanthan gum (Kelzan®, CP Kelco, U.S.A.), Rhodopol® 23 (Rhodia, France), Veegum® (R.T. Vanderbilt, U.S.A.) or Attaclay® (Engelhard Corp., NJ, USA).

Bactericides may be added for preservation and stabilization of the composition. Examples for suitable bactericides are those based on dichlorophene and benzylalcohol hemi formal (Proxel® from ICI or Acticide® RS from Thor Chemie and Kathon® MK from Rohm & Haas) and isothiazolinone derivatives such as alkylisothiazolinones and benzisothiazolinones (Acticide® MBS from Thor Chemie).

Examples for suitable anti-freezing agents are ethylene glycol, propylene glycol, urea and glycerin.

Examples for anti-foaming agents are silicone emulsions (such as e.g. Silikon® SRE, Wacker, Germany or Rhodorsil®, Rhodia, France), long chain alcohols, fatty acids, salts of fatty acids, fluoroorganic compounds and mixtures thereof.

Suitable colorants are pigments of low water solubility and water-soluble dyes. Examples to be mentioned and the designations rhodamin B, C. I. pigment red 112, C. I. solvent red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples for tackifiers or binders are polyvinylpyrrolidons, polyvinylacetates, polyvinyl alcohols and cellulose ethers (Tylose®, Shin-Etsu, Japan).

Powders, materials for spreading and dusts can be prepared by mixing or concomitantly grinding the compounds I and, if appropriate, further active substances, with at least one solid carrier.

Granules, e.g. coated granules, impregnated granules and homogeneous granules, can be prepared by binding the active substances to solid carriers. Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, e.g., ammonium sulfate, ammonium phosphate, ammonium nitrate, ureas, and products of vegetable origin, such as cereal meal, tree bark meal, wood meal and nutshell meal, cellulose powders and other solid carriers.

### Examples for Composition Types are:

1. Composition Types for Dilution with Water

### i) Water-Soluble Concentrates (SL, LS)

10 parts by weight of a carboxamide compounds according to the present invention are dissolved in 90 parts by weight of water or in a water-soluble solvent. As an alternative, wetting agents or other auxiliaries are added. The active substance dissolves upon dilution with water. In this way, a composition having a content of 10% by weight of active substance is obtained.

### ii) Dispersible Concentrates (DC)

20 parts by weight of a carboxamide compounds according to the present invention are dissolved in 70 parts by weight of cyclohexanone with addition of 10 parts by weight of a dispersant, e.g. polyvinylpyrrolidone. Dilution with water gives a dispersion. The active substance content is 20% by weight.

iii) Emulsifiable Concentrates (EC)

15 parts by weight of a carboxamide compounds according to the present invention are dissolved in 75 parts by weight of xylene with addition of calcium dodecylbenzenesulfonate and castor oil ethoxylate (in each case 5 parts by weight). Dilution with water gives an emulsion. The composition has an active substance content of 15% by weight.

### iv) Emulsions (EW, EO, ES)

25 parts by weight of carboxamide compounds according to the present invention are dissolved in 35 parts by weight of xylene with addition of calcium dodecylbenzenesulfonate and castor oil ethoxylate (in each case 5 parts by weight). This mixture is introduced into 30 parts by weight of water by means of an emulsifying machine (Ultraturrax) and made into a homogeneous emulsion. Dilution with water gives an emulsion. The composition has an active substance content of 25% by weight.

### v) Suspensions (SC, OD, FS)

In an agitated ball mill, 20 parts by weight of a carboxamide compounds according to the present invention are comminuted with addition of 10 parts by weight of dispersants and wetting agents and 70 parts by weight of water or an organic solvent to give a fine active substance suspension. Dilution with water gives a stable suspension of the active substance. The active substance content in the composition is 20% by weight.

### vi) Water-Dispersible Granules And Water-Soluble Granules (WG, SG)

50 parts by weight of a carboxamide compounds according to the present invention are ground finely with addition of 50 parts by weight of dispersants and wetting agents and prepared as water-dispersible or water-soluble granules by means of technical appliances (e.g. extrusion, spray tower, fluidized bed). Dilution with water gives a stable dispersion or solution of the active substance. The composition has an active substance content of 50% by weight.

vii) Water-Dispersible Powders And Water-Soluble Powders (WP, SP, SS, WS)

75 parts by weight of a carboxamide compounds according to the present invention are ground in a rotor-stator mill with addition of 25 parts by weight of dispersants, wetting agents and silica gel. Dilution with water gives a stable dispersion or solution of the active substance. The active substance content of the composition is 75% by weight.

viii) Gel (GF)

In an agitated ball mill, 20 parts by weight of a carboxamide compounds according to the present invention are comminuted with addition of 10 parts by weight of dispersants, 1 part by weight of a gelling agent wetters and 70 parts by weight of water or of an organic solvent to give a fine suspension of the active substance. Dilution with water gives a stable suspension of the active substance, whereby a composition with 20% (w/w) of active substance is obtained.

### 2. Composition Types to be Applied Undiluted

### ix) Dustable Powders (DP, DS)

5 parts by weight of a carboxamide compounds according to the present invention are ground finely and mixed intimately with 95 parts by weight of finely divided kaolin. This gives a dustable composition having an active substance content of 5% by weight.

### x) Granules (GR, FG, GG, MG)

0.5 parts by weight of a carboxamide compounds according to the present invention according to the invention is ground finely and associated with 99.5 parts by weight of carriers. Current methods are extrusion, spray-drying or the fluidized bed. This gives granules to be applied undiluted having an active substance content of 0.5% by weight.

### xi) ULV Solutions (UL)

10 parts by weight of a carboxamide compounds according to the present invention are dissolved in 90 parts by weight of an organic solvent, e.g. xylene. This gives a composition to be applied undiluted having an active substance content of 10% by weight.

The agrochemical compositions generally comprise between 0.01 and 95%, preferably between 0.1 and 90%, most preferably between 0.5 and 90%, by weight of active substance. The active substances are employed in a purity of from 90% to 100%, preferably from 95% to 100% (according to NMR spectrum). Water-soluble concentrates (LS), flowable concentrates (FS), powders for dry treatment (DS), water-dispersible powders for slurry treatment (WS), water-soluble powders (SS), emulsions (ES) emulsifiable concentrates (EC) and gels (GF) are usually employed for the purposes of treatment of plant propagation materials, particularly seeds. These compositions can be applied to plant propagation materials, particularly seeds, diluted or undiluted. The compositions in question give, after two-to-tenfold dilution, active substance concentrations of from 0.01 to 60% by weight, preferably from 0.1 to 40% by weight, in the ready-to-use preparations. Application can be carried out before or during sowing. Methods for applying or treating agrochemical compounds and compositions thereof, respectively, on to plant propagation material, especially seeds, are known in the art, and include dressing, coating, pelleting, dusting, soaking and in-furrow application methods of the propagation material. In a preferred embodiment, the compounds or the compositions thereof, respectively, are applied on to the plant propagation material by a method such that germination is not induced, e.g. by seed dressing, pelleting, coating and dusting.

In a preferred embodiment, a suspension-type (FS) composition is used for seed treatment. Typically, a FS composition may comprise 1-800 g/l of active substance, 1-200 g/l Surfactant, 0 to 200 g/l antifreezing agent, 0 to 400 g/l of binder, 0 to 200 g/l of a pigment and up to 1 liter of a solvent, preferably water. The carboxamide compounds according to the present invention can be used as such or in the form of their compositions, e.g. in the form of directly sprayable solutions, powders, suspensions, dispersions, emulsions, oil dispersions, pastes, dustable products, materials for spreading, or granules, by means of spraying, atomizing, dusting, spreading, brushing, immersing or pouring. The application forms depend entirely on the intended purposes; it is intended to ensure in each case the finest possible distribution of the active substances according to the invention.

Aqueous application forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. Alternatively, it is possible to prepare concentrates composed of active substance, wetter, tackifier, dispersant or emulsifier and, if appropriate, solvent or oil, and such concentrates are suitable for dilution with water.

The active substance concentrations in the ready-to-use preparations can be varied within relatively wide ranges. In general, they are from 0.0001 to 10%, preferably from 0.001 to 1% by weight of active substance.

The active substances may also be used successfully in the ultra-low-volume process (ULV), it being possible to apply compositions comprising over 95% by weight of active substance, or even to apply the active substance without additives.

The amounts of active substances applied are, depending on the kind of effect desired, from 0.001 to 2 kg per ha, preferably from 0.005 to 2 kg per ha, more preferably from 0.05 to 0.9 kg per ha, in particular from 0.1 to 0.75 kg per ha.

In treatment of plant propagation materials such as seeds, e.g. by dusting, coating or drenching seed, amounts of active substance of from 0.1 to 1000 g, preferably from 1 to 1000 g, more preferably from 1 to 100 g and most preferably from 5 to 100 g, per 100 kilogram of plant propagation material (preferably seed) are generally required.

Various types of oils, wetters, adjuvants, herbicides, bactericides, other fungicides and/or pesticides may be added to the active substances or the compositions comprising them, if appropriate not until immediately prior to use (tank mix). These agents can be admixed with the compositions according to the invention in a weight ratio of 1:100 to 100:1, preferably 1:10 to 10:1.

Adjuvants which can be used are in particular organic modified polysiloxanes such as Break Thru S 240®; alcohol alkoxylates such as Atplus 245®, Atplus MBA 1303®, Plurafac LF 300® and Lutensol ON 30®; EO/PO block polymers, e.g. Pluronic RPE 2035® and Genapol B®; alcohol ethoxylates such as Lutensol XP 80®; and dioctyl sulfosuccinate sodium such as Leophen RA®.

The compositions according to the invention can, in the use form as fungicides, also be present together with other active substances, e.g. with herbicides, insecticides, growth regulators, fungicides or else with fertilizers, as pre-mix or, if appropriate, not until immediately prior to use (tank mix).

In a preferred embodiment of the invention, the inventive mixtures are used for the protection of the plant propagation material, e.g. the seeds and the seedlings' roots and shoots, preferably the seeds.

Seed treatment can be made into the seedbox before planting into the field.

For seed treatment purposes, the weight ration in the binary, ternary and quaternary mixtures of the present invention generally depends from the properties of the carboxamide compounds according to the present invention.

Compositions, which are especially useful for seed treatment are e.g.:

A Soluble concentrates (SL, LS)

### D Emulsions (EW, EO, ES)

### E Suspensions (SC, OD, FS)

F Water-dispersible granules and water-soluble granules (WG, SG)
G Water-dispersible powders and water-soluble powders (WP, SP, WS)

### H Gel-Formulations (GF)

I Dustable powders (DP, DS)

These compositions can be applied to plant propagation materials, particularly seeds, diluted or undiluted. These compositions can be applied to plant propagation materials, particularly seeds, diluted or undiluted. The compositions in question give, after two-to-tenfold dilution, active substance concentrations of from 0.01 to 60% by weight, preferably from 0.1 to 40% by weight, in the ready-to-use preparations. Application can be carried out before or during sowing. Methods for applying or treating agrochemical compounds and compositions thereof, respectively, on to plant propagation material, especially seeds, are known in the art, and include dressing, coating, pelleting, dusting and soaking application methods of the propagation material (and also in furrow treatment). In a preferred embodiment, the compounds or the compositions thereof, respectively, are applied on to the plant propagation material by a method such that germination is not induced, e.g. by seed dressing, pelleting, coating and dusting.

In the treatment of plant propagation material (preferably seed), the application rates of the inventive mixture are generally for the formulated product (which usually comprises from 10 to 750 g/l of the active(s).

The invention also relates to the propagation products of plants, and especially the seed comprising, that is, coated with and/or containing, a mixture as defined above or a composition containing the mixture of two or more active ingredients or a mixture of two or more compositions each providing one of the active ingredients. The plant propagation material (preferably seed) comprises the inventive mixtures in an amount of from 0.1 g to 10 kg per 100 kg of plant propagation material (preferably seed).

The process of the present invention uses in one embodiment transgenic plants, parts thereof, cells or organelles.

For the purposes of the invention, “transgenic”, “transgene” or “recombinant” means with regard to, for example, a nucleic acid sequence, an expression cassette, gene construct or a vector comprising the nucleic acid sequence or an organism transformed with the nucleic acid sequences, expression cassettes or vectors, all those constructions brought about by recombinant methods in which either
(a) the nucleic acid sequences encoding proteins useful in the methods of the invention, or
(b) genetic control sequence(s) which is operably linked with the nucleic acid sequence according to the invention, for example a promoter, or
(c) a) and b)
   are not located in their natural genetic environment or have been modified by recombinant methods, it being possible for the modification to take the form of, for example, a substitution, addition, deletion, inversion or insertion of one or more nucleotide residues. The natural genetic environment is understood as meaning the natural genomic or chromosomal locus in the original plant and can be deduced from the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 2000/15815.

A transgenic plant for the purposes of the invention is thus understood as meaning, as above, that the nucleic acids are not at their natural locus in the genome of said plant, it being possible for the nucleic acids to be expressed homologously or heterologously. However, as mentioned, transgenic also means that, while the nucleic acids are at their natural position in the genome of a plant, the sequence has been modified with regard to the natural sequence, and/or that the regulatory sequences of the natural sequences have been modified. Transgenic is preferably understood as meaning the expression of the nucleic acids at an unnatural locus in the genome, i.e. homologous or, preferably, heterologous expression of the nucleic acids takes place. Preferred transgenic plants are mentioned herein.

These transgenic plants may be any listed in Table A, such as any of A-1 to A-156. Further, the trans-genic plants used in the process of the invention may comprise as transgene any one or several of the genes listed in Table B.

However, the present inventive process is not limited to transgenic plants, and not to these transgenic plants. Other transgenic plants suitable for the process of the present invention may be generated by methods known in the art. In the following section exemplary methods to produce transgenic plants suitable fort the process of the present invention are exemplified in a non-limiting fashion. The person skilled in the art is well aware that the methods used to produce the transgenic plants are not critical for the use of such plants in working the present invention.

The term “introduction” or “transformation” as referred to herein encompasses the transfer of an exogenous polynucleotide into a host cell, irrespective of the method used for transfer. In particular with respect to transgenic plants “transformation” or “transformed” preferably refers to the transfer of an exogenous polynucleotide into a host cell, irrespective of the method used for transfer.

Transformation methods include the use of liposomes, electroporation, chemicals that increase free DNA uptake, injection of the DNA directly into the plant, particle gun bombardment, transformation using viruses or pollen and microprojection. Methods may be selected from the calcium/polyethylene glycol method for protoplasts (Krens, F. A. et al., (1982) Nature 296, 72-74; Negrutiu I et al. (1987) Plant Mol Biol 8: 363-373); electroporation of protoplasts (Shillito R. D. et al. (1985) Bio/Technol 3, 1099-1102); microinjection into plant material (Crossway A et al., (1986) Mol. Gen. Genet. 202: 179-185); DNA or RNA-coated particle bombardment (Klein T M et al., (1987) Nature 327: 70) infection with (non-integrative) viruses and the like. Transgenic plants, including transgenic crop plants, are preferably produced via Agrobacterium-mediated transformation.

For example a suitable vector, e.g. a binary vector can be transformed into a suitable Agrobacterium strain e.g. LBA4044 according to methods well known in the art. Such a transformed Agrobacterium may then be used to transform plant cells, as disclosed in the following examples.

### EXAMPLE I

### Plant Transformation Examples

### Rice Transformation

The Agrobacterium containing the expression vector is used to transform Oryza sativa plants. Mature dry seeds of the rice japonica cultivar Nipponbare are dehusked. Sterilization is carried out by incubating for one minute in 70% ethanol, followed by 30 minutes in 0.2% HgCl₂, followed by a 6 times 15 minutes ish with sterile distilled water. The sterile seeds are then germinated on a medium containing 2,4-D (callus induction medium). After incubation in the dark for four weeks, embryogenic, scutellum-derived calli are excised and propagated on the same medium. After two weeks, the calli are multiplied or propagated by subculture on the same medium for another 2 weeks. Embryogenic callus pieces are sub-cultured on fresh medium 3 days before co-cultivation (to boost cell division activity).

Agrobacterium strain LBA4404 containing the expression vector is used for co-cultivation. Agrobacterium is inoculated on AB medium with the appropriate antibiotics and cultured for 3 days at 28° C. The bacteria are then collected and suspended in liquid co-cultivation medium to a density (OD₆₀₀) of about 1. The suspension is then transferred to a Petri dish and the calli immersed in the suspension for 15 minutes. The callus tissues are then blotted dry on a filter paper and transferred to solidified, co-cultivation medium and incubated for 3 days in the dark at 25° C. Co-cultivated calli are grown on 2,4-D-containing medium for 4 weeks in the dark at 28° C. in the presence of a selection agent. During this period, rapidly growing resistant callus islands developed. After transfer of this material to a regeneration medium and incubation in the light, the embryogenic potential is released and shoots developed in the next four to five weeks. Shoots are excised from the calli and incubated for 2 to 3 weeks on an auxin-containing medium from which they are transferred to soil. Hardened shoots are grown under high humidity and short days in a greenhouse.

Approximately 35 independent T0 rice transformants are generated for one construct. The primary transformants are transferred from a tissue culture chamber to a greenhouse. After a quantitative PCR analysis to verify copy number of the T-DNA insert, only single copy transgenic plants that exhibit tolerance to the selection agent are kept for harvest of T1 seed. Seeds are then harvested three to five months after transplanting. The method yielded single locus transformants at a rate of over 50% (Aldemita and Hodges 1996, Chan et al. 1993, Hiei et al. 1994).

Approximately 35 independent T0 rice transformants are generated. The primary transformants are transferred from a tissue culture chamber to a greenhouse for growing and harvest of T1 seed. Six events, of which the T1 progeny segregated 3:1 for presence/absence of the transgene, are retained. For each of these events, approximately 10 T1 seedlings containing the transgene (hetero- and homozygotes) and approximately 10 T1 seedlings lacking the transgene (nullizygotes) are selected by monitoring visual marker expression.

### Corn Transformation

Transformation of maize (Zea mays) is performed with a modification of the method described by Ishida et al. (1996) Nature Biotech 14(6): 745-50. Transformation is genotype-dependent in corn and only specific genotypes are amenable to transformation and regeneration. The inbred line A188 (University of Minnesota) or hybrids with A188 as a parent are good sources of donor material for transformation, but other genotypes can be used successfully as well. Ears are harvested from corn plant approximately 11 days after pollination (DAP) when the length of the immature embryo is about 1 to 1.2 mm. Immature embryos are cocultivated with Agrobacterium tumefaciens containing the expression vector, and transgenic plants are recovered through organogenesis. Excised embryos are grown on callus induction medium, then maize regeneration medium, containing the selection agent (for example imidazolinone but various selection markers can be used). The Petri plates are incubated in the light at 25° C. for 2-3 weeks, or until shoots develop. The green shoots are transferred from each embryo to maize rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Wheat Transformation

Transformation of wheat is performed with the method described by Ishida et al. (1996) Nature Biotech 14(6): 745-50. The cultivar Bobwhite (available from CIMMYT, Mexico) is commonly used in transformation. Immature embryos are co-cultivated with Agrobacterium tumefaciens containing the expression vector, and transgenic plants are recovered through organogenesis. After incubation with Agrobacterium, the embryos are grown in vitro on callus induction medium, then regeneration medium, containing the selection agent (for example imidazolinone but various selection markers can be used). The Petri plates are incubated in the light at 25° C. for 2-3 weeks, or until shoots develop. The green shoots are transferred from each embryo to rooting medium and incubated at 25° C. for 2-3 weeks, until roots develop. The rooted shoots are transplanted to soil in the greenhouse. T1 seeds are produced from plants that exhibit tolerance to the selection agent and that contain a single copy of the T-DNA insert.

### Soybean Transformation

Soybean is transformed according to a modification of the method described in the Texas A&M patent U.S. Pat. No. 5,164,310. Several commercial soybean varieties are amenable to transformation by this method. The cultivar Jack (available from the Illinois Seed foundation) is commonly used for transformation. Soybean seeds are sterilised for in vitro sowing. The hypocotyl, the radicle and one cotyledon are excised from seven-day old young seedlings. The epicotyl and the remaining cotyledon are further grown to develop axillary nodes. These axillary nodes are excised and incubated with Agrobacterium tumefaciens containing the expression vector. After the cocultivation treatment, the explants are ished and transferred to selection media. Regenerated shoots are excised and placed on a shoot elongation medium. Shoots no longer than 1 cm are placed on rooting medium until roots develop. The rooted shoots are transplanted to soil in the greenhouse. T1 seeds are produced from plants that exhibit tolerance to the selection agent and that contain a single copy of the T-DNA insert.

### Rapeseed/Canola Transformation

Cotyledonary petioles and hypocotyls of 5-6 day old young seedling are used as explants for tissue culture and transformed according to Babic et al. (1998, Plant Cell Rep 17: 183-188). The commercial cultivar Westar (Agriculture Canada) is the standard variety used for transformation, but other varieties can also be used. Canola seeds are surface-sterilized for in vitro sowing. The cotyledon petiole explants with the cotyledon attached are excised from the in vitro seedlings, and inoculated with Agrobacterium (containing the expression vector) by dipping the cut end of the petiole explant into the bacterial suspension. The explants are then cultured for 2 days on MSBAP-3 medium containing 3 mg/l BAP, 3% sucrose, 0.7% Phytagar at 23° C., 16 hr light. After two days of co-cultivation with Agrobacterium, the petiole explants are transferred to MSBAP-3 medium containing 3 mg/l BAP, cefotaxime, carbenicillin, or timentin (300 mg/l) for 7 days, and then cultured on MSBAP-3 medium with cefotaxime, carbenicillin, or timentin and selection agent until shoot regeneration. When the shoots are 5-10 mm in length, they are cut and transferred to shoot elongation medium (MSBAP-0.5, containing 0.5 mg/l BAP). Shoots of about 2 cm in length are transferred to the rooting medium (MS0) for root induction. The rooted shoots are transplanted to soil in the greenhouse. T1 seeds are produced from plants that exhibit tolerance to the selection agent and that contain a single copy of the T-DNA insert.

### Alfalfa Transformation

A regenerating clone of alfalfa (Medicago sativa) is transformed using the method of (McKersie et al., 1999 Plant Physiol 119: 839-847). Regeneration and transformation of alfalfa is genotype dependent and therefore a regenerating plant is required. Methods to obtain regenerating plants have been described. For example, these can be selected from the cultivar Rangelander (Agriculture Canada) or any other commercial alfalfa variety as described by Brown DCW and A Atanassov (1985. Plant Cell Tissue Organ Culture 4: 111-112). Alternatively, the RA3 variety (University of Wisconsin) has been selected for use in tissue culture (Walker et al., 1978 μm J Bot 65:654-659). Petiole explants are cocultivated with an overnight culture of Agrobacterium tumefaciens C58C1 pMP90 (McKersie et al., 1999 Plant Physiol 119: 839-847) or LBA4404 containing the expression vector. The explants are cocultivated for 3 d in the dark on SH induction medium containing 288 mg/L Pro, 53 mg/L thioproline, 4.35 g/L K2SO4, and 100 μm acetosyringinone. The explants are ished in half-strength Murashige-Skoog medium (Murashige and Skoog, 1962) and plated on the same SH induction medium without acetosyringinone but with a suitable selection agent and suitable antibiotic to inhibit Agrobacterium growth. After several weeks, somatic embryos are transferred to BOi2Y development medium containing no growth regulators, no antibiotics, and 50 g/L sucrose. Somatic embryos are subsequently germinated on half-strength Murashige-Skoog medium. Rooted seedlings are transplanted into pots and grown in a greenhouse. T1 seeds are produced from plants that exhibit tolerance to the selection agent and that contain a single copy of the T-DNA insert.

### Cotton Transformation

Cotton is transformed using Agrobacterium tumefaciens according to the method described in U.S. Pat. No. 5,159,135. Cotton seeds are surface sterilised in 3% sodium hypochlorite solution during 20 minutes and ished in distilled water with 500 μg/ml cefotaxime. The seeds are then transferred to SH-medium with 50 μg/ml benomyl for germination. Hypocotyls of 4 to 6 days old seedlings are removed, cut into 0.5 cm pieces and are placed on 0.8% agar. An Agrobacterium suspension (approx. 108 cells per ml, diluted from an overnight culture transformed with the gene of interest and suitable selection markers) is used for inoculation of the hypocotyl explants. After 3 days at room temperature and lighting, the tissues are transferred to a solid medium (1.6 g/l Gelrite) with Murashige and Skoog salts with B5 vitamins (Gamborg et al., Exp. Cell Res. 50:151-158 (1968)), 0.1 mg/l 2,4-D, 0.1 mg/l 6-furfurylaminopurine and 750 μg/ml MgCL2, and with 50 to 100 μg/ml cefotaxime and 400-500 μg/ml carbenicillin to kill residual bacteria. Individual cell lines are isolated after two to three months (with subcultures every four to six weeks) and are further cultivated on selective medium for tissue amplification (30° C., 16 hr photoperiod). Transformed tissues are subsequently further cultivated on non-selective medium during 2 to 3 months to give rise to somatic embryos. Healthy looking embryos of at least 4 mm length are transferred to tubes with SH medium in fine vermiculite, supplemented with 0.1 mg/l indole acetic acid, 6 furfurylaminopurine and gibberellic acid. The embryos are cultivated at 30° C. with a photoperiod of 16 hrs, and plantlets at the 2 to 3 leaf stage are transferred to pots with vermiculite and nutrients. The plants are hardened and subsequently moved to the greenhouse for further cultivation. #

### Arabidopsis Plant Transformation

Approximately 30-60 ng of prepared vector and a defined amount of prepared amplificate are mixed and hybridized at 65° C. for 15 minutes followed by 37° C. 0.1° C./1 seconds, followed by 37° C. 10 minutes, followed by 0.1° C./1 seconds, then 4-10° C.

The ligated constructs are transformed in the same reaction vessel by addition of competent E. coli cells (strain DH5alpha) and incubation for 20 minutes at 1° C. followed by a heat shock for 90 seconds at 42° C. and cooling to 1-4° C. Then, complete medium (SOC) is added and the mixture is incubated for 45 minutes at 37° C. The entire mixture is subsequently plated onto an agar plate with 0.05 mg/ml kanamycine and incubated overnight at 37° C.

The outcome of the cloning step is verified by amplification with the aid of primers which bind upstream and downstream of the integration site, thus allowing the amplification of the insertion. The amplifications are carried out as described in the protocol of Taq DNA polymerase (Gibco-BRL).

The amplification cycles are as follows:

1 cycle of 1-5 minutes at 94° C., followed by 35 cycles of in each case 15-60 seconds at 94° C., 15-60 seconds at 50-66° C. and 5-15 minutes at 72° C., followed by 1 cycle of 10 minutes at 72° C., then 4-16° C.

Several colonies are checked, but only one colony for which a PCR product of the expected size is detected is used in the following steps.

A portion of this positive colony is transferred into a reaction vessel filled with complete medium (LB) supplemented with kanamycin and incubated overnight at 37° C.

The plasmid preparation is carried out as specified in the Qiaprep or NucleoSpin Multi-96 Plus standard protocol (Qiagen or Macherey-Nagel).

### Generation of Transgenic Plants

1-5 ng of the plasmid DNA isolated is transformed by electroporation or transformation into competent cells of Agrobacterium tumefaciens, of strain GV 3101 pMP90 (Koncz and Schell, Mol. Gen. Gent. 204, 383 (1986)). Thereafter, complete medium (YEP) is added and the mixture is transferred into a fresh reaction vessel for 3 hours at 28° C. Thereafter, all of the reaction mixture is plated onto YEP agar plates supplemented with the respective antibiotics, e.g. rifampicine (0.1 mg/ml), gentamycine (0.025 mg/ml and kanamycine (0.05 mg/ml) and incubated for 48 hours at 28° C.

The agrobacteria that contains the plasmid construct are then used for the transformation of plants.

A colony is picked from the agar plate with the aid of a pipette tip and taken up in 3 ml of liquid TB medium, which also contained suitable antibiotics as described above. The preculture is grown for 48 hours at 28° C. and 120 rpm.

400 ml of LB medium containing the same antibiotics as above are used for the main culture. The preculture is transferred into the main culture. It is grown for 18 hours at 28° C. and 120 rpm. After centrifugation at 4 000 rpm, the pellet is resuspended in infiltration medium (MS medium, 10% sucrose).

In order to grow the plants for the transformation, dishes (Piki Saat 80, green, provided with a screen bottom, 30×20 ×4.5 cm, from Wiesauplast, Kunststofftechnik, Germany) are half-filled with a GS 90 substrate (standard soil, Werkverband E. V., Germany). The dishes are watered overnight with 0.05% Proplant solution (Chimac-Apriphar, Belgium). Arabidopsis thaliana C24 seeds (Nottingham Arabidopsis Stock Centre, UK; NASC Stock N906) are scattered over the dish, approximately 1 000 seeds per dish. The dishes are covered with a hood and placed in the stratification facility (8 h, 110 μmol/m2s1, 22° C.; 16 h, dark, 6° C.). After 5 days, the dishes are placed into the short-day controlled environment chamber (8 h, 130 μmol/m2s1, 22° C.; 16 h, dark, 20° C.), where they remained for approximately 10 days until the first true leaves had formed.

The seedlings are transferred into pots containing the same substrate (Teku pots, 7 cm, LC series, manufactured by Pöppelmann GmbH & Co, Germany). Five plants are pricked out into each pot. The pots are then returned into the short-day controlled environment chamber for the plant to continue growing.

After 10 days, the plants are transferred into the greenhouse cabinet (supplementary illumination, 16 h, 340 μE/m2s, 22° C.; 8 h, dark, 20° C.), where they are allowed to grow for further 17 days.

For the transformation, 6-week-old Arabidopsis plants, which had just started flowering are immersed for 10 seconds into the above-described agrobacterial suspension which had previously been treated with 10 μl Silwett L77 (Crompton S. A., Osi Specialties, Switzerland). The method in question is described by Clough J. C. and Bent A. F. (Plant J. 16, 735 (1998)).

The plants are subsequently placed for 18 hours into a humid chamber. Thereafter, the pots are returned to the greenhouse for the plants to continue growing. The plants remained in the greenhouse for another 10 weeks until the seeds are ready for harvesting.

Depending on the resistance marker used for the selection of the transformed plants the harvested seeds are planted in the greenhouse and subjected to a spray selection or else first sterilized and then grown on agar plates supplemented with the respective selection agent. Since the vector contained the bar gene as the resistance marker, plantlets are sprayed four times at an interval of 2 to 3 days with 0.02% BASTA® and transformed plants are allowed to set seeds.

The seeds of the transgenic A. thaliana plants are stored in the freezer (at −20° C.).

### EXAMPLE II

Application of a carboxamide compound selected from the group consisting of boscalid, (N-(3′,4′,5′-trifluorobiphenyl-2-yl)-3-difluoromethyl-1-methyl-1H-pyrazole-4-carboxamide), bixafen, penflufen (N-[2-(1,3-dimethylbutyl)-phenyl]-1,3-dimethyl-5-fluoro-1H-pyrazole-4-carboxamide), fluopyram, sedaxane, isopyrazam, penthiopyrad, benodanil, carboxin, fenfuram, flutolanil, furametpyr, mepronil, oxycarboxin and thifluzamide.

### II.A Seed Treatments

Control and cultivated corn seeds of the T2 generation are treated with deionized water (Blank), 10 grams to 200 grams of a carboxamide compound; all formulation rates are grams/100 kg seed. Every formulation is applied to approximately 80 seeds. The formulation is pipetted into a 125 ml flask along the sides and bottom of the flask before adding the seeds and shaking the flask for 30 seconds. The coated seeds are then removed from the flask and placed in a plastic dish for drying.

Seventy-five 3-L pots per treatment are filled with potting media, labeled with colored stakes, and given a unique barcode. One seed per pot is planted at a depth of approximately 2 cm and covered with media. The media is lightly watered to imbibe the seeds, while allowing for ample oxygen exchange and so that the chemical coatings on the seeds remained intact. After planting, the pots are randomly distributed into three replicate blocks (1 bench=1 block), each with 25 plants of every treatment.

The plants are maintained in a greenhouse under optimal, well-watered conditions (80-90% field capacity) upon emergence. Supplemental nutrients are administered every third day during watering. The greenhouse temperature is maintained at 30° C., relative humidity at 75%, and light at 350 μmol m⁻²s⁻¹, in a 15-hour day/9-hour night photoperiod. Supplemental lighting is provided using metal-halide lights. Once per week, the pots are randomly mixed within each block.

On day 21, the plants are imaged to collect the phenotypic data as described in the WO2008/129060.

### II.B Plant Treatments

The cultivation of plants, their treatment with fungicides and the evaluation of the fungicidal activity are known to experts in the field. The treatment of plants with carboxamide s and the determination of infection after treatment is described for example in EP0545099, WO200307075, WO2006087343, WO200435589, EP846416, DE19629828, WO2003010149, EP1313709, JP 2000-342183, EP1110956, WO200142223, WO2000/09482, WO200366609, WO200374491, WO200435555, WO200439799 and EP915868.

### III. Evaluation

### III.A Evaluation Procedure of Rice Plants Subject to the Process of the Present Invention

### 1 Evaluation Setup

The cultivated plants and the corresponding controls are grown side-by-side at random positions. Greenhouse conditions are of shorts days (12 hours light), 28° C. in the light and 22° C. in the dark, and a relative humidity of 70%. Plants grown under non-stress conditions are watered at regular intervals to ensure that water and nutrients are not limiting and to satisfy plant needs to complete growth and development.

From the stage of sowing until the stage of maturity the plants are passed several times through a digital imaging cabinet. At each time point digital images (2048×1536 pixels, 16 million colours) are taken of each plant from at least 6 different angles.

### 2 Statistical Analysis: F Test

A two factor ANOVA (analysis of variants) is used as a statistical model for the overall evaluation of plant phenotypic characteristics. An F test is carried out on all the parameters measured of all the plants of all the. The threshold for significance for a true global gene effect is set at a 5% probability level for the F test.

### 3 Parameters Measured

### Biomass-Related Parameter Measurement

From the stage of sowing until the stage of maturity the plants are passed several times through a digital imaging cabinet. At each time point digital images (2048×1536 pixels, 16 million colours) are taken of each plant from at least 6 different angles.

The plant aboveground area (or leafy biomass) is determined by counting the total number of pixels on the digital images from aboveground plant parts discriminated from the background. This value is averaged for the pictures taken on the same time point from the different angles and is converted to a physical surface value expressed in square mm by calibration. Experiments show that the aboveground plant area measured this way correlates with the biomass of plant parts above ground. The above ground area is the area measured at the time point at which the plant had reached its maximal leafy biomass. The early vigour is the plant (seedling) aboveground area three weeks post-germination. Increase in root biomass is expressed as an increase in total root biomass (measured as maximum biomass of roots observed during the lifespan of a plant); or as an increase in the root/shoot index (measured as the ratio between root mass and shoot mass in the period of active growth of root and shoot).

Early vigour is determined by counting the total number of pixels from aboveground plant parts discriminated from the background. This value is averaged for the pictures taken on the same time point from different angles and is converted to a physical surface value expressed in square mm by calibration.

### Seed-Related Parameter Measurements

The mature primary panicles are harvested, counted, bagged, barcode-labelled and then dried for three days in an oven at 37° C. The panicles are then threshed and all the seeds are collected and counted. The filled husks are separated from the empty ones using an air-blowing device. The empty husks are discarded and the remaining fraction is counted again. The filled husks are weighed on an analytical balance. The number of filled seeds is determined by counting the number of filled husks that remained after the separation step. The total seed yield is measured by weighing all filled husks harvested from a plant. Total seed number per plant is measured by counting the number of husks harvested from a plant. Thousand Kernel Weight (TKW) is extrapolated from the number of filled seeds counted and their total weight. The Harvest Index (HI) in the present invention is defined as the ratio between the total seed yield and the above ground area (mm²), multiplied by a factor 10⁶. The total number of flowers per panicle as defined in the present invention is the ratio between the total number of seeds and the number of mature primary panicles. The seed fill rate as defined in the present invention is the proportion (expressed as a %) of the number of filled seeds over the total number of seeds (or florets).

### EXAMPLE III:B

### Evaluation Procedure of Arabidopsis Plants Subject to the Process of the Present Invention

Plant screening for yield increase under standardised growth conditions In this experiment, a plant screening for yield increase (in this case: biomass yield increase) under standardised growth conditions in the absence of substantial abiotic stress can be performed. In a standard experiment soil is prepared as 3.5:1 (v/v) mixture of nutrient rich soil (GS90, Tantau, Wansdorf, Germany) and quarz sand. Alternatively, plants can be sown on nutrient rich soil (GS90, Tantau, Germany). Pots can be filled with soil mixture and placed into trays. Water can be added to the trays to let the soil mixture take up appropriate amount of water for the sowing procedure. The seeds for transgenic A. thaliana plants and their controls for example non-trangenic wild-type can be sown in pots (6 cm diameter). Stratification can be established for a period of 3-4 days in the dark at 4° C.-5° C. Germination of seeds and growth can be initiated at a growth condition of 20° C., and approx. 60% relative humidity, 16 h photoperiod and illumination with fluorescent light at approximately 200 μmol/m²s. In case the transgenic seed are not uniformly transgenic a selection step can be performed, e.g. BASTA selection. This can be done at day 10 or day 11 (9 or 10 days after sowing) by spraying pots with plantlets from the top. In the standard experiment, a 0.07% (v/v) solution of BASTA concentrate (183 g/l glufosinate-ammonium) in tap water can be sprayed once or, alternatively, a 0.02% (v/v) solution of BASTA can be sprayed three times. The wild-type control plants can be sprayed with tap water only (instead of spraying with BASTA dissolved in tap water) but can be otherwise treated identically.

Plants can be individualized 13-14 days after sowing by removing the surplus of seedlings and leaving one seedling in soil. Transgenic events and control plants can be evenly distributed over the chamber. Watering can be carried out every two days after removing the covers in a standard experiment or, alternatively, every day.

Treatment with formulations of active ingredients can be performed as described in this application or by any known method.

For measuring biomass performance, plant fresh weight can be determined at harvest time (24-29 days after sowing) by cutting shoots and weighing them. Plants can be in the stage prior to flowering and prior to growth of inflorescence when harvested. Transgenic plants can be compared to the non-transgenic wild-control plants, which can be harvested at the same day. Significance values for the statistical significance of the biomass changes can be calculated by applying the ‘student's’ t test (parameters: two-sided, unequal variance).

Two different types of experimental procedures are performed:
Procedure 1). Per transgenic construct 3-4 independent transgenic lines (=events) are tested (22-30 plants per construct) and biomass performance can be evaluated as described above.
Procedure 2.) Up to five lines per transgenic construct can be tested in successive experimental levels (up to 4). Only constructs that displayed positive performance are subjected to the next experimental level. Usually in the first level five plants per construct can be tested and in the subsequent levels 30-60 plants can be tested. Biomass performance can be evaluated as described above. Data from this type of experiment (Procedure 2) are shown for constructs that displayed increased biomass performance in at least two successive experimental levels.

Biomass production can be measured by weighing plant rosettes. Biomass increase can be calculated as ratio of average weight of transgenic plants compared to average weight of control plants from the same experiment. The mean biomass increase of transgenics can be given (significance value <0.3 and biomass increase >5% (ratio>1.05)).

Seed yield can be measured by collecting all seed form a plant and measuring the thousand kernel weight. Various methods are known in the art.

### IV. Evaluation Procedure for Pest Control

The person skilled in the art is aware of suitable methods of inoculation and assessing infections for different plant species and pathogen types. The following are examples not limiting the present invention.

IV.A. Fungicidal Control of Rice Blast Caused by Pyricularia oryzae (Protective Action)

Leaves of pot-grown rice seedlings are sprayed to run-off with an aqueous suspension, containing the concentration of the active ingredient as described above. The plants are allowed to air-dry. At the following day the plants are inoculated with an aqueous spore suspension of Pyricularia oryzae containing 1×106 spores/ml. The test plants are immediately transferred into a humid chamber. After 6 days at 22-24° C. and relative atmospheric humidity closed to 100% the extent of fungal attack on the leaves is visually assessed as % diseased leaf area.

### IV.B Evaluating the Susceptibility to Soybean Rust

The soybean rust fungus is a wild isolate from Brazil.

The plants are inoculated with P. pachyrhizi.

In order to obtain appropriate spore material for the inoculation, soybean leaves which had been infected with soybean rust 15-20 days ago, are taken 2-3 days before the inoculation and transferred to agar plates (1% agar in H2O). The leaves are placed with their upper side onto the agar, which allows the fungus to grow through the tissue and to produce very young spores. For the inoculation solution, the spores are knocked off the leaves and are added to a Tween-H2O solution. The counting of spores is performed under a light microscope by means of a Thoma counting chamber. For the inoculation of the plants, the spore suspension is added into a compressed-air operated spray flask and applied uniformly onto the plants or the leaves until the leaf surface is well moisturized. For the microscopy, a density of 10×105 spores/ml is used. The inoculated plants are placed for 24 hours in a greenhouse chamber with an average of 22° C. and >90% of air humidity. The inoculated leaves are incubated under the same conditions in a closed Petri dish on 0.5% plant agar. The following cultivation is performed in a chamber with an average of 25° C. and 70% of air humidity.

For the evaluation of the pathogen development, the inoculated leaves of plants are stained with aniline blue.

The aniline blue staining serves for the detection of fluorescent substances. During the defense reactions in host interactions and non-host interactions, substances such as phenols, callose or lignin accumulate or are produced and are incorporated at the cell wall either locally in papillae or in the whole cell (hypersensitive reaction, HR). Complexes are formed in association with aniline blue, which lead e.g. in the case of callose to yellow fluorescence. The leaf material is transferred to falcon tubes or dishes containing destaining solution II (ethanol/acetic acid 6/1) and is incubated in a water bath at 90° C. for 10-15 minutes. The destaining solution II is removed immediately thereafter, and the leaves are ished 2× with water. For the staining, the leaves are incubated for 1, 5-2 hours in staining solution II (0.05% aniline blue=methyl blue, 0.067 M di-potassium hydrogen phosphate) and analyzed by microscopy immediately thereafter.

The different interaction types are evaluated (counted) by microscopy. An Olympus UV microscope BX61 (incident light) and a UV Longpath filter (excitation: 375/15, Beam splitter: 405 LP) are used. After aniline blue staining, the spores appear blue under UV light. The papillae can be recognized beneath the fungal appressorium by a green/yellow staining. The hypersensitive reaction (HR) is characterized by a whole cell fluorescence.

IV.C Evaluating the Susceptibility to Phytophthora infestans

Phytophthora infestans resistance can be assessed for example in potato.

Three different P. infestans isolates are obtained from Plant Research International B. V. (Wageningen, the Netherlands).

### Disease Assays; Detached Leaves

For the detached leaf assay, leaves from plants grown for 6 to 12 weeks in the greenhouse are placed in pieces of water-saturated florists foam, approximately 35×4×4 cm, and put in a tray (40 cm width, 60 cm length and 6 cm height) with a perforated bottom. Each leaf is inoculated with two droplets (25 μl each) of sporangiospore solution on the abaxial side. Subsequently, the tray is placed in a plastic bag on top of a tray, in which a water-saturated filter paper is placed, and incubated in a climate room at 17° C. and a 16 h/8 h day/night photoperiod with fluorescent light (Philips TLD50W/84HF and OSRAM L58W/21-840). After 6 to 9 days, the leaves are evaluated for the development of P. infestans disease symptoms.

### Evaluation:

Plants with leaves that clearly showed sporulating lesions 6 to 9 days after inoculation are considered to have a susceptible phenotype, whereas plants with leaves showing no visible symptoms or necrosis at the side of inoculation in the absence of clear sporulation are considered to be resistant.

IV.D Evaluating the Susceptibility to Peronospora parasitica and Erysiphe cichoracearum

Control of pathogenic fungi can be measured in Arabidopsis plants, for example by inoculation with the biotrophic fungi Peronospora parasitica or Erysiphe cichoracearum.

a) Peronospora parasitica

Plants of 5 to 8 weeks of age are sprayed with a suspension of spores (conidial spores, approximately 106 spores/ml).

The inoculated plants are covered with a plastic bag and kept overnight moist and dark at 16 in a fridge. After one day the plastic bag is first opened and later, e.g. 6 hours later, removed completely. Six days post inoculation the plants are again put into a plastic bag overnight. this induced sporulation. On the following day the leaves are checked for the occurrence of Konidiophores. The growth of the fungi intracellularly results during the next days to weak chlorosis up to severe necrosis in the leaves. These symptoms are quantified and evaluated for their significance.

b) Erysiphe cichoracearum

This biotrophic fungus is being cultivated on Arabidopsis plants. To achieve infection, a soft, small brush is used to collect the Konidiophores of infected leaves and transfer these to the leaves of 4 week old plants. Then these plants are incubated for 7 days at 20° C. After this time, the new Konidiophores will be visible and during the next days chlorosis and necrosis will become visible. These symptoms are quantified and evaluated for their significance.

### V. Results:

The cultivated plants treated according to the method of the invention show increased plant health.

### VI. Evaluation Procedure of Plants Subject to the Process of the Present Invention

Experiments were conducted using carboxamide compounds BOSCALID and N-(3′,4′,5′-trifluorobiphenyl-2-yl)-3-difluoromethyl-1-methyl-1H-pyrazole-4-carboxamide, subsequently referred to as COMPOUND 2.

### Soja

Soybeans were grown in 2008 at the BASF experimental station in Campinas, San Antonio de Posse, Sao Paulo, Brazil. The soybeans were planted at a seeding rate of 300000 plants per ha. Row spacing was 45 cm. Plot size was 10 m².

COMPOUND 2 was applied twice at growth stage 55/61 (BBCH) and 65/71 (BBCH) as an experimental emulsion concentrate (EC) containing 62.5 g active ingredient per liter with a product rate of 0.48 l/ha and 0.8 l/ha. The formulation was applied in a total spray volume of 150 l/ha.

Infection with Asian Soybean Rust (Phakopsora pachyrhizi) was assessed 20 days after the last treatment by estimating the infected leaf area in 10 randomly chosen plants per plot (Tab. 1). The efficacy was calculated as % decrease of infected leaf area in the treatments compared to the untreated control:

E=(1−a/b)·100

a corresponds to the infected leaf area of the treated plants in % and b corresponds to the infected leaf area of the untreated (control) plants in %

An efficacy of 0 means the infected leaf area of the treated plants corresponds to that of the untreated control plants; an efficacy of 100 means the treated plants showed a reduction in infected leaf area by 100%, meaning no infection with Asian Soybean Rust could be detected.

In addition, the trial was harvested and the grain yield and thousand grain weight (TGW) were measured (Tab. 1).

TABLE Ex VI-1
Efficacy of COMPOUND 2 against soybean rust and yield effect

| | | | | Efficacy | | |
|---|---|---|---|---|---|---|
| | | | | against | | |
| | | | Application | soybean | Grain | |
| | Al rate | Formulation | time | rust | Yield | TGW |
| Product | (g Al/ha) | type | (BBCH) | (%)* | (dt/ha) | (g) |
| 1. Control | | | | 0 | 16.6 | 113.9 |
| 2. COMPOUND 2 | 30 | EC | 55/61 | 34 | 21.6 | 143.5 |
| | 30 | | 65/71 | | | |
| 3. COMPOUND 2 | 50 | EC | 19 | 46 | 22.9 | 144.1 |
| | 50 | | | | | |

*Infection in Control 95% (infected leaf area)

As shown in table 1 COMPOUND 2 has a good activity against Asian Soybean Rust. This activity is increased when treating a transgenic glyphosate tolerant soybean variety with COMPOUND 2 more than it can be expected from the single effects of COMPOUND 2 and the transgenic variety, respectively, on the control of soybean rust. In addition the treatment with COMPOUND 2 results in an increase in grain yield compared to the untreated control. As well, the grain weight of harvested grain of treated soybeans is increased versus the untreated. As for the efficacy against soybean rust, the increase in grain yield and in grain weight is much bigger when treating the transgenic soybean variety than can be expected from the combination of the single effects of both the COMPOUND 2 treatment and the transgenic variety. Hence, synergistic effects for disease control and grain yield can be observed in the combination of the COMPOUND 2 treatment with a transgenic soybean variety.

### Maize

Maize was grown in 2008 at the BASF experimental station in Campinas, San Antonio de Posse, Sao Paulo, Brazil. The variety DKB 390 was planted at a seeding rate of 60,000 plants per ha. Row spacing was 80 cm. Plot size was 30 m².

COMPOUND 2 was applied once at tassel emergence (growth stage 51/55, BBCH) as an experimental emulsion concentrate (EC) containing 62.5 g active ingredient per liter with a dose rate of 0.8 l/ha. The formulation was applied in a total spray volume of 200 l/ha.

Infection with common rust (Puccinia sorghi) 28 days after treatment with COMPOUND 2 was assessed (Tab. 2) by estimating the infected leaf area in 10 randomly chosen plants per plot. The efficacy was calculated as % decrease of infected leaf area in the treatments compared to the untreated control:

E=(1−a/b)·100

a corresponds to the infected leaf area of the treated plants in % and
b corresponds to the infected leaf area of the untreated (control) plants in %

An efficacy of 0 means the infected leaf area of the treated plants corresponds to that of the untreated control plants; an efficacy of 100 means the treated plants showed a reduction in infected leaf area by 100%, meaning no infection with common rust could be detected.

Green leaf retention was estimated in treated and control plants by estimating the green leaf area 28 days after treatment in 10 randomly chosen plants per plot.

At maturity, the plants were harvested and the grain yield and thousand grain weight (TGW) were measured (Tab. 2).

TABLE Ex VI-2
Efficacy of COMPOUND 2 against common rust, effect
on green leaf tissue retention, grain yield and grain weight.

| | | | | Efficacy | Green | | |
|---|---|---|---|---|---|---|---|
| | Al | | Application | against | Leaf | Grain | |
| | rate | Formulation | time | rust | Area | Yield | TGW |
| Product | (g/ha) | type | (BBCH) | (%)* | (%) | (dt/ha) | (g) |
| 1. Control | | | | 0 | 34 | 49.2 | 325 |
| 2. COMPOUND 2 | 50 | EC | 51/55 | 88.6 | 40 | 55.2 | 333 |

*infection in Control 8.8% (infected leaf area)

As shown in table 1 COMPOUND 2 has a good activity against common rust in maize. This activity is increased when treating transgenic glyphosate tolerant and/or insect resistant maize varieties with COMPOUND 2 more than it can be expected from the single effects of COMPOUND 2 and the transgenic varieties, respectively, on the control of common rust. COMPOUND 2 treated plants also show an increase in green leaf area compared to control plants. Similarly, transgenic plants treated with COMPOUND 2 show an increase in green leaf tissue that is bigger than can be expected from the combination of the effects that can observed in using either a transgenic variety or treating conventional maize plants with COMPOUND 2.

In addition the treatment with COMPOUND 2 results in an increase in grain yield compared to the untreated control. As well, the grain weight of harvested grain of treated maize is increased over the untreated control. The increase in grain yield and in grain weight is much bigger when treating the trans-genic maize variety than can be expected from the combination of the single effects of both the COMPOUND 2 treatment and the transgenic variety. Hence, synergistic effects for disease control and grain yield can be observed in the combination of the COMPOUND 2 treatment with a transgenic maize variety.

### Rice

Imidazolinone tolerant rice (Clearfield™) was grown in 2008 at Washington, 7033 Highway 103, LA, USA. The variety CL 161 was planted at a seeding rate of 134 kg/ha. Row spacing was 18 cm. Plot size was 27.5 m².

COMPOUND 2 was applied once at shooting (growth stage 32/34, BBCH) as an experimental emulsion concentrate (EC) containing 62.5 g active ingredient per liter with a dose rate of 0.8 l/ha. The formulation was applied in a total spray volume of 187 l/ha.

Infection with Rhizoctonia solani 77 days after treatment with COMPOUND 2 was assessed (Tab. 3) by estimating the infected leaf area and frequency of infection in 10 randomly chosen plants per plot. The efficacy was calculated as % decrease of infected leaf area in the treatments compared to the untreated control:

E=(1−a/b)·100

a corresponds to the infected leaf area of the treated plants in % and
b corresponds to the infected leaf area of the untreated (control) plants in %

An efficacy of 0 means the infected leaf area of the treated plants corresponds to that of the untreated control plants; an efficacy of 100 means the treated plants showed a reduction in infected leaf area by 100%, meaning no infection with Rhizoctonia solani could be detected.

At maturity, the plants were harvested and the grain yield was measured (Tab. 3).

TABLE Ex VI-3
Efficacy of COMPOUND 2 against Rhizoctonia and yield effect

| | | | | Efficacy against | | |
|---|---|---|---|---|---|---|
| | Al | | Application | Rhizctonia | | Grain |
| | rate | Formulation | time | (%)* | | Yield |
| Product | (g/ha) | type | (BBCH) | Infection | Frequency | (dt/ha) |
| 1. Control | | | | 0 | 0 | 37.76 |
| 2. COMPOUND 2 | 50 | EC | 32/34 | 50 | 45.6 | 54.28 |

*infection in Control 8% (infected leaf area)

As shown in table 3 COMPOUND 2 is active against Rhizoctonia in rice. This activity is higher in the Imidazolinone tolerant rice variety when treated with COMPOUND 2 than in a variety without this herbicide tolerance trait.

In addition the treatment with COMPOUND 2 results in an increase in grain yield compared to the untreated control. The increase in grain yield is bigger when treating the Clearfield™ variety than in a conventional variety.

The increase in disease control efficacy and in yield in the herbicide tolerant CL 161 variety is higher than can be expected from the effects of the COMPOUND 2 treatment in a conventional rice variety and the herbicide tolerance trait in the CL161 variety on disease control and yield. Hence, synergistic effects for disease control and grain yield can be observed in the combination of the COMPOUND 2 treatment with the Imidazolinone tolerance trait.

### Oilseed Rape

Oilseed rape was grown in 2002 at the Verde in France. The variety Colosse was planted at a seeding rate of 3 kg/ha. Row spacing was 17 cm. Plot size was 30 m². BOSCALID was applied once at growth stage 16 (BBCH) using the commercially available Cantus formulation (WG) containing 500 g active ingredient per kg with a dose rate of 0.5 kg/ha. The formulation was diluted in a total spray volume of 300 l/ha.

Infection with Leptosphaeria maculans 209 days after treatment with BOSCALID was assessed (Tab. 4) at crop growth stage 75 (BBCH). Stems of 50 plants were scored and the number of plants with no symptoms (H1), less severe symptoms (H2), severe symptoms (H3) and very severe symptoms (H4) counted. A disease index was calculated as the weighted mean number of plants across the four classes:

(1*No of plants in H1+2*No of plants in H2+3*No of plants in H3+No of plants in H4)/total No of plants assessed

Green leaf retention was estimated in treated and control plants by estimating the green leaf area 28 days after treatment in 10 randomly chosen plants per plot.

At maturity, the plants were harvested and the grain yield was measured (Tab. 4).

TABLE ExVI-4
Efficacy of BOSCALID against Leptoshaeria maculans
(LEPTMA), and effect on yield

| | Al | | Application | LEPTMA | Grain |
|---|---|---|---|---|---|
| | rate | Formulation | time | disease | Yield |
| Product | (g/ha) | type | (BBCH) | index* | (dt/ha) |
| 1. Control | | | | 3.17 | 33.9 |
| 2. BOSCALID | 50 | WG | 16 | 1.71 | 49.3 |

*infection rate in Control 49%

As shown in table 4 BOSCALID has a good activity against Leptosphaeria maculans in oilseed rape. This activity is increased when treating an herbicide tolerant oilseed rape variety with BOSCALID more than it can be expected from the combination of the effect of BOSCALID treatment and the herbicide tolerance trait (imidazolinone resistance), respectively, on Leptosphaeria control.

In addition the treatment with BOSCALID results in an increase in grain yield compared to the untreated control. The increase in grain yield is bigger when treating the herbicide tolerant oilseed rape variety than can be expected from the combination of the single effects of both the BOSCALID treatment and the herbicide tolerance trait. Hence, synergistic effects for disease control and grain yield can be observed in the combination of the BOSCALID treatment with an herbicide tolerant oilseed rape variety.

1. A method for controlling pests and/or increasing plant health of a cultivated plant as compared to the respective control, comprising applying a pesticide to a plant having at least one modification, parts of such plant, plant propagation material, or at its locus of growth, wherein the pesticide is selected from the group consisting of boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam, penthiopyrad, benodanil, carboxin, fenfuram, flutolanil, furametpyr, mepronil, oxycarboxin and thifluzamide.

2. The method according to claim 1, wherein the pesticide is selected from the group consisting of boscalid, N-(3′,4′,5′-trifluorobiphenyl-2-yl)-3-difluoromethyl-1-methyl-1H-pyrazole-4-carboxamide, bixafen, N-[2-(1,3-dimethylbutyl)-phenyl]-1,3-dimethyl-5-fluoro-1H-pyrazole-4-carboxamide, fluopyram, sedaxane, isopyrazam and penthiopyrad.

3. The method according to claim 1, wherein, increasing plant health is as an increase, compared to the respective control, in a trait selected from the group consisting of yield, plant vigor, early vigour, greening effect, quality, tolerance to environmental stress, herbicide tolerance, insect resistance, fungal resistance or viral resistance or bacterial resistance, and antibiotic resistance.

4. The method according claim 1, wherein the cultivated plant shows at least one of the following properties compared to the corresponding control plant respectively: herbicide tolerance, insect resistance, fungal resistance or viral resistance or bacterial resistance, stress tolerance, maturation alteration, content modification of chemicals present in the cultivated plant, modified nutrient uptake, antibiotic resistance and male sterility compared to the corresponding control plant respectively.

5. The method according to claim 1, wherein the cultivated plant is tolerant to the action of herbicides.

6. The method according to claim 1, wherein the cultivated plant is tolerant to the action of glyphosate.

7. The method according to claim 1, wherein the cultivated plant is tolerant to the action of glufosinate.

8. The method according to claim 1, wherein the cultivated plant is tolerant to the action of imidazolinone-herbicides.

9. The method according to claim 1, wherein the cultivated plant is tolerant to the action of dicamba.

10. The method according to claim 1, wherein the cultivated plant is capable of synthesizing at least one selectively acting toxin derived from the bacterial Bacillus spp.

11. The method according to claim 1, wherein the cultivated plant is capable of synthesizing at least one selectively acting toxin from Bacillus thuringiensis.

12. The method according to claim 1, wherein the cultivated plant is capable of synthesizing one or more selectively acting delta-endotoxins toxins from Bacillus thuringiensis.

13. The method according to claim 1, wherein the pesticide is applied to the plant propagation material of the cultivated plant.

14. The method according to claim 1, wherein the treatment(s) are carried out by applying at least one pesticide to the plant having at least one modification or to its habitat.

15. Seed of a cultivated plant treated with a pesticide selected from the group consisting of boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam, penthiopyrad, benodanil, carboxin, fenfuram, flutolanil, furametpyr, mepronil, oxycarboxin and thifluzamide wherein said seed exhibits herbicide tolerance, insect resistance, fungal resistance or viral resistance or bacterial resistance, stress tolerance, maturation alteration, content modification of chemicals present in the cultivated plant, modified nutrient uptake, antibiotic resistance and/or male sterility compared to the corresponding control plant, respectively.

16. A composition comprising a pesticide selected from the group consisting of boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam, penthiopyrad, benodanil, carboxin, fenfuram, flutolanil, furametpyr, mepronil, oxycarboxin and thifluzamide and a cultivated plant or parts or cells thereof.

17. The method according to claim 1, wherein the cultivated plant is a transgenic plant.

18. The method according to claim 1, wherein the cultivated plant is a modified plant.

19. A method for the production of an agricultural product comprising applying a pesticide selected from the group consisting of boscalid, N-(3′,4′,5′-trifluorobiphenyl-2-yl)-3-difluoromethyl-1-methyl-1H-pyrazole-4-carboxamide, bixafen, penflufen, fluopyram, sedaxane, isopyrazam, penthiopyrad, benodanil, carboxin, fenfuram, flutolanil, furametpyr, mepronil, oxycarboxin and thifluzamide to a cultivated plant having at least one modification, parts of such plant, plant propagation materials, or to its locus of growth, and producing the agricultural product from said plant or parts of such plant or plant propagation material.

20. The method according to claim 19, wherein the plant is, a transgenic plant or is a modified plant, or the plant is tolerant to the action of herbicides, glyphosate, glufosinate, imidazolinone-herbicides, or dicamba, or the plant is capable of synthesizing at least one selectively acting toxin derived from the bacterial Bacillus spp., at least one selectively acting toxin from Bacillus thuringiensis, or at least one selectively acting delta-endotoxins toxin from Bacillus thuringiensis.

21-23. (canceled)
